(19) United States
(12) Patent Application Publication
(10) Pub. No.: US 2004/0192686 A1
(43) Pub. Date: Sep. 30, 2004

(54) Benzoazine mono-N-oxides and benzoazine 1,4 dioxides and compositions therefrom for the therapeutic use in cancer treatments

(75) Inventors: William Robert Wilson, Waiuku (NZ); Frederik Bastiaan Pruijn, Auckland (NZ); Bronwyn Gae Siim, Auckland (NZ); Michael Patrick Hay, Auckland (NZ); William Alexander Denny, Auckland (NZ); Swarnalatha Akuratiya Gamage, Auckland (NZ)

Correspondence Address: NIXON & VANDERHYE, PC, 1100 N GLEBE ROAD, 8TH FLOOR, ARLINGTON, VA, 22201-4714, US

(73) Assignee: Auckland Uniservices Limited, Auckland (NZ)

(21) Appl. No.: 10/766,942

(22) Filed: Jan. 30, 2004

(30) Foreign Application Priority Data
Mar. 14, 2003 (NZ) NZ 524770

(51) Int. Cl. A61K 31/53; A61K 31/5377; A61K 31/498

(52) U.S. Cl. 514/234.2; 514/242; 514/249

(57) ABSTRACT

The present invention relates to a synergetistic composition comprising one or more benzoazine-mono-N-oxides, and one or more benzoazine 1,4 dioxides for use in cancer therapy.

The invention also provides a range of novel 1,2,4 benzoazine-mono-N-oxides and related analogues. These can be used as potentiators of the cytotoxicity of existing anticancer drugs and therapies for cancer treatment.

### TECHNICAL FIELD

The present invention relates generally to a cytotoxic synergistic composition comprising one or more benzoazine-mono-N-oxides, and an effective amount of one or more benzoazine 1,4 dioxides for use as anticancer drugs and as radiosensitizers for cancer therapy in combination with radiation and/or other anticancer drugs.

The present invention also relates to the provision of a range of novel 1,2,4-benzoazine-mono-N-oxides and related analogues, and to their use as potentiators of the cytotoxicity of anticancer drugs and as radiosensitizers for cancer therapy in combination with radiation and/or with other anticancer agents, or to at least provide the public with a useful choice.

The term benzoazine used throughout this specification is to be understood as a term used to encompass the mono and di N-oxide derivatives of benzotriazines and quinoxalines.

### BACKGROUND TO THE INVENTION

Hypoxic cells in tumours are resistant to ionising radiation, and are a major cause of treatment failure in radiation therapy (Movsas et al., Cancer, 2000, 89, 2018; Rudat et al., Radiother. Oncol., 2000, 57, 31). Hypoxic cells are also considered to compromise response of solid tumours to cytotoxic chemotherapy (Brown and Giaccia, Cancer Res., 1998, 58, 1408). The benzotriazine di-N-oxide tirapazamine (TPZ) is selectively toxic to hypoxic cells because of its metabolic activation to a cytotoxic species by one-electron reduction (Baker et al., Cancer Res., 1988, 48, 5947; Laderoute et al., Biochem. PharmacoL, 1988, 37, 1487; Brown, Br. J. Cancer 1993, 67, 1163). As shown below, the initial one-electron reduction product TPZ* is reoxidised to the starting compound by dioxygen, thereby preventing cytotoxicity in oxic cells.

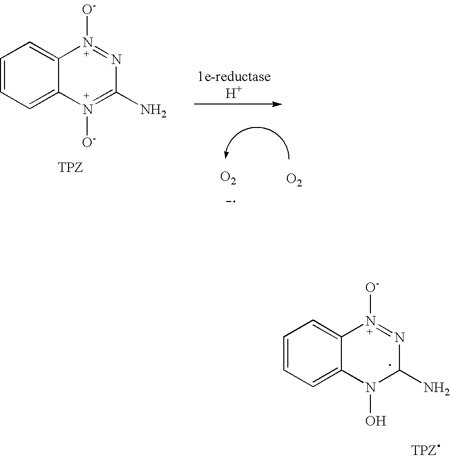

TPZ is therefore of interest for killing hypoxic cells in tumours, thereby improving overall response during radiation therapy. TPZ also has potential for combination with standard cytotoxic chemotherapy (Dorie and Brown, Cancer Res., 1993, 53, 4633; Langmuir et al., Cancer Res., 1994, 54, 2845; Dorie and Brown, Cancer Chemother. Pharmacol., 1997, 39, 361), with (at least) two mechanisms of therapeutic synergy. The first mechanism is the killing of resistant hypoxic cells (analogous to the mechanism of interaction with radiotherapy), and the second is the interference with repair of chemotherapy-induced DNA damage in hypoxic cells as has been demonstrated for cisplatin (Kovacs et al., Br. J. Cancer 1999, 80, 1245; Peters et al., Cancer Res., 2001, 61: 5425.

TPZ has already demonstrated significant antitumour activity in early phase human clinical trials in combination with ionising radiation and/or cisplatin chemotherapy (for a review, see Denny and Wilson, Exp. Opin. Invest. Drugs, 2000, 9, 2889), and a multicentre phase III trial of TPZ in combination with cisplatin and radiation for treatment of head and neck tumours is in progress. While TPZ shows promising indications of clinical activity, it also displays considerable toxicity, such as neutropenia, thrombocytopenia, nausea, vomiting, diarrhoea and muscle cramping. These toxicity limitations preclude administration of doses high enough to exploit hypoxia fully during cancer treatment. Although the mechanisms of TPZ toxicity to normal tissues are not fully understood, it is considered that the toxicity arises at least in part because of redox cycling. (Elwell et al., Biochem. PharmacoL, 1997, 54, 249; Wouters et al., Cancer Res., 2001, 61, 145) The mechanisms of TPZ toxicity are therefore considered to be distinct from the mechanism of hypoxic cell killing. There would be value in identifying agents capable of enhancing the hypoxic cytotoxic potency of TPZ, without increasing its toxicity to oxic cells, in order to improve its therapeutic selectivity for hypoxic tumour cells.

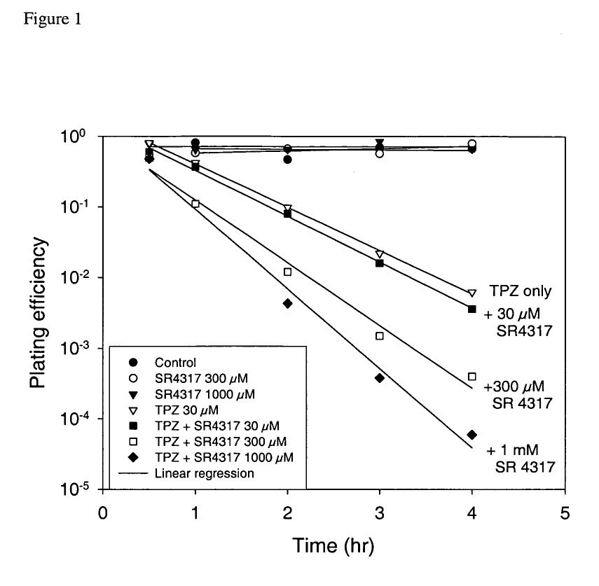

Recent studies indicate that the cytotoxic species arising from reduction of TPZ under hypoxia is an oxidising radical derived from the initial benzotriazine radical (TPZ*); the ultimate cytotoxin has been variously suggested to be the hydroxyl radical OH* (Daniels and Gates, J. Am. Chem. Soc, 1996, 118, 3380; Patterson and Taiwo, Biochem. Pharmacol., 2000, 60, 1933) or the benzotriazinyl radical TPZ⁻ shown in FIG. 1 below. (Anderson et al., J. Am. Chem. Soc, 2003, 125, 748). Whatever its identity, the oxidising radical generates DNA radicals which give rise to complex DNA lesions responsible for cytotoxicity in hypoxic cells (Wang et al., Cancer Res., 1992, 52, 4473; Siim et al., Br. J. Cancer 1996, 73, 952; Kotandeniya et al., Bioorg. Med. Chem. Lett., 2002, 12, 2325; Peters and Brown, Cancer Res., 2002, 62, 5248).

Published studies (Jones and Weinfeld, Cancer Res., 1996, 56, 1584; Daniels and Gates, Chem. Res. Toxicol., 1998, 11, 1254) have suggested that TPZ has a dual mechanism of action, with the parent drug being involved in two distinct steps in the generation of DNA damage as represented below in Scheme A.

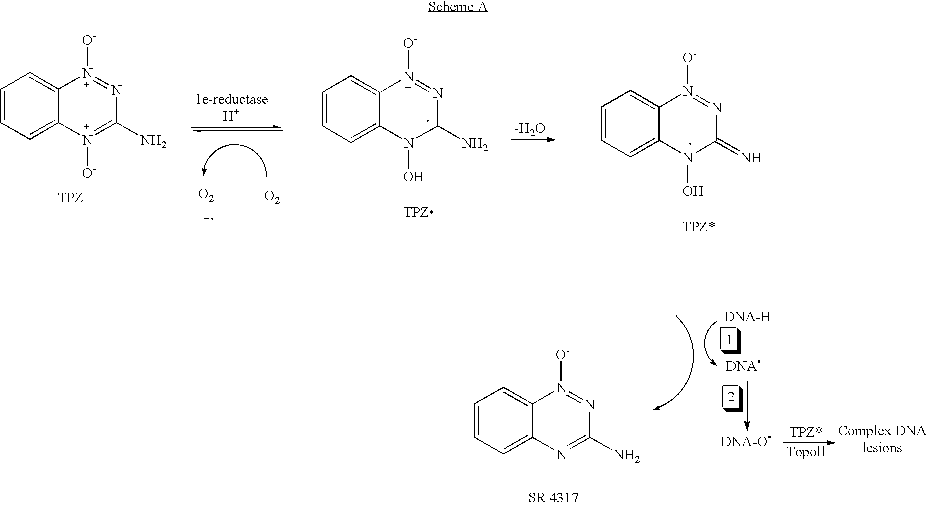

In the first step, the DNA-damaging species, TPZ* or OH*, is considered to generate DNA radicals by hydrogen abstraction. In the second step, TPZ itself can further oxidise the initial DNA radicals to generate a more cytotoxic lesion (DNA break). Certain other agents, such as the 1-N-oxide metabolite derived from TPZ (SR 4317, as illustrated in FIG. 1), have also been shown to be capable of oxidising DNA radicals of the kind formed by TPZ (Hwang et al., Biochemistry, 1999, 38, 14248).

It is an object of the present invention to provide a cytotoxic synergistic composition comprising one or more benzoazine-mono-N-oxides, and an effective amount of one or more benzoazine 1,4 dioxides for use as anticancer drugs and as radiosensitizers for cancer therapy in combination with radiation and/or other anticancer drugs, or to at least provide the public with a useful choice.

It is also an object of the present invention to provide a range of novel 1,2,4-benzoazine-mono-N-oxides and related analogues, and to their use as potentiators of the cytotoxicity of anticancer drugs and as radiosensitizers for cancer therapy in combination with radiation and/or with other anticancer agents, or to at least provide the public with a useful choice.

### SUMMARY OF THE INVENTION

In a first aspect, the present invention provides a cytotoxic synergistic composition, comprising an effective amount of a benzoazine N-mono oxide compound of Formula A or a pharmacologically acceptable salt thereof and an effective amount of a benzoazine 1,4 dioxide compound of Formula B or a pharmacologically acceptable salt thereof

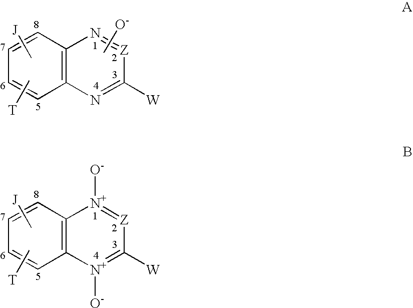

wherein in Formulae A or B

Z is selected from N or C—CN, and

wherein in Formula A when Z represents N, the N-oxide moiety occupies one of the 1-, 2-, or 4-positions; and when Z represents C—CN, the N-oxide moiety occupies one of the 1-, or 4-positions;

wherein J in Formulae A or B represents at one or more of the available carbons 5-8 on the benzo ring the following groups:

halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;

wherein each R is independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;

wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and

wherein W in Formulae A or B represents —X-A, wherein —X-A together may represent

H, or halogen; or

X can represent O, S, NH, NMe, CH₂, SO, SO₂, CONH, NHCO, CO or CO₂, and

A represents H, an optionally substituted C₁₋₁₂alkyl group wherein the optional substituents are each independently selected from OH, OR³, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂alkyl chain can be optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, or a pharmacologically acceptable salt thereof, or

W represents a group of Formula C

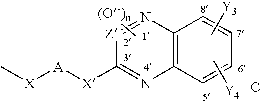

wherein in a group of Formula C

n represents either 1 or 2,

Z′ is selected from N or C—CN, and when Z′ represents N, and n represents 1 the N-oxide moiety may occupy one of the 1′-, 2′-, or 4‘-positions and when Z’ represents C—CN, the N-oxide moiety may occupy one of the 1′-, or 4′-positions; and when Z′ represents N or C—CN, and n represents 2 the N-oxide moieties occupy the 1′ and 4′-positions

Y₃ and Y₄ may each represent at one or more of the available carbons 5′-8′ on the benzo ring the following groups:

halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;

wherein each R is independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R may also represent an optionally substituted aryl or an optionally substituted heteroaryl group having up to 12 carbon atoms, and wherein the optional substituents are each independently selected from; halo, OH, OR¹, NH₂, NHR¹, NR¹ R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;

wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and

X¹ may represent O, NH, NMe, or CH₂,

A may represent an optionally substituted C₁₋₁₂alkyl group wherein the optional substituents are each independently selected from OH, OR³, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₂alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, or

W may represent a group of Formula D

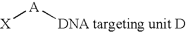

wherein X may represent NH, NMe, CH₂, or O;

A may represent an optionally substituted C₁₋₁₂alkyl group wherein the optional substituents are each independently selected from OH, OR, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂ alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, and

wherein the DNA-targeting unit is any moiety of a molecular weight below 700 Daltons that has an association constant (K) for binding to double-stranded random sequence DNA of >10³ M⁻¹ at an ionic strength of 0.01 M at 20° C.

wherein T in Formulae A or B, may represent at one of carbons 5-8 on the benzo ring the following groups: halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;

wherein each R is independently selected from an optionally substituted C₁₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R may also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;

wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, or

T may represent a group of Formula E

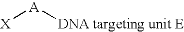

wherein X may represent O, S, NH, NMe, CH₂, SO, SO₂, CONH, NHCO, CO, CO₂, or O and

A may represent an optionally substituted C₁₋₁₂ alkyl group wherein the optional substituents are each independently selected from OH, OR, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₂alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, and wherein the DNA-targeting unit is any moiety of a molecular weight below 700 Daltons that has an association constant (K) for binding to double-stranded random-sequence DNA of >10³ M⁻¹ at an ionic strength of 0.01 M at 20° C.

Preferably, the DNA targeting agent defined above for a group of Formula D or Formula E is independently selected from any one of the following wherein the DNA-targeting unit is selected from one of formulae III-XVII,

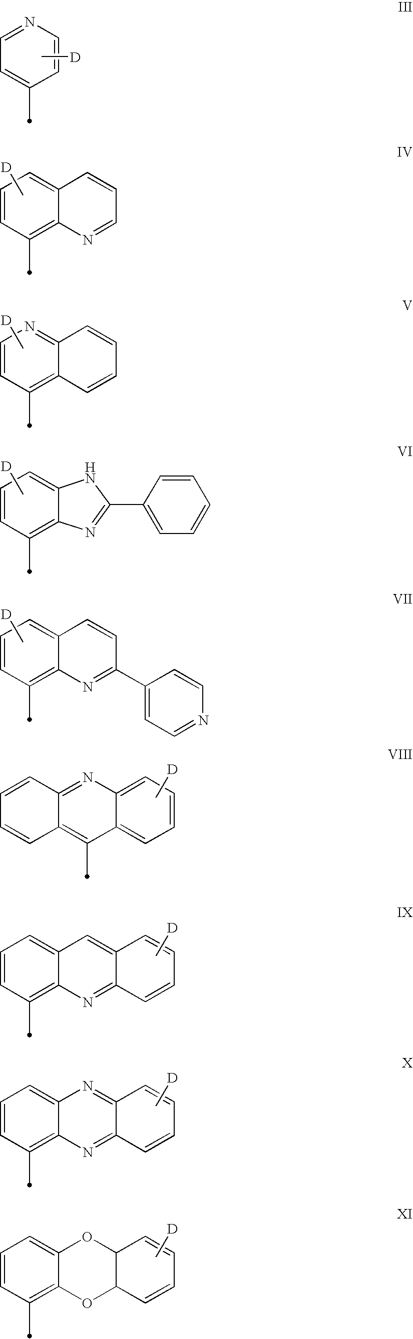

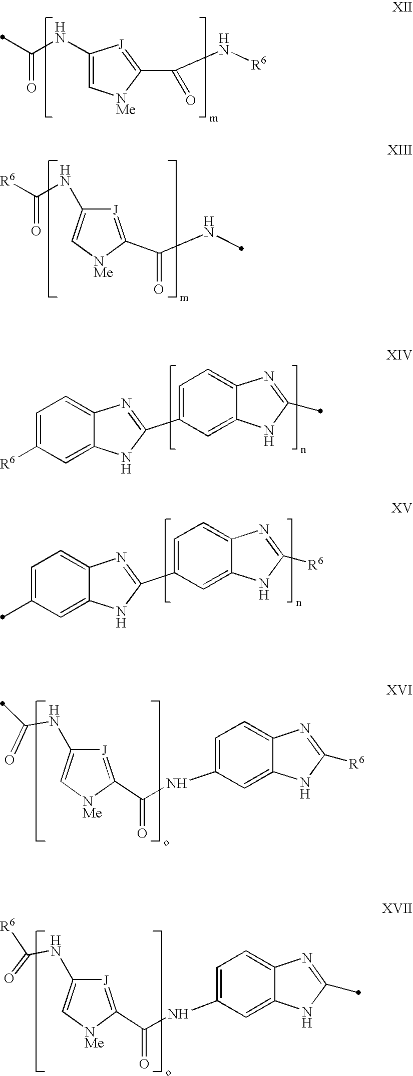

where in structures XII-XVII R⁶ is independently selected from an optionally substituted C₁₋₆ alicyclic or an optionally substituted C₃₋₆ cyclic alkyl group, and wherein the optional substituents are each independently selected from; OH, OR⁷ NO₂, NH₂, NHR⁷, NR⁷R⁷, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷;

R⁶ may also be represent an optionally substituted aryl or an optionally substituted heteroaryl group having up to 12 carbon atoms, and wherein the optional substituents are each independently selected from; halo, OH, OR⁷, NH₂, NHR⁷, NR⁷R⁷, SH, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷, and each heteroaryl group contains one or more heteroatoms in its ring system which are each independently selected from O, N or S;

wherein each R⁷ is independently selected from an optionally substituted C₁₋₄ alkyl or an optionally substituted C₂₋₄ alkenyl group and wherein the optional substituents are each independently selected from OH, OR⁸, NH₂, NHR⁸, NR⁸₂ or N(OH)R⁸ wherein each R⁸ is independently selected from C₁₋₄ alkyl, C₂₋₄ alkenyl, OH, NO₂, NH₂, CF₃, CN, CO₂H or SH;

D may represent up to four of the following groups as substituents at any available ring carbon position; H, R⁹, hydroxy, alkoxy, halogen, NO₂, NH₂, NHR⁹, NR⁹₂, SH, SR⁹, SO₂R⁹, CF₃, CN, CO₂H, CO₂R⁹, CHO, COR⁹, CONH₂, CONHR⁹ or CONR⁹R⁹, cyclic alkylamino, imidazolyl, alkylpiperazinyl, morpholino, wherein each R⁹ is independently selected from an optionally substituted C₁₋₄ alkyl or an optionally substituted C₂₋₄ alkenyl group and wherein the optional substituents are each independently selected from OH, OR¹⁰, NH₂, NHR¹⁰, NR¹⁰₂ or N(OH)R¹⁰ wherein each R¹⁰ is independently selected from C₁₋₄ alkyl, C₂₋₄ alkenyl, OH, NO₂, NH₂, CF₃, CN, CO₂H or SH;

and wherein any available ring carbon position of formulae III-XVII may also be optionally replaced by —N— when the valency and configuration of the formula allows, the point of attachment of formulae III-XVII to the A group defined above is represented by ♦; and

wherein in formulae XII, XIII, m is selected from 2, 3 or 4, and

wherein in formulae XII, XIII, XVI and XVII, J is selected from CH or N;

and wherein in formulae XIV and XV n is selected from 0, 1 or 2;

and wherein in formulae XVI and XVII o is selected from 1 and 2.

More preferably the DNA targeting unit is selected from one of formulae V, VI, VII, VIII, IX or X. Most preferably, D of the DNA targeting unit of Formulae III-XI is H or Me.

Preferably W in Formula A represents NH(C₁-C₁₂) optionally substituted alkyl or a O(C₁-C₁₂) optionally substituted alkyl.

Preferably W in the compound of Formula A represents NH₂, NHCH₂CH₂NHCH₃, NHCH₂CH₂N(CH₃)₂ or OCH₃.

Preferably, the composition includes a pharmaceutically acceptable excipient, adjuvant, carrier, buffer or stabiliser. The pharmaceutically acceptable excipient, adjuvant, carrier, buffer or stabiliser should be non-toxic and should not interfere with the efficacy of the active composition. The precise nature of the carrier or other material will depend on the intended route of administration, which may be oral, or by injection such as cutaneous, subcutaneous or by intravenous injection. Pharmaceutical compositions for oral administration may be in tablet, capsule, powder or liquid form. A tablet may comprise a solid carrier or an adjuvant. Liquid pharmaceutical compositions generally comprise a liquid such as water, petroleum, animal or vegetable oils, mineral oil or synthetic oil. Physiological saline solution, dextrose or other saccharide solution or glycols such as ethylene glycol, propylene glycol may be included. A capsule may comprise a solid carrier such as a gelatine. For intravenous, cutaneous or subcutaneous injection, the active composition will be in the form of a parenterally acceptable aqueous solution that is pyrogen-free and has a suitable pH, isotonicity and stability. Those of skill in the art would be able to prepare suitable solutions using for example, isotonic vehicles such as sodium chloride injection, Ringer's injection, lactated Ringer's injection. Preservatives, stabilisers, buffers, antioxidants and/or other additives may be included as required.

It is to be appreciated that the compounds of Formula B are to be taken as including all the DNA-targeted benzotriazine 1,4-dioxides and the methods disclosed for making these compounds as specified in co-pending PCT application PCT/NZ03/00210 to Auckland Uniservices and the Board of Trustees of the Leland Stanford Junior University. The disclosure of PCT/NZ03/00210 is hereby incorporated in its entirety.

In a second aspect, there is provided a method of treating a subject in need of cancer therapy, said method comprising the steps of administering to said subject a cytotoxic effective amount of a composition including an effective amount of one or more compounds of Formula A and one or more compounds of formula B as defined above to the tumour cells in said subject.

Preferably, the steps of administration of a compound of Formula A and B may be simultaneous or sequential.

Preferably the tumour cells are in a hypoxic environment.

Preferably, the method includes the further step of administering said composition defined above in combination with one or more other chemotherapeutic agents or treatments, including radiotherapy, either simultaneously, or sequentially, depending on the cancerous condition to be treated.

More preferably the method includes the step of administering radiotherapy to the tumour cells before, during or after the administration of the composition as defined above.

Preferably, the chemotherapeutic agents are selected from Cisplatin or other platinum-based derivatives, Temozolomide or other DNA methylating agents, cyclophosphamide or other DNA alkylating agents, doxorubicin, mitoxandrone, camptothecin or other topoisomerase inhibitors, methotrexate, gemcitabine or other antimetabolites.

While the compositions of the invention will typically be used in cancer therapy of human subjects, they may be used to target tumour cells in other warm blooded animal subjects such as other primates, farm animals such as cattle, and sports animals and pets such as horses, dogs, and cats.

A “cytotoxic effective amount”, is to be understood as an amount of the composition including one or more compounds of Formula A and one or more compounds of Formula B as defined above that is sufficient to show benefit to a patient. The actual amount, rate and time-course of administration, will depend on the nature and severity of the disease being treated. Prescription of treatment is within the responsibility of general practitioners and other medical doctors.

A hypoxic environment is to be understood as tissue environments at an oxygen concentration of <10 μM.

In a third aspect there is provided, the use in the manufacture of a medicament of an effective amount of a composition including an effective amount of one or more compounds of Formula A or one or more compounds of formula B as defined above for the treatment of a subject in need of cancer therapy.

In a fourth aspect, the present invention provides a compound of Formula I,

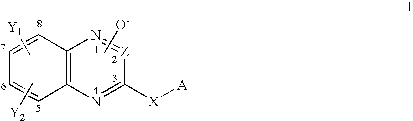

wherein

Z may be selected from N or C—CN, and when Z represents N, the N-oxide moiety occupies one of the 1-, 2-, or 4-positions; and when Z represents C—CN, the N-oxide moiety occupies one of the 1-, or 4-positions;

Y₁ and Y₂ may each represent at one or more of the available carbons 5-8 on the benzo ring the following groups: halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;

wherein each R may be independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R may also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;

wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and

wherein A and X together may represent H, or halogen; or

X may represent O, S, NH, NMe or CH₂ and

A may represent H, an optionally substituted C₁₋₁₂alkyl group wherein the optional substituents are each independently selected from OH, OR³, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, or a pharmacologically acceptable salt thereof,

with the proviso that the following compounds are excluded

3-Amino-1,2,4-benzotriazine-1-oxide,

3-Amino-7-trifluoromethyl-1,2,4-benzotriazine-1-oxide,

3-Amino-7-carbamyl-1,2,4-benzotriazine-1-oxide,

3-Amino-7-chloro-1,2,4-benzotriazine-1-oxide,

3-Amino-7-nitro-1,2,4-benzotriazine-1-oxide

3-Chloro-1,2,4-benzotriazine-1-oxide,

3-(3-N,N-Diethylaminopropylamino)-3-amino-1,2,4-benzotriazine-1-oxide,

3-Chloro-7-nitro-1,2,4-benzotriazine-1-oxide,

7-Nitro-(3-(2-N,N-diethylamino-ethylamino)-1,2,4-benzotriazine-1-oxide,

8-Methoxy-1,2,4-benzotriazin-3-amine 1-oxide,

8-Methyl-1,2,4-benzotriazin-3-amine 1-oxide,

8-Fluoro-1,2,4-benzotriazin-3-amine 1-oxide,

8-Chloro-1,2,4-benzotriazin-3-amine 1-oxide,

8-Trifluoromethyl-1,2,4-benzotriazin-3-amine 1-oxide,

8-(Methylsulfanyl)-1,2,4-benzotriazin-3-amine 1-oxide,

8-(Butylsulfanyl)-1,2,4-benzotriazin-3-amine 1-oxide,

3-Amino-1,2,4-benzotriazin-7-ol 1-oxide,

3-Amino-1,2,4-benzotriazin-7-ol 1-oxide,

7-Methyl-1,2,4-benzotriazin-3-amine 1-oxide,

7-Fluoro-1,2,4-benzotriazin-3-amine 1-oxide,

7-Chloro-1,2,4-benzotrazin-3-amine 1-oxide,

7-Trifluoromethyl-1,2,4-benzotriazin-3-amine 1-oxide,

7-(Methylsulfanyl)-1,2,4-benzotriazin-3-amine 1-oxide,

7-(Butylsulfanyl)-1,2,4-benzotriazin-3-amine 1-oxide,

7-Nitro-1,2,4-benzotriazin-3-amine 1-oxide,

6-Methoxy-1,2,4-benzotriazin-3-amine 1-oxide,

6-Methyl-1,2,4-benzotriazin-3-amine 1-oxide,

6-Phenyl-1,2,4-benzotriazin-3-amine 1-oxide,

6-Fluoro-1,2,4-benzotriazin-3-amine 1-oxide,

6-Chloro-1,2,4-benzotrazin-3-amine 1-oxide,

6-Trifluoromethyl-1,2,4-benzotriazin-3-amine 1-oxide,

6-(Methylsulfanyl)-1,2,4-benzotriazin-3-amine 1-oxide,

6-(Butylsulfanyl)-1,2,4-benzotriazin-3-amine 1-oxide,

5-Methoxy-1,2,4-benzotriazin-3-amine 1-oxide,

5-Methyl-1,2,4-benzotriazin-3-amine 1-oxide,

5-Chloro-1,2,4-benzotriazin-3-amine 1-oxide,

5-Fluoro-1,2,4-benzotriazin-3-amine 1-oxide,

N⁷, N⁷-Dimethyl-1,2,4-benzotriazine-3,7-diamine 1-oxide,

3-Chloro-1,2,4-benzotriazine 1-oxide,

3-Methyl-1,2,4-benzotriazine 1-oxide,

3-Ethyl-1,2,4-benzotriazine 1-oxide,

3-Phenyl-1,2,4-benzotriazine 1-oxide,

3-(4-Methoxyphenyl)-1,2,4-benzotriazine 1-oxide,

3-Vinyl-1,2,4-benzotriazine 1-oxide,

3-Allyl-1,2,4-benzotriazine 1-oxide,

3-(2-Hydroxyethyl)-1,2,4-benzotriazine 1-oxide,

3-(2-Methoxyethyl)-1,2,4-benzotriazine 1-oxide,

N-Phenyl-1,2,4-benzotriazin-3-amine 1-oxide,

3-Methoxy-1,2,4-benzotriazine 1-oxide,

3-Chloro-7-methyl-1,2,4-benzotriazine 1-oxide,

3-Chloro-7-methoxy-1,2,4-benzotriazine 1-oxide,

1,2,4-benzotriazine 1-oxide,

1,2,4-benzotriazin-3-amine 2-oxide, and

1,2,4-benzotriazin-3-amine 4-oxide.

Preferably, Z is N.

A preferred compound of Formula I is one in which X is NH or CH₂.

A more preferred compound of Formula I is one in which —X-A represents a NH(C₁-C₁₂) optionally substituted alkyl or an O(C₁-C₁₂) optionally substituted alkyl, such as NHCH₂CH₂NHCH₃, NHCH₂CH₂N(CH₃)₂ or OCH₃.

A further preferred compound of Formula I is one in which Y₁ and Y₂ each represent H.

A further preferred compound of Formula I′ is one in which the N-oxide moiety occupies the 1-position.

In a fifth aspect, there is provided a method of treating a subject in need of cancer therapy, said method comprising the steps of administering to said subject a cytotoxic effective amount of a compound of Formula I

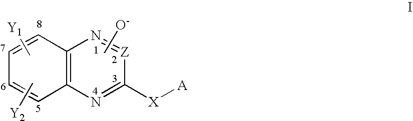

wherein

Z is selected from N or C—CN, and when Z represents N, the N-oxide moiety occupies one of the 1-, 2-, or 4-positions; and when Z represents C—CN, the N-oxide moiety occupies one of the 1-, or 4-positions;

Y₁ and Y₂ may each represent at one or more of the available carbons 5-8 on the benzo ring the following groups:

halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;

wherein each R is independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R may also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;

wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁ alkyl, C₂₋₄ alkenyl, OH, NO₂, NH₂, CF₃, CN, CO₂H or SH, and

wherein A and X together may represent H, or halogen; or

X may represent O, S, NH, NMe or CH₂ and

A may represent H, an optionally substituted C₁₋₁₂alkyl group wherein the optional substituents are each independently selected from OH, OR, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, or a pharmacologically acceptable salt thereof

to the tumour cells in said subject.

Preferably the tumour cells are in a hypoxic environment.

Preferably, the method includes the further step of administering the compound of Formula I in combination with one or more other chemotherapeutic agents or treatments, including radiotherapy, either simultaneously, or sequentially, depending on the cancerous condition to be treated.

More preferably the method includes the step of administering radiotherapy to the tumour cells before, during or after the administration of the composition as defined above.

Preferably, the chemotherapeutic agents are selected from Cisplatin or other platinum-based derivatives, Temozolomide or other DNA methylating agents, cyclophosphamide or other DNA alkylating agents, doxorubicin, mitoxandrone, camptothecin or other topoisomerase inhibitors, methotrexate, gemcitabine or other antimetabolites.

While the method of the invention will typically be used in cancer therapy of human subjects, they may be used to target tumour cells in other warm blooded animal subjects such as other primates, farm animals such as cattle, and sports animals and pets such as horses, dogs, and cats.

Preferably, the compound of Formula I is administered with a pharmaceutically acceptable excipient, adjuvant, carrier, buffer or stabiliser. The pharmaceutically acceptable excipient, adjuvant, carrier, buffer or stabiliser should be non-toxic and should not interfere with the efficacy of the active composition. The precise nature of the carrier or other material will depend on the intended route of administration, which may be oral, or by injection such as cutaneous, subcutaneous or by intravenous injection. Pharmaceutical compositions of Formula I for oral administration may be in tablet, capsule, powder or liquid form. A tablet may comprise a solid carrier or an adjuvant. Liquid pharmaceutical compositions generally comprise a liquid such as water, petroleum, animal or vegetable oils, mineral oil or synthetic oil. Physiological saline solution, dextrose or other saccharide solution or glycols such as ethylene glycol, propylene glycol may be included. A capsule may comprise a solid carrier such as a gelatine. For intravenous, cutaneous or subcutaneous injection, the active composition will be in the form of a parenterally acceptable aqueous solution that is pyrogen-free and has a suitable pH, isotonicity and stability. Those of skill in the art would be able to prepare suitable solutions using for example, isotonic vehicles such as sodium chloride injection, Ringer's injection, lactated Ringer's injection. Preservatives, stabilisers, buffers, antioxidants and/or other additives may be included as required.

A “cytotoxic effective amount”, is to be understood as an amount of a compound of Formula I defined above that is sufficient to show benefit to a patient. The actual amount, rate and time-course of administration, will depend on the nature and severity of the disease being treated. Prescription of treatment is within the responsibility of general practitioners and other medical doctors.

A hypoxic environment is to be understood as tissue environments at an oxygen concentration of <10 mM.

In a sixth aspect there is provided, the use in the manufacture of a medicament of an effective amount of a compound of Formula I as defined above for the treatment of a subject in need of cancer therapy.

In a seventh aspect the present invention provides a compound of Formula I′,

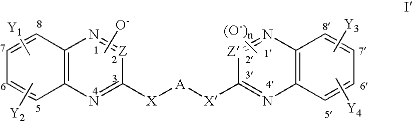

wherein

n may represent either 1 or 2,

Z or Z′ is selected from N or C—CN, and when Z or Z′ represents N, and n represents 1 each N-oxide moiety may occupy one of the 1-, 2-, or 4-positions or 1′-, 2′-, or 4′-positions respectively and when Z or Z′ represents C—CN, each N-oxide moiety may occupy one of the 1-, or 4-positions or 1′-, or 4′-positions respectively; and when Z′ represents N, and n represents 2, the N′-oxide moieties occupy the 1′- and 4‘-positions and when Z’ represents C—CN, and n represents 2 the N′-oxide moieties occupy the 1′-, and 4′-positions;

Y₁, Y₂, Y₃ and Y₄ may each represent at one or more of the available carbons 5-8 or one or more of the available carbons 5′-8′ on the respective benzo ring the following groups:

halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;

wherein each R is independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R may also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;

wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and

wherein X may represent NH, NMe, CH₂, or O;

A may represent an optionally substituted C₁₋₁₂alkyl group wherein the optional substituents are each independently selected from OH, OR³, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, or a pharmacologically acceptable salt thereof.

A preferred compound of Formula I′ is one in which X is NH or CH₂.

A further preferred compound of Formula I′ is one in which Y₁ and Y₂ each represent H.

A further preferred compound of Formula I′ is one in which A is —(CH₂)₂NMe(CH₂)₂—

A further preferred compound of Formula I′ is one in which the N-oxides are positioned at the 1-position.

In an eighth aspect, there is provided a method of treating a subject in need of cancer therapy, said method comprising the steps of administering to said subject a cytotoxic effective amount of a compound of Formula I′ as defined above to the tumour cells in said subject.

Preferably the tumour cells are in a hypoxic environment.

Preferably, the method includes the further step of administering the compound of Formula I′ in combination with one or more other chemotherapeutic agents or treatments, including radiotherapy, either simultaneously, or sequentially, depending on the cancerous condition to be treated.

More preferably the method includes the step of administering radiotherapy to the tumour cells before, during or after the administration of the composition as defined above.

Preferably, the chemotherapeutic agents are selected from Cisplatin or other platinum-based derivatives, Temozolomide or other DNA methylating agents, cyclophosphamide or other DNA alkylating agents, doxorubicin, mitoxandrone, camptothecin or other topoisomerase inhibitors, methotrexate, gemcitabine or other antimetabolites.

While the method of the invention will typically be used in cancer therapy of human subjects, they may be used to target tumour cells in other warm blooded animal subjects such as other primates, farm animals such as cattle, and sports animals and pets such as horses, dogs, and cats.

Preferably, the compound of Formula I′ is administered with a pharmaceutically acceptable excipient, adjuvant, carrier, buffer or stabiliser. The pharmaceutically acceptable excipient, adjuvant, carrier, buffer or stabiliser should be non-toxic and should not interfere with the efficacy of the active composition. The precise nature of the carrier or other material will depend on the intended route of administration, which may be oral, or by injection such as cutaneous, subcutaneous or by intravenous injection. Pharmaceutical compositions of Formula I′ for oral administration may be in tablet, capsule, powder or liquid form. A tablet may comprise a solid carrier or an adjuvant. Liquid pharmaceutical compositions generally comprise a liquid such as water, petroleum, animal or vegetable oils, mineral oil or synthetic oil. Physiological saline solution, dextrose or other saccharide solution or glycols such as ethylene glycol, propylene glycol may be included. A capsule may comprise a solid carrier such as a gelatine. For intravenous, cutaneous or subcutaneous injection, the active composition will be in the form of a parenterally acceptable aqueous solution that is pyrogen-free and has a suitable pH, isotonicity and stability. Those of skill in the art would be able to prepare suitable solutions using for example, isotonic vehicles such as sodium chloride injection, Ringer's injection, lactated Ringer's injection. Preservatives, stabilisers, buffers, antioxidants and/or other additives may be included as required.

A “cytotoxic effective amount”, is to be understood as an amount of a compound of Formula I′ defined above that is sufficient to show benefit to a patient. The actual amount, rate and time-course of administration, will depend on the nature and severity of the disease being treated. Prescription of treatment is within the responsibility of general practitioners and other medical doctors.

A hypoxic environment is to be understood as tissue environments at an oxygen concentration of <10 mM.

In a ninth aspect there is provided, the use in the manufacture of a medicament of an effective amount of a compound of Formula I′ as defined above for the treatment of a subject in need of cancer therapy.

In a tenth aspect, the present invention provides a compound of Formula II,

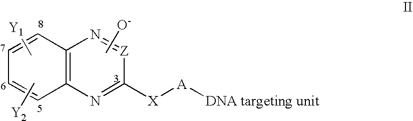

wherein

Z is selected from N or C—CN, and when Z represents N, the N-oxide moiety occupies one of the 1-, 2-, or 4-positions; and when Z represents C—CN, the N-oxide moiety occupies one of the 1-, or 4-positions;

Y₁ and Y₂ may each represent at one or more of the available carbons 5-8 on the benzo ring the following groups:

halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;

wherein each R is independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R may also represent an optionally substituted aryl or an optionally substituted heteroaryl group having up to 12 carbon atoms, and wherein the optional substituents are each independently selected from; halo, OH, OR¹, NH₂, NHR¹, NR¹ R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;

wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and

wherein X may represent NH, NMe, CH₂, or O;

A may represent an optionally substituted C₁₋₁₂alkyl group wherein the optional substituents are each independently selected from OH, OR³, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH; and

wherein the DNA-targeting unit is any moiety of a molecular weight below 700 Daltons that has an association constant (K) for binding to double-stranded random-sequence DNA of >10³ M-1 at an ionic strength of 0.01 M at 20° C.,

or a pharmacologically acceptable salt thereof.

The definition of the DNA targeting unit above refers to double-stranded random-sequence DNA. An example of such double-stranded random-sequence DNA is DNA extracted from calf thymus.

Preferably, Z is N.

A preferred compound of Formula II is one in which X is NH or CH₂.

A further preferred compound of Formula II is one in which the N-oxide is at the 1-position

A further preferred compound of Formula II is one in which Y₁ and Y₂ each represent H.

A further preferred compound of Formula II is one in which Y₁ represents Me

A preferred embodiment of Formula II are compounds wherein A is selected from —(CH₂)₆NH—, —(CH₂)₃NH(CH₂)₃NHCO—, —(CH₂)₃NMe(CH₂)₃NHCO—, —(CH₂)₃NH—, —(CH₂)₂NH(CH₂)₂NHCO— or —(CH₂)₂NMe(CH₂)₂NHCO—.

A further preferred embodiment of Formula II are compounds wherein the DNA-targeting unit is selected from one of formulae III-XVII,

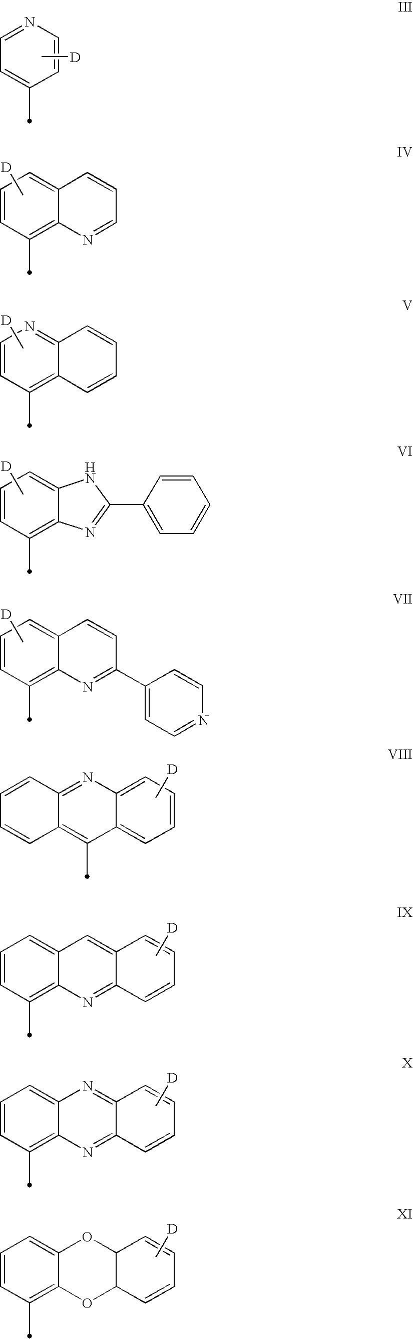

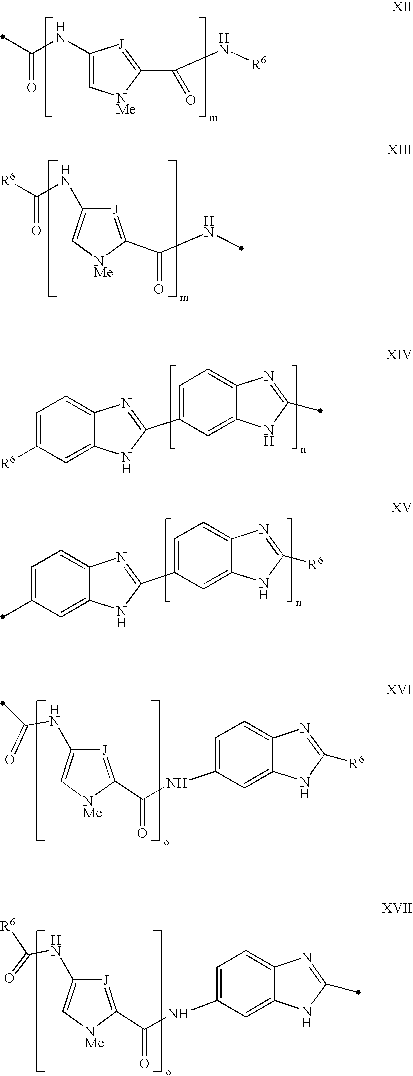

wherein in structures XII-XVII R⁶ is independently selected from an optionally substituted C₁₋₆ alicyclic or an optionally substituted C₃₋₆ cyclic alkyl group, and wherein the optional substituents are each independently selected from; halo, OH, OR⁷ NO₂, NH₂, NHR⁷, NR⁷R⁷, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷;

R⁶ may also be represent an optionally substituted aryl or an optionally substituted heteroaryl group having up to 12 carbon atoms, and wherein the optional substituents are each independently selected from; halo, OH, OR⁷, NH₂, NH⁷, NHR⁷, SH, SR⁷, imidazolyl, R⁷-iperazinyl, morpholino, SO₂R⁷ CF₃, CN, CO₂H, CO₂R⁷ CHO, COR⁷, CONH₂, CONHR⁷, CONR⁷R⁷, and each heteroaryl group contains one or more heteroatoms in its ring system which are each independently selected from O, N or S;

wherein each R⁷ is independently selected from an optionally substituted C₁₋₄ alkyl or an optionally substituted C₂₋₄ alkenyl group and wherein the optional substituents are each independently selected from OH, OR⁸, NH₂, NHR⁸, NR⁸₂ or N(OH)R⁸ wherein each R⁸ is independently selected from C₁₋₄ alkyl, C₂₋₄ alkenyl, OH, NO₂, NH₂, CF₃, CN, CO₂H or SH;

D may represent up to four of the following groups as substituents at any available ring carbon position; H, R⁹, hydroxy, alkoxy, halogen, NO₂, NH₂, NHR⁹, NR⁹₂, SH, SR⁹, SO₂R⁹, CF₃, CN, CO₂H, CO₂R⁹, CHO, COR⁹, CONH₂, CONHR⁹ or CONR⁹R⁹, cyclic alkylamino, imidazolyl, alkylpiperazinyl, morpholino, wherein each R⁹ is independently selected from an optionally substituted C₁₋₄ alkyl or an optionally substituted C₂₋₄ alkenyl group and wherein the optional substituents are each independently selected from OH, OR¹⁰, NH₂, NHR¹⁰, NR¹⁰₂ or N(OH)R¹⁰ wherein each R¹⁰ is independently selected from C₁₋₄ alkyl, C₂₋₄ alkenyl, OH, NO₂, NH₂, CF₃, CN, CO₂H or SH;

and wherein any available ring carbon position of formulae III-XVII may also be optionally replaced by —N— when the valency and configuration of the formula allows, the point of attachment of formulae III-XVII to the A group defined above is represented by ♦; and

wherein in formulae XII, XIII, m is selected from 2, 3 or 4, and

wherein in formulae XII, XIII, XVI and XVII, J is selected from CH or N;

and wherein in formulae XIV and XV n is selected from 0, 1 or 2;

and wherein in formulae XVI and XVII o is selected from 1 and 2.

A preferred embodiment of formula II is one in which the DNA targeting unit is selected from one of formulae V, VI, VII, VIII, IX or X.

A preferred embodiment of formula II is one in which D of the DNA targeting unit of Formulae III-XI is H or Me.

Further preferred compounds of formula II include the following

wherein X is NH—, Y is H, Z is N, position 1-oxide, A is —(CH₂)₂NH(CH₂)₂NHCO—, the DNA targeting unit represents formula IX and D is H;

wherein X is NH—, Y is H, Z is N, position 1-oxide, A is —(CH₂)₃NH(CH₂)₃NHCO—, the DNA targeting unit represents formula IX and D is H;

wherein X is NH—, Y is H, Z is N, position 1-oxide, A is —(CH₂)₂NMe(CH₂)₂NHCO—, the DNA targeting unit represents formula IX and D is H;

wherein X is NH—, Y is 6-Me, Z is N, position 1-oxide, A is —(CH₂)₂NMe(CH₂)₂NHCO—, the DNA targeting unit represents formula IX and D is H;

wherein X is NH—, Y is H, Z is N, position 1-oxide, A is —(CH₂)₃NMe(CH₂)₃NHCO—, the DNA targeting unit represents formula IX and D is H;

wherein X is NH—, Y is 6-Me, Z is N, position 1-oxide, A is —(CH₂)₃NMe(CH₂)₃NHCO—, the DNA targeting unit represents formula IX and D is H;

wherein X is NH—, Y is H, Z is N, position 1-oxide, A is —(CH₂)₂NMe(CH₂)₂NHCO—, the DNA targeting unit represents formula IX and D is Me;

wherein X is NH—, Y is H, Z is N, position 1-oxide, A is —(CH₂)₃NMe(CH₂)₃NHCO—, the DNA targeting unit represents formula IX and D is Me.

In an eleventh aspect, there is provided a method of treating a subject in need of cancer therapy, said method comprising the steps of administering to said subject a cytotoxic effective amount of a compound of Formula II as defined above to the tumour cells in said subject.

Preferably the tumour cells are in a hypoxic environment.

Preferably, the method includes the further step of administering the compound of Formula II in combination with one or more other chemotherapeutic agents or treatments, including radiotherapy, either simultaneously, or sequentially, depending on the cancerous condition to be treated.

More preferably the method includes the step of administering radiotherapy to the tumour cells before, during or after the administration of the composition as defined above.

Preferably, the chemotherapeutic agents are selected from Cisplatin or other platinum-based derivatives, Temozolomide or other DNA methylating agents, cyclophosphamide or other DNA alkylating agents, doxorubicin, mitoxandrone, camptothecin or other topoisomerase inhibitors, methotrexate, gemcitabine or other antimetabolites.

While the method of the invention will typically be used in cancer therapy of human subjects, they may be used to target tumour cells in other warm blooded animal subjects such as other primates, farm animals such as cattle, and sports animals and pets such as horses, dogs, and cats.

Preferably, the compound of Formula II is administered with a pharmaceutically acceptable excipient, adjuvant, carrier, buffer or stabiliser. The pharmaceutically acceptable excipient, adjuvant, carrier, buffer or stabiliser should be non-toxic and should not interfere with the efficacy of the active composition. The precise nature of the carrier or other material will depend on the intended route of administration, which may be oral, or by injection such as cutaneous, subcutaneous or by intravenous injection. Pharmaceutical compositions of Formula II for oral administration may be in tablet, capsule, powder or liquid form. A tablet may comprise a solid carrier or an adjuvant. Liquid pharmaceutical compositions generally comprise a liquid such as water, petroleum, animal or vegetable oils, mineral oil or synthetic oil. Physiological saline solution, dextrose or other saccharide solution or glycols such as ethylene glycol, propylene glycol may be included. A capsule may comprise a solid carrier such as a gelatine. For intravenous, cutaneous or subcutaneous injection, the active composition will be in the form of a parenterally acceptable aqueous solution that is pyrogen-free and has a suitable pH, isotonicity and stability. Those of skill in the art would be able to prepare suitable solutions using for example, isotonic vehicles such as sodium chloride injection, Ringer's injection, lactated Ringer's injection. Preservatives, stabilisers, buffers, antioxidants and/or other additives may be included as required.

A “cytotoxic effective amount”, is to be understood as an amount of a compound of Formula II defined above that is sufficient to show benefit to a patient. The actual amount, rate and time-course of administration, will depend on the nature and severity of the disease being treated. Prescription of treatment is within the responsibility of general practitioners and other medical doctors.

A hypoxic environment is to be understood as tissue environments at an oxygen concentration of <10 mM.

In a twelth aspect there is provided, the use in the manufacture of a medicament of an effective amount of a compound of Formula II as defined above for the treatment of a subject in need of cancer therapy.

In a thirteenth aspect, the present invention provides a compound of Formula II′.

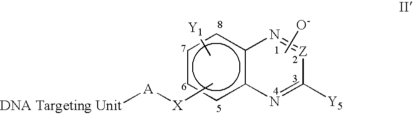

wherein

Z is selected from N or C—CN, and when Z represents N, the N-oxide moiety occupies one of the 1-, 2-, or 4-positions; and when Z represents C—CN, the N-oxide moiety occupies one of the 1-, or 4-positions;

Y₁ may represent at one or more of the available carbons 5-8 on the benzo ring the following groups:

halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;

Y₅ is selected from the following groups halo, H, R, OR, NH₂, NHR, NR₂, SO₂R, CF₃, CN, CO₂H, CO₂R, CHO, COR, CONH₂, CONHR or CONRR, cyclic alkylamino, imidazolyl, alkylpiperazinyl, morpholino;

wherein each R of groups Y₁ and Y₅ is independently selected from an optionally substituted C₁₋₆ alicyclic or an optionally substituted C₃₋₆ cyclic alkyl group, and wherein the optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R may also be represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S; wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and

wherein X may represent NH, NMe, CH₂, S, SO, SO₂, CONH, NHCO, CO, CO₂, or O;

A may represent an optionally substituted C₁₋₁₂alkyl group wherein the optional substituents are each independently selected from OH, OR³, NH₂, NHR³, NR³₂ or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₂₋₁₂alkyl chain is optionally interrupted by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH; and

wherein the DNA-targeting unit is any moiety of a molecular weight below 700 Daltons that has an association constant (K) for binding to double-stranded random-sequence DNA of >10³ M⁻¹ at an ionic strength of 0.01 M at 20° C.,

or a pharmacologically acceptable salt thereof.

The definition of the DNA targeting unit above refers to double-stranded random-sequence DNA. An example of such double-stranded random-sequence DNA is DNA extracted from calf thymus.

Preferably, Z is N.

A preferred compound of Formula II′ is one in which X is O or CH₂

A further preferred compound of Formula II′ is one in which the N-oxide is at the 1-position

A further preferred compound of Formula II′ is one in which Y₁ represents H

A further preferred compound of Formula II′ is one in which Y₅ represents NHR

A preferred embodiment of Formula II are compounds wherein A is selected from —(CH₂)₆NH—, —(CH₂)₃NH(CH₂)₃NHCO—, —(CH₂)₃NMe(CH₂)₃NHCO—, —(CH₂)₃NH—, —(CH₂)₂NH(CH₂)₂NHCO— or —(CH₂)₂NMe(CH₂)₂NHCO—.

A further preferred embodiment of Formula II are compounds wherein the DNA-targeting unit is selected from one of formulae III-XVII,

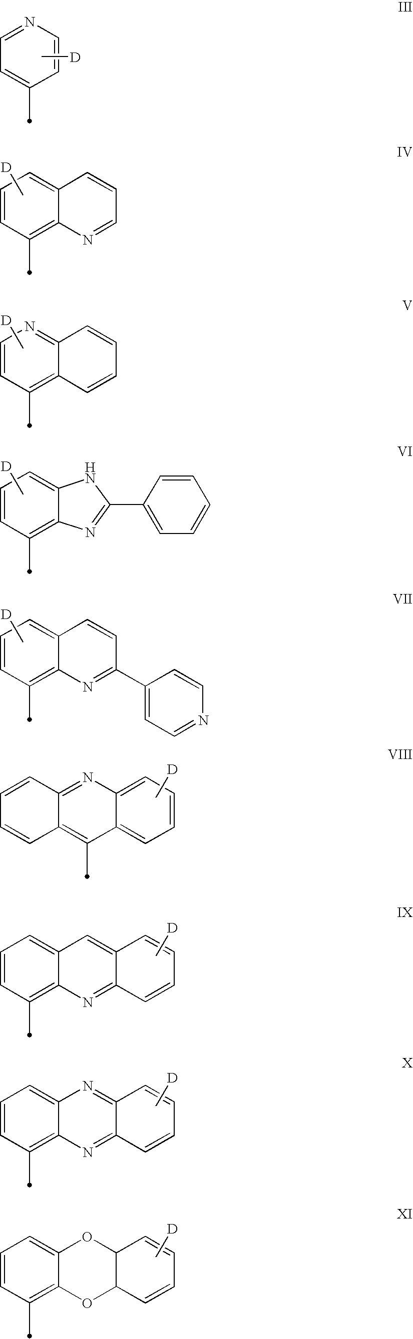

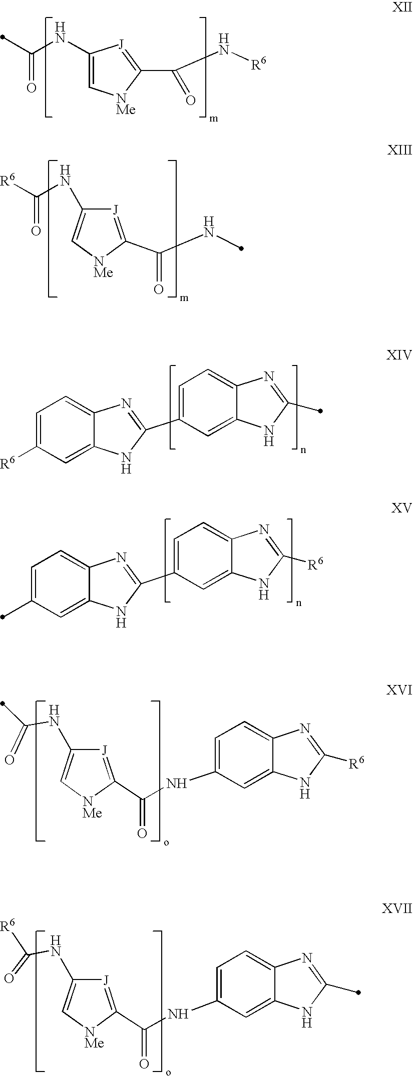

wherein in structures XII-XVII R⁶ is independently selected from an optionally substituted C₁₋₆ alicyclic or an optionally substituted C₃₋₆ cyclic alkyl group, and wherein the optional substituents are each independently selected from; halo, OH, OR⁷ NO₂, NH₂, NHR⁷, NR⁷R⁷, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷;

R⁶ may also be represent an optionally substituted aryl or an optionally substituted heteroaryl group having up to 12 carbon atoms, and wherein the optional substituents are each independently selected from; halo, OH, OR⁷, NH₂, NHR⁷, NR⁷R⁷, SH, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷, and each heteroaryl group contains one or more heteroatoms in its ring system which are each independently selected from O, N or S;

wherein each R⁷ is independently selected from an optionally substituted C₁₋₄ alkyl or an optionally substituted C₂₋₄ alkenyl group and wherein the optional substituents are each independently selected from OH, OR⁸, NH₂, NHR₃, NR⁸₂ or N(OH)R⁹⁸ wherein each R⁸ is independently selected from C₁₋₄ alkyl, C₂₋₄ alkenyl, OH, NO₂, NH₂, CF₃, CN, CO₂H or SH;

D may represent up to four of the following groups as substituents at any available ring carbon position; H, R⁹, hydroxy, alkoxy, halogen, NO₂, NH₂, NHR⁹, NR⁹₂, SH, SR⁹, SO₂R⁹, CF₃, CN, CO₂H, CO₂R⁹, CHO, COR⁹, CONH₂, CONHR⁹ or CONR⁹R⁹, cyclic alkylamino, imidazolyl, alkylpiperazinyl, morpholino, wherein each R⁹ independently selected from an optionally substituted C14 alkyl or an optionally substituted C₂₋₄ alkenyl group and wherein the optional substituents are each independently selected from OH, OR¹⁰, NH₂, NHR¹⁰, NR¹⁰₂ or N(OH)R¹⁰ wherein each R¹⁰ is independently selected from C₁₋₄ alkyl, C₂₋₄ alkenyl, OH, NO₂, NH₂, CF₃, CN, CO₂H or SH;

and wherein any available ring carbon position of formulae III-XVII may also be optionally replaced by —N— when the valency and configuration of the formula allows, the point of attachment of formulae III-XVII to the A group defined above is represented by ♦; and

wherein in formulae XII and XIII, m is selected from 2, 3 or 4,

and wherein in formulae XII, XIII, XVI or XVII J is selected from CH or N; and

wherein in formulae XIV and XV n is selected from 0, 1 or 2, and

wherein in formulae XVI and XVII o is selected from 1 or 2.

A preferred embodiment of formula II′ is one in which the DNA targeting unit is selected from one of formulae IV-X.

A preferred embodiment of formula 11′ is one in which D of the DNA targeting unit of Formulae III-XI is H or Me.

Preferred compounds of formula 11′ include the following

wherein X is CH₂—, Y₁ is H, Y₅ is NHCH₂CH₂OMe, Z is —N—, A is —(CH₂)NMe(CH₂)₂NHCO—, the DNA targeting unit represents formula IX and D is H;

wherein X is CH₂—, Y₁ is H, Y₅ is NHCH₂CH₂OMe, Z is —N—, A is —(CH₂)₂NH(CH₂)₃NHCO—, the DNA targeting unit represents formula IX and D is H;

wherein X is CH₂—, Y₁ is H, Y₅ is NHCH₂CH₂OMe, Z is —N—, A is —(CH₂)NMe(CH₂)₂NHCO—, the DNA targeting unit represents formula IX and D is Me;

wherein X is CH₂—, Y₁ is H, Y₅ is NHCH₂CH₂OMe, Z is —N—, A is —(CH₂)₂NMe(CH₂)₃NHCO—, the DNA targeting unit represents formula IX and D is Me;

wherein X is CH₂—, Y₁ is H, Y₅ is NHCH₂CH₂OMe, Z is —N—, A is —(CH₂)NMe(CH₂)₂NHCO—, the DNA targeting unit represents formula X and D is Me;

wherein X is CH₂—, Y₁ is H, Y₅ is NHCH₂CH₂OMe, Z is —N—, A is —(CH₂)₂NH(CH₂)₃NHCO—, the DNA targeting unit represents formula X and D is Me;

In a fourteenth aspect, there is provided a method of treating a subject in need of cancer therapy, said method comprising the steps of administering to said subject a cytotoxic effective amount of a compound of Formula II′ as defined above to the tumour cells in said subject.

Preferably the tumour cells are in a hypoxic environment.

Preferably, the method includes the further step of administering the compound of Formula II′ in combination with one or more other chemotherapeutic agents or treatments, including radiotherapy, either simultaneously, or sequentially, depending on the cancerous condition to be treated.

More preferably the method includes the step of administering radiotherapy to the tumour cells before, during or after the administration of the composition as defined above.

Preferably, the chemotherapeutic agents are selected from Cisplatin or other platinum-based derivatives, Temozolomide or other DNA methylating agents, cyclophosphamide or other DNA alkylating agents, doxorubicin, mitoxandrone, camptothecin or other topoisomerase inhibitors, methotrexate, gemcitabine or other antimetabolites.

While the method of the invention will typically be used in cancer therapy of human subjects, they may be used to target tumour cells in other warm blooded animal subjects such as other primates, farm animals such as cattle, and sports animals and pets such as horses, dogs, and cats.

Preferably, the compound of Formula II′ is administered with a pharmaceutically acceptable excipient, adjuvant, carrier, buffer or stabiliser. The pharmaceutically acceptable excipient, adjuvant, carrier, buffer or stabiliser should be non-toxic and should not interfere with the efficacy of the active composition. The precise nature of the carrier or other material will depend on the intended route of administration, which may be oral, or by injection such as cutaneous, subcutaneous or by intravenous injection. Pharmaceutical compositions of Formula II′ for oral administration may be in tablet, capsule, powder or liquid form. A tablet may comprise a solid carrier or an adjuvant. Liquid pharmaceutical compositions generally comprise a liquid such as water, petroleum, animal or vegetable oils, mineral oil or synthetic oil. Physiological saline solution, dextrose or other saccharide solution or glycols such as ethylene glycol, propylene glycol may be included. A capsule may comprise a solid carrier such as a gelatine. For intravenous, cutaneous or subcutaneous injection, the active composition will be in the form of a parenterally acceptable aqueous solution that is pyrogen-free and has a suitable pH, isotonicity and stability. Those of skill in the art would be able to prepare suitable solutions using for example, isotonic vehicles such as sodium chloride injection, Ringer's injection, lactated Ringer's injection. Preservatives, stabilisers, buffers, antioxidants and/or other additives may be included as required.

A “cytotoxic effective amount”, is to be understood as an amount of a compound of Formula II′ defined above that is sufficient to show benefit to a patient. The actual amount, rate and time-course of administration, will depend on the nature and severity of the disease being treated. Prescription of treatment is within the responsibility of general practitioners and other medical doctors.

A hypoxic environment is to be understood as tissue environments at an oxygen concentration of <10 mM.

In a fifteenth aspect there is provided, the use in the manufacture of a medicament of an effective amount of a compound of Formula II′ as defined above for the treatment of a subject in need of cancer therapy.

In a sixteenth aspect of the invention, there is provided a method of potentiating the cytotoxicity of an amount of a compound of Formula B or a composition including Formula B as defined above, which has been administered to a subject in need of cancer therapy, by administering to said subject a compound of Formula A or a composition including Formula A as defined above.

Preferably the method potentiates the hypoxic cytotoxicity of an amount of a compound of Formula B.

Preferably, the method includes the further step of administering to said subject the compound of Formula A or a composition including Formula A in combination with one or other chemotherapeutic agents or treatments defined above, including radiotherapy, either simultaneously, or sequentially depending on the cancerous condition to be treated.

More preferably, the method includes the step of administering radiotherapy to the subject, before, during or after the administration of said compound of Formula A or said composition including Formula A.

In a seventeenth aspect of the invention, there is provided a method of potentiating the cytotoxicity of one or more chemotherapeutic agents as defined above, administered to a subject, by further administering to said subject a compound of Formula A or a composition including Formula A as defined above.

Preferably the method potentiates the hypoxic cytotoxicity of the one or more chemotherapeutic agents.

Preferably, the method includes the further step of administering radiotherapy to said subject, either simultaneously, or sequentially depending on the cancerous condition to be treated.

More preferably, the method includes the step of administering radiotherapy to the subject, before, during or after the administration of said compound of Formula A or said composition including Formula A.

It is to be recognised that certain compounds of the present invention may exist in one of more different enantiomeric or diastereomeric forms. It is to be understood that the enantiomeric or diasteriomeric forms are included in the above aspects of the invention.

The term halo or halogen group used throughout the specification is to be taken as meaning a fluoro, chloro, bromo or iodo group.

Further aspects of the present invention will become apparent from the following description given by way of example only and with reference to the accompanying Figures and synthetic schemes, in which:

FIG. 1 shows the potentiation of the anoxic cytotoxicity of the benzotriazine di-N-oxide tirapazamine (TPZ; 30 μM) by the corresponding 1-oxide SR4317 in stirred single cell suspensions of HT29 human colon carcinoma cells at 5×10⁵ cells/ml. Cultures were maintained at <10 ppm O₂ under a continuously-flowing stream of 5% CO₂ in nitrogen and were sampled at intervals to determine plating efficiency. SR4317 alone was non-toxic up to its solubility limit (ca 1 mM).

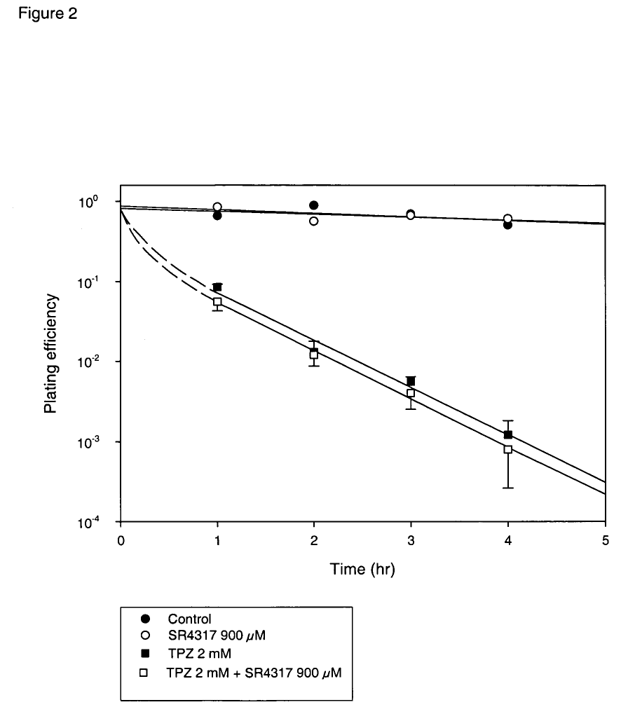

FIG. 2 shows lack of potentiation of the cytotoxicity of TPZ by SR4317 under aerobic conditions. Experimental conditions were as for FIG. 2, except that the gas phase was 5% CO₂ in air. Values are means and error bars are ranges for duplicate cultures.

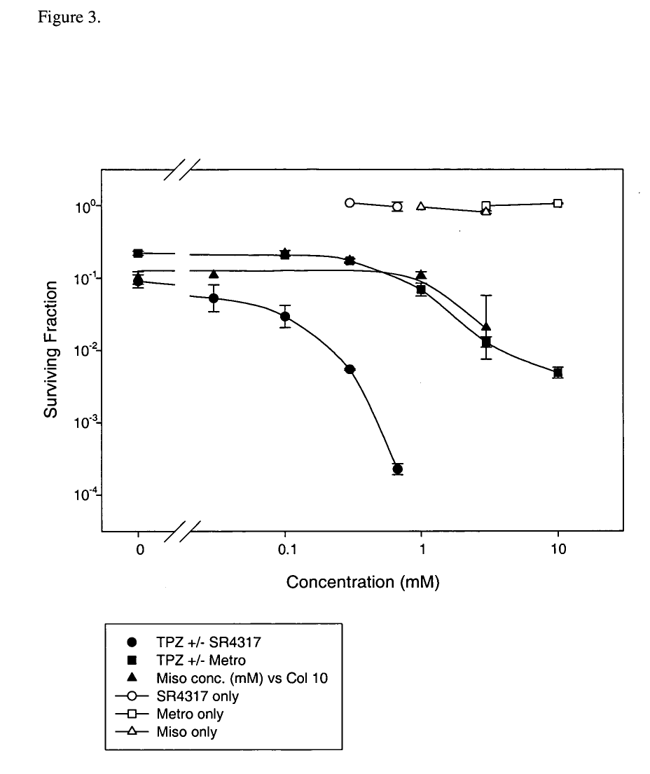

FIG. 3 shows potentiation of the cytotoxicity of TPZ (30 μM) against anoxic HT29 cells (5×10⁵/ml) by SR 4317, misonidazole and metronidazole. Drug exposure time was 1 hr. Error bars represent the range for duplicate determinations.

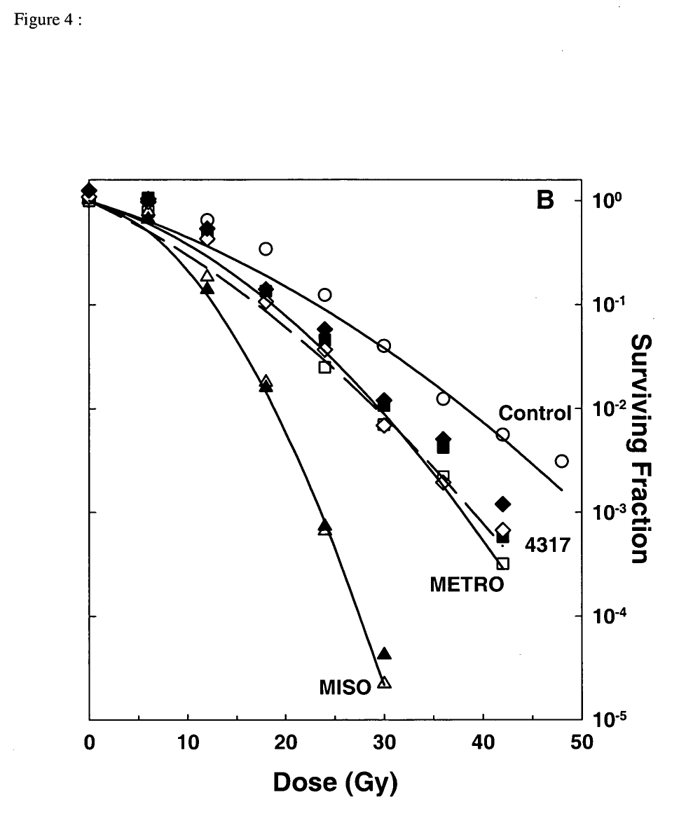

FIG. 4 shows radiosensitisation of anoxic HT29 cells (5×10⁵/ml) by 0.6 mM SR 4317 (squares), 0.6 mM misonidazole (triangles), and 0.6 mM metronidazole (diamonds). Anoxic cell suspensions were irradiated 5 min after addition of pre-equilibrated anoxic drug solutions. Data are shown for duplicate determinations, and are fitted using a linear-quadratic model

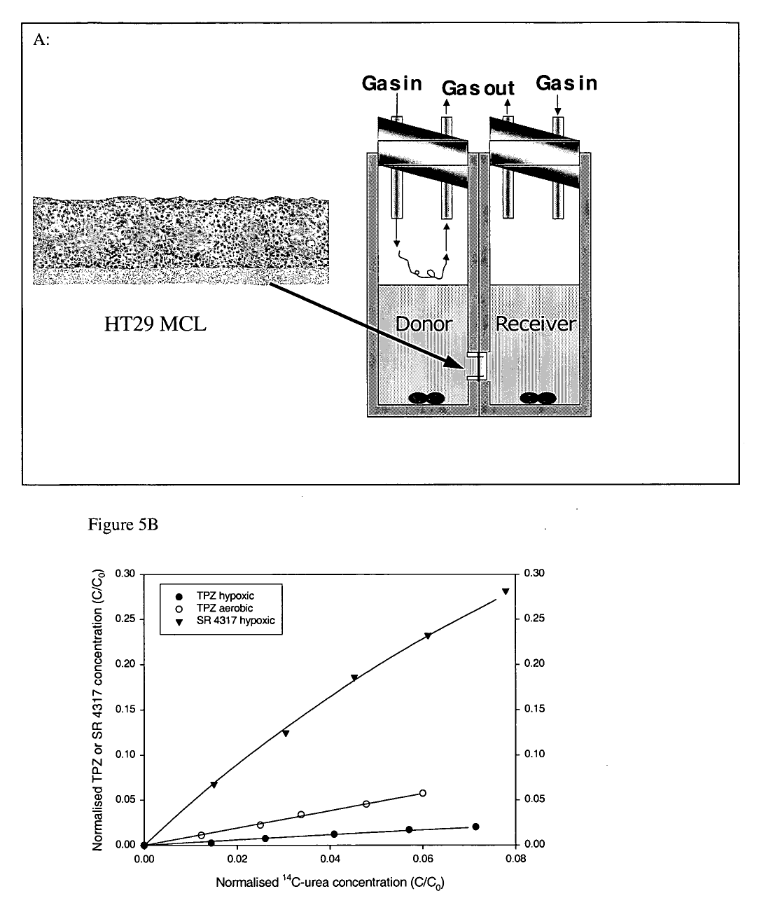

FIG. 5A shows histology of an HT29 MCL, stained with haematoxylin and eosin, and apparatus (diffusion chamber) for measurement of transport through MCLs. Compounds are added to the donor compartment, along with ¹⁴C-urea as an internal standard, and diffusion into the receiver compartment is monitored by HPLC and scintillation counting.

FIG. 5B shows transport of SR 4317 (200 μM) and TPZ (50 μM) through oxic and hypoxic HT-29 MCLs (ca 160 μm in thickness). The concentrations in the Receiver compartment (normalised against the initial concentration in the Donor compartment) are plotted against those of the flux marker ¹⁴C-urea to account for small differences in thickness of the MCLs. The fitted diffusion coefficients for TPZ (n=12) and SR 4317 (n=2) are: 3.97 and 32.7×10⁷ cm² sec⁻¹, respectively.

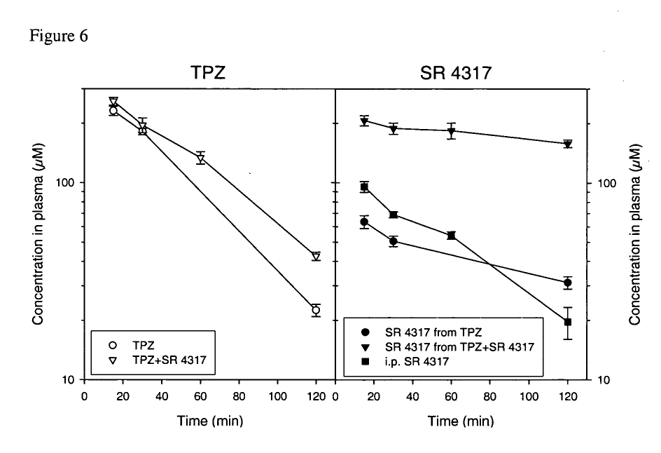

FIG. 6 shows plasma pharmacokinetics of TPZ (left panel) and SR 4317 (right panel) after intraperitoneal administration of TPZ (270 mmol/kg; circles), SR 4317 (750 μmol/kg; squares), or co-administration of TPZ+SR 4317 (triangles) at these doses to CD-1 mice bearing HT29 human tumour xenografts.

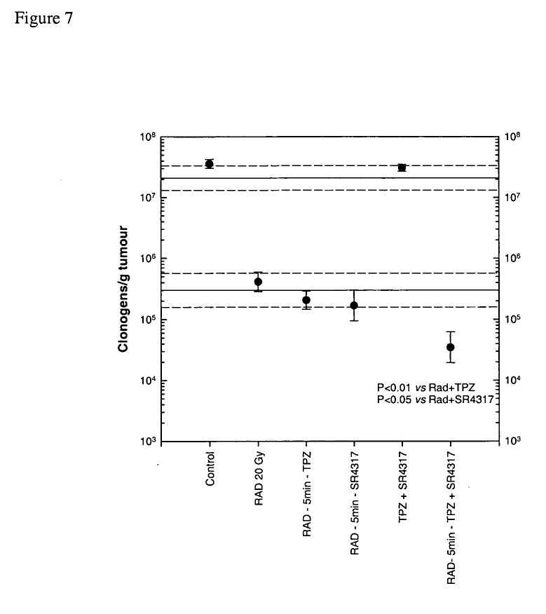

FIG. 7 shows potentiation of the cytotoxicity of TPZ (133 μmol/kg) against hypoxic (radioresistant) cells in HT29 tumours. Animals with subcutaneous tumours (ca 300 mg) received whole body radiation (RAD; 20 Gy) to sterilise oxygenated tumour cells. Activity of drugs against the hypoxic survivors was determined by intraperitoneal administration of solutions in 5% DMSO/saline 5 min after radiation, using TPZ alone (133 μmol/kg) or in combination with SR 4317 (1000 μmol/kg). Tumours were excised 18 hr after treatment, dissociated enzymatically, and plated to determine the number of clonogenic survivors. Values are geometric means and error bars are standard errors of the mean. Horizontal lines show historical values for untreated controls and radiation only (solid lines are means, dashed lines are 95% confidence limits. p values were determined by one-way ANOVA using only data within this experiment.

### DETAILED DESCRIPTION OF THE INVENTION

The inventors have demonstrated that the cytotoxicity of tirapazamine (TPZ) to hypoxic tumour cells can surprisingly be increased quite markedly by simultaneous exposure to SR 4317, as illustrated for HT29 tumour cells in culture in FIG. 1. Advantageously, SR 4317 does not potentiate the aerobic toxicity of TPZ (FIG. 2), and therefore can be used to increase the hypoxic selectivity of the latter.

This observation provides evidence that the second (DNA radical oxidation) step in the dual action of TPZ as illustrated in Scheme A in the background of the invention is a limiting factor for its hypoxic cytotoxicity. It also demonstrates that the therapeutic utility of TPZ and related analogues could, in principle, be improved by simultaneous exposure to SR 4317 or an analogous DNA radical oxidant.

The inventors have also shown that DNA radical oxidising agents other than TPZ (illustrated by the nitroimidazoles metronidazole and misonidazole) are able to potentiate the hypoxic cytotoxicity of TPZ, although with lower dose potency than SR 4317 (FIG. 4). Unexpectedly, comparison of the ability of these agents to radiosensitize hypoxic HT29 cells (FIG. 5), under the same conditions as for the TPZ potentiation experiments, shows that the structure-activity relationship for TPZ potentiation (potency SR 4317>metronidazole=misonidazole) is different from that for radiosensitization (misonidazole>metronidazole=SR4317). The inventors therefore consider that there is a special feature of the benzoazine N-oxide system for potentiation of the hypoxic cytotoxicity of TPZ and its related analogues.

The inventors have also investigated the extravascular transport properties (tissue diffusion characteristics) of SR 4317 to assess whether it can diffuse well enough to reach hypoxic cells in tumours efficiently. This study used the multicellular layer (MCL) assay (Hicks et al., Int. J. Radiat. Oncol., Biol., Phys., 1998, 42, 641; Hicks et al., J. Pharmacol. Exper. Ther., 2001, 297, 1088), developed in this laboratory. Transport of SR 4317 through hypoxic MCLs grown from HT29 cells was faster than for TPZ FIG. 5, which indicates its ability to reach hypoxic cells in tumours.

The plasma pharrmacokinetics of SR 4317 and TPZ were determined in C₃H mice, alone and in combination (FIG. 6), to assess whether therapeutic concentrations of SR 4317 can be achieved in mice. The maximum SR 4317 concentration in plasma, after co-administration of the two compounds, was ca 200 μM with a slow clearance over the first two hours. Based on the in vitro results in FIG. 3 (showing significant potentiation of TPZ cytotoxicity with SR 4317 at 100 μM for 1 hr), and the efficient tissue penetration of SR 4317 (FIG. 4), the plasma concentration profile would appear to be high enough to cause potentiation of TPZ hypoxic cytotoxicity in tumours.

The utility of SR 4317 as a potentiator of the hypoxic cytotoxicity of TPZ was assessed in an in vivo model (HT29 tumour xenografts) as illustrated in FIG. 7. In this experiment tumour response was determined by excising tumours 18 hr after treatment and quantifying the number of clonogenic survivors by plating in vitro. TPZ was administered at a sub-efficacious dose (0.133 mmol/kg), 5 minutes after whole body radiation (20 Gy). As anticipated from earlier experiments, this dose of TPZ did not result in statistically significant killing of the (hypoxic) cells surviving radiation. Adding SR 4317 (1 mmol/kg) to this combination provided activity that was now greater than radiation alone (p<0.01 by one way ANOVA), and the difference between radiation+TPZ vs radiation+TPZ+SR 4317 was also significant (p <0.05). No significant cytotoxicity was observed when the two compounds were administered in the absence of irradiation, indicating lack of activity against aerobic cells in tumours (FIG. 7). This experiment demonstrates selective potentiation of the hypoxic cytotoxicity of TPZ in tumours.

It is envisaged that further potentiation over and above that seen with SR 4317 could be readibly achieved. This is because SR4317 has only modest aqueous solubility (ca 800 μM) and provides relatively low plasma concentrations in mice.

The following table shows potentiation of anoxic cytotoxicity results using TPZ and benzotriazine-1-oxides.

TABLE 1
Potentiation of the anoxic cytotoxicity of tirapazamine by
benzotriazine-1-oxides as assessed using an IC50 assay (HT29 cell line)

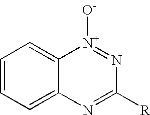

| | | | | TPZ | |
|---|---|---|---|---|---|
| | | Solu- | IC₅₀ of | potentiation test^{c} | |
| | | bility | potent- | Conc of | |
| Compound | | limit | iator | potentiator | |
| No (SN) | R | (mM)^{a} | (mM)^{b} | (mM) | TPR |
| 3 | NH₂ | 0.8 | >0.5 | 0.5 | 2.0 |
| (SR 4317) | | | | | |
| 25 | NHCH₂CN | 0.3 | >0.3 | 0.2 | 1.9 |
| 130 | NHCH₂CH₂NHCH₃ | 3 | 2.3 | 1.0 | 6.1 |
| 37 | NHCH₂CH₂N(CH₃)₂ | 1 | >1 | 1.0 | 13.9 |
| 44 | NH(CH₂)₃N(CH₃)₂ | 3.0 | 0.83 | 0.3 | 1.2 |
| 56 | Et | 3.0 | >3 | 3.0^{d} | 1.3 |
| 61 | CH₂CH₂OH | 3.0 | >3 | 3 | 1.2 |
| 71 | OCH₃ | 0.3 | >0.3 | 0.3 | 3.3 |

^{a}Approximate solubility limit in Alpha MEM culture medium containing 5% fetal calf serum, determined by dilution from DMSO stock solutions
^{b}Concentration of potentiator required to decrease density of HT29 colon carcinoma cells to 50% of controls on the same 96-well plate following 4 hr exposure under anoxic conditions (anaerobic chamber). Cell density was determined by staining with sulphorhodamine B five days after washing out the compounds.
^{c}The IC₅₀ of TPZ (4 hr anoxic exposure of HT29 cells) was determined in the presence and absence of the potentiator at the indicated concentration. Controls with potentiator only indicated that these concentrations were non-toxic. TPR (TPZ potentiation ratio) = TPZ IC₅₀ without potentiator/TPZ IC₅₀ with potentiator (on the same 96 well plate). The IC₅₀ for TPZ alone in this experiment was 0.0068 ± 0.0010 μM (mean ± sem, n = 4)
^{d}Approximate concentration (slight precipitation in the IC₅₀ assay).

Methods for Preparing Compounds of Formulas I, I′, II, and II′ of the Invention.

Reaction of the appropriately substituted 2-nitroanilines 1 with cyanamide, followed by cyclization of the intermediate guanidine with NaOH (Method A) gave corresponding 1-oxides 3. In several instances (7-CF₃, 7-NO₂, 5-Cl, 8-F), it was necessary to use an alternative method (Method B) involving reaction of 2-halonitrobenzenes (2) with guanidine, followed by cyclization under basic conditions. This gave 1-oxides 3 in modest yield (Scheme 1).

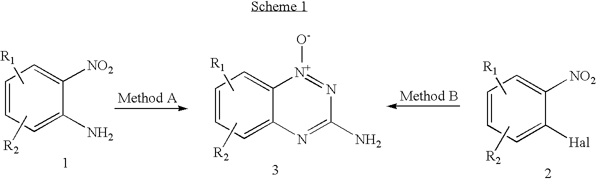

Method A Reagents:

a) NH₂CN, HCl;

b) NaOH;

Method B Reagents:

a) Guanidine.HCl, tBuOK.

The sulfides 1f and 1g were prepared by substitution of 3-chloro-2-nitroaniline 1d (Scheme 2).

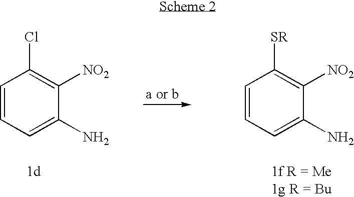

Reagents:

a) MeSLi, DMF;

b) BuSLi, DMF.

The sulfide 1n and 1o were prepared using the Newman-Kwart rearrangement (Newman & Karnes, J. Org. Chem. 1966, 31, 3980-3984) and vicarious nucleophilic substitution (VNS) (Seko, et al., J. Chem. Soc. Perkin Trans. 1 1999, 1437-1444; Makosza & Bialecki, J. Org. Chem. 1998, 63, 4878-4888) (Scheme 3). Thus, isomerisation of O-thiocarbamate 4 gave S-thiocarbamate 5, which was hydrolysed, and the intermediate thiol alkylated with MeI to give sulfide 6. V_{N}S Reaction of 6 with NH₂OMe.HCl gave nitroanilines 1 n and lad. A similar sequence from 5 gave butylsulfanylnitroaniline 1o as well as the isomeric 8 and 9.

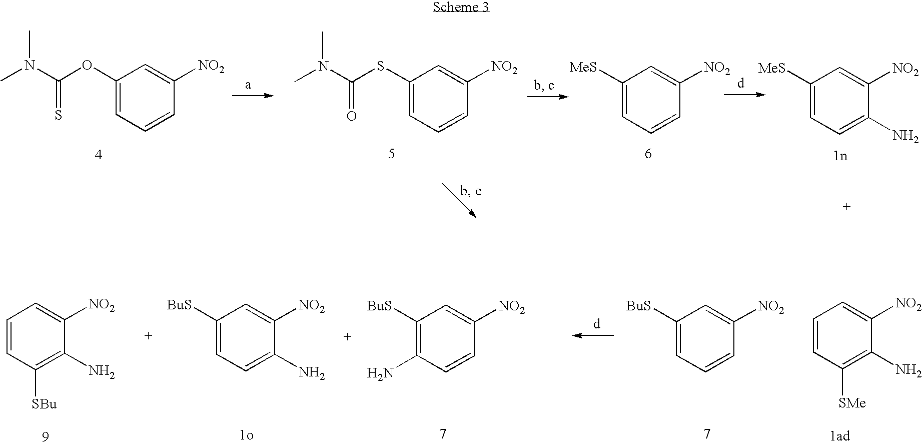

Reagents:

a) neat, 220° C.;

b) KOH, MeOH;

c) Me₂SO₄, KOH, MeOH;

d) NH₂OMe.HCl, KOtBu, CuCl, DMF;

e) nBuBr, K₂CO₃, DMF.

Nitroaniline 1q was prepared by Curtius rearrangement of 5-methoxy-2-nitrobenzoic acid (10) (Scheme 4). Nitroaniline 1x was prepared by nucleophilic displacement of 1 u (Scheme 4).

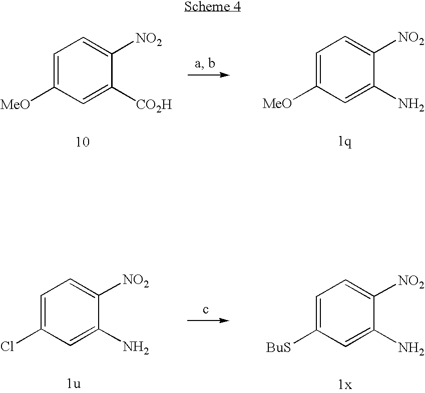

Reagents:

a) Diphenylphosphorylazide, Et₃N, tBuOH;

b) HCl, MeOH;

c) NBuSLi, DMF.

Displacement of the fluoride 3k with dimethylamine gave 1-oxide 11 (Scheme 5) and similarly reaction of fluoride 3t with either dimethylamine or diethylamine gave 12 and 13, respectively.

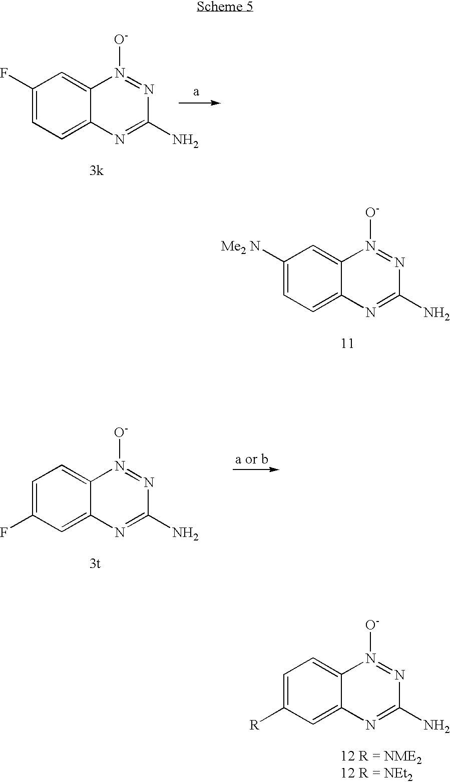

Reagents:

a) NMe₂, MeCN;

b) NEt₂, MeCN.

Alkylation of the 7-hydroxy-1-oxide 3h with bromides gave compounds 14-17 (Scheme 6).

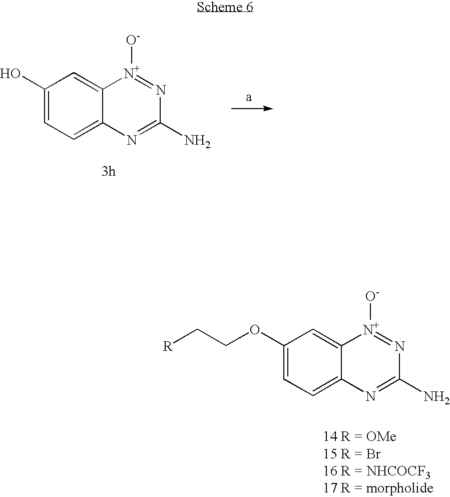

Reagents:

For 14: BrCH₂CH₂OMe, K₂CO₃, DMF;

For 15: BrCH₂CH₂Br, K₂CO₃, DMF;

For 16: BrCH₂CH₂NHCOCF₃, K₂CO₃, DMF;

For 17: BrCH₂CH₂morpholide, K₂CO₃, DMF.

1,2,4-Benzotriazin-3-amine 1-oxide (3) (Scheme 1: R₁═R₂═H) was synthesized from nitroaniline using Method A. Diazotisation gave the phenol 18 which was chlorinated to give chloride 19 (Scheme 7). Reaction of chloride 19 with glycine ethyl ester gave the ester 20. Base catalysed hydrolysis of the ester gave the acid 21.

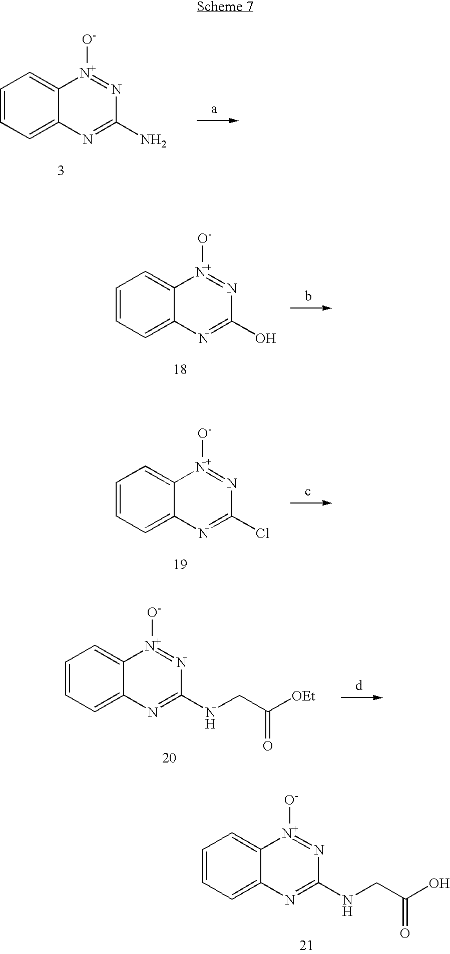

ents:

a) NaNO₂, HCl;

b) POCl₃, PhNMe₂;

c) NH₂CH₂CO₂Et, Et₃N, DME;

d) NaOH, MeOH.

Reaction of chloride 19 with various alkylamines in refluxing DME gave 1-oxides 22-29 and 32 in good yields (Scheme 8).

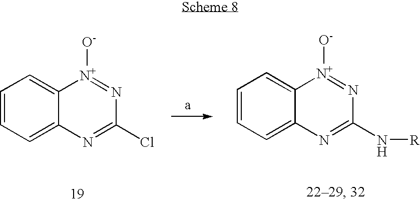

| Compound | Reagent | R = |
|---|---|---|
| 22 | NH₂(CH₂)₂OH | —(CH₂)₂OH |
| 23 | NH₂(CH₂)₂OMe | —(CH₂)₂OMe |
| 24 | NH₂(CH₂)₃OH | —(CH₂)₃OH |
| 25 | NH₂CH₂CN | —CH₂CN |
| 26 | NH₂(CH₂)₂CN | —(CH₂)₂CN |
| 27 | NH₂(CH₂)₃CN | —(CH₂)₃CN |
| 28 | NH₂(CH₂)₃N₃ | —(CH₂)₃N₃ |
| 29 | NH₂(CH₂)₃NHCO₂tBu | —(CH₂)₃NHCO₂tBu |
| 32 | NH₂(CH₂)₃N(Et)CO₂tBu | —(CH₂)₃N(Et)CO₂tBu |

Reaction of chloride 19 with 2-(aminoethoxy)ethanol gave 1-oxide 33 (Scheme 9) which was converted to the azide 34 by reaction with methanesulfonyl chloride and displacement with azide. Reduction with propanedithiol gave selective reduction of the azide 34 to an amine which was protected as carbamate 35. Deprotection of carbamate 35 under acidic conditions gave amine 36.

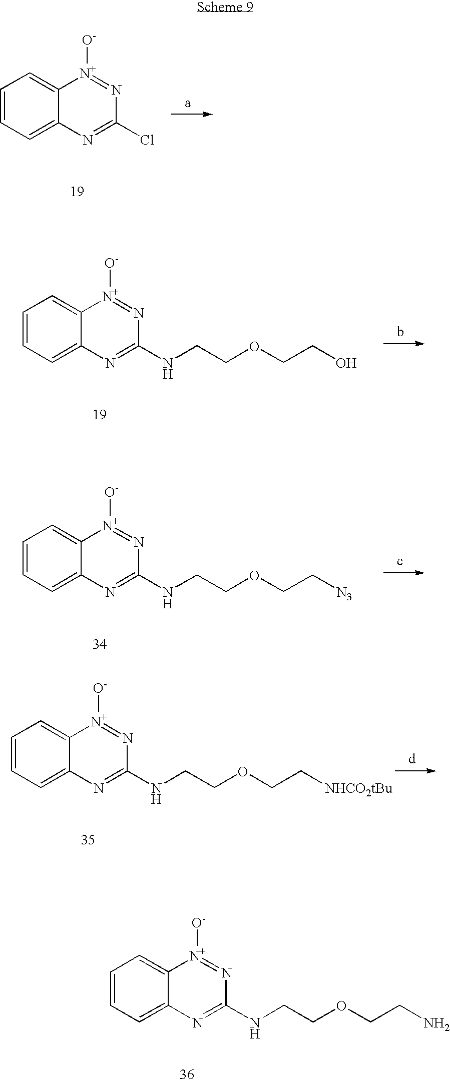

Reagents:

a) H₂(CH₂)₂O(CH₂)₂OH, Et₃N, DCM;

b) MsCl, Et₃N, DCM;

c) HS(CH₂)₃SH, Et₃N, MeOH; then di-t-butyidicarbonate, THF;

d) CI, MeOH.

Reaction of chloride 19 with a variety of amines gave 1-oxides 37, 38, 40-45 (Scheme 10). Reaction of alcohol 22 with methanesulfonyl chloride and displacement with di-n-propylamine gave 1-oxide 39.

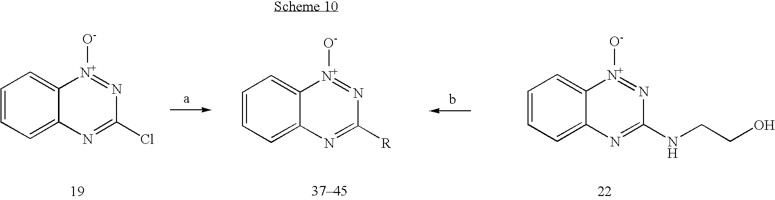

| Compound | Reagents | R = |
|---|---|---|
| 37 | NH₂(CH₂)₂NMe₂, DME | —NH(CH₂)₂NMe₂ |
| 38 | NHMe(CH₂)₂NMe₂, DME | —NMe(CH₂)₂NMe₂ |
| 39 | MsCl, Et₃N, DCM; then HNPr₂ | —NH(CH₂)₂NPr₂ |
| 40 | NH₂(CH₂)₂N-Pyrrolidine, DME | —NH₂(CH₂)₂N-Pyrrolidine |
| 41 | NH₂(CH₂)₂N-Morpholine, DME | —NH(CH₂)₂N-Morpholine |
| 42 | NH₂(CH₂)₂N-piperidine, DME | —NH(CH₂)₂N-piperidine |
| 43 | NH₂(CH₂)₂N-2,6- | —NH(CH₂)₂N-2,6-dimethylpiperidine |
| | dimethylpiperidine, DME | |
| 44 | NH₂(CH₂)₃NMe₂, DME | —NH(CH₂)₃NMe₂ |
| 45 | Aniline, HCl, DME | —NHPhenyl |

Aniline 48 was prepared by methylation of 3-nitrophenethyl alcohol 46 (Scheme 11) and reduction of the ether 47. Aniline 48 was coupled to chloride 19 to give 1-oxide 49.

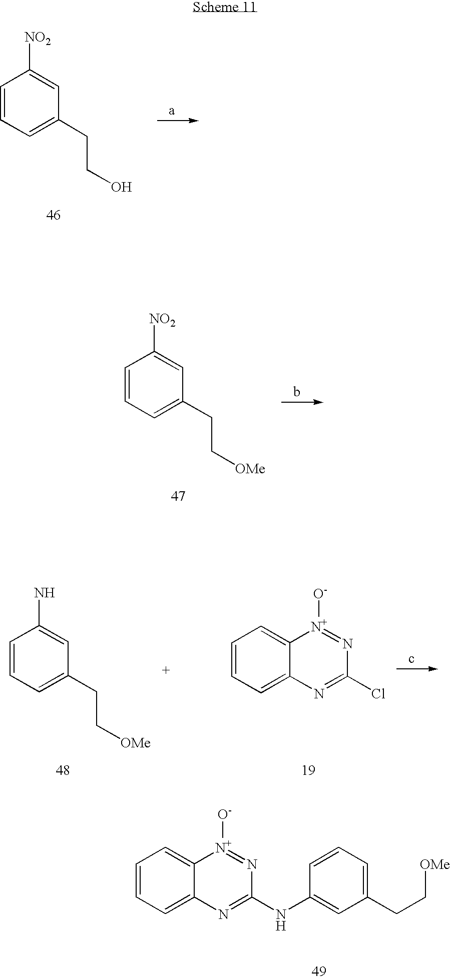

Reagents:

a) NaH, MeI, THF;

b) H₂, Pd/C, EtOH;

c) 19+48, DMSO.

Reaction of chloride 19 and aniline 50 gave ester 51 (Scheme 12). Hydrolysis of the ester 51 gave acid 52 which was condensed with methoxyethylamine and CDI to give amide 53. Condensation of 52 with N,N-dimethylethanediamine gave 54.

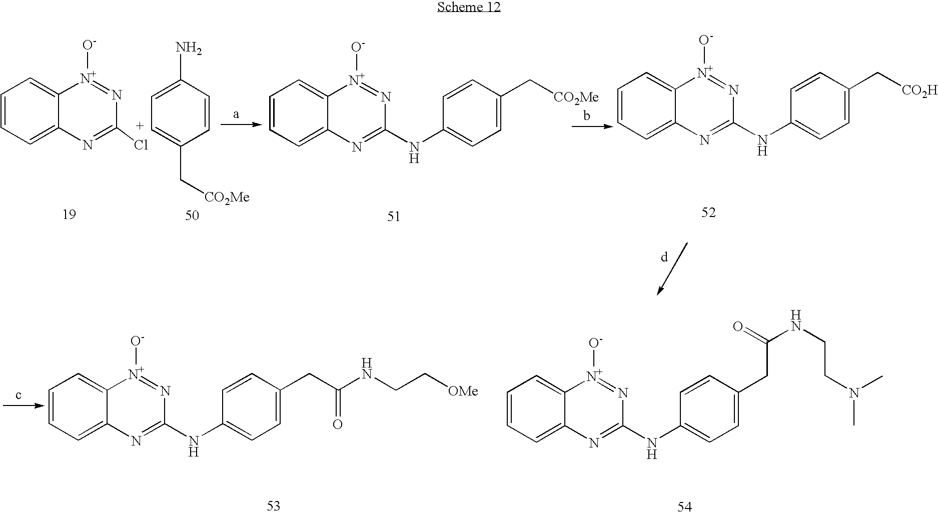

Reagents:

a) 19+50, DMSO;

b) aqueous NaOH, MeOH;

c) CDI, Methoxyethylamine, DMF;

d) CD₁, NH₂CH₂CH₂NMe₂, DMF.

Stille reaction of chloride 19 with tetramethyltin and Pd(PPh)₃ in refluxing DME gave the 3-methyl 1-oxide 55 (Scheme 13). Similarly, reaction of 19 with tetraethyltin gave 3-ethyl 1-oxide 56, reaction with vinyltributyltin gave 3-vinyl 1-oxide 59, and reaction with allyltributyltin gave 3-allyl 1-oxide 60. Suzuki reaction of chloride 19 with phenylboronic acid and 4-methoxyphenylboronic acid gave 3-aryl derivatives 57 and 58, respectively.

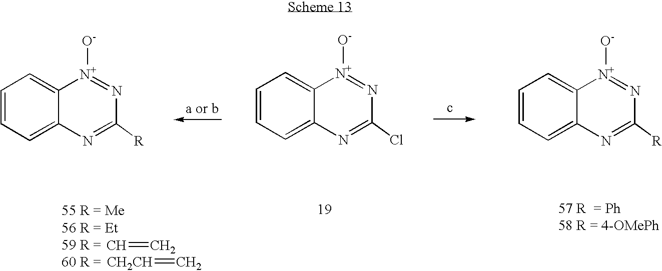

Reagents:

a) R₄Sn, Pd(PPh₃)₄, DME;

b) NBu₃SnR, Pd(PPh₃)₄, DME;

c) RB(OH)₂, Pd(PPH₃)₄, DME.

Oxidation of alkene 60 with MCPBA gave epoxide 62 (Scheme 14). Ozonolysis of 60, followed by a reductive workup gave 61. Treatment of alcohol 61 with TMS-diazomethane and HBF₄ gave the ether 63. Treatment of alcohol 61 with methanesulfonyl chloride followed by either dimethylamine or morpholine gave 1-oxides 64 and 65, respectively.

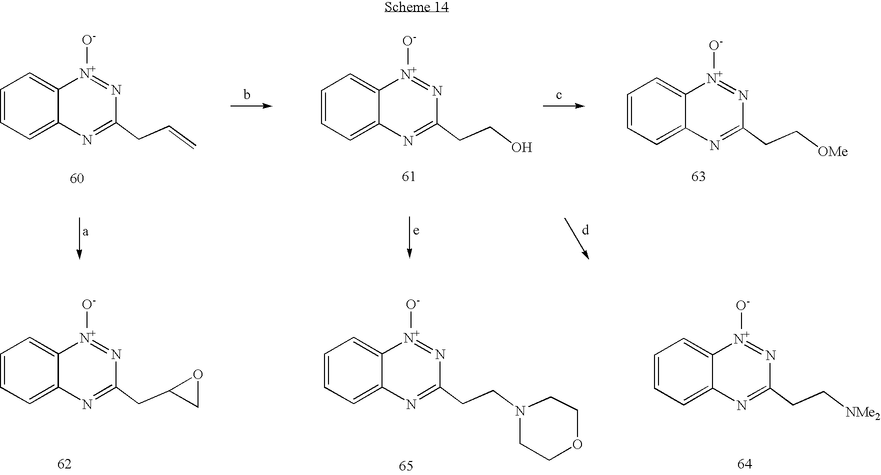

Reagents:

a) MCPBA, DCM;

b) O₃, DCM, MeOH; then NaBH₄;

c) TMSCH₂N₂, HBF₄, DCM;

d) MsCl, Et₃N, DCM; then HNMe₂.HCl, Et₃N, THF;

e) MsCl, Et₃N, DCM; then morpholine, THF.

Hydroboration and oxidation of alkene 60 gave alcohol 66 which was methylated with TMS-diazomethane and HBF₄ to give ether 67 (Scheme 15). Treatment of alcohol 66 with methanesulfonyl chloride and displacement with secondary amines gave 1-oxides 68-70.

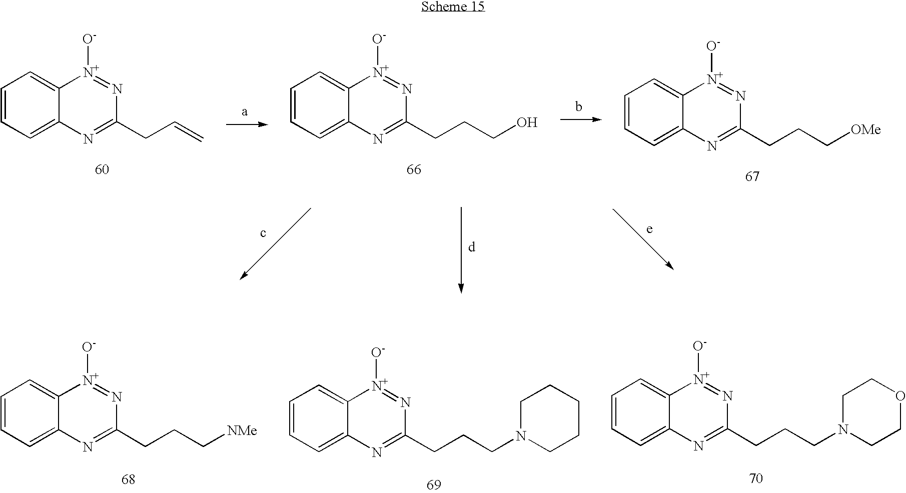

Reagents:

a) 9-BBN, THF; then 30% H₂O₂, NaOH;

b) TMSCH₂N₂, HBF₄, DCM;

c) MsCl, Et₃N, DCM; then HNMe₂, DMF;

d) MsCl, Et₃N, DCM; then piperidine, DMF;

e) MsCl, Et₃N, DCM; then morpholine, DMF.

Reaction of chloride 19 with sodium methoxide gave ether 71 (Scheme 16) and similarly reaction of 19 with sodium 2-methoxyethoxide gave ether 72.

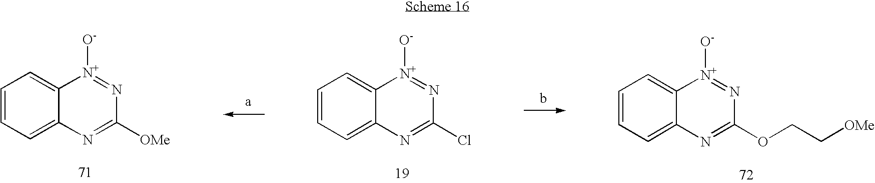

Reagents:

a) Na, MeOH;

b) Na, MeOCH₂CH₂OH;

Diazotisation of amine 3r in trifluoroacetic acid and chlorination of the intermediate phenol gave chloride 73 (Scheme 17). Nucleophilic displacement of chloride 73 with a variety of amines gave the 1-oxides 74-78.

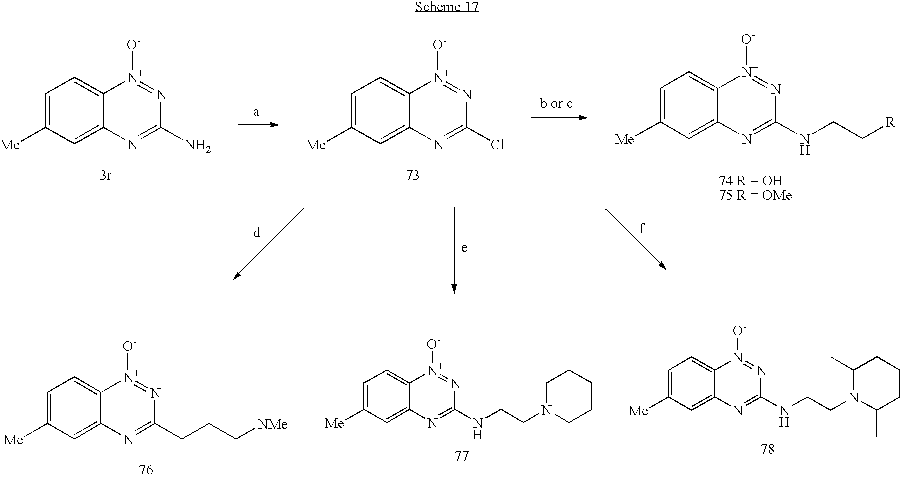

Reagents:

a) NaNO₂, TFA; then POCl₃, DMF;

b) NH₂CH₂CH₂OH, DME;

c) NH₂CH₂CH₂OMe, DME;

d) NH₂CH₂CH₂NMe₂, DME;

e) NH₂CH₂CH₂N-piperidine, DME;

f) NH₂CH₂CH₂N-2,6-dimethylpiperidine, DME.

Stille reaction of chloride 73 with tetraethyltin and Pd(PP₃)₄ gave 1-oxide 79, while reaction with allyltributyltin under similar conditions gave 1-oxide 80 (Scheme 18). Ozonolysis of 80 with a reductive workup gave alcohol 81 which was methylated with TMS-diazomethane and HBF₄ to give ether 82.

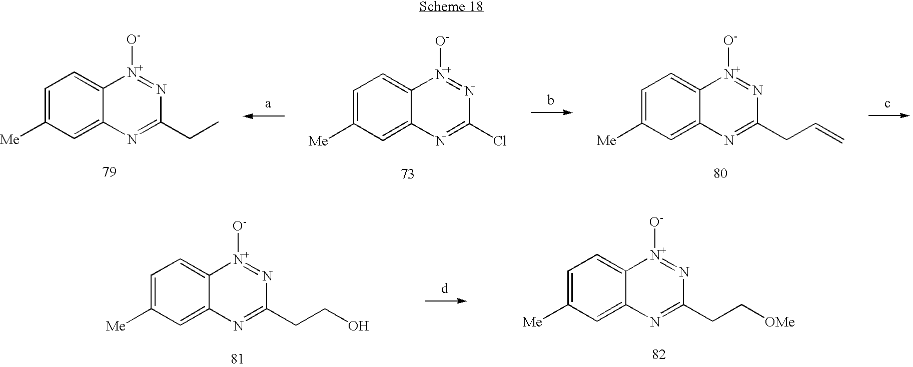

Reagents:

a) Et₄Sn, Pd(PPh₃)₄, DME;

b) nBu₃Snallyl, Pd(PPh₃)₄, DME;

c) O₃, DCM, MeOH, NaBH₄;

d) TMSCH₂N₂, HBF₄, DCM.

Diazotisation of amine 3q in trifluoroacetic acid and chlorination of the intermediate phenol gave chloride 83 (Scheme 19). Nucleophilic displacement of chloride 83 with a variety of amines gave the 1-oxides 84-86.

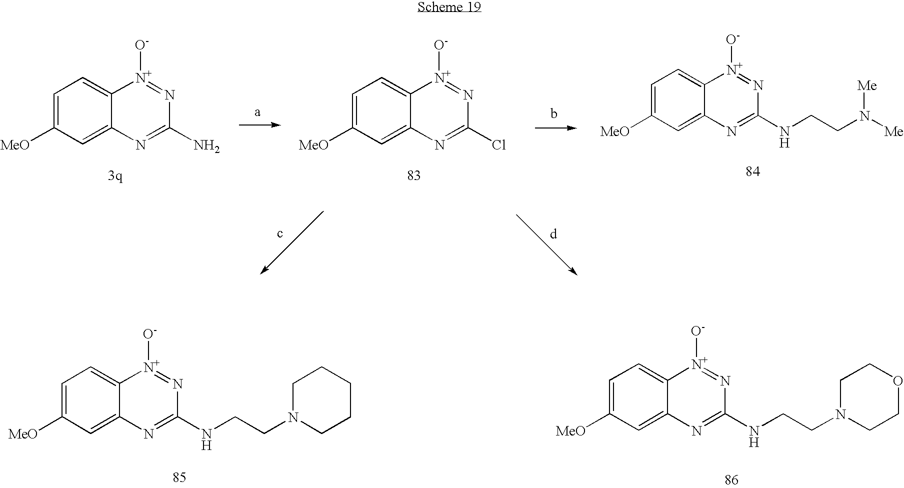

Reagents:

a) NaNO₂, TFA; then POCi₃, DMF;

b) NH₂CH₂CH₂NMe₂, DME;

c) NH₂CH₂CH₂N-piperidine, DME;

d) NH₂CH₂CH₂N-morpholine, DME.

Diazotisation of amine 3j and chlorination of the intermediate phenol gave chloride 87 (Scheme 20). Nucleophilic displacement of chloride 87 with a variety of amines gave the 1-oxides 88-90.

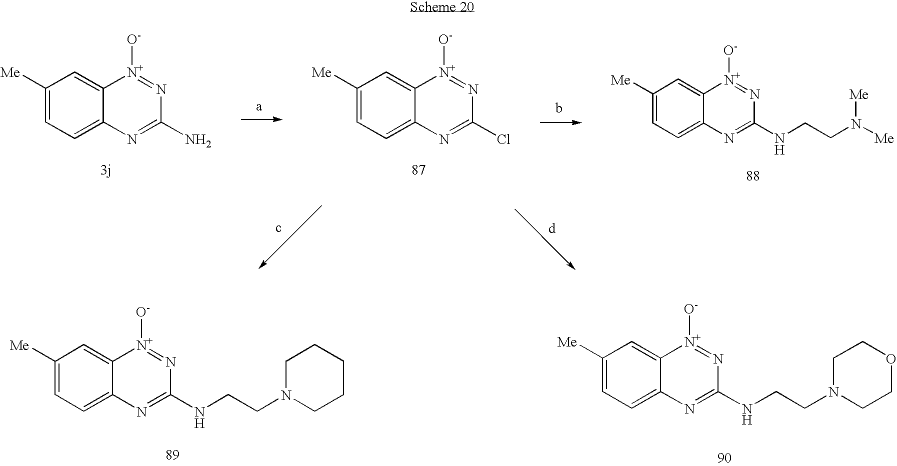

Reagents:

a) NaNO₂, aq. HCl; then POCl₃, DMF;

b) NH₂CH₂CH₂NMe₂, DME;

c) NH₂CH₂CH₂N-piperidine, DME;

d) NH₂CH₂CH₂N-morpholine, DME.

Diazotisation of amine 3i and chlorination of the intermediate phenol gave chloride 91 (Scheme 21). Nucleophilic displacement of chloride 91 with a variety of amines gave the 1-oxides 92-94.

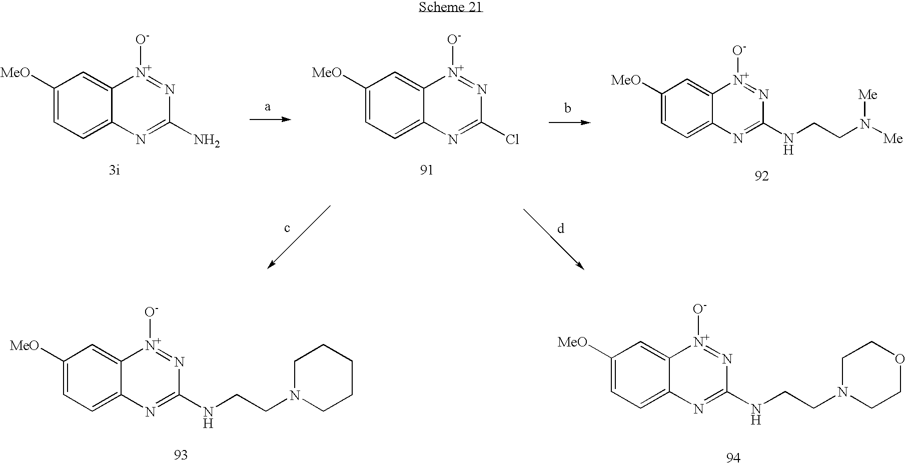

Reagents:

a) NaNO₂₁ aq. HCl; then POCl₃, DMF;

b) NH₂CH₂CH₂NMe₂, DME;

c) NH₂CH₂CH₂N-piperidine, DME;

d) NH₂CH₂CH₂N-morpholine, DME.

Diazotisation of amine 14 and chlorination of the intermediate phenol gave chloride 96 (Scheme 22). Stille reaction of chloride 96 with tetraethyltin and Pd(PPh₃)₄ in DMF gave the 1-oxide 97

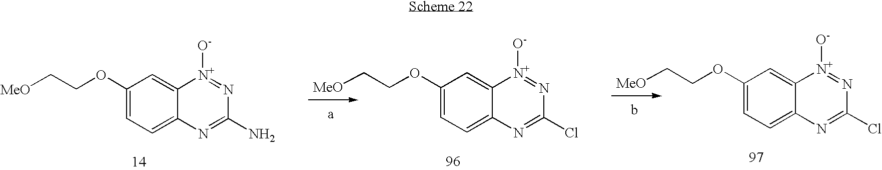

Reagents:

a) NaNO₂, aq. HCl; then POCl₃, DMF;

b) Et₄Sn, Pd(PPh₃)₄, DMF.

Similarly, diazotisation of amine 17 and chlorination of the intermediate phenol gave chloride 98 (Scheme 23). Stille reaction of chloride 98 with tetraethyltin and Pd(PPh₃)₄ in DMF gave the 1-oxide 99.

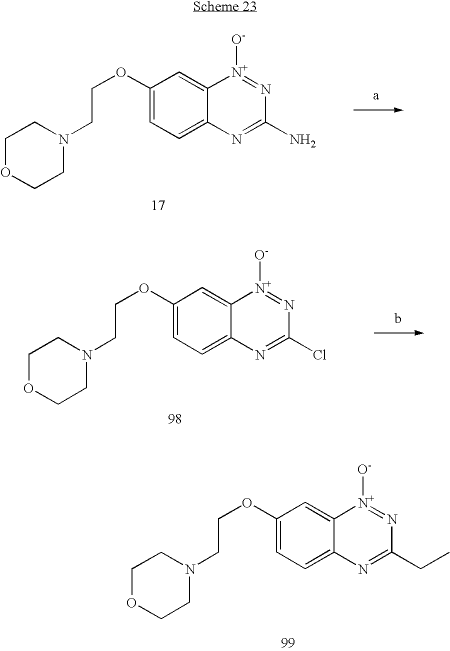

Reagents:

c) NaNO₂, aq. HCl; then POCl₃, DMF;

d) Et₄Sn, Pd(PPh₃)₄, DMF.

Diazotisation of amine 3b and chlorination of the intermediate phenol gave chloride 100 (Scheme 24). Nucleophilic displacement with amines gave 1-oxides 101 and 102.

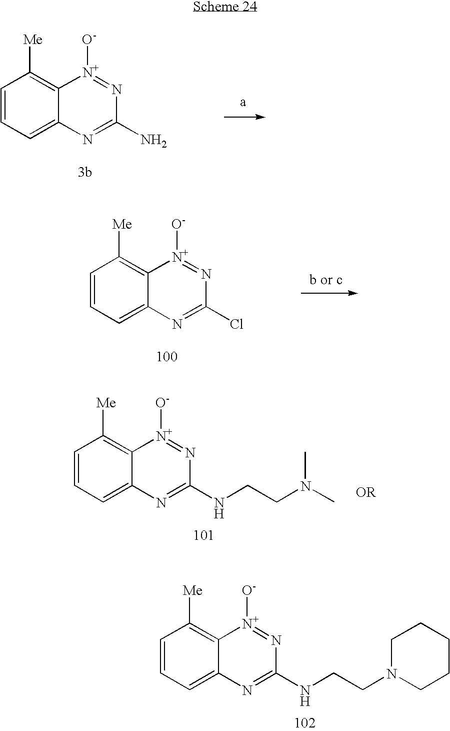

Reagents:

a) NaNO₂, TFA; then POCl₃, DMF;

b) NHMe(CH₂)₂NMe₂, DME;

c) NH₂(CH₂)₂N-piperidine, DME.

Reaction of nitroaniline 103 with cyanamide and condensation of the intermediate guanidine under basic conditions, followed by diazotisation and chlorination gave chloride 104 (Scheme 25). Displacement of 104 with amines gave 1-oxides 105 and 106.

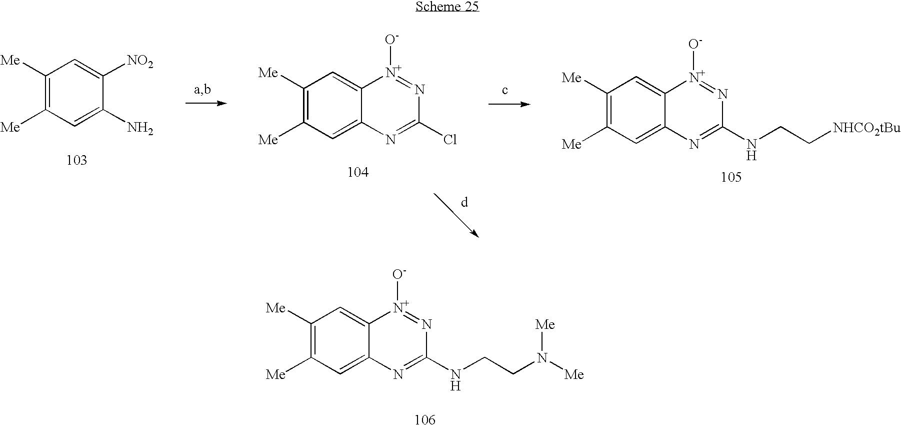

Reagents:

a) NH₂CN, HCl; then NaOH;

b) NaNO₂, TFA; then POCl₃, DMF;

c) NH₂CH₂CH₂N(Et)CO₂tBu, DME;

d) NHMe(CH₂)₂NMe₂, DME.

Reaction of nitroaniline 107 with cyanamide and condensation of the intermediate guanidine under basic conditions, followed by diazotisation and chlorination gave chloride 108 (Scheme 26). Displacement of 108 with amines gave 1-oxides 109 and 110.

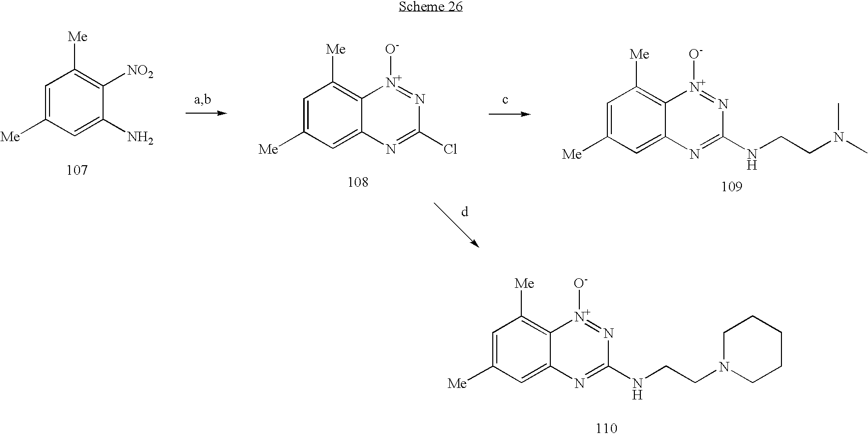

Reagents:

a) NH₂CN, HCl; then NaOH;

b) NaNO₂, TFA; then POCl₃, DMF;

c) NH₂CH₂CH₂NMe₂, DME;

d) NHMe(CH₂)₂Npiperidine, DME.

Reaction of nitroaniline 111 with cyanamide and condensation of the intermediate guanidine under basic conditions gave amine 112 (Scheme 27). Diazotisation and chlorination of 112 gave chloride 113. Displacement of 113 with N,N-dimethylethylenediamine gave 1-oxide 114.

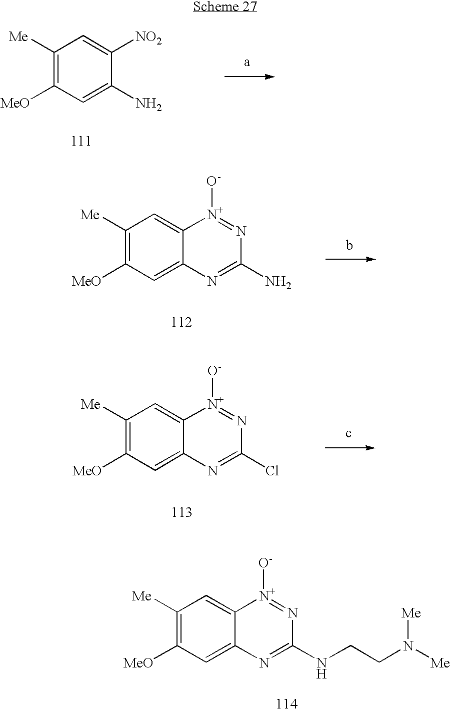

Reagents:

a) NH₂CN, HCl; then NaOH;

b) NaNO₂, TFA; then POCl₃, DMF;

c) NH₂CH₂CH₂NMe₂, DME.

Reaction of nitroaniline 115 with cyanamide and condensation of the intermediate guanidine under basic conditions gave amine 116 (Scheme 28). Diazotisation and chlorination of 116 gave chloride 117. Displacement of 117 with dimethylethylenediamine gave 1-oxide 118, while reaction of 117 with 2-(1-piperidinyl)ethylamine gave 1-oxide 119.

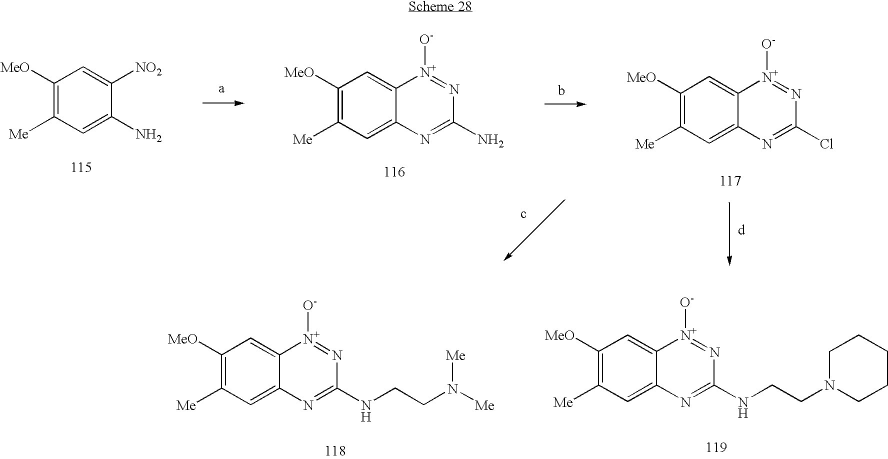

Reagents:

a) NH₂CN, HCl; then NaOH;

b) NaNO₂, TFA; then POCl₃, DMF;

c) NH₂CH₂CH₂NMe₂, DME;

d) NH₂CH₂CH₂Npiperidine, DME.

The imidazolide of 120 was formed using CDI in DMF and was coupled to amine 36 to give 1-oxide 121 (Scheme 29).

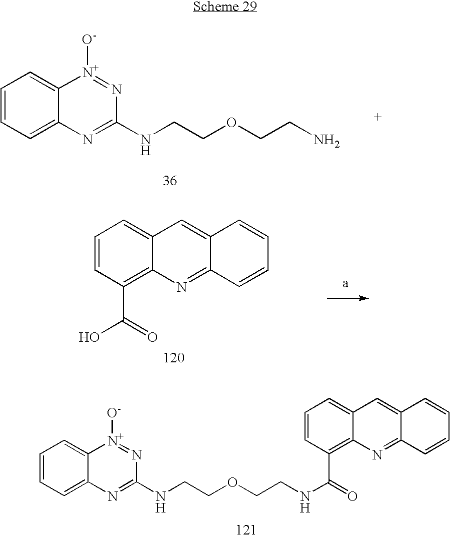

Reagents:

a) 120, CDI, DMF; then 36.

Reaction of the chloride 122 with methoxyethylamine gave 1-oxide 123 (Scheme 30).

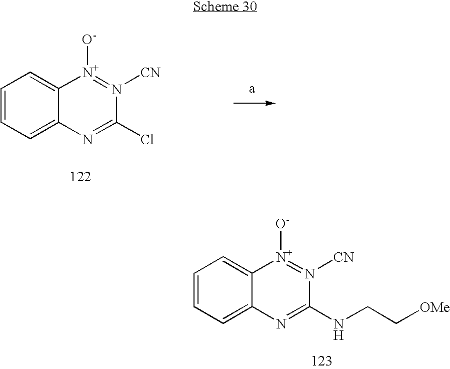

Reagents:

a) MeOCH₂CH₂NH₂, DME.

Amine 3 was deaminated with isoamyl nitrite in DMF to give 1-oxide 124 (Scheme 31). Reduction of amine 3 with sodium dithionite in aqueous ethanol gave benzotriazine 125 which was oxidised with MCPBA to 1-oxide 3,2-oxide 126, and 4-oxide 127.

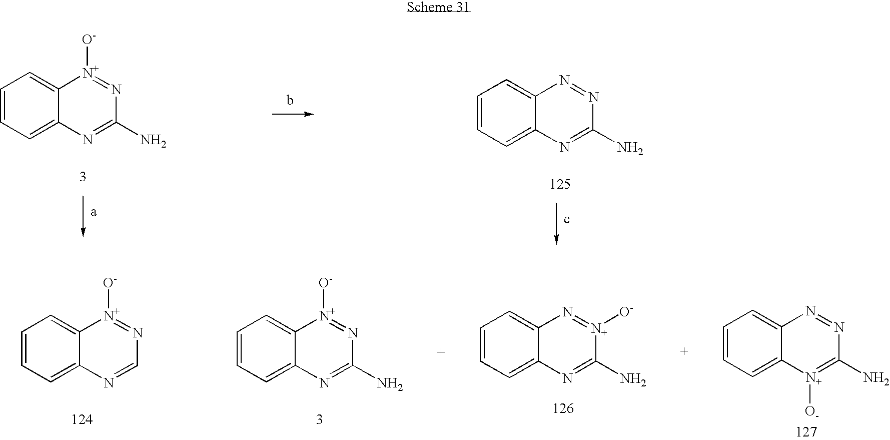

Reagents:

a) IsoamyInitrite, DMF;

b) Na₂S₂O₄, 70% EtOH;

c) MCPBA, DCM.

Reaction of chloride 19 with diamine 128 gave bis-1-oxide 129 (Scheme 32).

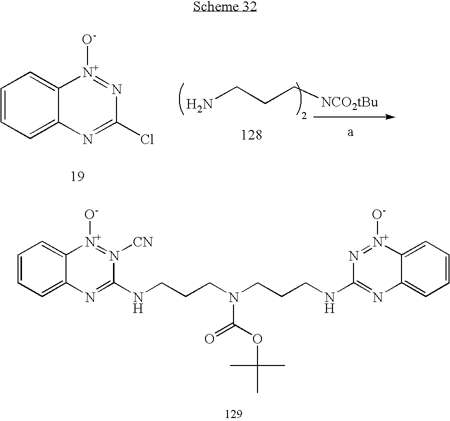

Reagents:

a) 19+128, Et₃N, DCM.

Reaction of chloride 19 with N-methylethylenediamine gave 1-oxide 130 (Scheme 33).

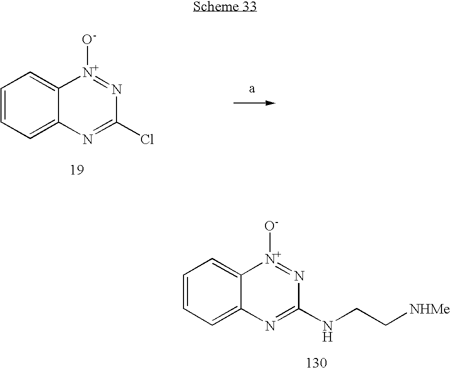

Reagents:

a) NH₂CH₂CH₂NHMe, Et₃N, DCM.

Treatment of 1-oxide 3t with NaNO₂ in trifluoroacetic acid, followed by chlorination in POCl₃ gave the chloride 131 (Scheme 34). Nucleophilic displacement of chloride 131 with N,N-dipropylethylenediamine gave the 1-oxide 132.

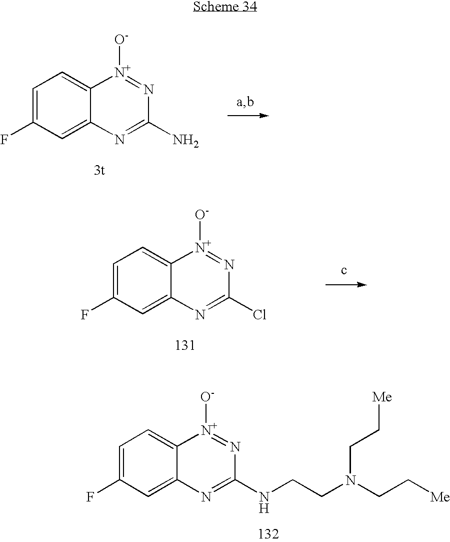

Reagents:

a) NaNO₂, TFA;

b) POCl₃, DMF;

c) NH₂CH₂CH₂NPr₂, DME.

Similarly, amine 3u was treated with NaNO₂ in trifluoroacetic acid and the intermediate chlorinated with POCl₃ to give chloride 133 (Scheme 35). Reaction of 133 with a range of amines gave 1-oxides 134-136.

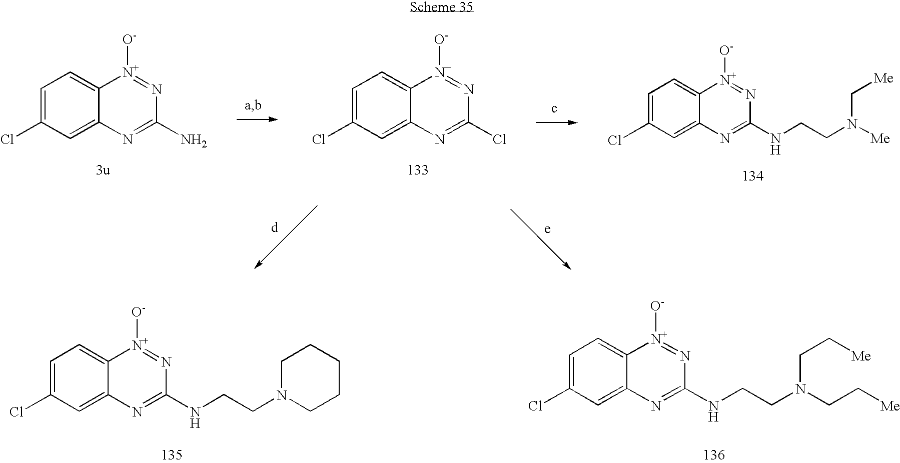

Reagents:

a) NaNO₂, TFA;

b) POCl₃, DMF;

c) NH₂CH₂CH₂NEt₂, DME;

d) NH₂CH₂CH₂Npiperidine, DME;

e) NH₂CH₂CH₂NPr₂, DME.

Reaction of chloride 73 with a range of amines gave 1-oxides 137-140 (Scheme 36).

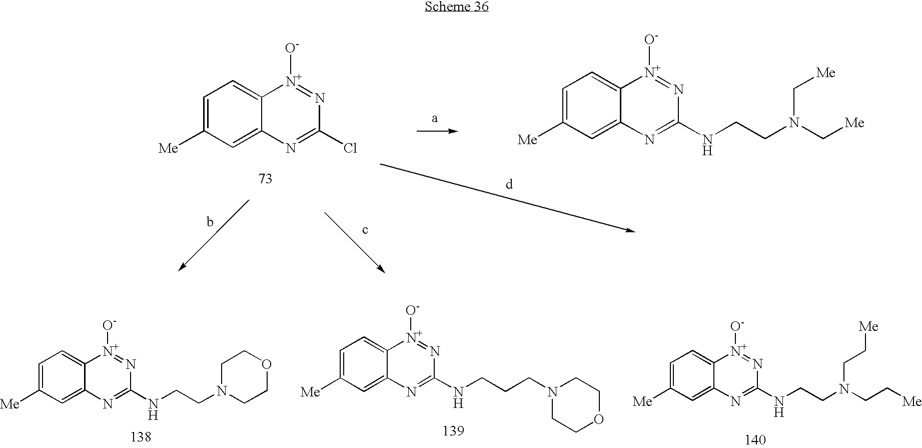

Reagents:

a) NH₂CH₂CH₂NEt₂, DME;

b) NH₂CH₂CH₂NMorpholine, DME;

c) NH₂CH₂CH₂CH₂NMorpholine, DME;

d) NH₂CH₂CH₂NPr₂, DME.

Reaction of the chloride 83 with the amine gave 1-oxide 141 (Scheme 37).

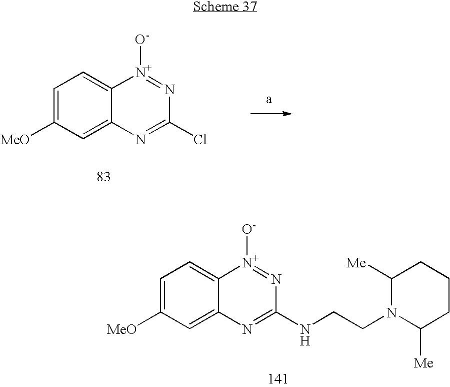

Reagents:

a) NH₂CH₂CH₂N-2,6-Me₂-piperidine, DME.

Treatment of 1-oxide 3v with NaNO₂ in trifluoroacetic acid, followed by chlorination in POCl₃ gave the chloride 142 (Scheme 38). Nucleophilic displacement of chloride 142 with N,N-dipropylethylenediamine gave the 1-oxide 143.

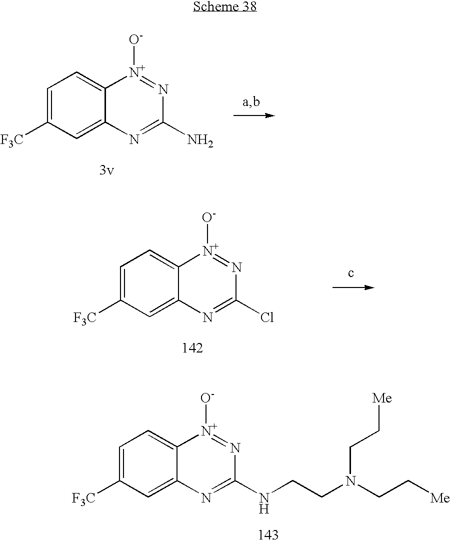

Reagents:

a) NaNO₂, TFA;

b) POCl₃, DMF;

c) NH₂CH₂CH₂NMe₂, DME.

Treatment of nitroaniline 144 with cyanamide under acidic conditions followed by cyclisation under basic conditions gave 1-oxide 145 (Scheme 39). Diazotization and chlorination of 145 gave chloride 146. Displacement of 146 with a range of amines gave 1-oxides 147-151.

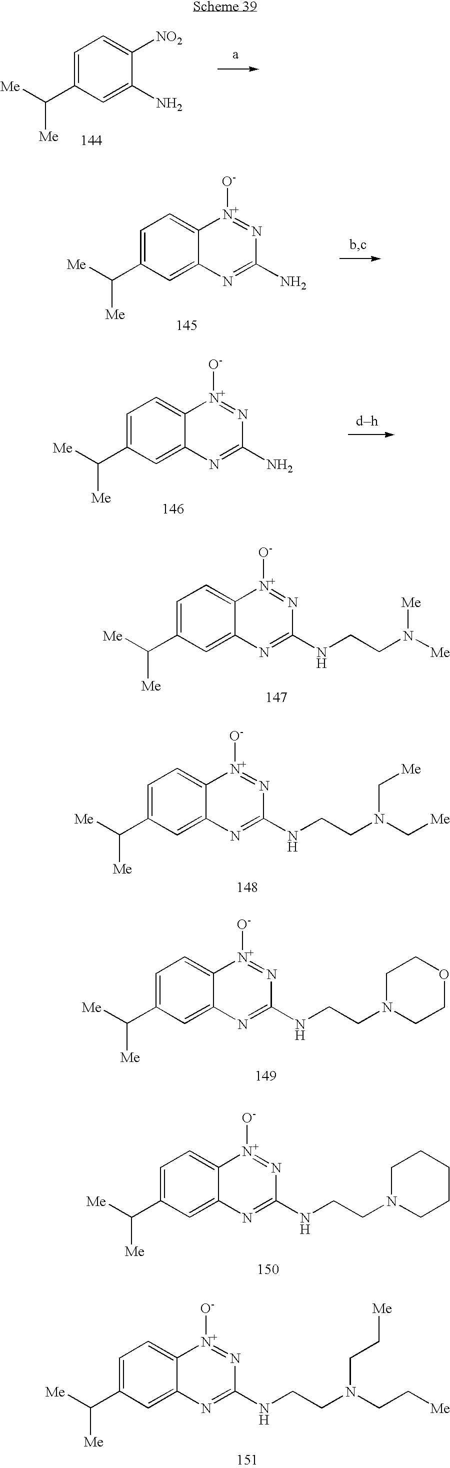

Reagents:

a) NH₂CN, HCl; then NaOH;

b) NaNO₂, TFA;

c) POCl₃, DMF;

d) NH₂CH₂CH₂NMe₂, DME;

e) NH₂CH₂CH₂NEt₂, DME;

f) NH₂CH₂CH₂Nmorpholine, DME;

g) NH₂CH₂CH₂Npiperidine, DME;

h) NH₂CH₂CH₂NPr₂, DME.

Treatment of nitroaniline 152 with cyanamide under acidic conditions followed by cyclisation under basic conditions gave 1-oxide 153 (Scheme 40). Diazotization and chlorination of 153 gave chloride 154. Displacement of 154 with a range of amines gave 1-oxides 155-159.

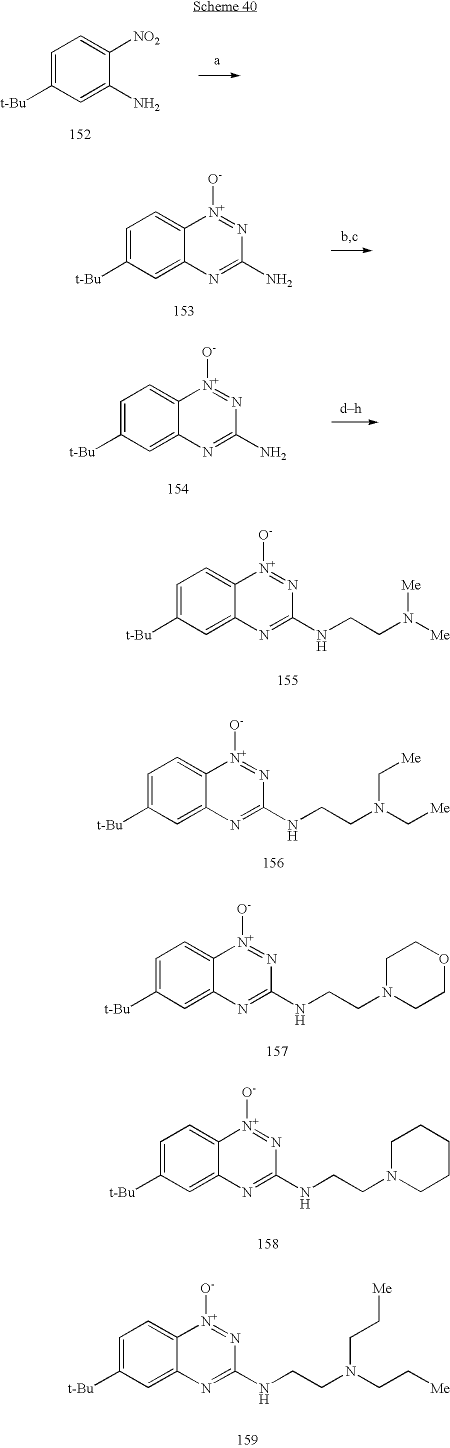

Reagents:

a) NH₂CN, HCl; then NaOH;

b) NaNO₂, TFA;

c) POCl₃, DMF;

d) NH₂CH₂CH₂NMe₂, DME;

e) NH₂CH₂CH₂NEt₂, DME;

f) NH₂CH₂CH₂Nmorpholine, DME;

g) NH₂CH₂CH₂Npiperidine, DME;

h) NH₂CH₂CH₂NPr₂, DME.

Reaction of chloride 131 under Stille conditions gave 1-oxide 160 which underwent nucleophilic displacement to give 1-oxide 161 (Scheme 41).

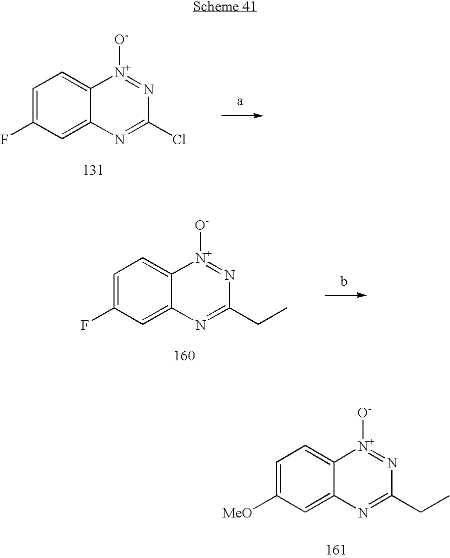

Reagents:

a) Et₄Sn, Pd(PPh₃)₄, DME;

b) NaOMe, MeOH.

Reaction of the chloride 100 with a range of amines gave 1-oxides 162-165 (Scheme 42).

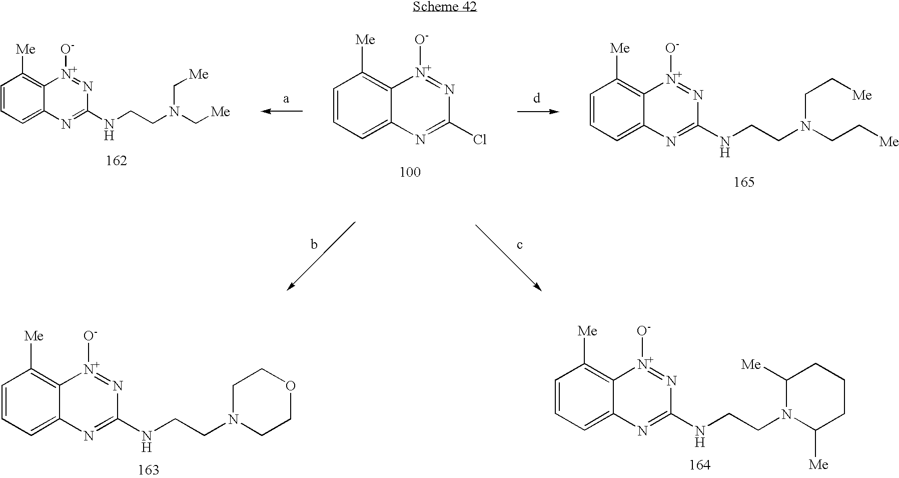

Reagents:

a) NH₂CH₂CH₂NEt₂, DME;

b) NH₂CH₂CH₂Nmorpholine, DME;

c) NH₂CH₂CH₂N(2,6-Me₂-piperidine), DME;

d) NH₂CH₂CH₂NPr₂, DME.

Reaction of the chloride 104 with a range of amines gave 1-oxides 166-172 (Scheme 43).

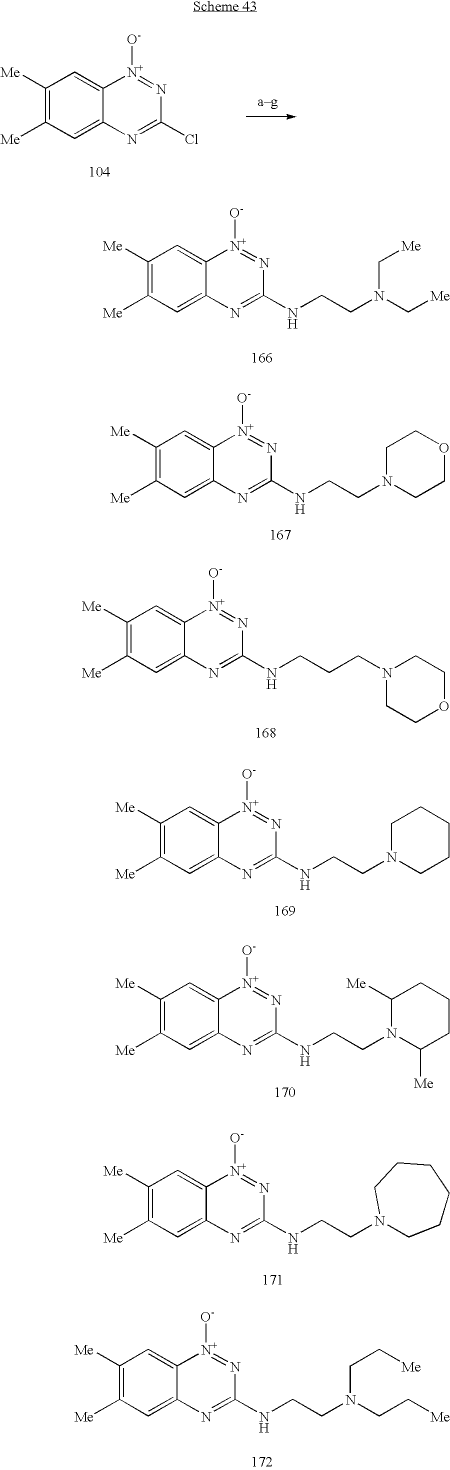

Reagents:

a) NH₂CH₂CH₂NEt₂, DME;

b) NH₂CH₂CH₂Nmorpholine, DME;

c) NH₂CH₂CH₂CH₂Nmorpholine, DME;

d) NH₂CH₂CH₂CH₂Npiperidine, DME;

e) NH₂CH₂CH₂N(2,6-Me₂-piperidine), DME;

f) NH₂CH₂CH₂CH₂Nazepane, DME;

g) NH₂CH₂CH₂NPr₂, DME.

Reaction of the chloride 108 with N¹,N¹-dipropyl-1,2-ethanediamine gave 1-oxide 173 (Scheme 44).

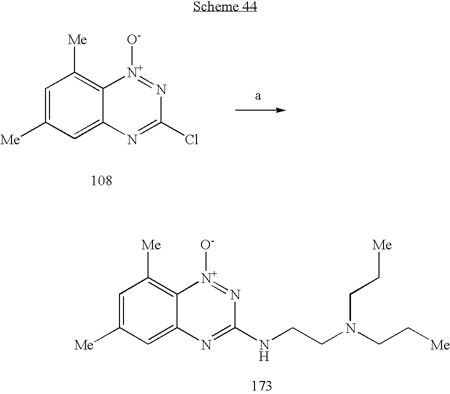

Reagents:

a) NH₂CH₂CH₂NPr₂, DME.

Examples of the Compounds of the Invention

The following examples are representative of the invention and the detailed methods for preparing them, however, the scope of the invention is not to be taken as being limited to these examples.

Analyses were carried out in the Microchemical Laboratory, University of Otago, Dunedin, NZ. Melting points were determined on an Electrothermal 2300 Melting Point Apparatus. NMR spectra were obtained on a Bruker AM-400 spectrometer at 400 MHz for ¹H and 100 MHz for ¹³C spectra. Spectra were obtained in CDCl₃ unless otherwise specified, and are referenced to Me₄Si. Chemical shifts and coupling constants were recorded in units of ppm and Hz, respectively. Assignments were determined using COSY, HSQC, and HMBC two-dimensional experiments. Mass spectra were determined on a VG-70SE mass spectrometer using an ionizing potential of 70 eV at a nominal resolution of 1000. High-resolution spectra were obtained at nominal resolutions of 3000, 5000, or 10000 as appropriate. All spectra were obtained as electron impact (El) using PFK as the reference unless otherwise stated. Solutions in organic solvents were dried with anhydrous Na₂SO₄. Solvents were evaporated under reduced pressure on a rotary evaporator. Thin-layer chromatography was carried out on aluminum-backed silica gel plates (Merck 60 F₂₅₄) with visualization of components by UV light (254 nm) or exposure to I₂. Column chromatography was carried out on silica gel, (Merck 230-400 mesh). All compounds designated for biological testing were analysed at >99% purity by reverse phase HPLC using a Philips PU4100 liquid chromatograph, a Phenomenex BondClone 10-C18 stainless steel column (300 mm×3.9 mm i.d.) and a Philips PU4120 diode array detector. Chromatograms were run using various gradients of aqueous (1 M NaH₂PO₄, 0.75 M heptanesulfonic acid, 0.5 M dibutylammonium phosphate, and MilliQ water in a 1:1:1:97 ratio) and organic (80% MeOH/MilliQ water) phases. DCM refers to dichloromethane; DME refers to dimethoxyethane, DMF refers to dry dimethylformamide; ether refers to diethyl ether; EtOAc refers to ethyl acetate; EtOH refers to ethanol; MeOH refers to methanol; pet. ether refers to petroleum ether, boiling range 40-60° C.; THF refers to tetrahydrofuran dried over sodium benzophenone ketyl. All solvents were freshly distilled.

### Example 1

Method A: Condensation of 2-nitroanilines (1) with cyanamide. 2-Nitroaniline (1) (4.3 mmol) and cyanamide (22 mmol) were melted together at 100° C., cooled to ca. 50° C., and cHCl (5 mL) added carefully. The mixture was stirred until the exotherm subsided then stirred at 100° C. for 2 h. If necessary, more cyanamide (22 mmol) was added and the mixture stirred at 100° C. for 4 h. The mixture was cooled to 20° C., made strongly basic with 7.5 M NaOH solution (ca. 50 mL) and the mixture heated at 100° C. for 1 h then cooled to 20° C. and diluted with water (100 mL). The precipitate was filtered, washed with water (2×10 mL), ether (2×10 mL) and dried. If necessary, the solid was chromatographed, eluting with a gradient (2-5%) of MeOH/CHCl₃, to give the corresponding 1,2,4-benzotriazin-3-amine 1-oxide (3).

### Example 2

Method B: Condensation of 2-nitrohalobenzenes (2) with guanidine. Guanidine hydrochloride (104 mmol) was added to a stirred solution of KOtBu (104 mmol) in abs. EtOH (80 mL) and the mixture stirred at 20° C. for 1 h. The mixture was filtered, and the filtrate added slowly to a stirred solution of 2-nitrohalobenzene (2) (26 mmol) in absolute EtOH (50 mL). The mixture was heated at reflux temperature for 72 h then cooled, acidified with cHCl and the solvent evaporated. The residue was suspended in 0.5 M HCl and the precipitate was filtered. The aqueous fraction was washed with CHCl₃, basified with aqueous NH₃, and extracted into EtOAc. The organic fraction was dried and the solvent evaporated. The residue was suspended in 10% aq. NaOH and heated at 100° C. for 2 h. The precipitate was filtered, washed with water (2×10 mL), ether (2×10 mL) and dried. If necessary, the solid was chromatographed, eluting with a gradient (2-5%) of MeOH/CHCl₃, to give the corresponding 1,2,4-benzotriazin-3-amine 1-oxide (3).

### Example 3

8-Methoxy-1,2,4-benzotriazin-3-amine 1-oxid (3a). Method A using 3-methoxy-2-nitroaniline (1a)(Shigyo et. al., Chem. Pharm. Bull.1993, 41, 1573) gave 3a (51%) as a yellow powder, mp (H₂O) 235-239° C.; ¹H NMR [(CD₃)₂SO] δ 7.62 (dd, J=8.3, 8.0 Hz, 1H, H-6), 7.15 (br s, 2H, NH₂), 7.02 (d, J=8.3 Hz, 1H, H-7), 6.80 (d, J=8.0 Hz, 1H, H-5), 3.83 (s, 3H, OCH₃); ¹³C NMR [(CD₃)₂SO] δ 160.0, 153.3, 151.4, 135.4, 122.6, 117.0, 105.1, 56.4; Anal. calc. for C₈H₈N₄O₂: C, 50.0; H, 4.2; N, 29.2; found C, 50.3; H, 4.1; N, 29.2%.

### Example 4

8-Methyl-1,2,4-benzotriazin-3-amine 1-oxide (3b). Method A using 3-methyl-2-nitroaniline (1b) gave 3b (100%) as a yellow powder, mp (DMF) 265° C. (dec.); ¹H NMR [(CD₃)₂SO] δ 7.59 (dd, J=8.3, 7.3 Hz, 1H, H-6), 7.35 (d, J=8.0 Hz, 1H, H-5), 7.18 (s, 2H, NH₂), 7.10 (dd, J=7.2, 0.8 Hz, 1H, H-7), 2.79 (s, 3H, CH₃); Anal. calc. for C₈H₈N₄O: C, 54.5; H, 4.6; N, 31.8; found C, 54.6; H, 4.7; N, 31.9%.

### Example 5

8-Fluoro-1,2,4-benzotriazin-3-amine 1-oxide (3c). Method B using 2,6-difluoroaniline (2c) gave 3c (49%) as a yellow powder, mp (DCM/pet. ether) 270-278° C. (dec.) [lit. (Suzuki & Kawakami, Synthesis 1977, 855) mp 271° C. (dec.)]; ¹H NMR [(CD₃)₂SO] δ 7.69 (ddd, J=10.9, 8.3, 5.2 Hz, 1H, H-6), 7.45 (br s, 2H, NH₂), 7.31 (dd, J=9.6, 1.0 Hz, 1H, H-5), 7.09 (ddd, J=12.0, 8.0, 1.0 Hz, 1H, H-7); ¹³C NMR [(CD₃)₂SO] δ 160.2 (d, J=5.3 Hz), 153.5 (d, J=264.3 Hz), 151.1 (d, J=3.2 Hz), 135.2 (d, J=4.4 Hz), 121.7 (d, J=4.5 Hz), 121.1, 110.0 (d, J=20.8 Hz); HRMS (EI⁺) calc. for C₇H₅FN₄O (M⁺) m/z 180.0360, found 180.0441.

### Example 6

8-Chloro-1,2,4-benzotriazin-3-amine 1-oxide (3d). Method A using 3-chloro-2-nitroaniline (1d) gave 3d (30%) as a yellow powder, mp (DMF) 280-290° C. (dec.); ¹H NMR [(CD₃)₂SO] δ 7.63 (dd, J=8.4, 7.8 Hz, 1H, H-6), 7.45 (dd, J=8.6, 1.0 Hz, 1H, H-7), 7.42 (br s, 2H, NH₂), 7.36 (dd, J=7.6, 1.1 Hz, 1H, H-5); HRMS (EI) calc. for C₇H₅N₄O³⁵Cl (M⁺) m/z 196.0152, found 196.0152; calc for C₇H₅N₄O³⁷Cl (M⁺) m/z 198.0122, found 198.0124.

### Exampl 7

8-Trifluoromethyl-1,2,4-benzotriazin-3-amine 1-oxid (3). Method A using 3-trifluoromethyl-2-nitroaniline (1e) gave 3 (14%) as a yellow powder, mp (DCM/pet. ether) 280-286° C. (dec.); ¹H NMR [(CD₃)₂SO] δ 7.78-7.87 (m, 3H, H-5, H-6, H-7), 7.55 (br s, 2H, NH₂); Anal. calc. for C₈H₅F₃N₄O: C, 41.8; H, 2.2; N, 24.3; F, 24.8; found C, 41.6; H, 2.1; N, 24.3; F, 24.9%.

### Example 8

8-(Methylsulfanyl)-1,2,4-benzotriazin-3-amine 1-oxide (3f).

3-(Methylsulfanyl)-2-nitroaniline (1f). A solution of LiSMe (1.19 g, 22.0 mmol) in DMF (20 mL) was added dropwise to a stirred solution of 3-chloro-2-nitroaniline (1d) (3.17 g, 18.4 mmol) in DMF (80 mL) at 20° C. and the mixture stirred for 2 h. The mixture was poured into water (300 mL) and extracted with EtOAc (2×150 mL). The combined organic fraction was washed with water (2×100 mL), brine (50 mL), dried, and the solvent evaporated. The residue was chromatographed, eluting with 20% EtOAc/pet. ether, to give (i) starting material (0.51 g, 16%) and (ii) sulfide (1f) (2.36 g, 70%) as red crystals, mp (EtOAc/pet. ether) 70-72° C.; ¹H NMR δ 7.21 (t, J=8.2 Hz, 1H, H-5), 6.55 (d, J=8.2 Hz, 2H, H-4, H-6), 5.92 (br s, 2H, NH₂), 2.42 (s, 3H, SCH₃); ¹³C NMR δ 146.0, 141.6, 133.3, 131.0, 113.9 (2), 17.0; Anal. calc. for C₇H₈N₂O₂S: C, 45.6; H, 4.4; N, 15.2; found C, 45.8; H, 4.4; N, 15.1%.

8-(Methylsulfanyl)-1,2,4-benzotriazin-3-amine 1-oxide (3f). Method A using 1f gave 3f (68%) as a yellow powder, mp (H₂O) 271-275° C.; ¹H NMR [(CD₃)₂SO] δ 7.63 (dd, J=8.3, 8.0 Hz, 1H, H-6), 7.28 (s, 2H, NH₂), 7.17 (d, J=8.3 Hz, 1H, H-5), 6.98 (d, J=8.0 Hz, 1H, H-7), 2.39 (s, 3H, SCH₃); ¹³C NMR [(CD₃)₂SO] 6159.9, 151.1, 137.1, 134.7, 127.9, 120.1, 118.7, 15.7; Anal. calc. for C₈H₈N₄OS: C, 46.1; H, 3.9; N, 26.9; found C, 45.9; H, 3.9; N, 26.7%.

### Example 9

8-(Butylsulfanyl)-1,2,4-benzotriazin-3-amine 1-oxide (3g).

3-(Butylsulfanyl)-2-nitroaniline (1g). A solution of LiSBu (1.39 g, 14.5 mmol) in DMF (10 mL) was added dropwise to a stirred solution of 3-chloro-2-nitroaniline (1d) (2.08 g, 12.05 mmol) in DMF (50 mL) at 20° C. and the mixture stirred for 2 h. The mixture was poured into water (300 mL) and extracted with EtOAc (2×150 mL). The combined organic fraction was washed with water (2×100 mL), brine (50 mL), dried, and the solvent evaporated. The residue was chromatographed, eluting with 20% EtOAc/pet. ether, to give sulfide 1g (2.47 g, 91%) as a red oil, ¹H NMR δ7.17 (dd, J=8.2, 8.0 Hz, 1H, H-5), 6.62 (d, J=8.0 Hz, 1H, H-4), 6.54 (dd, J=8.2, 1.0 Hz, 1H, H-6), 5.74 (br s, 2H, NH₂), 2.87 (t, J=7.4 Hz, 2H, CH₂S), 1.64-1.72 (m, 2H, CH₂), 1.45-1.53 (m, 2H, CH₂), 0.95 (s, 3H, CH₃); ¹³C NMR δ 145.3, 140.1, 133.0, 132.4, 115.0, 113.9, 33.0, 29.8, 22.2, 13.6; MS (EI) m/z 226 (M+, 35%), 106 (100); HRMS (EI) calc. for C₁₀H₁₄N₂O₂S (M+) m/z226.0776, found 226.0773.

8-(Butylsulfanyl)-1,2,4-benzotriazin-3-amine 1-oxide (3g). Method A using 1g gave 4i (26%) as a red/brown solid, mp (MeOH/CHCl₃) 233-236° C.; ¹H NMR [(CD₃)₂SO] δ 7.61 (dd, J=8.2, 7.4 Hz, 1H, H-6), 7.27 (s, 2H, NH₂), 7.16 (dd, J=8.2, 0.8 Hz, 1H, H-5), 7.04 (dd, J=7.4, 0.8 Hz, 1H, H-7), 2.86 (t, J=7.3 Hz, 2H, CH₂S), 1.62-1.70 (m, 2H, CH₂), 1.44-1.52 (m, 2H, CH₂), 0.93 (s, 3H, CH₃); ¹³C NMR [(CD₃)₂SO] δ 159.8, 151.1, 136.4, 134.7, 128.0, 120.1, 119.1, 31.0, 28.8, 21.7, 13.5; Anal. calc. for C₁₁H₁₄N₄OS: C, 52.8; H, 5.6; N, 22.4; found C, 52.7; H, 5.6; N, 22.6%.

### Example 10

3-Amino-1,2,4-benzotriazin-7-ol 1-oxide (3h). Method A using 4-amino-3-nitrophenol (1 h) gave 3h (97%) as a yellow powder, mp>300° C. [lit. (Friebe et. al., U.S. Pat. No. 5,856,325, Jan. 5, 1999) mp (HOAC)>270° C.]; ¹H NMR [(CD₃)₂SO] δ 10.37 (br s, 1H, OH), 7.48 (dd, J=7.7, 2.6 Hz, 1H, H-6), 7.40-7.37 (m, 2H, H-5, H-8), 6.96 (br s, 2H, NH₂).

### Example 11

3-Amino-1,2,4-benzotriazin-7-ol 1-oxide (3h). Method A using 4-hydroxy-2-nitroaniline (1 i) gave 3i (93%) as a yellow powder, mp (HOAC) 269-271° C. [lit. (Mason & Tennant, J. Chem. Soc. (B) 1970, 911) mp (HOAC) 271° C.]; ¹H NMR [(CD₃)₂SO] δ 7.48-7.53 (m, 3H, H-5, H-6, H-8), 7.10 (br s, 2H, NH₂), 3.88 (s, 3H, OCH₃); ¹³C NMR [(CD₃)₂SO] δ 159.3, 156.3, 144.9, 129.7, 128.3, 127.3, 97.9, 55.8.

### Example 12

7-Methyl-1,2,4-benzotriazin-3-amine 1-oxide (3j). Method A using 4-methyl-2-nitroaniline (1j) gave 3j (74%) as a yellow powder, mp (DMF) 270° C. (dec.) [lit. (Pazdera & Potacek, Chem. Papers 1988, 42, 527) mp (Methylcellosolve) 282° C.]; ¹H NMR [(CD₃)₂SO] δ 7.94 (s, 1H, H-8); 7.64 (dd, J=8.7, 1.9 Hz, 1H, H-6), 7.46 (d, J=8.6 Hz, 1H, H-5), 7.21 (s, 2H, NH₂), 2.42 (s, 3H, CH₃); Anal. calc. for C₈H₈N₄O: C, 54.5; H, 4.6; N, 31.8; found C, 54.8; H, 4.5; N, 31.9%.

### Exampl 13

7-Fluoro-1,2,4-benzotriazin-3-amin 1-oxide (3k). Method A using 4-fluoro-2-nitroaniline (1 k) gave 3k (78%) as a yellow powder, mp (DMF) 280-290° C. (dec.) [lit. (Suzuki & Kawakami, Synthesis 1977, 855) mp 290° C. (dec.)]; ¹H NMR [(CD₃)₂SO] δ 7.89 (dd, J=8.6, 2.9 Hz, 1H, H-8), 7.76 (ddd, J=9.3, 8.8, 2.9 Hz, 1H, H-6), 7.62 (dd, J=9.3, 5.2 Hz, 1H, H-5), 7.35 (br s, 2H, NH₂); Anal. calc. for C₇H₅FN₄O: C, 46.7; H, 2.8; N, 31.1; F, 10.6; found C, 46.7; H, 2.7; N, 31.1; F, 10.7%.

### Example 14

7-Chloro-1,2,4-benzotriazin-3-amine 1-oxide (31). Method A using 4-chloro-2-nitroaniline (1l) gave 31 (39%) as a yellow powder, mp (DCM/pet. ether) 309° C. (dec.) [lit. (Pazdera & Potacek, Chem. Papers 1988, 42, 527) mp (HOAc) 306-308° C.]; ¹H NMR [(CD₃)₂SO] δ 8.14 (d, J=1.7 Hz, 1H, H-8), 7.80 (dd, J=8.8, 1.9 Hz, 1H, H-6), 7.56 (d, J=9.0 Hz, 1H, H-5), 7.48 (br s, 2H, NH₂).

### Example 15

7-Trifluoromethyl-1,2,4-benzotriazin-3-amine 1-oxide (3m). Method B using 1-chloro-2-nitro-4-(trifluoromethyl)benzene (2m) gave 3m (30%) as a yellow powder, mp (DCM/pet. ether) 290° C. (dec.) [lit. (Suzuki & Kawakami, Synthesis 1977, 855) mp (acetone/toluene) 301-302° C.]; ¹H NMR [(CD₃)₂SO] δ 8.38 (br s, 1H, H-8), 8.01 (dd, J=8.9, 2.0 Hz, 1H, H-6), 7.72 (br s, 2H, NH₂), 7.68 (d, J=8.9 Hz, 1H, H-5);

¹³C NMR [(CD₃)₂SO] δ 161.1, 150.6, 130.7 (J=2.9 Hz), 129.3, 127.5, 123.4 (q, J=272.0 Hz), 123.6 (q, J=32.1 Hz), 118.0 (q, J=10.9 Hz).

### Example 16

7-(Methylsulfanyl)-1,2,4-benzotriazin-3-amine 1-oxide (3n).

4-(Methylsulfanyl)-2-nitroaniline (1 n) and 6-(methylsulfanyl)-2-nitroaniline (1ad). O-(3-Nitrophenyl)dimethylthiocarbamate (Newman & Karnes, J. Org. Chem. 1966, 31, 3980) (4) (14.05 g, 62.1 mmol) was heated at 235-240° C. for 3 h under N₂, cooled to 20° C. to give crude S-(3-nitrophenyl)dimethylthiocarbamate (5) which was heated at reflux temperature with KOH solution (7.5 M, 410 mL, 3.1 mol) and MeOH (200 mL) for 2 h. The mixture was cooled to 20° C. and Me₂SO₄ (59 mL, 0.62 mol) added dropwise and the mixture stirred at 20° C. for 16 h. The mixture was partitioned between EtOAc (300 mL) and water (300 mL), the organic fraction washed with water (3×100 mL), brine (100 mL), dried, and the solvent evaporated. The residue was chromatographed, eluting with 10% EtOAc/pet. ether, to give 1-(methylsulfanyl)-3-nitrobenzene (6) (9.6 g, 91%) as a soft solid, ¹H NMR δ 8.05 (dd, J=2.0, 1.9 Hz, 1H, H-2), 7.96 (dd, J=8.2, 2.0, 0.9 Hz, 1H, H-4), 7.53 (ddd, J=7.9, 1.9, 1.0 Hz, 1H, H-6), 7.43 (dd, J=8.2, 7.9 Hz, 1H, H-5), 2.56 (s, 3H, SCH₃); ¹³C NMR 8148.6, 141.6, 131.9, 129.4, 120.3, 119.6, 15.4.

A solution of NH₂OMe.HCl (2.83 g, 34.0 mmol) and nitrobenzene 6 (4.79 g, 28.3 mmol) in DMF (100 mL) was added dropwise to a stirred mixture of KOtBu (13.0 g, 116.6 mmol) and CuCl (0.28 g, 2.83 mmol) in DMF (50 mL) at 5° C. The mixture was stirred at 20° C. for 3 h, quenched with saturated aqueous NH₄Cl solution (100 mL). The mixture was extracted with EtOAc (3×100 mL), the combined organic fraction dried, and the solvent evaporated. The residue was chromatographed, eluting with 10% EtOAc/pet. ether, to give (i) 6-(methylsulfanyl)-2-nitroaniline (1ad) (2.11 g, 40%) as a red oil, ¹H NMR δ 8.11 (dd, J=8.7, 1.5 Hz, 1H, H-3), 7.66 (dd, J=7.4, 1.5 Hz, 1H, H-5), 6.92 (br s, 2H, NH₂), 6.66 (dd, J=8.7, 7.4 Hz, 1H, H-4), 2.38 (s, 3H, SCH₃); ¹³C NMR δ 145.0, 140.7, 132.5, 126.6, 124.5, 115.9, 18.2; MS (EI) m/z 184 (100, M⁺), 169 (10), 150 (30); HRMS calc. for C₇H₈N₂O₂S (M⁺) m/z 184.0307, found 184.0304; (ii) 4-(methylsulfanyl)-2-nitroaniline (1n) (0.8 g, 15%) as a red oil, ¹H NMR δ 8.05 (d, J=2.3 Hz, 1H, H-3), 7.34 (dd, J=8.7, 2.3 Hz, 1H, H-5), 6.76 (d, J=8.7 Hz, 1H, H-6), 6.05 (br s, 2H, NH₂), 2.46 (s, 3H, SCH₃); ¹³C NMR δ 143.1, 136.8, 132.2, 125.8, 125.1, 119.5, 18.0.

7-(Methylsulfanyl)-1,2,4-benzotriazin-3-amine 1-oxide (3n). Method A using 1 n gave 3n (56%) as a red solid, mp (MeOH/CHCl₃) 245-247° C.; ¹H NMR [(CD₃)₂SO] δ 7.79 (d, J=2.1 Hz, 1H, H-8), 7.67 (dd, J=8.9, 2.1 Hz, 1H, H-6), 7.47 (d, J=8.9 Hz, 1H, H-5), 7.28 (s, 2H, NH₂), 2.58 (s, 3H, SCH₃); ¹³C NMR [(CD₃)₂SO] δ 159.9, 147.0, 135.7, 134.6, 130.1, 126.5, 113.4, 14.6; Anal. calc. for C₈H₈N₄OS: C, 46.1; H, 3.9; N, 26.9; found C, 46.1; H, 3.8; N, 26.6%.

### Example 17

7-(Butylsulfanyl)-1,2,4-benzotriazin-3-amine 1-oxide (3o).

4-(Butylsulfanyl)-2-nitroaniline (1o) and 6-(butylsulfanyl)-2-nitroaniline (9). A mixture of crude 5 (7.0 g, 31 mmol) and KOH (7.5 M, 41 mL, 310 mmol) in MeOH (200 mL) was heated at reflux temperature for 2 h. The mixture was cooled to 5° C. and the pH adjusted to 2 with cHCl. The precipitate was collected, washed with water (20 mL), dissolved in EtOAc (200 mL), dried, and the solvent evaporated. The residue was dissolved in DMF (100 mL) and K₂CO₃ (5.15 g, 37.3 mmol) added and the mixture stirred at 20° C. for 30 min. n-Butylbromide (4.0 mL, 37.3 mmol) was added and the mixture stirred at 80° C. for 16 h. The mixture was cooled and the solvent evaporated. The residue was partitioned between EtOAc (300 mL) and water (300 mL), the organic fraction washed with water (2×100 mL), brine (100 mL), dried, and the solvent evaporated. The residue was chromatographed, eluting with 10% EtOAc/pet. ether, to give 3-(butylsulfanyl)nitrobenzene (7) (5.53 g, 84%) as a yellow oil, ¹H NMR δ 8.10 (dd, J=2.0, 2.0 Hz, 1H, H-2), 7.97 (ddd, J=8.1, 2.0, 0.9 Hz, 1H, H-4), 7.57 (ddd, J=8.1, 2.0, 0.9 Hz, 1H, H-6), 7.42 (dd, J=8.1, 8.1 Hz, 1H, H-5), 3.01 (dd, J=7.4, 7.3 Hz, 2H, CH₂S), 1.64-1.71 (m, 2H, CH₂), 1.43-1.53 (m, 2H, CH₂), 0.95 (t, J=7.3 Hz, 3H, CH₃); ¹³C NMR δ 148.6, 140.5, 133.5, 129.4, 121.9, 120.1, 32.6, 30.7, 21.9, 13.6; MS (EI) m/z211 (M⁺, 60%), 155 (100); HRMS (EI) calc. for C₁₀H₁₃NO₂S (M+) m/z 211.0667, found 211.0661.

A solution of NH₂OMe.HCl (2.61 g, 31.2 mmol) and 7 (5.5 g, 26.0 mmol) in DMF (40 mL) was added dropwise to a stirred mixture of KOtBu (12.0 g, 106.7 mmol) and CuCl (0.26 g, 2.6 mmol) in DMF (50 mL) at 5° C. The mixture was stirred at 20° C. for 6 h, quenched with saturated aqueous NH₄Cl solution (300 mL). The mixture was extracted with EtOAc (3×100 mL), the combined organic fraction dried, and the solvent evaporated. The residue was chromatographed, eluting with 5% EtOAc/pet ether, to give (i) 6-(butylsulfanyl)-2-nitroaniline (9) (2.34 g, 40%) as a red oil, ¹H NMR δ 8.12 (dd, J=8.7, 1.5 Hz, 1H, H-3), 7.66 (dd, J=7.3, 1.5 Hz, 1H, H-5), 7.00 (br s, 2H, NH₂), 6.64 (dd, J=8.7, 7.3 Hz, 1H, H-4), 2.75 (dd, J=7.5, 7.2 Hz, 2H, CH₂S), 1.51-1.58 (m, 2H, CH₂), 1.37-1.46 (m, 2H, CH₂), 0.90 (dd, 7.4, 7.2 Hz, 3H, CH₃);

¹³C NMR δ 145.8, 142.5, 132.5, 127.0, 122.7, 115.6, 35.0, 31.6, 21.7, 13.6; MS (EI) m/z226 (M+, 100%), 209 (15), 192 (25); HRMS (EI) calc. for C₁₀H₁₄N₂O₂S (M+) m/z 226.0776, found 226.0770; (ii) 4-(butylsulfanyl)-2-nitroaniline (10) (1.12 g, 19%) as a red oil, ¹H NMR δ 8.16 (d, J=2.2 Hz, 1H, H-3), 7.39 (dd, J=8.7, 2.2 Hz, 1H, H-5), 6.76 (d, J=8.7 Hz, 1H, H-6), 6.06 (br s, 2H, NH₂), 2.83 (dd, J=7.3, 7.2 Hz, 2H, CH₂S), 1.56-1.62 (m, 2H, CH₂), 1.38-1.48 (m, 2H, CH₂), 0.91 (dd, J=7.4, 7.3 Hz, 3H, CH₃); ¹³C NMR δ 143.5, 139.1, 132.2, 128.3, 123.9, 119.3, 35.3, 31.2, 21.8, 13.6; MS (EI) m/z226 (M⁺, 100%), 170 (80); HRMS (EI) calc. for C₁₀H₁₄N₂O₂S (M+) m/z 226.0776, found 226.0772; and (iii) 2-(butylsulfanyl)-4-nitroaniline (8) (0.76 g, 13%) as a red oil, ¹H NMR δ 8.16 (d, J=2.6 Hz, 1H, H-3), 8.00 (dd, J=9.1, 2.6 Hz, 1H, H-5), 6.68 (d, J=9.1 Hz, 1H, H-6), 5.07 (br s, 2H, NH₂), 2.79 (dd, J=7.2, 6.6 Hz, 2H, CH₂S), 1.52-1.60 (m, 2H, CH₂), 1.38-1.47 (m, 2H CH₂), 0.91 (t, J=7.3 Hz, 3H, CH₃); ¹³C NMR δ 153.5, 138.7, 131.4, 125.7, 118.1, 113.0, 34.7, 31.5, 21.7, 13.6.

7-(Butylsulfanyl)-1,2,4-benzotriazin-3-amine 1-oxid (3o). Method A using 10 gave 3O (68%) as a red solid, mp (MeOH/CHCl₃) 215-217° C.; ¹H NMR [(CD₃)₂SO] δ 7.87 (d, J=2.1 Hz, 1H, H-8), 7.68 (dd, J=8.9, 2.1 Hz, 1H, H-6), 7.46 (d, J=8.9 Hz, 1H, H-5), 7.07 (br s, 2H, NH₂), 3.04 (dd, J=7.3, 7.2 Hz, 2H, CH₂S), 1.55-1.63 (m, 2H, CH₂), 1.37-1.45 (m, 2H, CH₂), 0.88 (t, J=7.3 Hz, 3H, CH₃); ¹³C NMR [(CD₃)₂SO] δ 160.0, 147.3, 135.9, 133.9, 130.0, 126.4, 115.8, 31.6, 30.1, 21.2, 13.4; Anal. calc. for C₁₁H₁₄N₄OS: C, 52.8; H, 5.6; N, 22.4; found C, 52.9; H, 5.8; N, 22.2%.

### Example 18

7-Nitro-1,2,4-benzotriazin-3-amine 1-oxide (3p). Method B using 1-chloro-2,4-dinitrobenzene (2p) gave 3p (15%) as a yellow powder, mp (DMF) 269-272° C. [lit.(Pazdera & Potacek, Chem. Papers 1988, 42, 527-537) mp (pyridine/EtOH) 290° C.];

¹H NMR [(CD₃)₂SO] δ 8.82 (d, J=2.6 Hz, 1H, H-8), 8.44 (dd, J=9.4, 2.6 Hz, 1H, H-6), 8.03 (br s, 2H, NH₂), 7.64 (d, J=9.3 Hz, 1H, H-5).

### Example 19

6-Methoxy-1,2,4-benzotriazin-3-amine 1-oxide (3q).

5-Methoxy-2-nitroaniline (lq). A mixture of 5-methoxy-2-nitrobenzoic acid (10) (10 g, 50.7 mmol) diphenylphosphorylazide (DPPA) (11.5 mL, 53.3 mmol) and Et₃N (7.4 mL, 53.3 mmol) in t-BuOH (200 mL) was heated at reflux temperature for 16 h. The solution was cooled to 20° C. and the solvent evaporated. The residue was dissolved in DCM (300 mL) and washed with water (2×100 mL), saturated aqueous KHCO₃ (100 mL), brine (50 mL), dried, and the solvent evaporated. The residue was suspended in MeOH (250 mL), cHCl (50 mL) added, and the mixture stirred at 20° C. for 96 h. The solvent was evaporated and the residue suspended in saturated aqueous KHCO₃ (400 mL) and stirred for 30 min. The suspension was filtered, the solid washed with water (20 mL) and dried at 80° C. under reduced pressure. The solid was chromatographed, eluting with a gradient (20-30%) of EtOAc/pet. ether, to give lq (8.26 g, 98%); as a yellow solid, mp 128-130° C. [lit. (Seko et. al., J. Chem. Soc. Perkin Trans. 1 1999, 1437) mp 130-132° C.]; ¹H NMR δ 8.07 (d, J=9.5 Hz, 1H, H-3), 6.28 (dd, J=9.5, 2.6 Hz, 1H, H-4), 6.21 (brs, 2H, NH₂), 6.15 (d, J=2.6 Hz, 1H, H-6), 3.83 (s, 3H, OCH₃); ¹³C NMR 8165.4, 147.1, 128.5, 126.9, 106.7, 99.4, 55.7.

6-Methoxy-1,2,4-benzotriazin-3-amine 1-oxide (3q). Method A using 5-methoxy-2-nitroaniline (1q) gave 3q (63%) as a yellow powder, mp (CHCl₃) 265-270° C.; ¹H NMR [(CD₃)₂SO] δ 8.04 (d, J=9.5 Hz, 1H, H-8), 7.24 (br s, 2H, NH₂), 6.95 (dd, J=9.5, 2.6 Hz, 1H, H-7), 6.86 (d, J=2.6 Hz, 1H, H-5), 3.91 (s, 3H, OCH₃); ¹³C NMR [(CD₃)₂SO] δ 164.7, 160.7, 151.3, 125.0, 121.5, 117.0, 103.8, 56.0; MS (EI⁺) m/z 192 (M⁺, 100%), 176 (5); HRMS (EI) calc. for C₈H₈N₄O₂ (M⁺) m/z 192.0647, found 192.0653; Anal. calc. for C₈H₈N₄O₂: C, 50.0; H, 4.2; N, 29.2; found C, 50.0; H, 4.0; N, 29.0%.

### Example 20

6-Methyl-1,2,4-benzotriazin-3-amine 1-oxide (3r). Method A using 5-methyl-2-nitroaniline (1 r) gave 3r (87%) as a yellow powder, mp (DM F) 263° C. (dec.) [lit. (Friebe et. al., U.S. Pat. No. 5,856,325, January 1999) mp (HOAC) 284-286° C.]; ¹H NMR [(CD₃)₂SO] δ 8.02 (d, J=8.8 Hz, 1H, H-8), 7.33 (s, 1H, H-5), 7.27 (br s, 2H, NH₂), 7.18 (dd, J=8.8, 1.7 Hz, 1H, H-7), 2.42 (s, 3H, CH₃); Anal. calc. for C₈H₈N₄O: C, 54.5; H, 4.6; N, 31.8; found C, 54.9; H, 4.6; N, 31.9%.

### Example 21

6-Phenyl-1,2,4-benzotriazin-3-amine 1-oxide (3s). Method A using 4-nitro[1,1′-biphenyl]-3-amine (1 s) gave 3s (50%) as a yellow powder, mp (MeOH/DCM) 256-258° C.; ¹H NMR [(CD₃)₂SO] δ 8.11 (d, J=8.9 Hz, 1H, H-8), 7.82 (br d, J=7.2 Hz, 2H, H-2′, H-6′), 7.75 (d, J=1.9 Hz, 1H, H-5), 7.67 (dd, J=8.9, 1.8 Hz, 1H, H-7), 7.47-7.50 (m, 2H H-3′, H-5′), 7.47-7.50 (m, 1H, H-4′), 7.38 (br s, 2H, NH₂); ¹³C NMR [(CD₃)₂SO] δ 160.5, 149.1, 146.9, 138.0, 129.1 (2), 129.0, 128.9, 127.2 (2), 123.7, 122.5, 120.5; Anal. calc. for C₁₃H₁₀N₄O: C, 65.5; H, 4.2; N, 23.5; found C, 65.4; H, 4.2; N, 23.7%.

### Example 22

6-Fluoro-1,2,4-benzotriazin-3-amine 1-oxide (3t). Method A using 5-fluoro-2-nitroaniline (1t) gave 3t (61%) as a yellow powder, mp (DCM/pet. ether) 276-280° C. [lit. (Suzuki & Kawakami, Synthesis 1977, 855) mp 268° C.]; ¹H NMR [(CD₃)₂SO] δ 8.21 (dd, J=9.5, 5.9 Hz, 1H, H-8), 7.50 (br s, 2H, NH₂), 7.30 (dd, J=10.0, 2.6 Hz, 1H, H-5), 7.21 (ddd, J=8.8, 7.5, 2.6 Hz, 1H, H-7); ¹³C NMR [(CD₃)₂SO] δ 164.5 (d, J=254.6 Hz), 160.8, 150.6 (d, J=16.1 Hz), 127.4, 123.4 (d, J=11.1 Hz), 114.1 (d, J=26.2 Hz), 109.5 (d, J=23.1 Hz); Anal. calc. for C₇H₅FN₄O: C, 46.7; H, 2.8; N, 31.1; F, 10.6; found C, 46.5; H, 2.7; N, 31.3; F, 10.8%.

### Exampl 23

6-Chloro-1,2,4-benzotrazin-3-amine 1-oxide (3u). Method A using 5-fluoro-2-nitroaniline (1 u) gave 3u (53%) as a yellow solid, mp (DCM/pet. ether)>320° C. [lit. (Friebe et. al., U.S. Pat. No. 5,856,325, January 1999) mp (HOAc)>300° C.]; ¹H NMR [(CD₃)₂SO] δ 8.13 (d, J=9.2 Hz, 1H, H-8), 7.60 (d, J=2.11 Hz, 1H, H-5), 7.53 (br s, 2H, NH₂), 7.33 (dd, J=9.2, 2.1 Hz, 1H, H-7).

### Example 24

6-Trifluoromethyl-1,2,4-benzotriazin-3-amine 1-oxide (3v). Method A using 5-trifluoromethyl-2-nitroaniline (lv) gave 3v (29%) as a yellow solid, mp (DCM/pet. ether) 280-284° C. (dec.); ¹H NMR [(CD₃)₂SO] δ 8.31 (d, J=8.9 Hz, 1H, H-8), 7.85 (br s, 1H, H-5), 7.65 (br s, 2H, NH₂), 7.56 dd, J=8.9, 1.6 Hz, 1H, H-7); ¹³C NMR [(CD₃)₂SO] δ 160.8, 148.4, 134.8 (q, J=32.7 Hz), 131.5, 123.5 (q, J=4.0 Hz), 123.1 (q, J=273.1 Hz), 122.0, 119.3; Anal. calc. for C₈H₅F₃N₄O: C, 41.8; H, 2.2; N, 24.3; F, 24.8; found C, 41.1; H, 2.1; N, 24.3; F, 24.6%.

### Example 25

6-(Methylsulfanyl)-1,2,4-benzotriazin-3-amine 1-oxide (3w). Method A using 5-methylsulfanyl-2-nitroaniline (1w) (Seko et. al., J. Chem. Soc. Perkin Trans. 1 1999, 1437) gave 3w (55%) as a yellow powder, mp (MeOH/CHCl₃) 248-250° C.; ¹H NMR [(CD₃)₂SO] δ 7.99 (d, J=8.3 Hz, 1H, H-8), 7.30 (br s, 2H, NH₂), 7.16-7.19 (m, 2H, H-5, H-7), 2.59 (s, 3H, SCH₃); ¹³C NMR [(CD₃)₂SO] δ 160.7, 149.2, 149.1, 127.3, 122.9, 119.8, 118.0, 14.0; Anal. calc. for C₈H₈N₄OS: C, 46.1; H, 3.9; N, 26.9; found C, 46.0; H, 3.8; N, 27.0%.

### Example 26

6-(Butylsulfanyl)-1,2,4-benzotriazin-3-amine 1-oxide (3x).

5-(Butylsulfanyl)-2-nitroaniline (1x). A solution of LiSnBu (3.34 g, 34.8 mmol) in DMF (30 mL) was added dropwise to a stirred solution of 5-chloro-2-nitroaniline (1 u) (5.0 g, 30.0 mmol) in DMF (50 mL) at 20° C. and the mixture stirred for 2 h. The mixture was poured into water (300 mL) and extracted with EtOAc (2×150 mL). The combined organic fraction was washed with water (2×100 mL), brine (50 mL), dried, and the solvent evaporated. The residue was chromatographed, eluting with 20% EtOAc/pet. ether, to give 1x (6.12 g, 90%) as a red solid, mp (EtOAc/pet. ether) 91-93° C.; ¹H NMR δ 8.00 (d, J=7.5 Hz, 1H, H-3), 6.52-6.56 (m, 2H, H-4, H-6), 6.11 (br s, 2H, NH₂), 2.96 (dd, J=7.4, 7.3 Hz, 2H, CH₂S), 1.66-1.73 (m, 2H, CH₂), 1.44-1.53 (m, 2H, CH₂), 0.96 (s, 3H, CH₃); ¹³C NMR δ 149.0, 144.8, 129.6, 126.4, 115.2, 113.4, 31.3, 30.6, 22.0, 13.6; Anal. calc. for C₁₀H₁₄N₂O₂S: C, 53.1; H, 6.2; N, 12.4; found C, 53.2; H, 6.3; N, 12.4%.

6-(Butylsulfanyl)-1,2,4-benzotriazin-3-amin 1-oxide (3×). Method A using 1x gave (i) starting material (1x) (60%) and (ii) 3x (30%) as a red solid, mp (MeOH/CHCl₃) 180-182° C.; ¹H NMR [(CD₃)₂SO] δ 7.99 (d, J=9.1 Hz, 1H, H-8), 7.31 (br s, 2H, NH₂), 7.21 (d, J=2.0 Hz, 1H, H-5), 7.17 (dd, J=9.1, 2.0 Hz, 1H, H-7), 3.12 (dd, J=7.3, 7.2 Hz, 2H, CH₂S), 1.61-1.68 (m, 2H, CH₂), 1.41-1.49 (m, 2H, CH₂), 0.92 (s, 3H, CH₃); ¹³C NMR [(CD₃)₂SO] δ 160.6, 149.1, 147.9, 127.3, 123.4, 119.9, 118.8, 30.1, 29.9, 21.3, 13.4; Anal. calc. for C₁₁H₁₄N₄OS: C, 52.8; H, 5.6; N, 22.4; found C, 52.5; H, 5.6; N, 22.5%.

### Example 27

5-Methoxy-1,2,4-benzotriazin-3-amine 1-oxide (3y). Method A using 6-methoxy-2-nitroaniline (1y) (Seko et. al., J. Chem. Soc. Perkin Trans. 1 1999, 1437) gave 3y (66%) as a yellow powder, mp (HOAC) 267° C. (dec.) [lit. (Friebe et. al., U.S. Pat. No. 5,856,325, January 1999) mp (HOAC)>270° C.]; ¹H NMR [(CD₃)₂SO] δ 7.65-7.69 (m, 1H, H-7), 7.39 (br s, 2H, NH₂), 7.23-7.27 (m, 2H, H-6, H-8), 3.92 (s, 3H, OCH₃); ¹³C NMR [(CD₃)₂SO] δ 159.7, 153.3, 141.5, 130.0, 123.9, 113.4, 110.7, 55.9; Anal. calc. for C₈H₈N₄O₂: C, 50.0; H, 4.2; N, 29.2; found C, 50.2; H, 4.1; N, 29.1%.

### Example 28

5-Methyl-1,2,4-benzotriazin-3-amine 1-oxide (3z). Method A using 6-methyl-2-nitroaniline (1z) gave 3z (89%) as a yellow solid, mp (DCM/pet. ether) 253-255° C.; ¹H NMR [(CD₃)₂SO] δ 7.98 (d, J=8.6 Hz, 1H, H-8), 7.64 (d, J=7.1 Hz, 1H, H-6), 7.33 (br s, 2H, NH₂), 7.23 (dd, J=8.6, 7.1 Hz, 1H, H-7), 2.49 (s, 3H, CH₃); ¹³C NMR [(CD₃)₂SO] δ 156.7, 148.0, 135.1, 134.3, 129.8, 124.0, 117.4, 16.8; Anal. calc. for C₈H₈N₄O: C, 54.5; H, 4.6; N, 31.8; found C, 54.7; H, 4.7; N, 32.1%.

### Example 29

5-Chloro-1,2,4-benzotriazin-3-amine 1-oxide (3aa). Method B using 1,2-dichloro-3-nitrobenzene (2aa) gave 3aa (45%) as a yellow solid, mp (HOAC) 251-254° C.; ¹H NMR [(CD₃)₂SO] δ 8.11 (dd, J=8.7, 1.0 Hz, 1H, H-8), 7.95 (dd, J=7.6, 1.0 Hz, 1H, H-6), 7.67 (br s, 2H, NH₂), 7.29 (dd, J=8.7, 7.6 Hz, 1H, H-7); ¹³C NMR [(CD₃)₂SO] δ 160.1, 145.7, 135.1, 131.1, 128.6, 123.6, 119.1; Anal. calc. for C₇H₅ClN₄O: C, 42.8; H, 2.6; N, 28.5; Cl, 18.0; found C, 43.0; H, 2.5; N, 28.3; Cl, 17.0%.

### Exampl 30

5-Fluoro-1,2,4-benzotriazin-3-amine 1-oxide (3ab). Method A using 6-fluoro-2-nitroaniline (1ab) gave 3ab (43%) as a yellow powder, mp (DMF) 252-256° C. [lit. (Suzuki & Kawakami, Synthesis 1977, 855-857) mp 278° C.] ¹H NMR [(CD₃)₂SO] δ 7.96 (dd, J=8.7, 1.0 Hz, 1H, H-8), 7.66 (ddd, J=10.3, 7.9, 1.2 Hz, 1H, H-6), 7.61 (br s, 2H, NH₂), 7.28 (ddd, J=8.6, 8.0, 5.1 Hz, 1H, H-7); ¹³C NMR [(CD₃)₂SO] δ 160.1 (d, J=5.6 Hz), 154.9 (d, J=253.1 Hz), 139.7 (d, J=16.1 Hz), 131.1 (d, J=4.2 Hz), 122.8 (d, J=7.6 Hz), 11-9.4 (d, J=17.7 Hz), 115.8 (d, J=4.4 Hz); Anal. calc. for C₇H₅FN₄O: C, 46.7; H, 2.8; N, 31.1; F, 10.6; found C, 46.6; H, 2.7; N, 31.2; F, 10.3%.

### Example 31

5-Nitro-1,2,4-benzotriazin-3-amine-1-oxide (3ac). Method B using 2,6-dinitrofluorobenene (2ac) gave 1-oxide 3ac (27%) as a yellow powder, mp (DCM/pet. ether) 269-272° C.; ¹H NMR [(CD₃)₂SO] δ 8.38 (dd, J=8.5, 1.1 Hz, 1H, H-6), 8.32 (dd, J=7.7, 1.2 Hz, 1H, H-8), 7.88 (br s, 2H, NH₂), 7.39 (dd, J=8.5, 7.7 Hz, 1H, H-7); ¹³C NMR [(CD₃)₂SO] δ 160.5, 144 2, 141.5, 130.9, 129.7, 124.2, 122.0; HRMS (EI) calc. for C₇H₅N₅O₃ (M⁺) m/z207.0392, found 207.0393.

### Example 32

5-Methylsulfanyl-1,2,4-benzotriazin-3-amine 1-oxide (3ad). Method A using 6-methylsulfanyl-2-nitroaniline (1ad) gave 3ad (7%) as a yellow solid, mp (MeOH/DCM) 248-252° C.; ¹H NMR [(CD₃)₂SO] δ 7.85 (dd, J=8.7, 1.4 Hz, 1H, H-8), 7.45-7.48 (m, 3H, H-6, NH₂), 7.28 (dd, J=8.7, 7.7 Hz, 1H, H-7), 2.49 (s, 3H, SCH₃); ¹³C NMR [(CD₃)₂SO] δ 159.4, 145.9, 136.8, 129.4, 127.6, 124.3, 114.6, 13.5; Anal. calc. for C₈H₈N₄OS: C, 46.1; H, 3.9; N, 26.9; found C, 46.4; H, 3.8; N, 26.8%.

### Example 33

N⁷,N⁷-Dimethyl-1,2,4-benzotriazine-3,7-diamine 1-oxide (11). A solution of 7-fluoro-1,2,4-benzotriazine-3-amine 1-oxide (3k) (114 mg, 0.63 mmol) and 40% aqueous dimethylamine (5 mL) in CH₃CN (15 mL) was stirred at 90° C. for 4 days. The solvent was evaporated and the residue was partitioned between dilute aqueous NH₃ (10 mL) and DCM (10 mL). The aqueous fraction was extracted with DCM (3×15 mL), the combined organic fraction-dried, and the solvent evaporated. The residue was chromatographed, eluting with a gradient (0-2%) of MeOH/DCM, to give 11 (30 mg, 61%) as an orange powder, mp (DCM/hexane) 231-233° C.; ¹H NMR [(CD₃)₂SO]δ 7.58 (dd, J=9.4, 2.9 Hz, 1H, H-6), 7.45 (d, J=9.4 Hz, 1H, H-5), 7.02 (d, J=2.9 Hz, 1H, H-8), 6.97 (br s, 2H, NH₂), 3.05 [s, 6H, N(CH₃)₂]; ¹³C NMR [(CD₃)₂SO] δ 158.3, 147.6, 130.3, 126.5, 125.6, 95.3, 40.0; Anal. calc. for C₉H₁₁N₅O: C, 52.7; H, 5.4; N, 34.2; found, C, 52.4; H, 5.3; N, 34.2%.

### Example 34

N⁶, N⁶-Dimethyl-1,2,4-benzotriazine-3,6-diamine 1-oxide (12). A solution of 6-fluoro-1,2,4-benzotriazine-3-amine 1-oxide (3t) (0.1 g, 0.55 mmol) and 40% aqueous solution of dimethylamine (5 mL) in CH₃CN (15 mL) was stirred at 20° C. for 5 days. The solvent was evaporated and the residue was partitioned between dilute aqueous NH₃ (10 mL) and DCM (10 mL). The aqueous layer was extracted with DCM (3×15 mL), the combined organic extract dried, and the solvent evaporated. The residue was chromatographed, eluting with a gradient (0-3%) of MeOH/DCM, to give 12 (93 mg, 82%) as an orange powder, mp (DCM/hexane) 264-267° C.; ¹H NMR [(CD₃)₂SO] δ 7.92 (d, J=9.7 Hz, 1H, H-8), 6.97 (dd, J=9.7, 2.7 Hz, 1H, H-7), 6.88 (s, 2H, NH₂), 6.33 (d, J=2.7 Hz, 1H, H-5), 3.09 [s, 6H, N(CH₃)₂]; Anal. calc. for C₉H₁N₅O: C, 52.7; H, 5.4; N, 34.1; found C, 52.5; H, 5.40; N, 34.2%.

### Example 35

N⁶, N⁶-Diethyl-1,2,4-benzotriazine-3,6-diamine 1-oxide (13). 6-Fluoro-1,2,4-benzotriazine-3-amine 1-oxide (3t) (0.1 g, 0.55 mmol) and diethylamine (3 mL) in CH₃CN (15 mL) was heated at 90° C. for 2 days. The solvent was evaporated and the residue was stirred in dilute ammonia (10 mL) and the resulting precipitate was filtered and chromatographed eluting with a gradient of 0-2% MeOH/DCM, to give 13 (83 mg, 65%) as an orange powder, mp (DCM/Hexane) 247-251° C.; ¹H NMR [(CD₃)₂SO] δ 7.91 (d, J=9.8 Hz, 1H, H-8), 6.93 (dd, J=9.7, 2.8 Hz, 1H, H-7), 6.83 (s, 2H, NH₂), 6.31 (d, J=2.6 Hz, 1H, H-5), 3.47 (q, J=7.0 Hz, 4H, 2×CH₂) 1.14 (t, J=7.0 Hz, 6H, 2×CH₃); ¹³C NMR [(CD₃)₂SO] δ 160.6, 152.1, 150.9, 121.8, 121.2, 113.9, 99.0, 44.2, 12.3; Anal. calc. for C₁₁H₁₅N₅O: C, 56.6; H, 6.5; N, 30.0; found C, 56.6; H, 6.6; N, 30.1%.

### Example 36

7-(2-Methoxyethoxy)-1,2,4-benzotriazin-3-amine 1-oxide (14). A mixture of 7-hydroxy-1-oxide 3h (1.00 g, 5.8 mmol), dry K₂CO₃ (2.40 g, 17.4 mmol) and 2-bromoethylmethylether (2.42 g, 17.4 mmol) in DMF (20 mL) was heated at 80° C. for 2 h. The solvent was evaporated and the residue chromatographed, eluting with a gradient (0-3%) MeOH/DCM, to give compound 14 (1.06 g, 77%) as a yellow powder, mp (DCM/pet. ether) 201-203° C.; ¹H NMR [(CD₃)₂SO] δ 8.07 (d, J=9.5 Hz, 1H, H-5), 7.82 (br s, 2H, NH₂), 7.76 (dd, J=9.5, 2.6 Hz, 1H, H-6), 7.50 (d, J=2.6 Hz, 1H, H-8), 4.26, (t, J=4.3 Hz, 2H, CH₂), 3.72 (t, J=4.3 Hz, 2H, CH₂), 3.33 (s, 3H, OCH₃); Anal. calc. for C₁₀H₁₂N₄O₅: C, 50.8; H, 5.1; N, 23.7; found C, 51.1; H, 5.0; N, 23.7%.

### Example 37

7-(2-Bromoethoxy)-1,2,4-benzotriazin-3-amine 1-oxide (15). A mixture of 7-hydroxy-1-oxide 3h (1.23 g, 7.15 mmol), K₂CO₃ (1.97 g, 14.3 mmol) and 1,2-dibromoethane (4.0 ml) in DMF (20 ml) was heated at 80° C. for 20 h. The solvent was evaporated, and the residue stirred in water (100 mL). The resulting precipitate was filtered, washed with water (3×50 mL) and dried to give a yellow solid, which was chromatographed, eluting with 0-3% MeOH/DCM, to give 1-oxide 15 (1.0 g, 49%) as a yellow powder, mp (DCM/pet. ether) 228-230° C.; ¹H NMR δ 7.52-7.50 (m, 3H, H-5, H-6, H-8), 7.12 (br s, 2H, NH₂), 4.45 (t, J=5.2 Hz, 2H, CH₂), 3.85 (t, J=5.2 Hz, 2H, CH₂); HRMS (EI) calc. for C₉H₉⁷⁹BrN₄O₂(M+) m/z283.9909, found 283.9902; calc. for C₉H₉⁸¹BrN₄O₂ (M⁺) m/z 285.9888, found 285.9881.

### Example 38

N{2-[(3-Amino-1-oxido-1,2,4-benzotriazin-7-yl)oxy]ethyl}-2,2,2-trifluoroacetamide (16). A mixture of 7-hydroxy-1-oxide 3h (520 mg, 3.02 mmol), K₂CO₃ (883 mg, 6.04 mmol) and N-(2-bromoethyl)-2,2,2-trifluoroacetamide (1.25 g, 6.03 mmol) in DMF (20 ml) was heated at 80° C. for 3 h. The solvent was evaporated and residue stirred in water (100 mL) the resulted precipitate was filtered, washed with water (3×50 mL), and dried to give a yellow solid, which was chromatographed, eluting with a gradient (0-3%) of MeOH/DCM, to give 1-oxide 16 (639 mg, 66%) as a yellow powder, mp (DCM/pet. ether) 234-246° C.; ¹H NMR [(CD₃)₂SO] δ 9.66 (br s, 1H, CONH), 7.52 (d, J=9.2 Hz, 1H, H-5), 7.51 (d, J=2.3 Hz, 1H, H-8), 7.42 (dd, J=9.1, 2.9 Hz, 1H, H-6) 7.12 (br s, 2H, NH₂), 4.23 (t, J=5.5 Hz, 2H, CH₂), 3.63 (t, J=5.4 Hz, 2H, CH₂); ¹³C NMR [(CD₃)₂SO] δ 159.5, 156.7 (q, J=36.3 Hz), 155.1, 144.9, 129.7, 128.4, 127.4, 119.8, 115.8 (q, J=287.8 Hz), 99.0, 65.9; Anal. calc. for C₁₁H₁₀F₃N₅O₃: C, 41.7; H, 3.2; N, 22.2; F, 18.0; found C, 42.0; H, 3.0; N, 21.9; F, 17.5%.

### Example 39

7-[2-(4-Morpholinyl)ethoxy]-1,2,4-benzotriazin-3-amin 1-oxide (17). A mixture of 7-hydroxy-1-oxide 3h (1.15 g, 6.7 mmol), K₂CO₃ (3.77 g, 20.0 mmol) and 4-(2-chloroethyl)morpholine hydrochloride (2.49 g, 13.4 mmol) in DMF (25 ml) was heated at 80° C. for 2 h. The solvent was evaporated, the residue stirred in water (100 mL), the resulting precipitate filtered, washed with water (3×50 mL), and dried to give 1-oxide 17 (1.53 g, 79%) as a yellow solid, mp (DCM/pet. ether) 175-181° C.; ¹H NMR [(CD₃)₂SO] δ 7.52-7.46 (m, 3H, H-5, H-6, H-8), 7.09 (br s, 2H, NH₂), 4.20 (t, J=5.6 Hz, 2H, CH₂), 3.58 (t, J=4.7 Hz, 4H, 2×CH₂), 2.73 (t, J=5.6 Hz, 2H, CH₂), 2.49 (t, J=4.6 Hz, 4H, 2×CH₂); ¹³C NMR [(CD₃)₂SO] δ 159.5, 155.5, 144.8, 129.7, 128.5, 127.3, 98.8, 66.1 (2), 56.7, 53.5 (2); Anal. calc. for C₁₃H₁₇N₅O₃: C, 53.6; H, 5.9; N, 24.0; found C, 53.5; H, 6.0; N, 23.8%.

### Example 40

1,2,4-Benzotriazin-3-amine 1-oxide (3). A mixture of 2-nitroaniline (1) (10.0 g, 72.4 mmol) and cyanamide (15.2 g, 0.36 mmol) was melted at 100° C., cooled to ca. 40° C., cHCl (20 mL) added carefully. The exotherm was allowed to subside and the mixture was heated at 100° C. for 1 h. The mixture was cooled to ca. 40° C. and 30% NaOH solution (30 mL) added carefully. The mixture was stirred at 100° C. for 2 h, cooled to 25° C., diluted with water (50 mL) and stirred for 30 min. The suspension was filtered, washed with water (2×10 mL), ether (2×5 mL) and dried under vacuum to give 1-oxide 3 (10.3 g, 88%) as a yellow powder, mp (MeOH/EtOAc) 267-269° C. [lit. (Arndt, Ber. 1913, 46, 3522) mp (EtOH) 269° C.]; ¹H NMR δ 8.13 (d, J=8.7 Hz, 1H, H-8), 7.79 (dd, J=8.6, 7.0 Hz, 1H, H-6), 7.54 (d, J=8.6 Hz, 1H, H-5), 7.32-7.38 (m, 3H, H-7, NH₂).

### Example 41

3-Chloro-1,2,4-benzotriazine 1-oxide (19). A solution of NaNO₂ (10 g, 0.145 mol) in water (100 mL) added dropwise to a suspension of 1-oxide 3 (11.7 g, 72.2 mmol) in 2 M HCl (300 mL) at 5° C. and the mixture stirred vigorously until the foaming subsided (2 h). The resulting precipitate was filtered, dissolved in dilute aqueous NH₃, filtered, and acidified with cHCl. The precipitate was filtered, washed with water and dried to give 3-hydroxy-1,2,4-benzotriazine 1-oxide (18) (5.77 g, 49%) as a yellow powder, mp 209-212° C.; [lit. (Robbins et al., J. Chem. Soc. 1957, 3186) mp (H₂O) 219° C.]; ¹H NMR 88.14 (d, J=8.4 Hz, 1H, H-8), 7.77-7.81 (m, 1H, H-6), 7.54 (d, J=8.4 Hz, 1 H, H-5), 7.90 (m, 3H, H-7, NH₂); ¹³C NMR δ 160.2, 148.7, 135.6, 129.8, 125.8, 124.6, 119.8.

A mixture of alcohol 18 (5.7 g, 34.9 mmol), N,N-dimethylaniline (11 mL, 87.3 mmol), and POCl₃ (23 mL, 244 mmol) was heated at reflux temperature for 1 h then poured on to ice. The resulting solid was filtered and recrystallized to give chloride 19 (3.77 g, 59%) as a pale yellow powder, mp (MeOH) 119-119.5° C. [lit. (Robbins et al., J. Chem. Soc., 1957, 3186) mp (MeOH) 117-118° C.]; ¹H NMR δ 8.38 (dd, J=8.7, 1.0 Hz, 1H, H-8), 8.16 (ddd, J=8.3, 7.0, 1.3 Hz, 1H, H-6), 8.06 (dd, J=8.2, 1.0 Hz, 1H, H-5), 7.90 (ddd, J=8.7, 6.9, 1.3 Hz, 1H, H-7); ¹³C NMR δ 155.3, 146.9, 137.2, 133.9, 131.5, 128.0, 119.9.

### Example 42

Ethyl [(1-oxido-1,2,4-benzotriazin-3-yl)amino]acetate (20). A mixture of chloride 19 (2.02 g, 11.1 mmol), glycine ethyl ester hydrochloride (2.33 g, 16.7 mmol) and Et₃N (4.2 mL, 30 mmol) in DME (100 mL) was heated at reflux temperature for 6 h. The solvent was evaporated and the residue partitioned between DCM (100 mL) and water (100 mL), the aqueous fraction extracted with DCM (2×50 mL), the combined organic fraction dried, and the solvent evaporated. The residue was chromatographed, eluting with 10% EtOAc/DCM, to give ester 20 (2.75 g, 99%) as a yellow solid, mp (EtOAc/DCM) 136-138° C.; ¹H NMR δ 8.27 (dd, J=8.6, 1.0 Hz, 1H, H-8), 7.72 (ddd, J=8.5, 7.0, 1.4 Hz, 1H, H-6), 7.62 (dd, J=8.5, 1.0 Hz, 1H, H-5), 7.34 (ddd, J=8.6, 7.0, 1.0 Hz, 1H, H-7), 5.87 (br s, 1H, NH), 4.30 (d, J=5.7 Hz, 2H, CH₂N), 4.26 (q, J=7.2 Hz, 2H, CH₂O), 1.31 (t, J=7.2 Hz, 3H, CH₃); ¹³C NMR δ 169.9 (CO₂), 158.4 (C-3), 148.5 (C-4a), 135.6 (C-6), 131.2 (C-8a), 126.7 (C-5), 125.5 (C-7), 120.4 (C-8), 61.6 (CH₂O), 43.2 (CH₂N), 14.2 (CH₃); Anal. calc. for C₁₁H₁₂N₄O₃: C, 53.2; H, 4.9; N, 22.6; found C, 53.4; H, 5.0; N, 22.6%.

### Example 43

[(1-Oxido-1,2,4-benzotriazin-3-yl)amino]acetic acid (21). A solution of ester 20 (0.75 g, 3.0 mmol) and 1 M NaOH (15 mL, 15.0 mmol) in MeOH (50 mL) was stirred at 20° C. for 2 h. The solution was washed with ether (2×50 mL), the volume reduced to 20 mL, and the pH adjusted to 3 with 5 M HCl. The precipitate was filtered and recrystallized to give acid 21 (576 mg, 86%) as a pale yellow solid, mp (water) 217-219° C.; ¹H NMR [(CD₃)₂SO] δ 8.14 (d, J=8.6 Hz, 1H, H-8), 8.08 (br. s, 1H, NH), 7.80 (ddd, J=8.6, 7.1, 1.1 Hz, 1H, H-6), 7.59 (d, J=8.6 Hz, 1H, H-5), 7.37 (ddd, J=8.6, 7.1, 1.1 Hz, 1H, H-7), 4.01 (br s, 2H, CH₂N), CO₂H not observed; ¹³C NMR [(CD₃)₂SO] δ 171.3 (CO₂), 158.8 (C-3), 148.0 (C-4a), 135.8 (C-6), 130.2 (C-8a), 126.1 (C-5), 125.0 (C-7), 119.8 (C-8), 42.6 (CH₂N); Anal. calc. for C₉H₈N₄O₃.¹/4H₂O: C, 48.1; H, 3.8; N, 24.9; found C, 48.2; H, 3.8; N, 24.2%.

### Example 44

2-[(1-Oxido-1,2,4-benzotriazin-3-yl)amino]ethanol (22). A solution of chloride 19 (536 mg, 3.0 mmol) and ethanolamine (0.53 mL, 8.9 mmol) was heated at reflux temperature in DME (50 mL) for 1 h. The mixture was cooled to 20° C., the solvent evaporated, and the residue partitioned between dilute aqueous NH₃ (50 mL) and DCM (100 mL). The organic fraction was dried, and the solvent evaporated. The residue was chromatographed, eluting with 5% MeOH/DCM, to give alcohol 22 (533 mg, 88%) as a yellow solid, mp (MeOH/DCM) 214-218° C.; ¹H NMR [(CD₃)₂SO] δ 8.13 (dd, J=8.5, 1.0 Hz, 1H, H-8′), 7.82 (br s, 1H, NH), 7.78 (ddd, J=8.4, 7.0, 1.0 Hz, 1H, H-6′), 7.57 (d, J=8.4, Hz, 1H, H-5′), 7.34 (ddd, J=8.5, 7.0, 1.0 Hz, 1H, H-7′), 4.74 (t, J=5.6 Hz, 1H, OH), 3.56-3.61 (m, 2H, CH₂), 3.40-3.45 (m, 2H, CH₂N); ¹³C NMR [(CD₃)₂SO] δ 159.0 (C-3′), 148.2 (C-4a′), 135.7 (C-6′), 130.0 (C-8a′), 125.9 (C-5′), 124.4 (C-7′), 119.8 (C-8′), 59.2 (CH₂O), 43.2 (CH₂N); Anal. calc. for CgH,ON₄O₂: C, 52.4; H, 4.9; N, 27.2; found C, 52.3; H, 4.8; N, 26.6%.

### Example 45

N-(2-Methoxyethyl)-1,2,4-benzotriazin-3-amine 1-oxide (23). A solution of chloride 19 (783 mg, 4.3 mmol) and 2-methoxyethylamine (0.82 mL, 9.5 mmol) in DME (70 mL) was heated at reflux temperature for 5 h. The cooled solution was partitioned betwen EtOAc (100 mL) and water (100 mL). The aqueous fraction was extracted with EtOAc (50 mL), the combined organic fractioned dried, and the solvent evaporated. The residue was chromatographed, eluting with a gradient (10-30%) EtOAc/DCM, to give 1-oxide 23 (915 mg, 96%) as a yellow powder, mp (EtOAc/DCM) 154-156° C.; ¹H NMR δ 8.26 (dd, J=8.6, 1.3 Hz, 1H, H-8), 7.71 (ddd, J=8.4, 7.1, 1.3 Hz, 1H, H-6), 7.60 (br d, J=8.4 Hz, 1H, H-5), 7.30 (ddd, J=8.6, 7.1, 1.4 Hz, 1H, H-7), 5.71 (br s, 1H, NH), 3.72 (dt, J=5.5, 5.3 Hz, 2H, CH₂N), 3.61 (dd, J=5.3, 5.0 Hz, 2H, CH₂O), 3.40 (s, 3H, CH₃O); ¹³C NMR δ 158.8 (C-3), 148.5 (C-4a), 135.6 (C-6), 131.0 (C-8a), 126.3 (C-5), 125.80 (C-7), 120.5 (C-8),170.8 (CH₂O), 58.9 (OCH₃), 41.1 (CH₂N); Anal. calc. for C₁₀H₁₂N₄O₂: C, 54.5; H, 5.5; N, 25.4; found C, 54.8; H, 5.3; N, 25.3%.

### Exampl 46

3-[(1-Oxido-1,2,4-benzotriazin-3-yl)amino]propanol (24). A solution of chloride 19 (710 mg, 3.9 mmol) and 3-amino-1-propanol (0.75 mL, 9.8 mmol) was heated at reflux temperature in DME (50 mL) for 1 h. The mixture was cooled to 20° C., the solvent evaporated, and the residue partitioned between dilute aqueous NH₃ (50 mL) and DCM (100 mL). The organic fraction was dried, and the solvent evaporated. The residue was chromatographed, eluting with 5% EtOAc/DCM, to give alcohol 24 (828 mg, 96%) as a yellow solid, mp (MeOH/DCM) 155-156° C.; ¹H NMR [(CD₃)₂SO] δ 8.13 (dd, J=8.6, 1.1 Hz, 1H, H-8′), 7.87 (br s, 1H, NH), 7.78 (ddd, J=8.4, 7.1, 1.1 Hz, 1H, H-6′), 7.57 (d, J=8.4, Hz, 1H, H-5′), 7.34 (ddd, J=8.6, 7.1, 1.1 Hz, 1H, H-7′), 4.51 (t, J=5.1 Hz, 1H, OH), 3.50-3.55 (m, 2H, CH₂), 3.40-3.45 (m, 2H, CH₂N), 1.72-1.79 (m, 2H, CH₂); ¹³C NMR [(CD₃)₂SO] δ 158.9 (C-3′), 148.3 (C-4a′), 135.6 (C-6′), 129.9 (C-8a′), 125.9 (C-5′), 124.3 (C-7′), 119.8 (C-8′), 58.4 (CH₂O), 37.9 (CH₂N), 31.7 (CH₂); Anal. calc. for C₁₀H₁₂N₄O₂; C, 54.5; H, 5.5; N, 25.5; found C, 54.9; H, 5.6; N, 25.6%.

### Example 47

2-[(1-Oxido-1,2,4-benzotriazin-3-yl)amino]acetonitrile (25). A solution of chloride 19 (790 mg, 4.4 mmol), 2-aminoacetonitrile hydrochloride (0.81 g, 8.7 mmol) and Et₃N (1.2 mL, 8.7 mmol) in DME (80 mL) was stirred at reflux temperature for 6 h. The solution was partitioned between DCM (100 mL) and water (100 mL), the aqueous fraction washed with DCM (2×50 mL), the combined organic fraction dried and the solvent evaporated. The residue was chromatographed, eluting with a gradient (5-20%) of EtOAc/DCM, to give nitrile 25 (383 mg, 44%) as yellow needles, mp (EtOAc/DCM) 233-237° C.; ¹H NMR δ 8.33 (dd, J=8.5, 1.2 Hz, 1H, H-8′), 7.83 (ddd, J=8.4, 7.1, 1.2 Hz, 1H, H-6′), 7.76 (d, J=8.4 Hz, 1H, H-5′), 7.45 (ddd, J=8.5, 7.1, 1.2 Hz, 1H, H-7′), 5.68 (br s, 1H, NH), 4.49 (d, J=6.3 Hz, 2H, CH₂); Anal. calc for C₉H₇N₅O: C, 53.7; H, 3.5; N, 34.8; found C, 54.0; H, 3.2; N, 34.9%.

### Example 48

3-[(1-Oxido-1,2,4-benzotriazin-3-yl)amino]propanenitrile (26). A solution of chloride 19 (776 mg, 4.3 mmol), 3-aminopropanenitrile fumarate (2.74 g, 21.4 mmol) and Et₃N (3.6 mmol, 25.6 mmol) in DME (50 mL) was stirred at reflux temperature for 6 h. The solution was partitioned between DCM (100 mL) and water (100 mL), the aqueous fraction washed with DCM (2×50 mL), the combined organic fraction dried and the solvent evaporated. The residue was chromatographed, eluting with 10% EtOAc/DCM, to give nitrile 26 (771 mg, 84%) as yellow needles, mp (EtOAc/DCM) 191-193° C.; ¹H NMR δ 8.29 (dd, J=8.7, 1.1 Hz, 1H, H-8′), 7.76 (ddd, J=8.5, 7.0, 1.1 Hz, 1H, H-6′), 7.64 (dd, J=8.5, 1.0 Hz, 1H, H-5′) 7.37 (ddd, J=8.7, 7.0, 1.0 Hz, 1H, H-7′), 6.00 (br s, 1H, NH), 3.87 (q, J=6.5 Hz, 2H, H-3), 2.85 (t, J=6.5 Hz, 2H, H-2); ¹³C NMR δ 158 (C-3′), 148.4 (C-4a′), 135.9 (C-6′), 131.3 (C-8a′), 126.7 (C-5′), 120.4 (C-8′), 117.9 (C-1), 37.6 (C-3), 18.1 (C-2); Anal. calc. for C₁₀H₉N₅O: C, 55.8; H, 4.2; N, 32.6; found, C, 55.9; H, 4.3; N, 32.6%.

### Example 49

4-[(1-Oxido-1,2,4-benzotriazin-3-yl)amino]butanenitrile (27). A solution of chloride 19 (1.82 g, 10.0 mmol) and 4-aminobutanenitrile (2.11 g, 25.0 mmol) in DME (100 mL) was heated at reflux temperature for 4 h. The cooled solution was partitioned betwen EtOAc (200 mL) and water (200 mL). The aqueous fraction was extracted with EtOAc (100 mL), the combined organic fractioned dried, and the solvent evaporated. The residue was chromatographed, eluting with a gradient (5-10%) EtOAc/DCM, to give 1-oxide 27 (1.80 g, 79%) as a yellow powder, mp (EtOAc/DCM) 184-187° C.; ¹H NMR δ 8.26 (dd, J=8.7, 1.3 Hz, 1H, H-8′), 7.73 (ddd, J=8.4, 7.0, 1.5 Hz, 1H, H-6′), 7.63 (d, J=8.4 Hz, 1H, H-5′), 7.33 (ddd, J=8.7, 7.0, 1.3 Hz, 1H, H-7′), 6.00 (br s, 1H, NH), 3.72 (dd, J=6.6, 6.4 Hz, 2H, H-4), 2.51 (t, J=7.2 Hz, 2H, H-2), 2.07-2.14 (m, 2H, H-3); ¹³C NMR δ 158.7 (C-3′), 148.2 (C-4a′), 135.8 (C-6′), 131.0 (C-8a′), 126.4 (C-5′), 125.4 (C-7′), 120.4 (C-8′), 119.2 (C-1), 40.0 (C-4), 25.4 (C-2), 14.8 (C-3); Anal. calc. for C₁₁H₁₁N₅O: C, 57.6; H, 4.8; N, 30.6; found C, 57.8; H, 5.1; N, 30.8%.

### Example 50

N-(3-Azidopropyl)-1,2,4-benzotriazin-3-amine 1-oxide (28). A solution of chloride 19 (2.18 g, 12.0 mmol) and 3-azido-1-propanamine hydrochloride (2.46 g, 18.0 mmol) and Et₃N (5.0 mL, 36.0 mmol) in DCM (100 mL) was heated at reflux temperature for 16 h. The solvent was evaporated and the residue chromatographed, eluting with a gradient (5-10%) of EtOAc/DCM, to give 1-oxide 28 (2.49 g, 85%) as a yellow powder, mp (EtOAc/DCM) 128-130° C.; ¹H NMR δ 8.26 (dd, J=8.7, 1.4 Hz, 1H, H-8′), 7.71 (ddd, J=8.3, 7.1, 1.4 Hz, 1H, H-6′), 7.61 (d, J=8.3 Hz, 1H, H-5′), 7.30 (ddd, J=8.7, 7.1, 1.1 Hz, 1H, H-7′), 5.73 (br s, 1H, NH), 3.67 (dd, J=6.6, 6.4 Hz, 2H, CH₂N), 3.47 (t, J=6.5 Hz, 2H, CH₂N₃), 1.95-2.03 (m, 2H, CH₂); ¹³C NMR δ 158.9 (C-3′), 148.8 (C-4a′), 135.6 (C-6′), 130.9 (C-8a′), 126.6 (C-5′), 125.0 (C-7′), 120.4 (C-8′), 49.2 (CH₂N₃), 38.8 (CH₂N), 28.6 (CH₂); Anal. calc. for C₁₀H₁₁N₇O: C, 50.0; H, 4.5; N, 40.0; found C, 49.1; H, 4.6; N, 40.3%.

### Example 51

tert-Butyl 3-[(1-oxido-1,2,4-benzotriazin-3-yl)amino]propylcarbamate (29). A solution of chloride 19 (4.0 g, 22.0 mmol), tert-butyl 3-aminopropylcarbamate (5.76 g, 33.0 mmol) and Et₃N (4.6 mL, 33.0 mmol) in DCM (150 mL) was stirred at 20° C. for 5 days. The solvent was evaporated and the residue chromatographed, eluting with 20% EtOAc/DCM, to give 1-oxide 29 (5.21 g, 74%) as a yellow powder, mp (EtOAc/DCM) 145-147° C.; ¹H NMR [(CD₃)₂SO] δ 8.13 (dd, J=8.6, 1.1 Hz, 1H, H-8′), 7.84 (s, 1H, NH), 7.78 (ddd, J=8.4, 7.1, 1.1 Hz, 1H, H-6′), 7.56 (d, J=8.4 Hz, 1H, H-5′), 7.32 (ddd, J=8.6, 7.1, 1.1 Hz, 1H, H-7′), 6.83 (t, J=5.3 Hz, 1H, NHCO₂), 3.32-3.36 (m, 2H, H-1), 2.99-3.04 (m, 2H, H-3), 1.66-1.73 (m, 2H, H-2), 1.37 [s, 9H, C(CH₃)₃]; ¹³C NMR [(CD₃)₂SO] δ 158.9, 155.6, 148.2, 135.7, 130.0, 125.9, 124.4, 119.9, 77.4, 38.2, 37.5, 28.9, 28.2 (3); Anal. calc. for C₁₅H₂₁N₅O₃: C, 56.4; H, 6.6; N, 21.9; found: C, 56.4; H, 6.6; N, 22.1%.

### Example 52

tert-Butyl 3-[(1-oxido-1,2,4-benzotriazin-3-yl)amino]propyl(ethyl)carbamate (32). tert-Butyl 2-cyanoethyl(ethyl)carbamate (30). A solution of ethylamine (6.1 mL, 76 mmol) was added dropwise to stirred acrylonitrile (10 mL) at 5° C. and the mixture allowed to warm to 20° C. over 1 h. The excess acrylonitrile was evaporated and the residue dissolved in DCM (100 mL). A solution of di-tert-butyldicarbonate (18.3 g, 84 mmol) in DCM (50 mL) added dropwise at 5° C. and then stirred at 20° C. for 16 h. The solution was diluted with DCM (100 mL), washed with dilute Na₂CO₃ solution (100 mL), 0.1 M HCl (100 mL), water (2×100 mL), and brine (50 mL). The organic fraction was dried and the solvent evaporated. The residue was chromatographed, eluting with 20% EtOAc/pet. ether, to give nitrile 30 (15.0 g, 99%) as an oil, ¹H NMR δ 3.47 (t, J=6.7 Hz, 2H, CH₂N), 3.20 (q, J=7.1 Hz, 2H, CH₂), 2.61 (br s, 2H, CH₂), 1.48 [s, 9H, C(CH₃)₃], 1.14 (t, J=7.1 Hz, 3H, CH₃); MS (EI) m/z198 (M⁺, 1%), 158 (2), 143 (5), 125 (20), 57 (100); HRMS (EI) calc. for C₁₀H₁₈N₂O₂ (M⁺) m/z 198.1368, found 198.1367.

tert-Butyl 3-[(1-oxido-1,2,4-benzotriazin-3-yl)amino]propyl(ethyl)carbamate (32). A mixture of nitrile 30 (4.60 g, 23.2 mmol) and freshly prepared Raney Nickel (3 mL) in EtOH saturated with NH₃ was stirred under H₂ (60 psi) for 16 h. The mixture was filtered through celite, washed with EtOH (4×10 mL), and the solvent evaporated to give tert-butyl 3-aminopropyl(ethyl)carbamate (31) (4.65 g, 99%) as an oil which was used without further characterization. Amine 31 (2.5 g, 12.3 mmol) was added to a stirred solution of chloride 19 (0.89 g, 4.9 mmol) in DME (50 mL) and the solution heated at 100° C. for 6 h. The solvent was evaporated and the residue partitioned between DCM (150 mL) and water (100 mL). The organic fraction was dried and the solvent evaporated. The residue was chromatographed, eluting with a gradient (20-50%) of EtOAc/pet. ether, to give 1-oxide 32 (1.43 g, 84%) as a yellow solid, mp (EtOAc/pet ether) 64-66° C.; ¹H NMR δ 8.24 (d, J=8.6 Hz, 1H, H-8), 7.65-7.70 (m, 1H, H-6), 7.57 (d, J=8.4 Hz, 1H, H-5), 7.24-7.28 (m, 1H, H-7), 6.32 (br s, 1H, NH), 3.50-3.55 (m, 2H, CH₂N), 3.32-3.35 (m, 2H, CH₂N), 3.19-3.25 (m, 2H, CH₂N), 1.82-1.86 (m, 2H, CH₂), 1.48 [s, 9H, C(CH₃)₃], 1.12 (t, J=7.0 Hz, 3H, CH₃); ¹³C NMR δ 159.0 (NCO₂), 156.2 (C-3), 148.9 (C-4a), 135.4 (C-6), 130.8 (C-8a), 126.4 (C-5), 124.6 (C-7), 120.4 (C-8), 79.6 [C(CH₃)₃], 43.5 (CH₂N), 42.0 (CH₂N), 37.9 (CH₂N), 27.9 [C(CH₃)₃], 27.4 (CH₂), 13.8 (CH₃); Anal. calc. for C₁₇H₂₅N₅O₃: C, 58.8; H, 7.3; N, 20.2; found C, 59.0; H, 7.3; N, 20.4%.

### Example 53

3-{[2-(2-Hydroxyethoxy)ethyl]amino}-1,2,4-benzotriazine 1-oxide (33). A solution of chloride 19 (3.0 g, 16.5 mmol) in DCM (50 mL) was added to a stirred solution of 2-(aminoethoxy)ethanol (2.49 mL, 24.8 mmol) and Et₃N (3.45 mL, 24.8 mmol) in DCM (80 mL) and the solution stirred at 20° C. for 16 h. The solvent was evaporated and the residue chromatographed, eluting with 40% EtOAc/DCM, to give 1-oxide 33 (2.62 g, 63%) as a yellow powder, mp (DCM/EtOAc) 131-131.5° C.; ¹H NMR δ 8.25 (dd, J=8.7, 1.2 Hz, 1H, H-8), 7.68 (ddd, J=8.4, 7.2, 1.5 Hz, 1H, H-6), 7.57 (d, J=8.4 Hz, 1H, H-5), 7.28 (ddd, J=8.7, 7.2, 1.3 Hz, 1H, H-7), 6.02 (br s, 1H, NH), 3.74-3.80 (m, 6H, 3×CH₂O), 3.64-3.67 (m, 2H, CH₂N), 2.71 (t, J=5.9 Hz, 1H, OH); ¹³C NMR δ 158.9, 149.7, 135.5, 130.9, 126.4, 124.9, 120.4, 72.4, 69.5, 61.7, 41.9; Anal. calc. for C₁₁H₁₄N₄O₃: C, 52.8; H, 5.6; N, 22.4; found C, 52.9; H, 5.7; N, 22.6%.

### Example 54

3-{[2-(2-Azidoethoxy)ethyl]amino}-1,2,4-benzotriazine 1-oxide (34). Methanesulfonyl chloride (0.82 mL, 10.6 mmol) was added dropwise to a stirred solution of alcohol 33 (2.41 g, 9.63 mmol) and Et₃N (1.74 mL, 12.5 mmol) in DCM (100 mL) at 5° C. and the solution stirred at 20° C. for 1 h. The solution was diluted with DCM (100 mL) and washed with water (3×50 mL), brine (50 mL), dried and the solvent evaporated. The residue was dissolved in DMF (50 mL) and NaN₃ (0.69 g, 10.6 mmol) added. The mixture was heated at 100° C. for 2 h, cooled to 30° C. and the solvent evaporated. The residue was partitioned between EtOAc (100 mL) and water (100 mL). The organic fraction was washed with brine (50 mL), dried, and the solvent evaporated. The residue was chromatographed, eluting with 50% EtOAc/pet. ether, to give azide 34 (2.35 g, 89%) as yellow crystals, mp (EtOAc/pet. ether) 102-104° C.; ¹H NMR δ 8.27 (dd, J=8.7, 1.4 Hz, 1H, H-8), 7.70 (ddd, J=8.6, 7.1, 1.5 Hz, 1H, H-6), 7.59 (d, J=8.6 Hz, 1H, H-5), 7.29 (ddd, J=8.6, 7.1, 1.4 Hz, 1H, H-7), 5.70 (br s, 1H, NH), 3.71-3.78 (m, 4H, 2×CH₂O), 3.69 (dd, J=5.3, 4.8 Hz, 2H, CH₂N₃), 3.41 (dd, J=5.1, 4.9 Hz, 2H, CH₂N); ¹³C NMR 6158.9, 148.7, 135.5, 131.1, 126.5, 125.0, 120.4, 70.0, 69.6, 50.7, 41.1; Anal. calc. for C₁₁H₁₃N₇O₂; C, 48.0; H, 4.8; N, 35.6; found: C, 48.3; H, 4.6; N, 35.7%.

### Example 55

3-{[2-(2-tert-Butyloxycarbamoylethoxy)ethyl]amino}-1,2,4-benzotriazine 1-oxide (35). Propane-1,3-dithiol (5.7 mL, 57.0 mmol) was added dropwise to a stirred solution of azide 34 (1.57 g, 5.70 mmol) and Et₃N (7.95 mL, 57 mmol) in MeOH (100 mL) under N₂ and the solution heated at reflux temperature for 8 h. The solution was cooled to 30° C. and partitioned between 1 M HCl (100 mL) and Et₂O (100 mL). The aqueous fraction was adjusted to pH 12 with 7 M NaOH solution and extracted with DCM (3×50 mL). The organic fraction was dried and the solvent evaporated. The residue was dissolved in THF (100 mL) and a solution of di-tert-butyldicarbonate (1.87 g, 8.55 mmol) in THF (50 mL) added dropwise. The solution was stirred at 20° C. for 16 h, the solvent evaporated and the residue chromatographed, eluting with 40% EtOAc/pet. ether, to give carbamate 35 (1.85 g, 93%) as a yellow solid, mp (EtOAc/pet. ether) 134-137° C.; ¹H NMR δ 8.26 (dd, J=8.4, 0.9 Hz, 1H, H-8), 7.71 (ddd, J=8.3, 7.1, 1.4 Hz, 1H, H-6), 7.59 (d, J=8.3 Hz, 1H, H-5), 7.29 (ddd, J=8.4, 7.1, 1.3 Hz, 1H, H-7), 5.74 (br s, 1H, NH), 4.93 (br s, 1H, NH), 3.67-3.73 (m, 4H, 2×CH₂O), 3.56 (t, J=5.2 Hz, 2H, CH₂N), 3.29-3.36 (m, 2H, CH₂N), 1.45 [s, 9H, C(CH₃)₃]; ¹³C NMR δ 159.9, 155.9, 148.7, 135.5, 131.0, 126.5, 125.0, 120.4, 79.4, 70.2, 69.2, 41.1, 40.4, 28.4 (3); Anal. calc. for C₁₆H₂₃N₅O₄: C, 55.0; H, 6.6; N, 20.1; found C, 55.3; H, 6.8; N, 20.1%.

### Example 56

N-[2-(2-Aminoethoxy)ethyl]-1,2,4-benzotriazin-3-amin 1-oxide (36).

A solution of carbamate 35 (0.99 g, 2.8 mmol) in MeOH (30 mL) was treated with HCl gas and stirred at 20° C. for 2 h. The solvent was evaporated and the residue partioned between dilute NH₄OH (50 mL) and CHCl₃ (50 mL). The organic fraction was dried and the solvent evaporated to give amine 36 (618 mg, 88%) as a red solid, mp 116-118° C.; ¹H NMR δ 8.25 (dd, J=8.7, 1.1 Hz, 1H, H-8), 7.70 (ddd, J=8.4, 7.1, 1.1 Hz, 1H, H-6), 7.58 (d, J=8.4 Hz, 1H, H-5), 7.28 (ddd, J=8.7, 7.1, 1.1 Hz, 1H, H-7), 6.04 (br s, 1H, NH), 3.68-3.76 (m, 4H, 2×CH₂O), 3.54 (t, J=5.1 Hz, 2H, CH₂N), 2.90 (t, J=5.1 Hz, 2H, CH₂N), 1.82 (br s, 2H, NH₂); ¹³C NMR δ 158.9 (C-3), 148.8 (C-4a), 135.5 (C-6), 130.9 (C-8a), 126.4 (C-5), 124.9 (C-7), 120.4 (C-8), 73.1 (CH₂O), 69.2 (CH₂O), 41.7 (CH₂N), 41.2 (CH₂N); Anal. calc. for C₂₅H₂₂N₆O₃.½H₂O: C, 51.15; H, 6.2; N, 27.1; found C, 51.6; H, 6.1; N, 26.8%.

### Example 57

N¹,N¹-Dimethyl-N²-(1-oxido-1,2,4-benzotriazin-3-yl)-1,2-ethanediamine (37). N,N-Dimethylethanediamine (0.66 mL, 6.0 mmol) was added to a stirred solution of chloride 19 (438 mg, 2.4 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 37 (514 mg, 91%) as a yellow solid, mp (MeOH/EtOAc) 121-123° C.; ¹H NMR [(CD₃)₂SO] δ 8.13 (dd, J=8.6, 1.1 Hz, 1H, H-8′), 7.78 (ddd, J=8.5, 7.0, 1.1 Hz, 1H, H-6′), 7.72 (br s, 1H, NH), 7.57 (br d, J=8.5 Hz, 1H, H-5′), 7.33 (ddd, J=8.6, 7.0, 1.3 Hz, 1H, H-7′), 3.41-3.45 (m, 2H, CH₂N), 2.45-2.50 (m, 2H, CH₂N), 2.20 [s, 6H, N(CH₃)₂]; ¹³C NMR [(CD₃)₂SO] δ 158.8 (C-3′), 148.3 (C-4a′), 135.6 (C-6′), 129.9 (C-8a′), 125.9 (C-5′), 124.4 (C-7′), 119.8 (C-8′), 57.6 (CH₂N), 45.1 [N(CH₃)₂], 38.6 (CH₂N); Anal. calc. for C₁₁H₁₅N₅O; C, 56.6; H, 6.5; N, 30.0; found C, 56.8: H, 6.6; N, 30.4%.

### Example 58

N¹,N¹,N²-Trimethyl-N²-(1-oxido-1,2,4-benzotriazin-3-yl)-1,2-ethanediamine (38).

N¹,N¹,N²-Trimethyl-1,2-ethanediamine (0.45 mL, 3.5 mmol) was added to a stirred solution of chloride 19 (210 mg, 1.2 mmol) in DME (50 mL) and the solution stirred at reflux temperature for 16 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 38 (277 mg, 96%) as a yellow solid which was recrystallized as the hydrochloride salt, mp (MeOH/EtOAc) 220-223° C.; ¹H NMR [(CD₃)₂SO] δ 10.64 (br s, 1H, NH⁺Cl⁻), 8.16 (dd, J=8.7, 1.3 Hz, 1H, H-8′), 7.84 (ddd, J=8.6, 7.1, 1.3 Hz, 1H, H-6′), 7.64 (d, J=8.6 Hz, 1H, H-5′), 7.40 (ddd, J=8.7, 7.1, 1.2 Hz, 1H, H-7′), 4.04 (t, J=6.3 Hz, 2H, CH₂N), 3.37-3.42 (m, 2H, CH₂N), 3.21 (s, 3H, NCH₃), 2.85 [d, J=4.5 Hz, 6H, N(CH₃)₂]; ¹³C NMR [(CD₃)₂SO] δ 158.2 (C-3′), 148.1 (C-4a′), 136.0 (C-6′), 129.4 (C-8a′), 126.2 (C-5′), 125.3 (C-7′), 119.8 (C-8′), 53.5 (CH₂N), 43.7 (NCH₃), 42.5 [N(CH₃)₂], 35.0 (CH₂N); Anal. calc. for C₁₂H₁₈ClN₅O: C, 50.8; H, 6.4; N, 24.7; Cl, 12.5; found C, 51.3: H, 6.7; N, 24.8; Cl, 12.7%.

### Example 59

N¹-(1-Oxido-1,2,4-benzotriazin-3-yl)-N²,N²-dipropyl-1,2-ethanediamine hydrochloride (39). MsCI (125 μL, 1.6 mmol) was added to a stirred solution of alcohol 22 (277 mg, 1.3 mmol) and Et₃N (280 μL, 2.0 mmol) in dry DCM (50 mL) at 5° C. and the solution stirred for 2 h at 20° C. The solution was diluted with DCM (30 mL), washed with water (2×20 mL), the organic fraction dried and the solvent evaporated. The residue was dissolved in DMF (5 mL) and di-n-propylamine (9.2 mL, 67 mmol) added and the solution heated at 50° C. for 2 h. The solvent was evaporated and the residue partitioned between EtOAc (50 mL) and water (50 mL). The organic fraction was extracted with water (2×25 mL), dried and the solvent evaporated. The residue was chromatographed, eluting with a gradient (2-5%) of MeOH/DCM, to give the amine 39 (152 mg, 39%) which was dissolved in HCl saturated MeOH, the solvent evaporated and the residue crystallized as a tan solid, mp (MeOH/EtOAc) 159-161° C.; ¹H NMR [(CD₃)₂SO] δ 10.70 (br s, 1H, NH⁺Cl⁻), 8.17 (dd, J=8.6, 1.0 Hz, 1H, H-8), 8.14 (br s, 1H, NH), 7.84 (ddd, J=8.4, 7.0, 1.3 Hz, 1H, H-6′), 7.59 (d, J=8.4 Hz, 1H, H-5′), 7.40 (ddd, J=8.6, 7.0, 1.3 Hz, 1H, H-7′), 3.74-3.81 (m, 2H, CH₂N), 3.29-3.33 (m, 2H, CH₂N), 3.03-3.13 (m, 4H, 2×CH₂N), 1.70-1.79 (m, 4H, 2×CH₂), 0.93 (t, J=7.3 Hz, 6H, 2×CH₃); ¹³C NMR [(CD₃)₂SO] δ 158.5 (C-3′), 147.8 (C-4a′), 135.9 (C-6′), 130.3 (C-8a′), 126.0 (C-5′), 125.1 (C-7′), 119.8 (C-8′), 53.6 (2×CH₂N), 50.1 (CH₂N), 35.3 (CH₂N), 16.3 (2×CH₂), 10.8 (2×CH₃); Anal. calc. for C₁₅H₂₃N₅0.2HCl; C, 49.7; H, 7.0; N, 19.3; Cl, 19.6; found C, 50.1; H, 7.0; N, 19.4; Cl, 19.4%.

### Example 60

N-[2-(1-Pyrrolidinyl)ethyl]-1,2,4-benzotriazin-3-amin 1-oxid (40). 2-(1-Pyrrolidinyl)ethylamine (1.25 mL, 9.9 mmol) was added to a stirred solution of chloride 19 (599 mg, 3.3 mmol) in DME (50 mL) and the solution stirred at reflux temperature for 4 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 40 (806 mg, 94%) as a yellow solid, mp (DCM) 141-143° C.; ¹H NMR [(CD₃)₂SO] δ 8.13 (dd, J=8.6, 1.0 Hz, 1H, H-8), 7.81 (br s, 1H, NH), 7.78 (ddd, J=8.4, 7.0, 1.0 Hz, 1H, H-6), 7.57 (d, J=8.4 Hz, 1H, H-5), 7.33 (ddd, J=8.6, 7.0, 1.0 Hz, 1H, H-7), 3.42-3.48 (m, 2H, CH₂N), 2.63 (t, J=6.8 Hz, 2H, CH₂N), 2.47-2.53 (m, 4H, 2×CH₂N), 1.64-1.71 (m, 4H, 2×CH₂); ¹³C NMR [(CD₃)₂SO] δ 158.8 (C-3),148.3 (C-4a), 135.6 (C-6),128.0 (C-8a), 126.0 (C-5), 124.4 (C-7), 119.8 (C-8), 54.2 (CH₂N), 53.5 (2×CH₂N), 39.8 (CH₂N), 23.0 (2×CH₂); Anal. calc. for C₁₃H₁₇N₅O; C, 60.2; H, 6.6; N, 27.0; found C, 60.2: H, 6.9; N, 27.3%.

### Example 61

N-[2-(4-Morpholinyl)ethyl]-1,2,4-benzotriazin-3-amine 1-oxide (41). 2-(4-Morpholinyl)ethylamine (1.2 mL, 8.9 mmol) was added to a stirred solution of chloride 19 (541 mg, 3.0 mmol) in DME (50 mL) and the solution stirred at reflux temperature for 4 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 41 (802 mg, 98%) as a yellow solid, mp (DCM) 170-172° C.; ¹H NMR [(CD₃)₂SO] δ 8.13 (dd, J=8.6, 1.2 Hz, 1H, H-8), 7.79 (ddd, J=8.4, 7.0, 1.2 Hz, 1H, H-6), 7.76 (br s, 1H, NH), 7.57 (d, J=8.4 Hz, 1H, H-5), 7.34 (ddd, J=8.6, 7.0, 1.2 Hz, 1H, H-7), 3.55-3.58 (m, 4H, 2×CH₂O), 3.45-3.50 (m, 2H, CH₂N), 2.51-2.56 (m, 2H, CH₂N), 2.41-2.45 (m, 4H, 2×CH₂N);

¹³C NMR [(CD₃)₂SO] δ 158.9 (C-3), 148.3 (C-4a), 135.7 (C-6), 130.0 (C-8a), 126.0 (C-5), 124.5 (C-7), 119.8 (C-8), 66.1 (2×CH₂O),56.8 (CH₂N), 53.2 (2×CH₂N), 37.7 (CH₂N); Anal. calc. for C₁₃H₁₇N₅O₂: C, 56.7; H, 6.2; N, 25.4; found C, 56.5; H, 6.3; N, 25.1%.

### Exampl 62

N-[2-(1-Piperidinyl)ethyl]-1,2,4-benzotriazin-3-amin 1-oxid (42). 2-(1-Piperidinyl)ethylamine (1.2 mL, 8.2 mmol) was added to a stirred solution of chloride 19 (499 mg, 2.7 mmol) in DME (50 mL) and the solution stirred at reflux temperature for 1 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-4%) of MeOH/DCM, to give 1-oxide 42 (644 mg, 86%) as a yellow solid, mp (DCM) 141-144° C.; ¹H NMR δ 8.24 (d, J=8.6, Hz, 1H, H-8), 7.69 (ddd, J=8.4, 7.0, 1.4 Hz, 1H, H-6), 7.58 (d, J=8.4 Hz, 1H, H-5), 7.27 (ddd, J=8.6, 7.0, 1.3 Hz, 1H, H-7), 5.30 (br s, 1H, NH), 3.57-3.64 (m, 2H, CH₂N), 2.63-2.69 (m, 2H, CH₂N), 2.47-2.54 (m, 4H, 2×CH₂N), 1.60-1.68 (m, 4H, 2×CH₂), 1.44-1.50 (m, 2H, CH₂);

¹³C NMR δ 158.8 (C-3), 148.9 (C-4a), 135.5 (C-6), 130.8 (C-8a), 126.4 (C-5), 124.7 (C-7), 120.5 (C-8), 56.8 (CH₂N), 54.3 (2×CH₂N), 37.8 (CH₂N), 25.7 (2×CH₂), 24.2 (CH₂); Anal. calc. for C₁₄H₁₉N₅O: C, 61.5; H, 7.0; N, 25.6; found C, 61.4: H, 7.1; N, 25.6%.

### Example 63

N[2-(2,6-Dimethyl-1-piperidinyl)ethyl]-1,2,4-benzotriazin-3-amine 1-oxide (43). 2-(2,6-Dimethyl-1-piperidinyl)ethylamine (636 mg, 4.1 mmol) was added to a stirred solution of chloride 19 (493 mg, 2.7 mmol) and Et₃N (0.57 mL, 4.1 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 43 (484 mg, 59%) as a yellow solid, mp (MeOH/DCM) 160-163° C.; ¹H NMR δ 8.25 (dd, J=8.6, 1.4 Hz, 1H, H-8), 7.68 (ddd, J=8.5, 7.0, 1.4 Hz, 1H, H-6), 7.57 (dd, J=8.5, 1.3 Hz, 1H, H-5), 7.27 (ddd, J=8.6, 7.0, 1.3 Hz, 1H, H-7), 5.68 (b 2.59 (m, 2H, 2×CH), 1.65-1.70 (m, 1H, CH₂), 1.54-1.59 (m, 2H, CH₂), 1.35-1.40 (m, 1H, CH₂), 1.25-1.33 (m, 2H, CH₂), 1.20 (d, J=6.3 Hz, 6H, 2×CH₃); ¹³C NMR δ 159.0 (C-3), 148.9 (C-4a), 135.4 (C-6), 130.9 (C-8a), 126.5 (C-5), 124.7 (C-7), 120.5 (C-8), 57.3 (2×CH), 47.4 (CH₂N), 39.5 (CH₂N), 34.2 (CH₂), 24.4 (2×CH₂), 21.6 (2×CH₃); Anal. calc. for C₁₆H₂₃N₅O: C, 63.8; H, 7.7; N, 23.2; found C, 63.6; H, 7.6; N, 23.3%.

### Example 64

N¹-(1-Oxido-1,2,4-benzotriazin-3-yl)-N³,N³-dimethyl-1,3-propan diamin (44). N,N-dimethylpropylenediamine (0.9 mL, 6.9 mmol) was added dropwise to a stirred solution of chloride 19 (500 mg, 2.75 mmol) in DME (50 mL) and the solution stirred at reflux temperature for 8 h. The solution was cooled to 20° C., the solvent evaporated and the residue partitioned between aqueous NH₄OH solution (100 mL) and EtOAc (100 mL). The organic fraction was dried, and the solvent evaporated. The residue was chromatographed, eluting with a gradient (0-10%) of MeOH/DCM, to give 1-oxide 44 (629 mg, 92%) as a yellow solid, mp 137-138° C.; ¹H NMR [(CD₃)₂SO] δ 8.13 (dd, J=8.6, 1.1 Hz, 1H, H-8′), 7.92 (br s, 1H, NH), 7.77 (ddd, J=8.4, 7.1, 1.1 Hz, 1H, H-6′), 7.56 (d, J=8.4 Hz, 1H, H-5′), 7.32 (ddd, J=8.6, 7.1, 1.1 Hz, 1H, H-7′), 3.37 (br s, 2H, H-1), 2.30 (t, J=7.0 Hz, 2H, H-3), 2.15 [s, 6H, N(CH₃)₂], 1.70-1.76 (m, 2H, H-2); ¹³C NMR [(CD₃)₂SO] δ 158.8 (C-3′), 148.3 (C-4a′), 135.6 (C-6′), 129.9 (C-8a′), 125.9 (C-5′), 124.3 (C-7′), 119.8 (C-8′), 56.6 (CH₂N), 45.1 [N(CH₃)₂], 39.0 (CH₂N), 26.3 (CH₂); Anal. calc. for C₁₂H₁₇N₅O: C, 58.3; H, 6.9; N, 28.3; found C, 58.3; H, 7.0; N, 28.5%.

### Example 65

N-Phenyl-1,2,4-benzotriazin-3-amine 1-oxide (45). Two drops of cHCl were added to a solution of chloride 19 (0.52 g, 2.86 mmol) and aniline (0.78 mL, 8.59 mmol) in DME (10 mL) and the solution stirred at reflux temperature for 16 h. The solvent was evaporated and the residue chromatographed, eluting with 10% EtOAc/pet. ether, to give 1-oxide 45 (334 mg, 49%) as a yellow powder, mp 197-198.5° C. [lit. (Pazdera & Potacek, Chem. Papers, 1989, 43, 107) mp 199-201° C.]; ¹H NMR δ 8.32 (d, J=9.0 Hz, 1H, H-8), 7.70-7.77 (m, 4H, H-5, H-6, H-2′, H-6′), 7.37-7.42 (m, 3H, H-7, H-3′, H-5′), 7.22 (br s, 1H, NH), 7.13 (dt, J=7.5, 0.9 Hz, 1H, H-4′); ¹³C NMR δ 156.3 (C-3), 148.1 (C-4a), 138.1 (C-1′), 135.8 (C-6), 131.6 (C-8a), 129.1 (C-3′, C-5′), 127.1 (C-5), 126.1 (C-4′), 123.8 (C-7), 120.4 (C-8), 119.7 (C-2′, C-6′).

### Example 66

N[3-(2-Methoxyethyl)phenyl]-1,2,4-benzotriazin-3-amine 1-oxide (49).

1-(2-Methoxyethyl)-3-nitrobenzene (47). A solution of 3-nitrophenethyl alcohol (46) (1.05 g, 6.3 mmol) in THF (10 mL) was addded dropwise to a stirred suspension of NaH (325 mg, 8.1 mmol) in THF (30 mL) at 5° C. and the mixture warmed to 20° C. and stirred 30 min. Iodomethane (3.9 mL, 62.5 mmol) was added and the mixture stirred at 20° C. for 16 h. The solvent was evaporated and the residue partitioned between EtOAc (100 mL) and water (100 mL). The organic fraction was washed with water (2×30 mL), brine (30 mL), dried, and the solvent evaporated. The residue was chromatographed, eluting with 20% EtOAc/pet. ether, to give ether 47 (981 mg, 87%) as a clear oil, (Norman & Radda, J. Chem. Soc. 1961, 3030) ¹H NMR δ 8.06-8.11 (m, 2H, H-2, H-4), 7.57 (d, J=7.6 Hz, 1H, H-6), 7.47 (dd, J=7.9, 7.6 Hz, 1H, H-5), 3.65 (t, J=6.5 Hz, 2H, CH₂O), 3.36 (s, 3H, OCH₃), 2.98 (t, J=6.5 Hz, 2H, CH₂);

¹³C NMR δ 148.3 (C-3), 141.3 (C-1), 135.2 (C-6), 129.2 (C-5), 123.7 (C-2), 121.4 (C-4), 72.5 (CH₂O), 58.8 (OCH₃), 35.8 (CH₂).

3-(2-Methoxyethyl)aniline (48). A solution of ether 47 (928 mg, 5.1 mmol) in EtOH (50 mL) with Pd/C (100 mg) was stirred under H₂ (60 psi) for 2 h. The mixture was filtered through celite, washed with EtOH (2×10 mL) and the solvent evaporated to give aniline 48 (718 mg, 93%) as a pale pink oil, ¹H NMR δ 7.08 (dd, J=7.7, 7.3 Hz, 1H, H-5), 6.62 (br d, J=7.3 Hz, 1H, H-4), 6.51-6.55 (m, 2H, H-2, H-6), 3.50 (br s, 2H, NH₂), 3.58 (t, J=7.2 Hz, 2H, CH₂O), 3.35 (s, 3H, OCH₃), 2.80 (t, J=7.2 Hz, 2H, CH₂); ¹³C NMR δ 146.4 (C-1), 140.1 (C-3), 129.3 (C-5), 119.1 (C-4), 115.7 (C-2), 113.1 (C-6), 73.6 (CH₂O), 58.6 (OCH₃), 36.2 (CH₂); MS (EI⁺) m/z 151 (M⁺, 90%), 136 (20), 106 (100); HRMS (EI⁺) calc. for C₉H₁₃NO (M⁺) m/z 151.0997, found 151.0995.

N-[3-(2-Methoxyethyl)phenyl]-1,2,4-benzotriazin-3-amine 1-oxide (49). A solution of chloride 19 (376 mg, 2.07 mmol) and aniline 48 (688 mg, 4.55 mmol) in DMSO (20 mL) was heated at 100° C. for 16 h. The solution was partitioned between EtOAc (100 mL) and water (100 mL), the organic fraction washed with water (2×50 mL), brine (50 mL), dried, and the solvent evaporated. The residue was chromatographed, eluting with a gradient (20-50%) of EtOAc/pet. ether, to give 1-oxide 49 (590 mg, 96%) as an orange powder, mp (EtOAc/Et₂O) 122-124° C.; ¹H NMR [(CD₃)₂SO] δ 10.18 (s, 1H, NH), 8.22 (dd, J=8.6, 1.0 Hz, 1H, H-8), 7.87 (ddd, J=8.5, 7.1, 1.3 Hz, 1H, H-6), 7.70-7.76 (m, 3H, H-5, H-2′, H-6′), 7.47 (ddd, J=8.6, 7.1, 1.3 Hz, 1H, H-7), 7.27 (dd, J=7.9, 7.8 Hz, 1H, H-5′), 6.94 (d, J=7.8 Hz, 1H, H-4′), 3.58 (t, J=6.8 Hz, 2H, CH₂O), 3.27 (s, 3H, OCH₃), 2.82 (t, J=6.8 Hz, 2H, CH₂); ¹³C NMR [(CD₃)₂SO] δ 156.3 (C-3′), 147.5 (C-3), 139.5 (C-1′), 139.1 (C-4a), 135.9 (C-6), 130.9 (C-8a), 128.4 (C-5′), 126.6 (C-5), 125.8 (C-4′), 123.2 (C-7), 119.8 (C-8), 119.7 (C-2′), 117.3 (C-6′), 72.6 (CH₂O), 57.8 (OCH₃), 35.5 (CH₂); Anal. calc. for C₁₆H₁₆N₄O₂: C, 64.9; H, 5.4; N, 18.9; found C, 65.0; H, 5.5; N, 19.2%.

### Example 67

M thyl {4-[(1-oxido-1,2,4-benzotriazin-3-yl)amino]phenyl}ac tat (51). A solution of chloride 19 (992 mg, 5.5 mmol) and aniline 50 (1.99 g, 12.0 mmol) in DMSO (30 mL) was heated at 100° C. for 6 h and then 20° C. for 16 h. The solution was partitioned between EtOAc (200 mL) and water (200 mL), the organic fraction washed with water (2×100 mL), dried, and the solvent evaporated. The residue was chromatographed, eluting with 10% EtOAc/DCM, to give the 1-oxide 51 (1.05 g, 61%) as a yellow solid, mp (EtOAc/DCM) 216-218° C.; ¹H NMR [(CD₃)₂SO] δ 10.00 (s, 1H, NH), 8.24 (d, J=8.3 Hz, 1H, H-8″), 7.82 (d, J=8.4 Hz, 2H, H-2′, H-6′), 7.79 (dd, J=8.2, 7.3 Hz, 1H, H-6″), 7.70 (d, J=8.2 Hz, 1H, H-5″), 7.40 (dd, J=8.3, 7.3 Hz, 1H, H-7″), 7.22 (d, J=8.4 Hz, 2H, H-3′, H-5′), 3.67 (s, 3H, OCH₃), 3.60 (s, 2H, H-2); ¹³C NMR [(CD₃)₂SO] δ 171.7 (C-1), 156.3 (C-3″), 147.9 (C-4a″), 138.1 (C-4′), 135.6 (C-6″), 131.9 (C-8a″), 129.4 (C-2′, C-6′), 128.3 (C-1′), 126.7 (C-5″), 125.5 (C-7″), 119.9 (C-8″), 119.7 (C-3′, C-5′), 51.7 (OCH₃), 40.1 (C-2); Anal. calc. for C₁₆H₁₄N₄O₃: C, 61.9; H, 4.6; N, 18.1; found C, 62.3; H, 4.8; N, 18.1%.

### Example 68

{4-[(1-Oxido-1,2,4-benzotriazin-3-yl)amino]phenyl}acetic acid (52). A solution of NaOH (1 M, 5.2 mL, 5.2 mmol) was added to a stirred suspension of ester 51 (323 mg, 1.0 mmol) in MeOH (30 mL) and the mixture stirred at 20° C. for 2 h. The volume was reduced to ca. 10 mL and the remaining solution washed with Et₂O (2×10 mL). The solution was adjusted to pH 1 with 2 M HCl and the suspension extracted with EtOAc (3×50 mL), the combined organic fraction dried, and the solvent evaporated. Recrystallization gave the acid 52 (306 mg, 99%) as a yellow powder, mp (EtOAc) 243-245° C.; ¹H NMR [(CD₃)₂SO] δ 12.27 (s, 1H, CO₂H), 10.21 (s, 1H, NH), 8.22 (dd, J=8.6, 0.8 Hz, 1H, H-8″), 7.88 (ddd, J=8.4, 7.1, 1.0 Hz, 1H, H-6″), 7.79 (d, J=8.5 Hz, 2H, H-2′, H-6′), 7.75 (d, J=8.4 Hz, 1H, H-5″), 7.46 (ddd, J=8.6, 7.1, 1.0 Hz, 1H, H-7″), 7.25 (d, J=8.5 Hz, 2H, H-3′, H-5′), 3.54 (s, 2H, H-2); ¹³C NMR [(CD₃)₂SO] δ 172.7 (C-1), 156.3 (C-3″), 147.6 (C-4a″), 137.7 (C-4′), 135.9 (C-6″), 130.9 (C-8a″), 129.5 (C-2′, C-6′), 129.2 (C-1′), 126.5 (C-5″), 125.8 (C-7″), 119.8 (C-8″), 119.3 (C-3′, C-5′), 40.0 (C-2); Anal. calc. for C₁₅H₁₂N₄O₃: C, 60.8; H, 4.1; N, 18.9; found C, 61.0; H, 4.0; N, 19.1%.

### Example 69

2-{4-[(1-Oxido-1,2,4-benzotriazin-3-yl)amino]phenyl}-N-(2-methoxyethyl)acetamide (53). A solution of acid 52 (259 mg, 0.87 mmol) and CDI (213 mg, 1.3 mmol) in DMF (10 mL) was stirred at 50° C. for 10 min. 2-Methoxyethylamine (152 μL, 1.75 mmol) was added dropwise and the solution stirred at 20° C. for 16 h. The solvent was evaporated and the residue suspended in water (100 mL). The precipitate was filtered, dried, and recrystallized from MeOH to give amide 53 (239 mg, 78%) as a yellow powder, mp (MeOH) 216-218° C.; ¹H NMR [(CD₃)₂SO] δ 10.19 (s, 1H, NH), 8.21 (dd, J=8.6, 1.0 Hz, 1H, H-8″), 8.09 (dd, J=5.6, 5.3 Hz, 1H, CONH), 7.88 (ddd, J=8.3, 7.1, 1.0 Hz, 1H, H-6″), 7.73-7.78 (m, 3H, H-2′, H-6′, H-5″), 7.47 (ddd, J=8.6, 7.1, 1.0 Hz, 1H, H-7″), 7.25 (d, J=8.5 Hz, 2H, H-3′, H-5′), 3.40 (br s, 2H, H-2), 3.32-3.37 (m, 2H, CH₂O), 3.25 (s, 3H, OCH₃), 3.23 (q, J=5.7 Hz, 2H, CH₂N); ¹³C NMR [(CD₃)₂SO] δ 170.3 (C-1), 156.4 (C-3″), 147.6 (C-4a″), 137.5 (C-4′), 135.9 (C-6″), 130.9 (C-8a″), 130.7 (C-1′), 129.1 (C-2′, C-6′), 128.5 (C-5″), 125.8 (C-7″), 119.8 (C-8″), 119.4 (C-3′, C-5′), 70.6 (CH₂O), 57.8 (OCH₃), 41.6 (CH₂N), 38.4 (C-2); Anal. calc. for C₁₈H₁₉N₅O₃: C, 61.2; H, 5.4; N, 19.8; found C, 61.6; H, 5.3; N, 19.9%.

### Example 70

N-[2-(Dimethylamino)ethyl]-2-{4-[(1-oxido-1,2,4-benzotriazin-3-yl)amino]phenyl}acetamide (54). A solution of acid 52 (476 mg, 1.6 mmol) and CDI (391 mg, 2.4 mmol) in DMF (10 mL) was stirred at 50° C. for 10 min. N,N-Dimethylaminoethylamine (353 μL, 3.2 mmol) was added dropwise and the solution stirred at 20° C. for 16 h. The solvent was evaporated and the residue suspended in EtOAc (200 mL). The precipitate was filtered and dried. The mother liquor was evaporated and the residue suspended in water (50 mL), the precipitate filtered, and combined with the previous crop to give amide 54 (562 mg, 95%) as a yellow powder, mp (EtOAc) 225-226° C.; ¹H NMR [(CD₃)₂SO] δ 10.18 (s, 1H, NH), 8.22 (dd, J=8.6, 1.0 Hz, 1H, H-8″), 7.92 (t, J=5.4 Hz, 1H, CONH), 7.87 (ddd, J=8.4, 7.1, 1.0 Hz, 1H, H-6″), 7.72-7.78 (m, 3H, H-2′, H-6′, H-5″), 7.46 (ddd, J=8.6, 7.1, 1.0 Hz, 1H, H-7″), 7.24 (d, J=8.5 Hz, 2H, H-3′, H-5′), 3.16 (dt, J=6.7, 5.4 Hz, 2H, CH₂N), 2.28 (t, J=6.7 Hz, 2H, CH₂N), 2.15 [s, 6H, N(CH₃)₂]; ¹³C NMR [(CD₃)₂SO) δ 170.1 (C-1), 156.4 (C-3″), 147.6 (C-4a″), 137.5 (C-4′), 135.7 (C-6″), 130.9 (C-8a″), 130.8 (C-1′), 129.1 (C-2′, C-6′), 128.5 (C-5″), 125.8 (C-7″), 119.8 (C-8″), 119.4 (C-3′, C-5′), 58.2 (CH₂N), 45.1 [N(CH₃)₂], 41.7 (CH₂N), 36.8 (C-2); Anal. calc. for C₁₉H₂₂N₆O₂: C, 62.3; H, 6.1; N, 22.9; found C, 62.6; H, 6.2; N, 22.7%.

### Example 71

3-M thyl-1,2,4-benzotriazin 1-oxide (55). Pd(PPh₃)₄ (82 mg, 71 μmol) was added to a stirred, degassed solution of chloride 19 (258 mg, 1.42 mmol) and Me₄Sn (0.39 mL, 2.8 mmol) in DME (20 mL) and the solution stirred under N₂ at reflux temperature for 48 h. The solvent was evaporated and the residue chromatographed, eluting with 20% EtOAc/pet. ether, to give an oil which was chromatographed, eluting with 5% EtOAc/DCM, to give (i) starting material 19 (164 mg, 64%) and (ii) 1-oxide 55 (55 mg, 24%) as a white solid, mp (EtOAc/pet. ether) 99-101° C. [lit. (Atallah & Nazar, Tetrahedron Lett., 1982, 38, 1793) mp (benzene/pet.ether) 101-102° C.]; ¹H NMR δ 8.44 (d, J=8.6 Hz, 1H, H-8), 7.90-7.97 (m, 2H, H-5, H-6), 7.70 (ddd, J=8.6, 6.8, 1.8 Hz, 1H, H-7), 2.80 (s, 3H, CH₃).

### Example 72

3-Ethyl-1,2,4-benzotriazine 1-oxide (56). Pd(PPh₃)₄ (340 mg, 0.30 mmol) was added to a stirred, degassed solution of chloride 19 (539 mg, 2.97 mmol) and Et₄Sn (0.54 mL, 2.7 mmol) in DME (20 mL) and the solution stirred under N₂ at reflux temperature for 4 h. The solvent was evaporated and the residue chromatographed, eluting with 20% EtOAc/pet. Ether, to give an oil which was chromatographed, eluting with 5% EtOAc/DCM, to give 1-oxide 56 (448 mg, 86%) as a white solid, mp (EtOAc/pet. ether) 78-80° C.; ¹H NMR δ 8.45 (dd, J=8.7, 1.1 Hz, 1H, H-8), 7.99 (dd, J=8.5, 1.1 Hz, 1H, H-5), 7.93 (ddd, J=8.5, 7.1, 1.3 Hz, 1H, H-6), 7.69 (ddd, J=8.7, 7.1, 1.2 Hz, 1H, H-7), 3.06 (q, J=7.6 Hz, 2H, CH₂), 1.45 (t, J=7.6 Hz, 3H, CH₃); ¹³C NMR δ 168.1 (C-3), 147.6 (C-4a), 135.5 (C-6), 133.2 (C-8a), 129.8 (C-5), 128.7 (C-7), 120.1 (C-8), 30.7 (CH₂), 12.2 (CH₃); Anal. calc. for C₉H₉N₃O₃: C, 61.7; H, 5.2; N, 24.0; found C, 62.0; H, 5.0; N, 24.6%.

### Example 73

3-Phenyl-1,2,4-benzotriazine 1-oxide (57). Pd(PPh₃)₄ (314 mg, 0.27 mmol) was added to a stirred, degassed solution of chloride 19 (986 mg, 5.43 mmol) and phenylboronic acid (0.73 g, 5.97 mmol) in DME (50 mL) and Cs₂CO₃ (5.3 g, 16.3 mmol) in water (10 mL) and the mixture stirred under N₂ at reflux temperature for 2 h. The mixture was partitioned between EtOAc (100 mL) and water (100 mL), the organic fraction washed with water (2×50 mL), dried, and the solvent was evaporated. The residue was chromatographed, eluting with a gradient (20-50%) of EtOAc/pet. ether, to give an oil which was chromatographed, eluting with 5% EtOAc/DCM, to give 1-oxide 57 (743 mg, 61%) as a white solid, mp (EtOAc/pet. ether) 125-127° C.; ¹H NMR δ 8.49-8.54 (m, 3H, H-8, H-2′, H-6′), 8.09 (d, J=8.6 Hz, 1H, H-5), 7.94 (ddd, J=8.6, 7.1, 1.4 Hz, 1H, H-6), 7.70 (ddd, J=8.7, 7.1, 1.4 Hz, 1H, H-7), 7.51-7.57 (m, 3H, H-3′, H-4′, H-5′); ¹³C NMR δ 160.7 (C-3), 147.7 (C-4a), 135.6 (C-6), 134.1 (C-1′), 133.5 (C-8a), 131.9 (C-5), 130.5 (C-7), 129.4 (C-4′), 128.8 (C-2′, C-6′), 128.5 (C-3′, C-5′), 120.3 (C-8); Anal. calc. for C₁₃H₉N₃O: C, 69.9; H, 4.1; N, 18.8; found C, 69.9; H, 4.0; N, 18.7%.

### Example 74

3-(4-Methoxyphenyl)-1,2,4-benzotriazine 1-oxide (58). Pd(PPh₃)₄ (162 mg, 0.14 mmol) was added to a stirred, degassed solution of chloride 19 (510 mg, 2.8 mmol) and 4-methoxyphenylboronic acid (0.47 g, 3.1 mmol) in DME (50 mL) and Cs₂CO₃ (3.0 g, 8.4 mmol) in water (8 mL) and the mixture stirred under N₂ at reflux temperature for 2 h. The mixture was partitioned between EtOAc (100 mL) and water (100 mL), the organic fraction washed with water (2×50 mL), dried, and the solvent was evaporated. The residue was chromatographed, eluting with a gradient (20-50%) of EtOAc/pet. ether, to give an oil which was chromatographed, eluting with 5% EtOAc/DCM, to give 1-oxide 58 (408 mg, 57%) as a white solid, mp (EtOAc/pet. ether) 168-170° C.; ¹H NMR δ 8.44-8.49 (m, 3H, H-8, H-2′, H-6′), 8.02 (d, J=8.7 Hz, 1H, H-5), 7.90 (ddd, J=8.7, 7.2, 1.4 Hz, 1H, H-6), 7.64 (ddd, J=8.5, 7.2, 1.4 Hz, 1H, H-7), 7.02 (ddd, J=9.0, 2.9, 2.1 Hz, 2H, H-3′, H-5′), 3.90 (s, 3H, OCH₃); ¹³C NMR δ 162.8 (C-4′), 160.5 (C-3), 147.8 (C-4a), 135.5 (C-6), 133.2 (C-8a), 130.3 (C-3′, C5′), 129.5 (C-5), 129.1 (C-7), 126.5 (C-1′), 120.3 (C-8), 114.3 (C-2′, C-6′), 55.4 (OCH₃); Anal. calc. for C₁₄H₁₁N₃O₂: C, 66.4; H, 4.4; N, 16.6; found C, 66.5; H, 4.4; N, 16.7%.

### Example 75

3-Vinyl-1,2,4-benzotriazine 1-oxide (59). Pd(PPh₃)₄ (204 mg, 0.18 mmol) was added to a stirred solution of chloride 19 (320 mg, 1.8 mmol) and vinyltributyltin (0.77 mL, 2.6 mmol) in DME (20 mL), the solution degassed and stirred under N₂ at reflux temperature for 6 h. The solvent was evaporated and the residue chromatographed, eluting with 20% EtOAc/pet. ether to give an oil which was chromatographed, eluting with 5% EtOAc/DCM, to give 1-oxide 59 (177 mg, 58%) as a white solid, mp (EtOAc/pet. ether) 85-86° C.; ¹H NMR δ 8.46 (dd, J=8.9, 1.4 Hz, 1H, H-8), 8.10 (d, J=8.5 Hz, 1H, H-5), 7.92 (ddd, J=8.5, 7.1, 1.4 Hz, 1H, H-6), 7.69 (ddd, J=8.9, 7.1, 1.4 Hz, 1H, H-7), 6.86 (dd, J=17.4, 9.4 Hz, 2H, H-1′), 6.79 (dd, J=17.4, 2.2 Hz, 1 H, H-2′), 5.92 (dd, J=9.4, 2.2 Hz, 1H, H-2′); ¹³C NMR δ 160.2 (C-3), 147.4 (C-4a), 135.6 (C-6), 133.6 (C-8a), 133.0 (C-2′), 130.2 (C-5), 129.1 (C-7), 126.6 (C-1′), 120.2 (C-8); Anal. calc. for C₉H₇N₃O: C, 62.4; H, 4.1; N, 24.3; found C, 61.8; H, 4.0; N, 24.4%.

### Example 76

3-Allyl-1,2,4-benzotriazine 1-oxide (60). Pd(PPh₃)₄ (340 mg, 0.29 mmol) was added to a stirred solution of chloride 19 (1.1 g, 5.9 mmol) and allyltributyltin (2.0 mL, 6.5 mmol) in DME (60 mL), the solution degassed and stirred under N₂ at reflux temperature for 6 h. The solvent was evaporated and the residue chromatographed, eluting with 20% EtOAc/pet. ether, to give an oil which was chromatographed, eluting with 5% EtOAc/DCM, to give 1-oxide 60 (1.00 g, 90%) as a white solid, mp (EtOAc/pet. ether) 57-58° C.; ¹H NMR δ 8.45 (dd, J=8.6, 1.4 Hz, 1H, H-8), 8.10 (dd, J=8.4, 1.4 Hz, 1H, H-5), 7.94 (ddd, J=8.4, 7.1, 1.4 Hz, 1H, H-6), 7.70 (ddd, J=8.6, 7.1, 1.4 Hz, 1H, H-7), 6.15-6.24 (m, 1H, H-2′), 5.31 (dq, J=17.0, 1.5 Hz, 1H, H-3′), 5.24 (dq, J=10.1, 1.5 Hz, 1H, H-3′), 3.80 (dq, J=6.8, 1.5 Hz, 2H, H-1′); ¹³C NMR δ 165.2 (C-3), 147.5 (C-4a), 135.6 (C-6), 133.3 (C-8a), 132.7 (C-2′), 130.1 (C-5), 128.8 (C-7), 120.8 (C-8), 118.5 (C-3′), 41.8 (C-1′); Anal. calc. for C₁₀H₉N₃O: C, 64.2; H, 4.9; N, 22.5; found C, 63.9; H, 4.9; N, 22.7%.

### Example 77

3-(2-Hydroxyethyl)-1,2,4-benzotriazine 1-oxide (61). Ozone was bubbled into a solution of 60 (548 mg, 2.9 mmol) in DCM/MeOH (1:1, 50 mL) at −78° C. until a blue colour persisted. The solution was purged with N₂ to remove excess ozone and a solution of NaBH₄ (111 mg, 2.9 mmol) in EtOH (10 mL) added dropwise and the solution allowed to warm to 20° C. over 1 h. HOAc (1 mL) was added and the solution stirred at 20° C. for 30 min. The solvent was evaporated and the residue partitioned between DCM (50 mL) and water (3×50 mL). The organic fraction was dried and the solvent evaporated. The residue was chromatographed, eluting with 50% EtOAc/pet. ether, to give alcohol 61 (392 mg, 70%) as pale yellow needles, mp 105-107° C.; ¹H NMR δ 8.45 (dd, J=8.6, 1.3 Hz, 1H, H-8), 7.99 (dd, J=8.5, 1.6 Hz, 1H, H-5), 7.96 (ddd, J=8.5, 6.7, 1.3 Hz, 1H, H-6), 7.72 (ddd, J=8.5, 6.7, 1.6 Hz, 1H, H-7), 4.18-4.20 (m, 2H, CH₂O), 3.29 (t, J=5.6 Hz, 2H, CH₂), 3.11 (t, J=5.5 Hz, 1H, OH); ¹³C NMR δ 165.4 (C-3), 147.0 (C-4a), 135.8 (C-6), 133.6 (C-8a), 130.2 (C-5), 128.6 (C-7), 120.1 (C-8), 60.0 (CH₂O), 39.0 (CH₂); Anal. calc. for C₉H₉N₃O₂: C, 56.5; H, 4.7; N, 22.0; found C, 56.8; H, 4.7; N, 21.8%.

### Example 78

3-(2-Oxiranylmethyl)-1,2,4-benzotriazine 1-oxide (62). MCPBA (0.96 g, 3.9 mmol) was added to a stirred solution of alkene 60 (484 mg, 2.6 mmol) in DCM (50 mL) at 20° C. and the mixture stirred for 16 h. The solution was diluted with DCM (100 mL), washed with dilute aqeous NH₃ (3×50 mL). The organic fraction was dried and the solvent evaporated. The residue was chromatographed, eluting with 50% EtOAc/pet. ether, to give (i) starting material 60 (173 mg, 36%) and (ii) epoxide 62 (251 mg, 48%) as white crystals, mp (EtOAc/pet. ether) 105-107° C.; ¹H NMR δ 8.46 (dd, J=8.7, 1.2 Hz, 1H, H-8), 8.03 (dd, J=8.5, 1.1 Hz, 1H, H-5), 7.95 (ddd, J=8.5, 7.1, 1.2 Hz, 1H, H-6), 7.73 (ddd, J=8.7, 7.1, 1.1 Hz, 1H, H-7), 3.55-3.60 (m, 1H, H-2′), 3.27 (dd, J=5.8, 1.9 Hz, 2H, H-1′), 2.93 (dd, J=4.7, 4.1 Hz, 1H, H-3′), 2.76 (dd, J=4.7, 2.6 Hz, 1H, H-3′); ¹³C NMR δ 163.4 (C-3), 147.4 (C-4a), 135.7 (C-6), 133.6 (C-8a), 130.4 (C-5), 128.9 (C-7), 120.1 (C-8), 50.0 (C-2′), 47.1 (C-3′), 40.5 (C-1′); Anal. calc. for C₁₀H₉N₃O₂: C, 59.1; H, 4.5; N, 20.7; found C, 59.2; H, 4.6; N, 20.4%.

### Example 79

3-(2-Methoxyethyl)-1,2,4-benzotriazine 1-oxide (63). Five aliquots of TMSCH₂N₂ (3 mL, 6.0 mmol) were added to a stirred solution of alcohol 61 (1.14 g, 6.0 mmol) and HBF₄ (1.5 mL, 12 mmol) in DCM (50 mL) over 3 h. The solution was stirred at 20° C. for 16 h, the solvent evaporated and the residue chromatographed, eluting with 30% EtOAc/pet. ether, to give (i) methyl ether 63 (375 mg, 30%) as a yellow powder, mp (EtOAC/pet. ether) 56-58° C.; ¹H NMR δ 8.45 (dd, J=8.7, 1.2, 1H, H-8), 8.03 (d, J=8.4 Hz, 1H, H-5), 7.92 (ddd, J=8.4, 7.0, 1.2 Hz, 1H, H-6), 7.71 (ddd, J=8.7, 7.0, 1.1 Hz, 1H, H-7), 3.97 (dd, J=6.5, 6.3 Hz, 2H, CH₂O), 3.38 (s, 3H, OCH₃), 3.31 (dd, J=6.5, 6.3 Hz, 2H, CH₂); ¹³C NMR δ 164.7 (C-3), 147.3 (C-4a), 135.6 (C-6), 130.3 (C-8a), 130.1 (C-5), 128.7 (C-7), 120.1 (C-8), 70.0 (CH₂O), 58.8 (OCH₃), 37.6 (CH₂); Anal. calc. for C₁₀H₁₁N₃O₂: C, 58.5; H, 5.4; N, 20.5; found: C, 58.8; H, 5.4; N, 20.6%; and (ii) starting material 61 (334 mg, 24%), spectroscopically identical to sample prepared above.

### Example 80

2-(1-Oxido-1,2,4-benzotriazin-3-yl)-N,N-dimethylethanamine (64). MsCI (246 μL, 3.1 mmol) was added to a stirred solution of alcohol 61 (496 mg, 2.6 mmol) and Et₃N (470 μL, 3.4 mmol) in dry DCM (50 mL) at 20° C. and the solution stirred for 2 h. The solution was diluted with DCM (50 mL), washed with water (2×10 mL), brine (20 mL), the organic fraction dried and the solvent evaporated. The residue was dissolved in THF (50 mL) and Et₃N (9.0 mL, 64.9 mmol) and dimethylamine hydrochloride (5.3 g, 64.9 mmol) added and the solution heated at reflux temperature for 3 h, then stirred at 20° C. for 16 h. The solvent was evaporated and the residue partitioned between EtOAc (100 mL) and water (100 mL). The organic fraction was extracted with water (2×25 mL), dried and the solvent evaporated. The residue was chromatographed, eluting with a gradient (0-20%) of MeOH/EtOAc then 1% Et₃N/20% MeOH/EtOAc, to give amine 64 (528 mg, 93%) as a yellow/orange solid, mp (MeOH/EtOAc) 47-49° C., ¹H NMR [(CD₃)₂SO] δ 8.36 (d, J=8.5 Hz, 1H, H-8′), 8.02-8.10 (m, 2H, H-5′, H-6′), 7.83 (ddd, J=8.5, 6.7, 1.6 Hz, 1H, H-7′), 3.09 (dd, J=7.5, 7.2 Hz, 2H, H-1), 2.81 (d, J=7.5, 7.2 Hz, 2H, H-2), 2.22 [s, 6H, N(CH₃)₂]; ¹³C NMR [(CD₃)₂SO] δ 165.1 (C-3′), 146.8 (C-4a′), 136.1 (C-6′), 132.7 (C-8a′), 130.5 (C-5′), 128.3 (C-7′), 119.5 (C-8′), 56.9 (C-1), 44.7 [N(CH₃)₂], 34.6 (C-1); Anal. calc. for C₁₁H₁₄N₄O: C, 60.5; H, 6.5; N, 25.7; found C, 60.7; H, 6.7; N, 25.6%.

### Example 81

3-[2-(4-Morpholinyl)ethyl]-1,2,4-benzotriazine 1-oxide hydrochloride (65). MsCl (381 μL, 4.8 mmol) was added to a stirred solution of alcohol 61 (769 mg, 4.0 mmol) and Et₃N (729 μL, 5.2 mmol) in dry DCM (50 mL) at 20° C. and the solution stirred for 2 h. The solution was diluted with DCM (50 mL), washed with water (2×30 mL), brine (50 mL), the organic fraction dried and the solvent evaporated. The residue was dissolved in THF (50 mL) and morpholine (8.8 mL, 100 mmol) added and the solution heated at reflux temperature for 3 h, then stirred at 20° C. for 16 h. The solvent was evaporated and the residue partitioned between EtOAc (100 mL) and water (100 mL). The aqueous fraction was extracted with EtOAc (3×50 mL), the combined organic fraction dried and the solvent evaporated. The residue was chromatographed, eluting with a gradient (0-10%) of MeOH/EtOAc, to give the morpholide 65 (840 mg, 80%) which was dissolved in HCl saturated MeOH, the solvent evaporated and the residue crystallized as an yellow/orange solid, mp (MeOH/EtOAc) 213-215° C.; ¹H NMR [(CD₃)₂SO] δ 11.37 (br s, 1H, NH⁺Cl⁻), 8.40 (d, J=8.7 Hz, 1H, H-8), 8.13 (ddd, J=8.5, 6.9, 1.2 Hz, 1H, H-6), 8.06 (dd, J=8.5, 1.2 Hz, 1H, H-5), 7.87 (ddd, J=8.7, 6.9, 1.2 Hz, 1H, H-7), 3.96-4.01 (m, 2H, CH₂O), 3.80-3.86 (m, 2H, CH₂O), 3.63-3.68 (m, 2H, CH₂N), 3.52-3.58 (m, 4H, 2×CH₂N), 3.15-3.25 (m, 2H, CH₂); Anal. calc. for C₁₃H₁₇ClN₄O₂: C, 52.6; H, 5.8; N, 18.9; Cl, 12.0; found C, 52.6; H, 5.5; N, 18.9; Cl, 12.0%.

### Example 82

3-(3-Hydroxypropyl)-1,2,4-benzotriazine 1-oxid (66). A solution of 9-BBN in THF (13.7 mL, 6.8 mmol) was added to a stirred solution of alkene 60 (1.07 g, 5.7 mmol) in THF (50 mL) and the solution stirred at 20° C. for 1 h. A solution of NaOH (3 M; 2.9 ml, 8.5 mmol), followed by 35% H₂O₂ (2.6 mL, 25.6 mmol) were carefully added and the mixture stirred at 20° C. for 1 h. The mixture was diluted with brine (100 mL), extracted with EtOAc (3×100 mL), the combined organic fraction dried, and the solvent evaporated. The residue was chromatographed, eluting with a gradient (10-50%) of EtOAc/DCM, to give alcohol 66 (1.02 g, 87%) as a white solid, mp (EtOAc/pet. ether) 99-100° C.; ¹H NMR δ 8.46 (dd, J=8.7, 1.0 Hz, 1H, H-8), 7.99 (dd, J=8.5, 1.2 Hz, 1H, H-5), 7.93 (ddd, J=8.5, 7.0, 1.0 Hz, 1H, H-6), 7.70 (ddd, J=8.7, 7.0, 1.2 Hz, 1H, H-7), 3.80 (t, J=6.1 Hz, 2H, CH₂O), 3.18 (t, J=7.3 Hz, 2H, CH₂), 2.15-2.22 (m, 2H, CH₂), (OH not observed); ¹³C NMR δ 166.9, 147.3, 135.7, 133.3, 130.1, 128.6, 120.1, 62.1, 34.1, 30.5; Anal. calc. for C₁₀H₁₁N₃O₂: C, 58.5; H, 5.4; N, 20.5; found C, 58.6; H, 5.5; N, 20.5%.

### Example 83

3-[3-Methoxypropyl]-1,2,4-benzotriazine 1-oxide (67). TMSCH₂N₂ (1.1 mL, 2.1 mmol) was added to a stirred solution of alcohol 66 (437 mg, 2.1 mmol) and HBF₄ (0.53 mL, 4.3 mmol) in DCM (20 mL) at 20° C. and the solution stirred for 2 h at 20° C. More TMSCH₂N₂ (5×1.1 mL) was added at hourly intervals and the solution stirred vigorously for 16 h. The solvent was evaporated and the residue chromatographed, eluting with a gradient (20-35%) of EtOAc/DCM, to give methyl ether 67 (310 mg, 66%) as a tan oil, ¹H NMR δ 8.45 (dd, J=8.7, 1.1 Hz, 1H, H-8), 8.03 (d, J=8.5 Hz, 1H, H-5), 7.94 (ddd, J=8.5, 7.0, 1.1 Hz, 1H, H-6), 7.70 (ddd, J=8.7, 7.0, 1.1 Hz, 1H, H-7), 3.52 (t, J=6.3 Hz, 2H, CH₂O), 3.33 (s, 3H, OCH₃), 3.10-3.14 (m, 2H, CH₂N), 2.16-2.23 (m, 2H, CH₂); ¹³C NMR δ 166.8 (C-3), 147.0 (C-4a), 135.7 (C-6), 133.3 (C-8a), 130.0 (C-5), 128.4 (C-7), 120.1 (C-8), 71.7 (CH₂O), 58.5 (OCH₃), 34.0 (CH₂), 32.5 (CH₂); MS (EI) m/z219 (M+, 25%), 202 (90), 101 (100); HRMS (EI) calc. for C₁₁H₁₃N₃O₂(M+) m/z219.1008, found 219.1006.

### Example 84

N,N-Dimethyl-3-(1-oxido-1,2,4-benzotriazi n-3-yl)-1-propanami ne hydrochloride (68). Methanesulfonyl chloride (175 μL, 2.3 mmol) was added to a stirred solution of alcohol 66 (386 mg, 1.9 mmol) and Et₃N (393 μL, 2.8 mmol) in dry DCM (30 mL) at 5° C. and the solution stirred for 2 h at 20° C. The solution was diluted with DCM (30 mL), washed with water (2×20 mL), the organic fraction dried and the solvent evaporated. The residue was dissolved in DMF (5 mL) and 40% aqueous dimethylamine (12 mL, 94 mmol) added and the solution heated at 50° C. for 2 h. The solvent was evaporated and the residue partitioned between EtOAc (50 mL) and water (50 mL). The organic fraction was extracted with water (2×25 mL), dried and the solvent evaporated. The residue was chromatographed, eluting with a gradient of (0-1%) Et₃N/(0-5%) MeOH/DCM, to give amine 68 (348 mg, 80%) which was dissolved in HCl saturated MeOH, the solvent evaporated and the residue crystallized as a tan solid, mp (MeOH/EtOAc) 228-230° C.; ¹H NMR [(CD₃)₂SO] δ 10.72 (br s, 1H, NH⁺Cl⁻), 8.39 (d, J=8.6 Hz, 1H, H-8), 8.10 (ddd, J=8.4, 7.0, 1.4 Hz, 1H, H-6), 8.06 (dd, J=8.4, 1.5 Hz, 1H, H-5), 7.86 (ddd, J=8.6, 7.0, 1.5 Hz, 1H, H-7), 3.04-3.09 (m, 2H, CH₂N), 3.04 (t, J=7.4 Hz, 2H, CH₂), 2.74 [s, 6H, N(CH₃)₂], 2.19-2.27 (m, 2H, CH₂); ¹³C NMR [(CD₃)₂SO] δ 164.7 (C-3), 146.8 (C-4a), 136.1 (C-6), 133.0 (C-8a), 130.7 (C-5), 128.4 (C-7), 119.5 (C-8), 55.5 (CH₂N), 41.8 [N(CH₃)₂], 33.2 (CH₂), 21.6 (CH₂); Anal. calc. for C₁₂H₁₇ClN₄O; C, 53.6; H, 6.4; N, 20.9; found C, 53.9; H, 6.3; N, 21.0%.

### Example 85

3-[3-(1-Piperidinyl)propyl]-1,2,4-benzotriazine 1-oxide hydrochloride (69). MsCI (133 μL, 1.7 mmol) was added to a stirred solution of alcohol 66 (293 mg, 1.4 mmol) and Et₃N (300 μL, 2.1 mmol) in dry DCM (20 mL) at 5° C. and the solution stirred for 1 h at 20° C. The solution was diluted with DCM (30 mL), washed with water (2×20 mL), the organic fraction dried and the solvent evaporated. The residue was dissolved in DMF (10 mL) and piperidine (7 mL, 70 mmol) added and the solution heated at 50° C. for 2 h. The solvent was evaporated and the residue partitioned between EtOAc (50 mL) and water (50 mL). The organic fraction was extracted with water (2×25 mL), dried and the solvent evaporated. The residue was chromatographed, eluting with a gradient (0-5%) of MeOH/DCM, to give amine 69 (291 mg, 75%) which was dissolved in HCl saturated MeOH, the solvent evaporated and the residue crystallized as a white solid, mp (MeOH/EtOAc) 151-153° C.; ¹H NMR [(CD₃)₂SO] δ 10.71 (br s, 1H, NH⁺Cl⁻), 8.39 (d, J=8.7 Hz, 1H, H-8), 8.10 (ddd, J=8.5, 6.9, 1.2 Hz, 1H, H-6), 8.05 (dd, J=8.5, 1.3 Hz, 1H, H-5), 7.86 (ddd, J=8.7, 6.9, 1.3 Hz, 1H, H-7), 3.37-3.43 (m, 2H, CH₂N), 3.10-3.17 (m, 2H, CH₂N), 3.03 (t, J=7.4 Hz, 2H, CH₂), 2.80-2.89 (m, 2H, CH₂N), 2.25-2.33 (m, 2H, CH₂), 1.67-1.83 (m, 6H, 3×CH₂); ¹³C NMR [(CD₃)₂SO] δ 164.8 (C-3), 146.8 (C-4a), 136.1 (C-6), 133.0 (C-8a), 130.7 (C-5), 128.4 (C-7), 119.5 (C-8), 54.8 (CH₂N), 51.8 (2×CH₂N), 33.42 (CH₂), 22.1 (2×CH₂), 21.3 (CH₂), 21.0 (CH₂); Anal. calc. for C₁₅H₂₀N₄O.2HCl; C, 52.2; H, 6.4; N, 16.3; found C, 52.2; H, 6.4; N, 16.4%.

### Example 86

3-[3-(4-Morpholinyl)propyl]-1,2,4-benzotriazine 1-oxide hydrochloride (70). MsCl (137 μL, 1.8 mmol) was added to a stirred solution of alcohol 66 (303 mg, 1.5 mmol) and Et₃N (309 μL, 2.2 mmol) in dry DCM (20 mL) at 5° C. and the solution stirred for 2 h at 20° C. The solution was diluted with DCM (30 mL), washed with water (2×20 mL), the organic fraction dried and the solvent evaporated. The residue was dissolved in DMF (5 mL) and morpholine (6.4 mL, 74 mmol) added and the solution heated at 50° C. for 2 h. The solvent was evaporated and the residue partitioned between EtOAc (50 mL) and water (50 mL). The organic fraction was extracted with water (2×25 mL), dried and the solvent evaporated. The residue was chromatographed, eluting with a gradient (0-5%) of MeOH/30% EtOAc/DCM, to give the morpholide 70 (330 mg, 81%) which was dissolved in HCl saturated MeOH, the solvent evaporated and the residue crystallized as a tan solid, mp (MeOH) 175-177° C.; ¹H NMR [(CD₃)₂SO] δ 11.54 (br s, 1H, NH⁺Cl⁻), 8.39 (dd, J=8.7, 1.0 Hz, 1H, H-8), 8.10 (ddd, J=8.4, 6.9, 1.0 Hz, 1H, H-6), 8.06 (dd, J=8.4, 1.0 Hz, 1H, H-5), 7.86 (ddd, J=8.7, 6.9, 1.0 Hz, 1H, H-7), 3.82-3.96 (m, 4H, 2×CH₂O), 3.38-3.43 (m, 2H, CH₂), 3.18-3.23 (m, 2H, CH₂N), 2.98-3.08 (m, 4H, 2×CH₂N), 2.26-2.33 (m, 2H, CH₂); ¹³C NMR [(CD₃)₂SO] δ 164.7 (C-3), 146.8 (C-4a), 136.2 (C-6), 133.0 (C-8a), 130.7 (C-5), 128.4 (C-7), 119.5 (C-8), 63.0 (2×CH₂O), 55.0 (CH₂N), 50.8 (2×CH₂N), 33.4 (CH₂), 20.7 (CH₂).

### Example 87

3-Methoxy-1,2,4-benzotriazine 1-oxide (71). A solution of NaOMe [prepared from the dissolution of Na (57 mg, 2.5 mmol) in dry MeOH (10 mL)] and chloride 19 (298 mg, 1.6 mmol) was stirred at 20° C. for 3 h. The solvent was evaporated and the residue partitioned between DCM (100 mL) and water (100 mL). The organic fraction was dried and the solvent evaporated. The residue was chromatographed, eluting with 5% EtOAc/DCM, to give 1-oxide 71 (257 mg, 88%) as pale yellow needles, mp (EtOAc/pet. ether) 123-124° C. [lit (Ergener, Istanbul Univ. Fen. Fak. Mecm. Seri. A, 1950, 15, 91) mp (MeOH) 121-122° C.]; ¹H NMR δ 8.38 (dd, J=8.4, 0.7 Hz, 1H, H-8), 7.83-7.88 (m, 2H, H-5, H-6), 7.54 (ddd, J=8.4, 6.2, 2.2 Hz, 1H, H-7), 4.16 (s, 3H, OCH₃); ¹³C NMR δ 162.6 (C-3), 147.4 (C-4a), 135.9 (C-6), 132.4 (C-8a), 127.6 (C-5), 127.5 (C-7), 120.5 (C-8), 55.6 (OCH₃).

### Exampl 88

3-(2-Methoxyethoxy)-1,2,4-benzotriazin 1-oxide (72). Na (123 mg, 5.3 mmol) was added to a solution of chloride 19 (645 mg, 3.6 mmol) in 2-methoxyethanol (20 mL) at 5° C. The mixture was stirred at 20° C. for 2 h, diluted with water (80 mL), extracted with EtOAc (3×80 mL), the organic fraction dried, and the solvent evaporated. The residue was chromatographed, eluting with 10% EtOAc/DCM, to give 1-oxide 72 (618 mg, 79%) as white needles, mp (EtOAc/pet. ether) 74-76° C.; ¹H NMR δ 8.37 (dd, J=8.7, 1.3 Hz, 1H, H-8), 7.81-7.88 (m, 2H, H-5, H-6), 7.53 (ddd, J=8.7, 6.7, 1.8 Hz, 1H, H-7), 4.65-4.68 (m, 2H, CH₂O), 3.81-3.84 (m, 2H, CH₂O), 3.46 (s, 3H, OCH₃);

¹³C NMR δ 162.1 (C-3), 147.4 (C-4a), 135.9 (C-6), 132.5 (C-8a), 127.6 (C-5), 127.5 (C-7), 120.4 (C-8), 70.2 (CH₂O), 67.6 (CH₂O), 59.2 (OCH₃); Anal. calc. for C₁₀H₁₁N₃O₃: C, 54.3; H, 5.0; N, 19.0; found C, 54.4; H, 4.9; N, 19.0%.

### Example 89

3-Chloro-6-methyl-1,2,4-benzotriazine 1-oxide (73). Sodium nitrite (7.09 g, 103 mmol) was added in small portions to a stirred solution of 6-methyl-1,2,4-benzotriazin-3-amine 1-oxide (3r) (9.05 g, 51.4 mmol) in trifluoroacetic acid (80 mL) at 5° C. and the solution stirred at 20° C. for 3 h. The solution was poured into ice/water, stirred 30 minutes, filtered, washed with water (3×30 mL) and dried. The solid was suspended in POCl₃ (100 mL) and DMF (0.5 mL) and stirred at 100° C. for 1 h. The solution was cooled, poured into ice/water, stirred for 0.30 minutes, filtered, washed with water (3×30 mL) and dried. The solid was suspended in DCM (150 mL), dried and the solvent evaporated. The residue was chromatographed, eluting with 5% EtOAc/DCM, to give chloride 73 (7.86 g, 78%) as a pale yellow solid, mp (EtOAc/DCM) 156-158° C.; ¹H NMR δ 8.29 (d, J=8.8 Hz, 1H, H-8), 7.74 (d, J=1.7 Hz, 1H, H-5), 7.56 (dd, J=8.8, 1.7 Hz, 1H, H-7), 2.61 (s, 3H, CH₃); Anal. calc. for C₈H₆ClN₃O: C, 49.1; H, 3.1; N, 21.5; found C, 49.2; H, 3.4; N, 21.5%.

### Example 90

2-[(6-Methyl-1-oxido-1,2,4-benzotriazin-3-yl)amino]ethanol (74). 2-Aminoethanol (0.61 mL, 10.1 mmol) was added to a stirred solution of chloride 73 (657 mg, 3.4 mmol) in DME (50 mL) and the solution stirred at reflux temperature for 16 h. The solvent was evaporated and the residue partitioned between DCM (100 mL) and dilute aqueous NH₃ (50 mL). The organic fraction was dried and the solvent evaporated. The residue was chromatographed, eluting with a gradient (5-10%) of MeOH/DCM, to give 1-oxide 74 (700 mg, 95%) as a yellow powder, mp (MeOH) 198-202° C.; ¹H NMR δ 8.00 (d, J=8.8 Hz, 1H, H-8), 7.70 (br s, 1H, NH), 7.35 (d, J=1.7 Hz, 1H, H-5), 7.15 (dd, J=8.8, 1.7 Hz, 1H, H-7), 4.70 (t, J=5.6 Hz, 1H, OH), 3.54-3.60 (m, 2H, CH₂O), 3.38-3.43 (m, 2H, CH₂N), 2.47 (s, 3H, CH₃); ¹³C NMR δ 159.1 (C-3), 148.4 (C-4a), 146.6 (C-6), 128.2 (C-8a), 126.4 (C-5), 125.7 (C-7), 119.5 (C-8), 59.2 (CH₂O), 43.2 (CH₂N), 21.3 (CH₃); Anal. calc. for C₁₀H₁₂N₄O₂: C, 54.5; H, 5.5; N, 25.4; found C, 54.7; H, 5.4; N, 25.7%.

### Example 91

N(2-Methoxyethyl)-6-methyl-1,2,4-benzotriazin-3-amine 1-oxide (75). 2-Methoxyethylamine (0.44 mL, 5.0 mmol) was added to a stirred solution of chloride 73 (329 mg, 1.7 mmol) in DME (50 mL) and the solution stirred at reflux temperature for 2 h. The solvent was evaporated and the residue partitioned between DCM (100 mL) and dilute aqueous NH₃ (50 mL). The organic fraction was dried and the solvent evaporated. The residue was chromatographed, eluting with a gradient (5-40%) of EtOAc/DCM, to give 1-oxide 75 (368 mg, 93%) as a yellow powder, mp (MeOH) 157-158° C.; ¹H NMR 88.14 (d, J=8.8 Hz, 1H, H-8), 7.36 (d, J=1.7 Hz, 1H, H-5), 7.10 (dd, J=8.8, 1.7 Hz, 1H, H-7), 5.58 (br s, 1H, NH), 3.68-3.72 (m, 2H, CH₂N), 3.59-3.62 (m, 2H, CH₂O), 3.39 (s, 3H, OCH₃), 2.47 (s, 3H, CH₃); ¹³C NMR δ 159.1 (C-3), 149.1 (C-4a), 147.0 (C-6), 129.3 (C-8a), 127.2 (C-5), 125.4 (C-7), 120.1 (C-8), 70.9 (CH₂O), 58.8 (OCH₃), 41.1 (CH₂N), 22.0 (CH₃); Anal. calc. for C₁₁H₁₄N₄O₂: C, 56.4; H, 6.0; N, 23.9; found C, 56.4; H, 5.9; N, 23.8%.

### Example 92

N¹,N¹-Dimethyl-N²-(6-methyl-1-oxido-1,2,4-benzotriazin-3-yl)-1,2-ethanediamine (76). N,N-Dimethylethanediamine (705 μL, 6.6 mmol) was added to a stirred solution of chloride 73 (518 mg, 2.7 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 76 (603 mg, 92%) as a yellow solid, mp (MeOH/EtOAc) 143-145° C.; ¹H NMR δ 8.11 (d, J=8.8 Hz, 1H, H-8), 7.35 (d, J=1.7 Hz, 1H, H-5), 7.07 (dd, J=8.8, 1.7 Hz, 1H, H-7), 5.89 (br s, 1H, NH), 3.50-3.56 (m, 2H, CH₂N), 2.52-2.56 (m, 2H, CH₂N), 2.45 (s, 3H, CH₃), 2.26 [s, 6H, N(CH₃)₂]; ¹³C NMR δ 159.2 (C-3), 149.1 (C-4a), 146.9 (C-6), 129.2 (C-8a), 126.9 (C-5), 125.3 (C-7), 120.1 (C-8), 57.5 (CH₂N), 45.1 [N(CH₃)₂], 38.7 (CH₂N), 22.0 (CH₃); Anal. calc. for C₁₂H₁₇N₅O: C, 58.3; H, 6.9; N, 28.3; found C, 58.5: H, 7.1; N, 28.6%.

### Exampl 93

6-Methyl-N-[2-(1-piperidinyl)ethyl]-1,2,4-benzotriazin-3-amine 1-oxide (77). 2-(1-Piperidinyl)ethylamine (0.87 mL, 6.1 mmol) was added to a stirred solution of chloride 73 (476 mg, 2.4 mmol) in DME (50 mL) and the solution stirred at reflux temperature for 2 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give 1-oxide 77 (656 mg, 94%) as a yellow powder, mp (MeOH/EtOAc) 156-158° C.; ¹H NMR δ 8.13 (d, J=8.7 Hz, 1H, H-8), 7.36 (d, J=1.7 Hz, 1H, H-5), 7.08 (dd, J=8.7, 1.7 Hz, 1H, H-7), 5.98 (br s, 1H, NH), 3.51-3.56 (m, 2H, CH₂N), 2.54-2.58 (m, 2H, CH₂N), 2.47 (s, 3H, CH₃), 2.39-2.45 (m, 4H, 2×CH₂N), 1.55-1.61 (m, 4H, 2×CH₂), 1.42-1.48 (m, 2H, CH₂); ¹³C NMR δ 159.1 (C-3), 149.1 (C-4a), 146.9 (C-6), 129.1 (C-8a), 126.9 (C-7), 125.3 (C-5), 120.1 (C-8), 56.9 (CH₂N), 54.3 (2×CH₂N), 37.9 (CH₂N), 26.0 (2×CH₂), 24.4 (CH₂), 22.0 (CH₃); Anal. calc. for C₁₅H₂₁N₅O: C, 62.7; H, 7.4; N, 24.4; found C, 62.8; H, 7.7; N, 24.5%.

### Example 94

N-[2-(2,6-Dimethyl-1-piperidinyl)ethyl]-6-methyl-1,2,4-benzotriazin-3-amine 1-oxide (78). 2-(2,6-Dimethyl-1-piperidinyl)ethylamine (834 mg, 5.3 mmol) was added to a stirred solution of chloride 73 (418 mg, 2.1 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 78 (597 mg, 93%) as a yellow solid, mp (MeOH/EtOAc) 162-165° C.; ¹H NMR δ 8.12 (d, J=8.9 Hz, 1H, H-8), 7.35 (d, J=1.7 Hz, 1H, H-5), 7.09 (dd, J=8.9, 1.7 Hz, 1H, H-7), 5.57 (br s, 1H, NH), 3.50-3.56 (m, 2H, CH₂N), 2.87-2.91 (m, 2H, CH₂N), 2.49-2.57 (m, 2H, 2×CH), 2.47 (s, 3H, CH₃), 1.65-1.69 (m, 1H, CH₂), 1.53-1.58 (m, 2H, CH₂), 1.25-1.41 (m, 3H, CH₂), 1.19 (d, J=6.3 Hz, 6H, 2×CH₃); ¹³C NMR δ 159.2 (C-3), 149.1 (C-4a), 146.9 (C-6), 129.2 (C-8a), 127.0 (C-5), 125.4 (C-7), 120.8 (C-8), 57.3 (2×CH), 47.4 (CH₂N), 39.5 (CH₂N), 34.2 (CH₂), 24.4 (2×CH₂), 22.0 (2×CH₃), 21.6 (CH₃); Anal. calc. for C₁₇H₂₅N₅O: C, 64.7; H, 8.0; N, 22.2; found C, 64.3: H, 7.3; N, 22.0%.

### Exampl 95

3-Ethyl-6-methyl-1,2,4-benzotriazine 1-oxide (79). Pd(PPh₃)₄ (410 mg, 0.35 mmol) was added to a stirred solution of chloride 73 (728 mg, 3.6 mmol) and tetraethyltin (1.4 mL, 7.1 mmol), the solution degassed, and stirred under N₂ at reflux temperature for 16 h. The solvent was evaporated and the residue chromatographed, eluting with 20% EtOAc/pet. ether to give an oil which was chromatographed, eluting with 5% EtOAc/DCM, to give (i) starting material 73 (412 mg, 56%) and (ii) 1-oxide 79 (250 mg, 37%) as a white solid, mp (EtOAc/DCM) 68-70° C.; ¹H NMR δ 8.33 (d, J=8.8 Hz, 1H, H-8), 7.74 (br s, 1H, H-5), 7.49 (dd, J=8.8, 1.7 Hz, 1H, H-7), 3.02 (q, J=7.6 Hz, 2H, CH₂), 2.59 (s, 3H, CH₃), 1.44 (t, J=7.6 Hz, 3H, CH₃); ¹³C NMR δ 168.2 (C-3), 147.8 (C-4a), 147.1 (C-6), 132.0 (C-5), 131.6 (C-8a), 127.5 (C-7), 119.8 (C-8), 30.7 (CH₂), 22.1 (CH₃), 12.2 (CH₃); Anal. calc. for C₁₀H₁₁N₃O: C, 63.5; H, 5.9; N, 22.2; found C, 63.5; H, 6.0; N, 22.3%.

### Example 96

3-Allyl-6-methyl-1,2,4-benzotriazine 1-oxide (80). Pd(PPh₃)₄ (370 mg, 0.32 mmol) was added to a stirred solution of chloride 73 (1.24 g, 6.3 mmol) and allyltributyltin (2.2 mL, 7.0 mmol), the solution degassed, and stirred under N₂ at reflux temperature for 6 h. The solvent was evaporated and the residue chromatographed, eluting with 20% EtOAc/pet. ether, to give an oil which was chromatographed, eluting with 5% EtOAc/DCM, to give alkene 80 (0.97 g, 74%) as a white solid, mp (EtOAc/pet. ether) 65-67° C., ¹H NMR δ 8.32 (d, J=8.8 Hz, 1H, H-8), 7.76 (d, J=1.7 Hz, 1H, H-5), 7.50 (dd, J=8.8, 1.7 Hz, 1H, H-7), 6.13-6.21 (m, 1H, H-2′), 5.30 (dq, J=17.0, 1.5 Hz, 1H, H-3′), 5.22 (dq, J=10.1, 1.5 Hz, 1H, H-3′), 3.76 (dq, J=6.9, 1.5 Hz, 2H, H-1′), 2.58 (s, 3H, CH₃); ¹³C NMR δ 165.3 (C-3), 147.8 (C-4a), 147.3 (C-6), 132.8 (C-2′), 132.5 (C-5), 131.6 (C-8a), 127.6 (C-7), 119.7 (C-8), 118.4 (C-3′), 41.8 (C-1′), 22.1 (CH₃); Anal. calc. for C₁₁H₁₁N₃O: C, 65.7; H, 5.5; N, 20.9; found C, 65.8; H, 5.5; N, 21.0%.

### Example 97

2-(6-Methyl-1-oxido-1,2,4-benzotriazin-3-yl)ethanol (81). Ozone was bubbled into a solution of alkene 80 (1.12 g, 5.6 mmol) in DCM/MeOH (1:1, 80 mL) at −78° C. until a blue colour persisted. The solution was purged with N₂ to remove excess ozone, then a solution of NaBH₄ (210 mg, 5.6 mmol) in EtOH (10 mL) added dropwise and the solution allowed to warm to 20° C. over 1 h. HOAc (2 mL) was added and the solution stirred at 20° C. for 30 min. The solvent was evaporated and the residue partitioned between DCM (50 mL) and water (3×50 mL). The organic fraction was dried and the solvent evaporated. The residue was chromatographed, eluting with a gradient (50-100%) of EtOAc/pet. ether, to give alcohol 81 (780 mg, 68%) as pale yellow prisms, mp (EtOAc/pet. ether) 121-123° C.; ¹H NMR δ 8.32 (d, J=8.8 Hz, 1H, H-8), 7.74 (d, J=1.7 Hz, 1H, H-5), 7.50 (dd, J=8.8, 1.7 Hz, 1H, H-7), 4.04-4.10 (m, 2H, CH₂O), 3.27 (t, J=5.6 Hz, 2H, CH₂), 3.17 (t, J=6.3 Hz, 1H, OH), 2.60 (s, 3H, CH₃); ¹³C NMR δ 165.5 (C-3), 147.6 (C-4a), 147.3 (C-6), 132.4 (C-5), 131.9 (C-8a), 127.4 (C-7), 119.8 (C-8), 60.1 (CH₂O), 39.0 (CH₂), 22.1 (CH₃); Anal. calc for C₁₀H₁₁N₃O₂: C, 58.5; H, 5.4; N, 20.5; found C, 58.8; H, 5.5; N, 20.5%.

### Example 98

3-(2-Methoxyethyl)-6-methyl-1,2,4-benzotriazine 1-oxide (82). Three aliquots of TMSCH₂N₂ (1.1 mL, 2.1 mmol) were added to a stirred solution of alcohol 81 (433 mg, 2.1 mmol) and HBF₄ (0.26 mL, 2.1 mmol) in DCM (30 mL) over 3 h. The solution was stirred at 20° C. for 16 h, the solvent evaporated and the residue chromatographed, eluting with 50% EtOAc/pet. ether, to give (i) methyl ether 82 (119 mg, 19%) as a yellow powder, mp (EtOAC/pet. ether) 77-79° C.; ¹H NMR δ 8.32 (d, J=8.8 Hz, 1H, H-8), 7.56 (d, J=1.7 Hz, 1H, H-5), 7.50 (dd, J=8.8, 1.7 Hz, 1H, H-6), 3.95 (t, J=6.5 Hz, 2H, CH₂O), 3.37 (s, 3H, OCH₃), 3.27 (t, J=6.5 Hz, 2H, CH₂), 2.58 (s, 3H, CH₃); ¹³C NMR δ 164.7 (C-3), 147.7 (C-4a), 147.2 (C-6), 132.2 (C-5), 131.8 (C-8a), 127.6 (C-7), 119.7 (C-8), 70.1 (CH₂O), 58.7 (OCH₃), 37.6 (CH₂), 22.1 (CH₃); and (ii) starting material 81 (360 mg, 62%), spectroscopically identical to sample prepared above.

### Example 99

3-Chloro-6-methoxy-1,2,4-benzotriazine 1-oxide (83). Sodium nitrite (7.14 g, 103.4 mmol) was added in portions to a stirred solution of 6-methoxy-1,2,4-benzotriazin-3-amine 1-oxide 3q (9.94 g, 51.7 mmol) in trifluoroacetic acid (50 mL) at 5° C. and the solution stirred at 20° C. for 1 h. The solution was poured into ice/water, filtered, washed with water (2×50 mL) and dried. The solid was suspended in POCl₃ (80 mL), DMF (2 drops) added, and the mixture stirred at 100° C. for 3 h. The solution was poured into ice/water, stirred for 20 minutes and filtered. The solid was dissolved in DCM (150 mL), dried, and the solvent evaporated. The residue was chromatographed, eluting with 5% EtOAc/DCM, to give chloride 83 (7.42 g, 68%) as a pale yellow solid, mp (EtOAc/DCM) 196-199° C.; ¹H NMR δ 8.30 (d, J=9.6 Hz, 1H, H-8), 7.32 (dd, J=9.6, 2.7 Hz, 1H, H-7), 7.19 (d, J=2.7 Hz, 1H, H-5), 4.01 (s, 3H, OCH₃); ¹³C NMR δ 166.3 (C-6), 157.8 (C-3), 150.2 (C-4a), 128.9 (C-8a), 123.9 (C-5), 121.9 (C-7), 105.7 (C-8), 56.5 (OCH₃); Anal. calc. for C₈H₆ClN₃O₂: C, 45.4; H, 2.9; N, 19.9; Cl, 16.8; found C, 45.2; H, 2.6; N, 19.9; Cl, 16.9%.

### Example 100

N#-(6-Methoxy-1-oxido-1,2,4-benzotriazin-3-yl)-N²,N²-dimethyl-1,2-ethanediamine (84). N,N-Dimethyl-1,2-ethanediamine (1.33 mL, 12.1 mmol) was added to a stirred solution of chloride 83 (0.85 g, 4.04 mmol) in DME (50 mL) and the solution stirred at reflux temperature for 16 h. The solvent was evaporated and the residue partitioned between DCM (100 mL) and dilute aqueous NH₃ (50 mL). The organic fraction was dried and the solvent evaporated. The residue was chromatographed, eluting with a gradient (0-5%) of MeOH/DCM, to give amine 84 (0.72 g, 68%) which was dissolved in HCl saturated MeOH, the solvent evaporated and the residue crystallized as a tan solid, mp (MeOH/EtOAc) 236-239° C.; ¹H NMR [(CD₃)₂SO] δ 10.68 (br s, 1H, NH⁺Cl⁻), 8.07 (d, J=9.3 Hz, 1H, H-8), 8.03 (br s, 1H, NH), 6.95-6.99 (m, 2H, H-5, H-7), 3.92 (s, 3H, OCH₃), 3.70-3.76 (m, 2H, CH₂N), 3.30-3.35 (m, 2H, CH₂N), 2.81 [d, J=4.9 Hz, 6H, N(CH₃)₂]; ¹³C NMR [(CD₃)₂SO] δ 164.9 (C-6), 159.0 (C-3), 150.4 (C-4a), 125.4 (C-8a), 121.6 (C-8), 117.3 (C-5), 104.3 (C-7), 55.2 (OCH₃), 55.2 (CH₂N), 42.3 [N(CH₃)₂], 35.8 (CH₂N); Anal. calc. for C₁₂H₁₈ClN₅O₂: C, 48.1; H, 6.1; N, 23.4; Cl, 11.8; found C, 48.3; H, 6.1; N, 23.6; Cl, 11.9%.

### Example 101

6-Methoxy-N-[2-(1-piperidinyl)ethyl]-1,2,4-benzotriazin-3-amine 1-oxide (85). 2-(1-Piperidinyl)ethylamine (0.9 mL, 6.0 mmol) was added to a stirred solution of chloride 83 (509 mg, 2.4 mmol) in DME (50 mL) and the solution stirred at reflux temperature for 2 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give 1-oxide 85 (736 mg, 100%) as a yellow powder, mp (MeOH) 133-135° C.; ¹H NMR δ 8.15 (d, J=9.8 Hz, 1H, H-8), 6.85-6.88 (m, 2H, H-5, H-7), 6.00 (br s, 1H, NH), 3.92 (s, 3H, OCH₃), 3.52-3.56 (m, 2H, CH₂N), 2.56-2.60 (m, 2H, CH₂N), 2.38-2.44 (m, 4H, 2×CH₂N), 1.56-1.62 (m, 4H, 2×CH₂), 1.42-1.48 (m, 2H, CH₂); ¹³C NMR 6165.4 (C-7), 159.5 (C-3), 151.5 (C-4a), 126.0 (C-8a), 122.0 (C-7), 117.6 (C-8), 104.5 (C-5), 56.8 (CH₂N), 56.0 (OCH₃), 54.3 (2×CH₂N), 37.9 (CH₂N), 26.0 (2×CH₂), 24.4 (CH₂); Anal. calc. for C₁₅H₂₁N₅O₂.H₂O: C, 56.1; H, 7.2; N, 21.8; found C, 55.9; H, 7.0; N, 21.7%.

### Example 102

6-Methoxy-N-[2-(4-morpholinyl)ethyl]-1,2,4-benzotriazin-3-amine 1-oxide (86). 2-(4-Morpholinyl)ethylamine (1.92 mL, 14.6 mmol) was added to a stirred solution of chloride 83 (1.03 g, 4.9 mmol) in DME (50 mL) and the solution stirred at reflux temperature for 16 h. The solvent was evaporated and the residue partitioned between DCM (100 mL) and dilute aqueous NH₃ (50 mL). The organic fraction was dried and the solvent evaporated. The residue was chromatographed, eluting with a gradient (0-5%) of MeOH/DCM, to give amine 86 (0.89 g, 60%) as a yellow powder, mp (MeOH/EtOAc) 186-188° C.; ¹H NMR [(CD₃)₂SO] δ 8.03 (d, J=9.5 Hz, 1H, H-8), 7.63 (br s, 1H, NH), 6.90-6.95 (m, 2H, H-5, H-7), 3.91 (s, 3H, OCH₃), 3.55-3.58 (m, 4H, 2×CH₂O), 3.45-3.49 (m, 2H, CH₂N), 2.50-2.55 (m, 2H, CH₂N), 2.41-2.44 (m, 4H, 2×CH₂N); ¹³C NMR [(CD₃)₂SO] δ 164.8 (C-6), 159.4 (C-3), 150.9 (C-4a), 125.0 (C-8a), 121.5 (C-8), 116.8 (C-7), 104.2 (C-5), 66.1 (2×CH₂O), 56.8 (CH₂N), 56.1 (OCH₃), 53.2 (2×CH₂N), 37.7 (CH₂N); Anal. calc. for C₁₄H₁₉N₅O₃.¼H₂O: C, 54.3; H, 6.3; N, 22.6; found C, 54.5; H, 6.2; N, 22.8%.

### Example 103

3-Chloro-7-methyl-1,2,4-benzotriazine 1-oxide (87). A solution of NaNO₂ (3.9 g, 56.3 mmol) in water (15 mL) was added dropwise to a stirred suspension of amine 3j (4.95 g, 28.1 mmol) in 2 M HCl (200 mL) at 5° C. and the mixture stirred vigorously for 2 h at 20° C. The suspension was filtered, the solid dissolved in dilute aqueous NH₃ (150 mL), filtered and the filtrate acidified with cHCl. The suspension was cooled, filtered and the solid washed with water (2×10 mL) and dried. The solid (3.76 g, 21.2 mmol) was suspended in dimethylaniline (6.7 mL, 53 mmol) and POCl₃ (14 mL, 149 mmol). The mixture was stirred at relux temperature for 1 h, the resulting solution poured on to ice (300 mL). The suspension was filtered, washed with water (2×20 mL), dissolved in EtOAc (200 mL), dried and the solvent evaporated. The residue was chromatographed, eluting with 5% EtOAc/DCM, to give chloride 87 (2.99 g, 72%) as a yellow solid, mp 176.5-177° C. [lit (Foye et. al., J. Het. Chem. 1982, 19, 497) mp (toluene) 177-179° C.]; ¹H NMR δ 8.21 (d, J=2.0 Hz, 1H, H-8), 7.89 (d, J=8.6 Hz, 1H, H-5), 7.81 (dd, J=8.6, 2.0 Hz, 1H, H-6), 2.61 (s, 3H, CH₃).

### Example 104

7-Methyl-N[2-(di methylamino)ethyl]-1,2,4-benzotriazi n-3-amin 1-oxide (88). 2-(Dimethylamino)ethylamine (1.0 mL, 9.0 mmol) was added to a stirred solution of chloride 87 (700 mg, 3.6 mmol) in DME (50 mL) and the solution stirred at reflux temperature for 8 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) MeOH/DCM, to give 1-oxide 88 (781 mg, 88%) as a yellow solid, mp (DCM) 143-144° C.; ¹H NMR [(CD₃)₂SO] δ 7.93 (br s, 1H, H-8), 7.60-7.64 (m, 2H, NH, H-6), 7.48 (d, J=8.6 Hz, 1H, H-5), 3.37-3.45 (m, 2H, CH₂N), 2.46-2.52 (m, 2H, CH₂N), 2.41 (s, 3H, CH₃), 2.21 μm, 6H, N(CH₃)₂]; ¹³C NMR [(CD₃)₂SO] δ 158.6 (C-3), 146.8 (C-4a), 137.6 (C-6), 134.6 (C-7), 129.6 (C-8a), 125.8 (C-5), 118.4 (C-8), 57.6 (CH₂N), 45.1 [N(CH₃)₂], 39.0 (CH₂N), 20.6 (CH₃); Anal. calc. for C₁₂H₁₇N₅O: C, 58.3; H, 6.9; N, 28.3; found C, 58.5: H, 7.2; N, 28.6%

### Example 105

7-Methyl-N-[2-(1-piperidinyl)ethyl]-1,2,4-benzotriazin-3-amine 1-oxide (89). 2-(1-Piperidinyl)ethylamine (0.83 mL, 5.8 mmol) was added to a stirred solution of chloride 87 (453 mg, 2.3 mmol) in DME (50 mL) and the solution stirred at reflux temperature for 2 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give 1-oxide 89 (635 mg, 95%) as a yellow powder, mp (MeOH) 166-168° C.; ¹H NMR δ 8.04-8.06 (m, 1H, H-8), 7.52 (dd, J=8.7, 1.8 Hz, 1H, H-6), 7.49 (d, J=8.7 Hz, 1H, H-5), 5.95 (br s, 1H, NH), 3.52-3.56 (m, 2H, CH₂N), 2.56-2.59 (m, 2H, CH₂N), 2.45 (s, 3H, CH₃), 2.40-2.44 (m, 4H, 2×CH₂N), 1.55-1.61 (m, 4H, 2×CH₂), 1.42-1.47 (m, 2H, CH₂); ¹³C NMR δ 158.7 (C-3), 147.7 (C-4a), 137.6 (C-6), 135.3 (C-7), 130.4 (C-8a), 126.1 (C-5), 119.2 (C-8), 56.9 (CH₂N), 54.3 (2×CH₂N), 37.9 (CH₂N), 26.0 (2×CH₂), 24.4 (CH₂), 21.3 (CH₃); Anal. calc. for C₁₅H₂₁N₅O: C, 62.7; H, 7.4; N, 24.4; found C, 62.8; H, 7.1; N, 24.7%.

### Example 106

7-Methyl-N-[3-(4-morpholinyl)propyl]-1,2,4-benzotriazin-3-amine 1-oxide (90). 3-(4-Morpholinyl)propylamine (1.4 mL, 9.4 mmol) was added to a stirred solution of chloride 87 (738 mg, 3.8 mmol) in DME (50 mL) and the solution stirred at reflux temperature for 8 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 90 (1.12 g, 98%) as a yellow powder, mp 158-160° C.; ¹H NMR [(CD₃)₂SO] δ 7.94 (d, J=1.7 Hz, 1H, H-8), 7.80 (br s, 1H, NH), 7.63 (dd, J=8.6, 1.7 Hz, 1H, H-6), 7.47 (d, J=8.6 Hz, 1H, H-5), 3.53-3.57 (m, 4H, 2×CH₂O), 3.36-3.39 (m, 2H, CH₂N), 2.41 (s, 3H, CH₃), 2.31-2.38 (m, 6H, 3×CH₂N), 1.71-1.77 (m, 2H, CH₂); ¹³C NMR [(CD₃)₂SO] δ 158.6 (C-3), 146.8 (C-4a), 137.6 (C-6), 134.5 (C-7), 129.6 (C-8a), 125.7 (C-5), 118.4 (C-8), 66.1 (2×CH₂O), 55.8 (CH₂N), 53.2 (2×CH₂N), 38.9 (CH₂N), 25.3 (CH₂), 20.6 (CH₃); Anal. calc. for C₁₅H₂₁H₅O₂: C, 59.4; H, 7.0; N, 23.1; found C, 59.7: H, 7.2; N, 23.2%

### Example 107

3-Chloro-7-methoxy-1,2,4-benzotriazine 1-oxide (91). A solution of NaNO₂ (6.45 g, 93.6 mmol) in water (25 mL) was added dropwise to a stirred suspension of amine 3i (9.0 g, 46.8 mmol) in 2 M HCl (500 mL) at 5° C. and the foaming suspension stirred vigourously for 2 h. The solid was filtered, dissolved in dilute aqueous NH₃, filtered, the filtrate acidified with conc. HCl, and cooled. The resulting precipitate was filtered, washed with water and dried. The solid was dissolved in POCl₃ (100 mL) and DMF (0.5 mL) and heated at 100° C. for 1 h. The solution was cooled, poured into ice/water, stirred for 30 minutes, filtered, washed with water (3×30 mL) and dried. The solid was suspended in DCM (150 mL), dried and the solvent evaporated. The residue was chromatographed, eluting with 5% EtOAc/DCM, to give chloride 87 (7.10 g, 71%) as a pale yellow solid, mp (EtOAc/DCM) 177-179° C. [lit (Sasse et. al, Ger. Offen. 2, 740, 887, 1979) mp (benzene) 164-165° C.]; ¹H NMR δ 7.91 (d, J=9.2 Hz, 1H, H-5), 7.69 (d, J=2.9 Hz, 1H, H-8), 7.64 (dd, J=9.2, 2.9 Hz, 1H, H-6), 4.03 (s, 3H, OCH₃); ¹³C NMR δ 161.7 (C-7), 154.7 (C-3), 143.6 (C-4a), 130.0 (C-8, C-8a), 129.6 (C-6), 97.8 (C-5), 56.6 (OCH₃).

### Example 108

N¹-(7-Methoxy-1-oxido-1,2,4-benzotriazin-3-yl)-N²,N²-dimethyl-1,2-ethanediamine (92). N,N-dimethylethylenediamine (1.0 mL, 9.3 mmol) was added to a stirred solution of chloride 91 (659 mg, 3.1 mmol) in DME (50 mL) and the solution stirred at reflux temperature for 16 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give the amine 92 (820 mg, 90%) as a yellow powder, which was recrystallized as the hydrochloride salt, mp (MeOH/EtOAc) 231-235° C.; ¹H NMR [(CD₃)₂SO] δ 10.61 (br s, 1H, NH⁺Cl⁻), 7.89 (br s, 1H, NH), 7.59 (d, J=8.9 Hz, 1H, H-5), 7.49-7.53 (m, 2H, H-6, H-8), 3.88 (s, 3H, OCH₃), 3.69-3.73 (m, 2H, CH₂N), 3.28-3.32 (m, 2H, CH₂N), 2.82 [d, J=4.9 Hz, 6H, N(CH₃)₂]; ¹³C NMR [(CD₃)₂SO] δ 157.8 (C-7), 156.7 (C-3), 144.0 (C-4a), 130.3 (C-8a), 128.3 (C-5), 127.6 (C-6), 98.1 (C-8), 55.9 (OCH₃), 55.3 (CH₂N), 42.3 [N(CH₃)₂], 35.8 (CH₂N); Anal. calc. for C₁₂H₁₈ClN₅O₂: C, 48.1; H, 6.1; N, 23.4; Cl, 11.8; found C, 48.1; H, 6.2; N, 23.5; Cl, 12.0%.

### Example 109

7-Methoxy-N-[2-(1-piperidi nyl)ethyl]-1,2,4-benzotriazin-3-amine 1-oxide (93). 2-(1-Piperidinyl)ethylamine (0.8 mL, 5.4 mmol) was added to a stirred solution of chloride 91 (453 mg, 2.1 mmol) in DME (50 mL) and the solution stirred at reflux temperature for 16 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give 1-oxide 93 (625 mg, 96%) as a yellow powder, mp (MeOH) 162-165° C.; ¹H NMR δ 7.58 (d, J=2.8 Hz, 1H, H-8), 7.53 (d, J=9.2 Hz, 1H, H-5), 7.38 (dd, J=9.2, 2.8 Hz, 1H, H-6), 5.90 (br s, 1H, NH), 3.91 (s, 3H, OCH₃), 3.52-3.56 (m, 2H, CH₂N), 2.56-2.60 (m, 2H, CH₂N), 2.46-2.56 (m, 4H, 2×CH₂N), 1.56-1.62 (m, 4H, 2×CH₂), 1.43-1.48 (m, 2H, CH₂); ¹³C NMR δ 158.4 (C-7), 157.2 (C-3), 145.3 (C-4a), 130.7 (C-8a), 128.8 (C-5), 127.7 (C-6), 98.2 (C-8), 56.9 (CH₂N), 56.0 (OCH₃), 54.3 (2×CH₂N), 38.2 (CH₂N), 26.0 (2×CH₂), 24.4 (CH₂); Anal. calc. for C₁₅H₂₁N₅O₂: C, 59.4; H, 7.0; N, 23.1; found C, 59.1; H, 6.7; N, 23.2%.

### Example 110

7-Methoxy-N-[2-(4-morpholinyl)ethyl]-1,2,4-benzotriazin-3-amine 1-oxide (94). 2-(4-Morpholinyl)ethylamine (1.2 mL, 9.0 mmol) was added to a stirred solution of chloride 91 (633 mg, 3.0 mmol) in DME (50 mL) and the solution stirred at reflux temperature for 16 h. The solvent was evaporated and the residue partitioned between DCM (100 mL) and dilute aqueous NH₃ (50 mL). The organic fraction was dried and the solvent evaporated. The residue was chromatographed, eluting with a gradient (0-5%) of MeOH/DCM, to give 1-oxide 94 (820 mg, 90%) as a yellow powder, mp (MeOH) 208-212° C.; ¹H NMR δ 7.58 (d, J=2.7 Hz, 1H, H-8), 7.53 (d, J=9.3 Hz, 1H, H-5), 7.38 (dd, J=9.3, 2.7 Hz, 1H, H-6), 5.77 (br s, 1H, NH), 3.91 (s, 3H, OCH₃), 3.70-3.73 (m, 4H, 2×CH₂O), 3.55-3.59 (m, 2H, CH₂N), 2.63-2.67 (m, 2H, CH₂N), 2.48-2.52 (m, 4H, 2×CH₂N); ¹³C NMR δ 158.3 (C-7), 157.3 (C-3), 145.2 (C-4a), 130.8 (C-8a), 128.9 (C-5), 127.7 (C-6), 98.2 (C-8), 66.9 (2×CH₂O), 56.8 (CH₂N), 56.0 (OCH₃), 53.3 (2×CH₂N), 37.5 (CH₂N); Anal. calc. for C₁₄H₁₉N₅O₃.¹/4H₂O: C, 54.3; H, 6.3; N, 22.6; found C, 54.1; H, 6.2; N, 22.8%.

### Example 111

3-Chloro-7-(2-methoxyethoxy)-1,2,4-benzotriazine 1-oxide (96).

3-Hydroxy-7-(2-methoxyethoxy)-1,2,4-benzotriazine 1-oxide (95). A suspension of amine 14 (1.00 g, 4.2 mmol) in 2 N HCl (32 mL) was cooled to 5° C. and a solution of NaNO₂ (0.58 g, 8.5 mmol) in water (1.5 mL) was added over 1 h. More NaNO₂ (0.58 g, 8.5 mmol) in water (1.5 mL) was added and the suspension stirred 72 h at room temperature. The precipitate was filtered and washed with water. The solid was dissolved in 5% aqueous NH₃ and filtered. The filtrate was acidified with conc. HCl to give a precipitate which was filtered, dried and chromatographed, eluting with a gradient (0-5%) of MeOH/DCM, to give compound 95 (0.68 g, 68%) as a yellow solid, mp (DCM/pet.ether) 190-192° C.; ¹H NMR [(CD₃)₂SO] δ 12.52 (br, 1H, OH), 7.69 (br s, 1H, H-8), 7.53 (dd, J=8.8, 2.8 Hz, 1H, H-6), 7.33 (d, J=8.8 Hz, 1H, H-5), 4.19 (t, J=4.4 Hz, 2H, CH₂), 3.68 (t, J=4.4 Hz, 2H, CH₂), 3.33 (s, 3H, OCH₃); ¹³C NMR [(CD₃)₂SO] δ 154.6, 152.9, 131.8, 129.5, 127.4, 117.8, 101.8, 70.0, 67.9, 58.1; Anal. calc. for C₁₀H₁₁N₃O₄: C, 50.6; H, 4.2; N, 17.7; found: C, 50.5; H, 4.7; N, 17.7%. 3-Chloro-7-(2-methoxyethoxy)-1,2,4-benzotriazine 1-oxide (96). A mixture of 95 (1.00 g, 4.3 mmol) in POCl₃ (8 mL) was refluxed for 2 h. Excess reagent was evaporated under vacuum, and ice cold water (50 mL) was added to the residue, then solid Na₂CO₃ (1.0 g) was added portionwise. The resulting precipitate was filtered and chromatographed, eluting with a gradient (50-100%) of DCM/pet. ether, to give chloride 96 (0.90 g, 83%) as a pale yellow solid, mp (DCM/pet. ether) 121-125° C.; ¹H NMR [(CD₃)₂SO] δ 8.00 (d, J=9.2 Hz, 1H, H-5), 7.81 (dd, J=9.2, 2.9 Hz, 1H, H-6), 7.68 (d, J=2.8 Hz, 1H, H-8), 4.35 (t, J=4.4 Hz, 2H, CH₂), 3.74 (t, J=4.4 Hz, 2H, CH₂), 3.33 (s, 3H, OCH₃); Anal. calc. for C₁₀H₁₀ClN₃O₃: C, 47.0; H, 3.9; N, 16.4, Cl, 13.9; found C, 46.9; H, 4.3; N, 16.4; Cl, 13.7%.

### Example 112

3-Ethyl-7-(2-methoxyethoxy)-1,2,4-benzotriazine 1-oxid (97).

Pd(PPh₃)₄ (92 mg, 0.08 mmol) was added to a N₂ purged solution of chloride 96 (260 mg, 1.0 mmol) and tetraethyltin (0.4 mL, 2.0 mmol) in DMF (15 mL). The purged reaction mixture was heated at reflux temperature for 20 h under N₂. The solvent was evaporated and the residue chromatographed, eluting with 50% DCM/pet. ether, to give 1-oxide 97 (142 mg, 56%) as a white powder, mp (DCM/pet. ether) 95-97° C.; ¹H NMR [(CD₃)₂SO] δ 7.97 (d, J=9.2 Hz, 1H, H-5), 7.74 (dd, J=9.2, 2.9 Hz, 1H, H-6), 7.68 (d, J=2.8 Hz, 1H, H-8), 4.33 (t, J=4.4 Hz, 2H, CH₂), 3.74 (t, J=4.4 Hz, 2H, CH₂), 3.32 (s, 3H, OCH₃), 2.75 (q, J=7.5 Hz, 2H, CH₂), 1.33 (t, J=7.6 Hz, 3H, CH₃); ¹³C NMR [(CD₃)₂SO] δ 164.9, 159.6, 143.1, 133.3, 129.8, 128.7, 98.1, 69.8, 68.2, 58.1, 29.5, 11.9; Anal. calc. for C₁₂H₁₅N₃O₃C, 57.8; H, 6.1; N, 16.9; found C, 57.7; H, 6.1; N, 16.6%.

### Example 113

3-Chloro-7-[2-(4-morpholinyl)ethoxy]-1,2,4-benzotriazine 1-oxide (98). A solution of NaNO₂ (286 mg, 4.1 mmol) in water (2 mL) was added slowly to a solution of amine 17 (610 mg, 2.09 mmol) in 1 N HCl (16 mL) at 5° C. The reaction mixture was stirred at 5° C. for a further 2 h, neutralized with NaHCO₃ and solvent was evaporated. The residue was filtered through a short column of silica, eluting with MeOH. The filtrate was evaporated and the residue heated at reflux temperature in POCl₃ (2 mL) and dimethylaniline (0.6 mL, 2.5 eq) for 2 h. The reaction mixture was cooled and poured into ice/water (50 mL). The cold slurry was neutralized with solid NaHCO₃ (2 g), extracted with EtOAc (3×50 mL), dried and the solvent evaporated. The residue was chromatographed, eluting with a gradient (0-20%) of EtOAc/DCM, to give chloride 98 (346 mg, 53%) as a white crystals, mp (DCM/pet. ether) 144-146° C.; ¹H NMR [(CD₃)₂SO] δ 7.99 (d, J=9.2 Hz, 1H, H-5), 7.80 (dd, J=9.1, 2.8 Hz, 1H, H-6), 7.70 (d, J=2.8 Hz, 1H, H-8), 4.34 (t, J=5.8 Hz, 2H, CH₂), 3.58 (t, J=4.6 Hz, 4H, 2×CH₂), 2.77 (t, J=5.5 Hz, 2H, CH₂), 2.52-2.58 (m, 4H, 2×CH₂); ¹³C NMR [(CD₃)₂SO] δ 160.3, 153.1, 143.1, 134.6, 129.7, 129.4, 98.7, 66.8, 66.1 (2), 56.5, 53.4 (2); Anal. calc. for C₁₃H₁₅ClN₄O₃: C, 50.3; H, 4.9; N, 18.0; found C, 50.7; H, 5.0; N, 18.1%.

### Example 114

3-Ethyl-7-[2-(4-morpholinyl)ethoxy]-1,2,4-benzotriazine 1-oxide (99). Pd(PPh₃)₄ (92 mg, 0.08 mmol) was added to a N₂ purged solution of chloride 96 (315 mg, 1.0 mmol) and tetraethyltin (0.4 mL, 2.0 mmol) in DMF (15 mL). The purged reaction mixture was heated at reflux temperature for 20 h under N₂. The solvent was evaporated and the residue chromatographed, eluting with a gradient (0-1%) of MeOH/DCM, to give 1-oxide 99 (204 mg, 67%) as a white powder, mp (DCM/pet. ether) 99-101° C.; ¹H NMR [(CD₃)₂SO] δ 7.96 (d, J=9.0 Hz, 1H, H-5), 7.72 (dd, J=9.0, 2.8 Hz, 1H, H-6), 7.70 (d, J=2.6 Hz, 1H, H-8), 4.36 (t, J=5.8 Hz, 2H, CH₂), 3.59 (t, J=4.6 Hz, 4H, 2×CH₂), 2.94 (q, J=7.6 Hz, 2H, CH₂), 2.77 (t, J=5.5 Hz, 2H, CH₂), 2.50 (t, J=4.2 Hz, 4H, 2×CH₂), 1.34 (t, J=7.6 Hz, 3H, CH₃), ¹³C NMR [(CD₃)₂SO] δ 164.8, 159.5, 143.0, 133.3, 129.8, 128.8, 98.3, 66.6, 66.1 (2), 55.6, 53.5 (2), 29.5, 11.9; Anal. calc. for C₁₅H₂₀N₄O₃: C, 59.2; H, 6.6; N, 18.4; found C, 59.3; H, 6.5; N, 18.4%.

### Example 115

3-Chloro-8-methyl-1,2,4-benzotriazine 1-oxide (100). Sodium nitrite (6.15 g, 89.1 mmol) was added in small portions to a stirred solution of 8-methyl-1,2,4-benzotriazin-3-amine 1-oxide (3b) (7.85 g, 44.6 mmol) in trifluoroacetic acid (80 mL) at 5° C. and the solution stirred at 20° C. for 3 h. The solution was poured into ice/water, stirred 30 minutes, filtered, washed with water (3×30 mL) and dried. The solid was suspended in POCl₃ (100 mL) and DMF (0.5 mL) and stirred at 100° C. for 1 h. The solution was cooled, poured into ice/water, stirred for 30 minutes, filtered, washed with water (3×30 mL) and dried. The solid was dissolved in DCM (150 mL), dried and the solvent evaporated. The residue was chromatographed, eluting with 5% EtOAc/DCM, to give chloride 100 (4.25 g, 49%) as a pale yellow solid, mp (EtOAc/DCM) 170-173° C.; ¹H NMR δ 7.78-7.82 (m, 2H, H-5, H-7), 7.47-7.51 (m, 1H, H-6), 2.98 (s, 3H, CH₃); ¹³C NMR δ 156.4 (C-3), 149.1 (C-4a), 135.7 (C-6), 134.5 (C-8), 133.9 (C-8a), 133.1 (C-5), 126.7 (C-7), 23.6 (CH₃); Anal. calc. for C₈H₆ClN₃O: C, 49.1; H, 3.1; N, 21.5; found: C, 49.4; H, 2.9; N, 21.6%.

### Example 116

N¹,N¹-Dimethyl-N²-(8-methyl-1-oxido-1,2,4-benzotriazin-3-yl)-1,2-ethanediamine (101). N,N-Dimethylethanediamine (530 μL, 4.9 mmol) was added to a stirred solution of chloride 100 (316 mg, 1.6 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 101 (341 mg, 85%) as a yellow solid, mp (MeOH/EtOAc) 121-123° C.; ¹H NMR δ 7.48 (dd, J=8.1, 7.1 Hz, 1H, H-6), 7.41 (d, J=8.1 Hz, 1H, H-5), 7.00 (d, J=7.1 Hz, 1H, H-7), 5.86 (br s, 1H, NH), 3.51-3.57 (m, 2H, CH₂N), 2.88 (s, 3H, CH₃), 2.56-2.60 (m, 2H, CH₂N), 2.29 [s, 6H, N(CH₃)₂]; ¹³C NMR δ 158.5 (C-3), 150.7 (C-4a), 134.4 (C-6), 134.2 (C-8), 131.1 (C-8a), 127.5 (C-5), 124.7 (C-7), 57.6 (CH₂N), 45.0 [N(CH₃)₂], 38.6 (CH₂N), 24.0 (CH₃); Anal. calc. for C₁₂H₁₇N₅O; C, 58.3; H, 6.9; N, 28.3; found C, 58.0: H, 7.2; N, 28.1%.

### Example 117

8-Methyl-N-[2-(1-piperidinyl)ethyl]-1,2,4-benzotriazin-3-amine 1-oxide (102). 2-(1-Piperidinyl)ethylamine (1.26 mL, 8.9 mmol) was added to a stirred solution of chloride 100 (578 mg, 3.0 mmol) in DME (50 mL) and the solution stirred at reflux temperature for 2 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give 1-oxide 102 (764 mg, 90%) as a yellow powder, mp (MeOH/EtOAc) 137-140° C.; ¹H NMR δ 7.48 (dd, J=7.8, 7.8 Hz, 1H, H-6), 7.41 (br d, J=7.8 Hz, 1H, H-5), 7.00 (d, J=7.1 Hz, 1H, H-7), 5.90 (br s, 1H, NH), 3.52-3.56 (m, 2H, CH₂N), 2.90 (s, 3H, CH₃), 2.55-2.59 (m, 2H, CH₂N), 2.40-2.45 (m, 4H, 2×CH₂N), 1.55-1.61 (m, 4H, 2×CH₂), 1.41-1.48 (m, 2H, CH₂); ¹3C NMR δ 158.4 (C-3), 150.7 (C-4a), 134.5 (C-6), 134.2 (C-8), 131.1 (C-8a), 127.4 (C-5), 124.7 (C-7), 57.0 (CH₂N), 54.3 (2×CH₂N), 37.9 (CH₂N), 26.0 (2×CH₂), 24.4 (CH₂), 24.0 (CH₃).

### Example 118

3-Chloro-6,7-dimethyl-1-oxido-1,2,4-benzotriazine (104). A mixture of 4,5-dimethyl-2-nitroaniline 103 (5.0 g, 30.1 mmol) and cyanamide (5.06 g, 120 mmol) were mixed together at 100° C. The mixture was cooled to ca. 50° C. and cHCl (15 mL) added (CAUTION: exotherm) and the resulting solution heated at 100° C. for 1 h. The solution was cooled to ca. 50° C. and 7.5 M NaOH solution (50 mL) added carefully. The suspension was stirred at 100° C. for 2 h, cooled to 20° C. and diluted with water (100 mL). The suspension was filtered, washed with water (2×10 mL), washed with ether (2×10 mL) and dried. The yellow solid (4.50 g, 23.7 mmol) was suspended in 2 M HCl (250 mL), cooled to 5° C., and a solution of NaNO₂ (3.27 g, 47.3 mmol) in water (20 mL) added dropwise. The mixture was stirred vigorously for 2 h at 20° C. The suspension was filtered, the solid suspended in dilute aqueous NH₃ (200 mL) and filtered. The filtrate was acidified with cHCl, cooled at 5° C. for 16 h and the precipitate collected. The solid was washed with water (2×15 mL) and dried to give the 3-hydroxy-6,7-dimethyl-1,2,4-benzotriazine 1-oxide (1.21 g, 21%) which was used without further characterization. A mixture of the 3-hydroxide (1.21 g, 6.3 mmol), dimethylaniline (2.0 mL, 15.8 mmol) and POCl₃ (4.1 mL, 44.3 mmol) was heated at reflux temperature for 1 h. The solution was poured onto ice, stirred and filtered. The solid was dissolved in EtOAc (200 mL), dried, and the solvent evaporated. The residue was chromatographed, eluting with 5% EtOAc/DCM, to give the chloride 104 (1.07 g, 81%) as colourless plates, mp 148-149° C.; ¹H NMR δ 8.16 (s, 1H, H-8), 7.72 (s, 1H, H-5), 2.51 (s, 3H, CH₃), 2.50 (s, 3H, CH₃); ¹³C NMR δ 156.1 (C-3), 148.9 (C-6), 146.3 (C-4a), 142.5 (C-7), 132.0 (C-8a), 127.4 (C-5), 119.8 (C-8), 20.8 (CH₃), 20.5 (CH₃); Anal. calc. for C₉H₈ClN₃O: C, 51.6; H, 3.85; N, 20.0; found C, 51.8; H, 3.7; N, 20.2%

### Example 119

tert-Butyl 2-[(6,7-dimethyl-1-oxido-1,2,4-benzotriazi n-3-yl)amino]ethylcarbamate (105). A solution of chloride 104 (842 mg, 4.0 mmol) and tert-butyl 2-aminoethylcarbamate (1.4 g, 8.8 mmol) in DME (50 mL) was heated at reflux temperature for 3 h. The solvent was evaporated and the residue partitioned between EtOAc (100 mL) and water (100 mL). The organic fraction was dried and the solvent evaporated. The residue was chromatographed, eluting with a gradient (20-50%) of EtOAc/DCM, to give 1-oxide 105 (1.04 g, 78%) as a yellow solid, mp (EtOAc/DCM) 226-228° C.; ¹H NMR [(CD₃)₂SO] δ 7.90 (s, 1H, H-8′), 7.66 (br s, 1H, NH), 7.39 (s, 1H, H-5′), 6.88 (t, J=5.3 Hz, 1H, NH), 3.33-3.38 (m, 2H, CH₂N), 3.14-3.18 (m, 2H, CH₂N), 2.36 (s, 3H, CH₃), 2.33 (s, 3H, CH₃), 1.37 [C(CH₃)₃]; ¹³C NMR [(CD₃)₂SO] δ 155.8 (NHCO₂), 155.6 (C-3′), 147.1 (C-4a′), 147.8 (C-7′), 134.8 (C-6′), 128.2 (C-8a′), 125.2 (C-5′), 118.5 (C-8′), 77.6 [C(CH₃)₃], 40.9 (CH₂N), 39.0 (CH₂N), 28.1 [C(CH₃)₃], 19.9 (CH₃), 19.2 (CH₃); Anal. calc. for C₁₆H₂₃N₅O₃: C, 57.6; H, 7.0; N, 21.0; found C, 57.9; H, 7.0; N, 20.8%.

### Example 120

N¹-(6,7-Dimethyl-1-oxido-1,2,4-benzotriazin-3-yl)-N²,N²-dimethyl-1,2-ethanediamine (106). N,N-Dimethyl-1,2-ethanediamine (0.3 mL, 2.7 mmol) was added to a stirred solution of chloride 104 (190 mg, 0.9 mmol) in DME (30 mL) and the solution stirred at reflux temperature for 16 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give amine 106 (181 mg, 76%), mp (MeOH) 175-178° C.; ¹H NMR δ 8.00 (s, 1H, H-8), 7.36 (s, 1H, H-5), 5.82 (br s, 1H, NH), 3.52-3.56 (m, 2H, CH₂N), 2.55-2.59 (m, 2H, CH₂N), 2.38 (s, 3H, CH₃), 2.36 (s, 3H, CH₃), 2.27 [s, 6H, N(CH₃)₂]; ¹³C NMR [(CD₃)₂SO] δ 158.9 (C-3), 147.8 (C-4a), 146.9 (C-6), 135.4 (C-7), 129.1 (C-8a), 125.7 (C-5), 119.4 (C-8), 57.6 (CH₂N), 45.1 [N(CH₃)₂], 38.7 (CH₂N), 20.5 (CH₃), 19.8 (CH₃); MS (EI) m/z261 (M⁺, 5%), 224 (3), 217 (1), 58 (100); HRMS calc. for C₁₃H₁₉N₅O (M+) m/z261.1590, found 261.1587.

### Example 121

3-Chloro-6,8-dimethyl-1,2,4-benzotriazine 1-oxide (108). A mixture 3,5-dimethyl-2-nitroaniline (107) (Andrews et. al., Aust. J. Chem. 1972, 25, 639) (6.61 g, 39.8 mmol) and cyanamide (6.7 g, 159 mmol) were mixed together at 100° C., cooled to 50° C., cHCl (30 mL) added carefully and the mixture heated at 100° C. for 4 h. The mixture was cooled to 50° C., 7.5 M NaOH solution added until the mixture was strongly basic and the mixture stirred at 100° C. for 3 h. The mixture was cooled, diluted with water (100 mL), filtered, washed with water (3×30 mL), washed with ether (3×20 mL) and dried to give crude 1-oxide (2.62 g, 35%) as a yellow powder. Sodium nitrite (1.55 g, 22.5 mmol) was added in small portions to a stirred solution of 1-oxide (2.14 g, 11.3 mmol) in trifluoroacetic acid (20 mL) at 5° C. and the solution stirred at 20° C. for 3 h. The solution was poured into ice/water, stirred 30 minutes, filtered, washed with water (3×30 mL) and dried. The solid was suspended in POCl₃ (50 mL) and DMF (0.2 mL) stirred at 100° C. for 1 h. The solution was cooled, poured into ice/water, stirred for 30 minutes, filtered, washed with water (3×30 mL) and dried. The solid was suspended in DCM (150 mL), dried and the solvent evaporated. The residue was chromatographed, eluting with 5% EtOAc/DCM, to give chloride 108 (1.58 g, 67%) as a pale yellow solid, mp (EtOAc/DCM) 120-122° C.; ¹H NMR δ 7.55 (s, 1H, H-5), 7.30 (s, 1H, 1H, H-7), 2.93 (s, 3H, CH₃), 2.52 (s, 3H, CH₃); ¹³C NMR δ 156.4 (C-3), 149.4 (C-4a), 147.7 (C-6), 135.1 (C-5), 133.9 (C-8), 132.2 (C-8a), 125.5 (C-7), 23.4 (CH₃), 21.9 (CH₃); Anal. calc. for C₉H₈ClN₃O: C, 51.6; H, 3.9; N, 20.0; Cl, 16.9; found C, 51.8; H, 3.6; N, 20.2; Cl, 16.6%.

### Example 122

N¹-(6,8-Dimethyl-1-oxido-1,2,4-benzotriazi n-3-yl)-N²,N²-dimethyl-1,2-ethanediamine (109). N,N-Dimethylethanediamine (0.64 mL, 5.9 mmol) was added to a stirred solution of chloride 108 (494 mg, 2.4 mmol) in DME (80 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 109 (561 mg, 91%) as a yellow solid, mp (MeOH) 176-179° C.; ¹H NMR δ 7.20 (br s, 1H, H-5), 6.84 (br s, 1H, H-7), 5.76 (br s, 1H, NH), 3.50-3.54 (m, 2H, CH₂N), 2.85 (s, 3H, CH₃), 2.52-2.56 (m, 2H, CH₂N), 2.38 (s, 3H, CH₃), 2.26 [s, 6H, N(CH₃)₂]; ¹³C NMR δ 158.7 (C-3), 150.9 (C-4a), 145.6 (C-6), 133.7 (C-8), 129.6 (C-5), 129.4 (C-8a), 123.7 (C-7), 57.6 (CH₂N), 45.1 [N(CH₃)₂], 38.7 (CH₂N), 23.8 (CH₃), 21.6 (CH₃); Anal. calc. for C₁₃H₁₉N₅O; C, 59.8; H, 7.3; N, 26.8; found C, 60.0: H, 7.6; N, 27.0%.

### Example 123

6,8-Dimethyl-N-[2-(1-piperidinyl)ethyl]-1,2,4-benzotriazin-3-amine 1-oxide (110). 2-(1-Piperidinyl)ethylamine (0.67 mL, 4.7 mmol) was added to a stirred solution of chloride 108 (395 mg, 1.9 mmol) in DME (50 mL) and the solution stirred at reflux temperature for 2 h. The solvent was evaporated and the residue partitioned between DCM (100 mL) and dilute aqueous NH₃ (50 mL). The organic fraction was dried and the solvent evaporated. The residue was chromatographed, eluting with a gradient (0-5%) of MeOH/DCM, to give 1-oxide 110 (517 mg, 91%) as a yellow powder, mp (MeOH) 177-178° C.; ¹H NMR δ 7.20 (s, 1H, H-5), 6.84 (s, 1H, H-7), 5.84 (br s, 1H, NH), 3.49-3.55 (m, 2H, CH₂N), 2.85 (s, 3H, CH₃), 2.54-2.57 (m, 2H, CH₂N), 2.39-2.44 (m, 4H, 2×CH₂N), 2.37 (s, 3H, CH₃), 1.55-1.61 (m, 4H, 2×CH₂), 1.41-1.47 (m, 2H, CH₂); ¹³C NMR δ 158.6 (C-3), 150.9 (C-4a), 145.6 (C-6), 133.7 (C-8), 129.5 (C-5), 129.4 (C-8a), 123.7 (C-7), 57.0 (CH₂N), 54.3 (2×CH₂N), 37.9 (CH₂N), 26.0 (2×CH₂), 24.4 (CH₂), 23.8 (CH₃), 21.7 (CH₃); Anal. calc. for C₁₆H₂₃N₅O: C, 63.8; H, 7.7; N, 23.2; found C, 63.9; H, 8.0; N, 23.5%.

### Example 124

6-Methoxy-7-methyl-1,2,4-benzotriazin-3-amine 1-oxide (112). A mixture of 5-methoxy-4-methyl-2-nitroaniline (111) (James & Felix, U.S. Pat. No. 5,360,986) (8.9 g, 49 mmol) and cyanamide (8.2 g, 196 mmol) were mixed together at 100° C., cooled to 50° C., cHCl (50 mL) added carefully and the mixture heated at 100° C. for 4 h. The mixture was cooled to 50° C., 7.5 M NaOH solution added until the mixture was strongly basic and the mixture stirred at 100° C. for 3 h. The mixture was cooled, diluted with water (200 mL), filtered, washed with water (3×50 mL), washed with ether (3×30 mL) and dried. The solid was recrystallized from MeOH to give 1-oxide 112 (6.0 g, 59%) as a yellow powder, mp (MeOH) 289-292° C.; ¹H NMR [(CD₃)₂SO] δ 7.91 (d, J=1.1 Hz, 1H, H-8), 7.10 (br s, 2H, NH₂), 6.84 (s, 1H, H-5), 3.94 (s, 3H, OCH₃), 2.23 (s, 3H, CH₃); ¹³C NMR [(CD₃)₂SO] δ 163.7 (C-6), 160.4 (C-3), 150.3 (C-4a), 127.3 (C-7), 124.4 (C-8a), 120.0 (C-8), 102.7 (C-5), 56.3 (OCH₃), 16.1 (CH₃); Anal. calc. for C₉H₁₀N₄O₂.¹/4MeOH: C, 51.9; H, 5.2; N, 26.2; found C, 52.1; H, 4.8; N, 26.4%.

### Example 125

3-Chloro-6-methoxy-7-methyl-1,2,4-benzotriazine 1-oxide (113). Sodium nitrite (3.38 g, 49.0 mmol) was added in small portions to a stirred solution of 1-oxide 112 (5.05 g, 24.5 mmol) in trifluoroacetic acid (30 mL) at 5° C. and the solution stirred at 20° C. for 3 h. The solution was poured into ice/water, stirred 30 minutes, filtered, washed with water (3×30 mL) and dried. The solid was suspended in POCl₃ (50 mL) and DMF (0.2 mL) stirred at 100° C. for 1 h. The solution was cooled, poured into ice/water, stirred for 30 minutes, filtered, washed with water (3×30 mL) and dried. The solid was suspended in DCM (150 mL), dried and the solvent evaporated. The residue was chromatographed, eluting with 5% EtOAc/DCM, to give chloride 113 (2.72 g, 49%) as a pale yellow solid, mp (EtOAc) 180-182° C.; ¹H NMR δ 8.44 (d, J=0.9 Hz, 1H, H-8), 7.14 (s, 1H, 1H, H-5), 4.03 (s, 3H, OCH₃), 2.40 (d, J=0.9 Hz, 3H, CH₃); ¹³C NMR δ 165.4 (C-6), 156.8 (C-3), 149.2 (C-4a), 135.5 (C-7), 128.4 (C-8a), 120.4 (C-8), 104.3 (C-5), 56.6 (OCH₃), 17.2 (CH₃); Anal. calc. for C₉H₈ClN₃O₂: C, 47.9; H, 3.6; N, 18.6; Cl, 15.7; found C, 48.0; H, 3.5; N, 18.6; Cl, 15.7%.

### Example 126

N¹-(6-Methoxy-7-methyl-1-oxido-1,2,4-benzotriazi n-3-yl)-N²,N²-dimethyl-1,2-ethanediamine (114). N,N-Dimethylethanediamine (0.70 mL, 6.3 mmol) was added to a stirred solution of chloride 113 (474 mg, 2.1 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-15%) of MeOH/DCM, to give 1-oxide 114 (529 mg, 90%) as a yellow solid, mp (MeOH) 167-169° C.; ¹H NMR δ 7.99 (d, J=1.0 Hz, 1H, H-8′), 6.79 (s, 1H, H-5′), 5.84 (br s, 1H, NH), 3.94 (s, 3H, OCH₃), 3.49-3.54 (m, 2H, CH₂N), 2.52-2.56 (m, 2H, CH₂N), 2.27 (d, J=1.0 Hz, 3H, CH₃), 2.26 [s, 6H, N(CH₃)₂]; ¹³C NMR δ 164.5 (C-6′), 159.3 (C-3′), 150.5 (C-4a′), 128.5 (C-7′), 125.4 (C-8a′), 120.7 (C-8′), 120.7 (C-5′), 57.5 (CH₂N), 56.1 (OCH₃), 45.1 [N(CH₃)₂], 38.7 (CH₂N), 16.5 (CH₃); Anal. calc. for C₁₃H₁₉N₅O₂: C, 56.3; H, 6.9; N, 25.3; found C, 56.5: H, 7.2; N, 25.7%.

### Example 127

7-Methoxy-6-m thyl-1,2,4-benzotriazin-3-amine 1-oxid (116). A mixture of 4-methoxy-5-methyl-2-nitroaniline (115) (Arnold & McCool, J. Am. Chem. Soc. 1942, 64, 1315) (2.3 g, 12.6 mmol) and cyanamide (2.0 g, 50 mmol) were mixed together at 100° C., cooled to 50° C., cHCl (20 mL) added carefully and the mixture heated at 100° C. for 4 h. The mixture was cooled to 50° C., 7.5 M NaOH solution added until the mixture was strongly basic and the mixture stirred at 100° C. for 3 h. The mixture was cooled, diluted with water (100 mL), filtered, washed with water (3×30 mL), washed with ether (3×20 mL) and dried to give crude 1-oxide 116 (2.52 g, 97%) as a yellow powder, mp (MeOH)>320° C.; ¹H NMR [(CD₃)₂SO] δ 7.42 (s, 1H, H-8), 7.39 (d, J=1.0 Hz, 1H, H-5), 6.99 (br s, 2H, NH₂), 3.90 (s, 3H, OCH₃), 2.30 (s, 3H, CH₃); ¹³C NMR [(CD₃)₂SO] δ 159.7 (C-7), 155.4 (C-3), 144.8 (C-4a), 139.3 (C-6), 128.4 (C-8a), 126.2 (C-5), 96.6 (C-8), 56.0 (OCH₃), 16.7 (CH₃).

### Example 128

3-Chloro-7-methoxy-6-methyl-1,2,4-benzotriazine 1-oxide (117). Sodium nitrite (1.7 g, 24.4 mmol) was added in small portions to a stirred solution of 1-oxide 116 (2.50 g, 12.2 mmol) in trifluoroacetic acid (20 mL) at 5° C. and the solution stirred at 20° C. for 3 h. The solution was poured into ice/water, stirred 30 minutes, filtered, washed with water (3×30 mL) and dried. The solid was suspended in POCl₃ (50 mL) and DMF (0.2 mL) stirred at 100° C. for 1 h. The solution was cooled, poured into ice/water, stirred for 30 minutes, filtered, washed with water (3×30 mL) and dried. The solid was suspended in DCM (150 mL), dried and the solvent evaporated. The residue was chromatographed, eluting with 5% EtOAc/DCM, to give chloride 117 (1.38 g, 50%) as a pale yellow solid, mp (EtOAc/DCM) 200-202° C.; ¹H NMR δ 7.70 (d, J=0.9 Hz, 1H, H-5), 7.59 (s, 1H, 1H, H-8), 4.03 (s, 3H, OCH₃), 2.44 (d, J=0.9 Hz, 3H, CH₃); ¹³C NMR δ 160.7 (C-7), 154.7 (C-3), 143.5 (C-4a), 142.0 (C-6), 133.1 (C-8a), 128.4 (C-5), 96.4 (C-8), 56.6 (OCH₃), 17.4 (CH₃); Anal. calc. for C₉H₈ClN₃O₂: C, 47.9; H, 3.6; N, 18.6; found C, 48.1; H, 3.4; N, 18.7%.

### Example 129

N¹,N¹-Dimethyl-N²-(7-methoxy-6-methyl-1-oxido-1,2,4-benzotriazin-3-yl)-1,2-ethanediamine (118). N,N-Dimethylethanediamine (0.48 mL, 4.3 mmol) was added to a stirred solution of chloride 117 (391 mg, 1.7 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 118 (432 mg, 90%) as a yellow solid, mp (MeOH/EtOAc) 182-184° C.; ¹H NMR δ 7.49 (s, 1H, H-8′), 7.36 (s, 1H, H-5′), 5.72 (br s, 1H, NH), 3.92 (s, 3H, OCH₃), 3.49-3.54 (m, 2H, CH₂N), 2.53-2.56 (m, 2H, CH₂N), 2.34 (s, 3H, CH₃), 2.26 [s, 6H, N(CH₃)₂]; ¹³C NMR δ 158.6 (C-7′), 156.4 (C-3′), 145.2 (C-4a′), 140.2 (C-6′), 129.4 (C-8a′), 124.9 (C-5′), 96.9 (C-8′), 57.6 (CH₂N), 56.0 (OCH₃), 45.1 [N(CH₃)₂], 38.8 (CH₂N), 17.2 (CH₃); Anal. calc. for C₁₃H₁₉N₅O₂: C, 56.3; H, 6.9; N, 25.3; found C, 56.5: H, 6.7; N, 25.5%.

### Example 130

7-Methoxy-6-methyl-N-[2-(1-piperidinyl)ethyl]-1,2,4-benzotriazin-3-amine 1-oxide (119). 2-(1-Piperidinyl)ethylamine (0.74 mL, 5.2 mmol) was added to a stirred solution of chloride 117 (467 mg, 2.1 mmol) in DME (50 mL) and the solution stirred at reflux temperature for 4 h. The solvent was evaporated and the residue partitioned between DCM (100 mL) and dilute aqueous NH₃ (50 mL). The organic fraction was dried and the solvent evaporated. The residue was chromatographed, eluting with a gradient (0-5%) of MeOH/DCM, to give 1-oxide 119 (574 mg, 87%) as a yellow powder, mp (MeOH/EtOAc) 195-197° C.; ¹H NMR δ 7.50 (s, 1H, H-8), 7.36 (d, J=0.8 Hz, 1H, H-5), 5.83 (br s, 1H, NH), 3.92 (s, 3H, OCH₃), 3.49-3.54 (m, 2H, CH₂N), 2.54-2.59 (m, 2H, CH₂N), 2.39-2.43 (m, 4H, 2×CH₂N), 2.33 (s, 3H, CH₃), 1.54-1.60 (m, 4H, 2×CH₂), 1.41-1.47 (m, 2H, CH₂); ¹³C NMR δ 158.6 (C-7), 156.3 (C-3), 145.2 (C-4a), 140.2 (C-6), 129.4 (C-8a), 126.8 (C-5), 96.9 (C-8), 57.0 (CH₂N), 56.0 (OCH₃), 54.3 (2×CH₂N), 38.0 (CH₂N), 26.0 (2×CH₂), 24.4 (CH₂), 17.2 (CH₃); Anal. calc. for C₁₆H₂₃N₅O₂: C, 60.6; H, 7.3; N, 22.1; found C, 60.6; H, 7.1; N, 22.3%.

### Example 131

N-(2-{2-[(1-Oxido-1,2,4-benzotriazin-3-yl)amino]ethoxy}ethyl)-4-acridinecarboxamide (121). A solution of amine 36 (0.53 g, 2.1 mmol) in DCM (10 mL) was added dropwise to a stirred solution of imidazolide of acridine-4-carboxylic acid 120 (0.58 g, 2.1 mmol) in THF (25 mL) and the solution stirred at 20° C. for 72 h. The solvent was evaporated and the residue chromatographed, eluting with a gradiant (0-4%) of MeOH/DCM, to give 121 (642 mg, 66%) as a yellow powder, mp 178-182° C.; ¹H NMR [(CD₃)₂SO] δ 11.75 (t, J=5.0 Hz, 1H, NH), 9.27 (s, 1H, H-9), 8.74 (dd, J=8.4, 1.5 Hz, 1H, H-3), 8.36 (dd, J=8.4, 1.4 Hz, 1H, H-1), 8.18 (m, 2H, H-5, H-8), 8.02 (dd, J=8.6, 1.2 Hz, 1H, H-8′), 7.90 (br s, 1H, NH), 7.83 (t, J=7.5 Hz, 1H, H-6), 7.75 (dd, J=8.6, 7.2 Hz, 1H, H-2), 7.70 (ddd, J=8.6, 7.1, 1.2 Hz, 1H, H-6′), 7.61 (ddd, J=8.9, 7.1, 0.7 Hz, 1H, H-7), 7.42 (br d, J=8.6 Hz, 1H, H-5′), 7.28 (ddd, J=8.4, 7.1, 1.4 Hz, 1H, H-7′), 3.80-3.84 (m, 4H, 2×CH₂O), 3.73-3.77 (m, 2H, CH₂N), 3.60-3.65 (m, 2H, CH₂N); ¹³C NMR [(CD₃)₂SO] δ 164.6 (CONH), 158.8 (C-3′), 148.0 (C-4a′), 146.8 (C-4b), 145.3 (C-4a), 138.5 (C-9), 135.5 (C-6′), 134.5 (C-3), 132.8 (C-1), 131.7 (C-6), 129.9 (C-9a, C-8a′), 128.3 (C-8), 128.2 (C-5), 127.9 (C-7), 126.3 (C-5′), 125.8 (C-4), 125.5 (C-8a), 125.1 (C-7′), 124.4 (C-2), 119.7 (C-8′), 69.0 (CH₂O), 68.4 (CH₂O), 40.5 (CH₂N), 39.1 (CH₂N); Anal. calc. for C₂₅H₂₂N₆O₃.½H₂O: C, 64.8; H, 5.0; N, 18.1; found C, 65.2; H, 4.8; N, 18.4%.

### Example 132

3-[(2-Methoxyethyl)amino]-2-quinoxalinecarbonitrile 1-oxide (123). 2-Methoxyethylamine (0.32 mL, 3.0 mmol) was added to a stirred solution of 3-chloro-2-quinoxalinecarbonitrile 1-oxide 122 (Yoshida & Otomasu, Chem. Pharm. Bull. 1984, 32, 3361) (203 mg, 1.0 mmol) in DME (50 mL) and the solution stirred at reflux temperature for 2 h. The solvent was evaporated and the residue partitioned between DCM (100 mL) and dilute aqueous NH₃ (50 mL). The organic fraction was dried and the solvent evaporated. The residue was chromatographed, eluting with 5% EtOAc/DCM, to give 1-oxide 123 (209 mg, 86%) as a yellow powder, mp (MeOH) 124-126° C.; ¹H NMR δ 8.28 (br d, J=8.5 Hz, 1H, H-8), 7.66-7.71 (m, 2H, H-5, H-6), 7.36-7.42 (m, 1H, H-7), 5.60 (br s, 1H, NH), 3.78-3.84 (m, 2H, CH₂N), 3.63-3.66 (m, 2H, CH₂O), 3.42 (s, 3H, OCH₃); ¹³C NMR δ 154.2 (C-3), 144.6 (C-4a), 134.1 (C-6), 132.6 (C-8a), 127.6 (C-5), 125.7 (C-7), 118.9 (C-8), 111.9 (C-2), 108.2 (CN), 70.4 (CH₂O), 59.0 (OCH₃), 41.2 (CH₂N); Anal. calc. for C₁₂H₁₂N₄O₂: C, 59.0; H, 5.0; N, 22.9; found C, 59.2; H, 5.2; N, 22.6%.

### Example 133

1,2,4-Benzotriazine 1-oxide (124). Isoamyl nitrite (1.05 mL, 7.8 mmol) was added to a stirred solution of 1-oxide 3 (254 mg, 1.6 mmol) in DMF (10 mL) and the solution stirred at 60° C. for 2 h. The solvent was evaporated and the residue partitioned between EtOAc (50 mL) and water (50 mL). The organic fraction was washed with water (2×25 mL), brine (20 mL), dried, and the solvent evaporated. The residue was chromatographed, eluting with a gradient (0-10%) of EtOAc/DCM, to give 124 (120 mg, 52%) as a pale yellow solid, mp (EtOAc/DCM) 138-139° C. [lit. (Robbins & Schofield, J. Chem. Soc. 1957, 3186) mp (MeOH) 138-140° C.]; ¹H NMR δ 9.19 (s, 1 H, H-3), 8.41 (d, J=8.6 Hz, 1H, H-8), 8.10-8.13 (m, 2H, H-5, H-6), 7.90-7.94 (m, 1H, H-7).

### Examples 134 and 135

1,2,4-Benzotriazin-3-amine 2-oxide (126) and 1,2,4-benzotriazin-3-amine 4-oxide (127).

1,2,4-Benzotriazin-3-amine (125). A solution of 1-oxide 3 (1.98 g, 14.3 mmol) and Na₂S₂O₄ (4.99 g, 28.7 mmol) in 70% aqueous EtOH (100 mL) was heated at reflux temperature for 3 h. The hot suspension was filtered and the filtrate extracted with CHCl₃ (3×50 mL). The combined organic fraction was dried and the solvent evaporated. The combined solid and extracts were chromatographed, eluting with a gradient (0-2%) of MeOH/CHCl₃, to give benzotriazine 125 (1.58 g, 67%) as a yellow solid, mp (CHCl₃/MeOH) 200-203° C. [lit. (Mason & Tennant, J. Chem. Soc. (B) 1970, 911) mp 207° C.]; ¹H NMR δ 8.19 (dd, J=8.3, 0.9 Hz, 1H, H-8), 7.78-7.83 (m, 1H, H-6), 7.62 (br s, 2H, NH₂), 7.54 (d, J=8.4 Hz, 1H, H-5), 7.43-7.48 (m, 1H, H-7).

1,2,4-Benzotriazin-3-amine 2-oxide (126) and 1,2,4-benzotriazin-3-amine 4-oxide (127). A solution of MCPBA (0.89 g, 3.6 mmol) in DCM (5 mL) was added dropwise to a stirred solution of 125 (0.50 g, 3.4 mmol) in 10% MeOH/DCM (50 mL) at 20° C. and the solution stirred at 20° C. for 3 h. The solution was washed with dilute aqueous NH₃ solution (50 mL), dried, and the solvent evaporated. The residue was chromatographed, eluting with a gradient (0-5%) of MeOH/CHCl₃, to give (i) 1,2,4-benzotriazin-3-amine 2-oxide (126) (405 mg, 70%) as a yellow powder, mp (MeOH/DCM) 175-180° C. [lit. (Mason & Tennant, J. Chem. Soc. (B) 1970, 911) mp (HOAc) 200° C.]; ¹H NMR δ 8.20 (br s, 2H, NH₂), 7.68 (d, J=8.2 Hz, 1H, H-8), 7.60-7.65 (m, 1H, H-6), 7.53 (d, J=7.6 Hz, 1H, H-5), 7.45-7.59 (m, 1H, H-7); followed by (ii) 1,2,4-benzotriazin-3-amine 1-oxide (3) (65 mg, 11%) as a yellow powder, mp 266-268° C. [lit. (Arndt, Ber. 1913, 46, 3522) mp (EtOH) 269° C.]; spectroscopically identical with the sample prepared above; and (iii) 1,2,4-benzotriazin-3-amine 4-oxide (127) (51 mg, 9%) as pale yellow solid, [lit. (Fuchs, et. al., J. Org. Chem. 2001, 66, 107)]; ¹H NMR δ 8.29 (d, J=8.6 Hz, 1H, H-8), 8.20 (br s, 2H, NH₂), 8.16 (d, J=8.6 Hz, 1H, H-5), 7.91 (ddd, J=8.6, 7.0, 1.2 Hz, 1H, H-6), 7.65 (ddd, J=8.6, 7.0, 1.1 Hz, 1H, H-7); MS (EI) 162 (M⁺, 100%), 146 (10); HRMS (EI) calc. for C₇H₆N₄O (M⁺) m/z 162.0542, found 162.0540.

### Example 136

tert-Butyl bis{3-[(1-oxido-1,2,4-benzotriazi n-3-yl)amino]propyl}carbamat (129). A solution of chloride 19 (0.86 g, 4.8 mmol), Et₃N (1.0 mL, 7.1 mmol) and tert-butyl bis(3-aminopropyl)carbamate (1.1 g, 4.8 mmol) in DCM was stirred at 20° C. for 3 days. The solvent was evaporated and the residue chromatographed, eluting with 20% EtOAc/DCM, to give bis-1-oxide 129 (457 mg, 18%) as a yellow oil, ¹H NMR δ 8.24 (d, J=8.4 Hz, 2H, 2×H-8′), 7.65-7.69 (m, 2H, 2×H-6′), 7.56 (br d, J=8.5 Hz, 2H, 2×H-5′), 7.23-7.28 (m, 2H, 2×H-7′), 6.26 (br s, 2H, 2×NH), 3.54-3.57 (m, 4H, 2×CH₂N), 2.32-2.39 (m, 4H, 2×CH₂N), 1.85-1.93 (m, 4H, 2×2×CH₂), 1.49 [s, 9H, C(CH₃)₃]; ¹³C NMR δ 158.9 (2×C-3′), 156.1 (NCO₂), 148.9 (2×C-4a′), 135.4 (2×C-6′), 130.9 (2×C-8a′), 126.4 (2×C-5′), 124.9 (2×C-7′), 120.4 (2×C-8′), 80.2 [OC(CH₃)₃], 60.4 (2×CH₂N), 46.0 (2×NCH₂), 28.5 (2×CH₂), 28.4 [OC(CH₃)₃]; Anal. calc. for C₂₅H₃₁N₉O₄: C, 57.6; H, 6.0; found C, 57.1; H, 6.1%.

### Example 137

N¹-Methyl-#f-(1-oxido-1,2,4-benzotriazin-3-yl)-1,2-ethanediamine (130). N-Methylethanediamine (0.73 mL, 8.3 mmol) was added to a stirred solution of chloride 19 (1.0 g, 5.5 mmol) and Et₃N (1.2 mL, 8.3 mmol) in DCM (50 mL) and the solution stirred at 20° C. for 2 days. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 130 (600 mg, 50%) as a yellow solid, mp (MeOH/EtOAc) 226-228° C.; ¹H NMR [(CD₃)₂SO] δ 8.40 (br s, 1H, NH), 8.17 (dd, J=8.6, 1.0 Hz, 1H, H-8′), 7.84 (ddd, J=8.4, 7.0, 1.0 Hz, 1H, H-6′), 7.64 (br d, J=8.4 Hz, 1H, H-5′), 7.40 (ddd, J=8.6, 7.0, 1.0 Hz, 1H, H-7′), 3.93-3.97 (m, 2H, CH₂N), 3.53 [br s, 1H, NH), 3.21 (s, 3H, NCH3), 3.13-3.17 (m, 2H, CH₂N); ¹³C NMR [(CD₃)₂SO] δ 158.3 (C-3′), 148.2 (C-4a′), 135.9 (C-6′), 129.3 (C-8a′), 126.2 (C-5′), 125.2 (C-7′), 119.8 (C-8′), 45.5 (CH₂N), 36.7 (CH₂N), 35.6 (NCH₃).

### Example 138

3-Chloro-6-fluoro-1,2,4-benzotriazine 1-Oxide (131). NaNO₂ (4.26 g, 61.7 mmol) was added in small portions to a stirred solution of amine 3t (5.56 g, 30.9 mmol) in trifluoroacetic acid (60 mL) at 5° C. and the solution stirred at 20° C. for 3 h. The solution was poured into ice/water, stirred 30 minutes, filtered, washed with water (3×30 mL) and dried. The solid was suspended in POCl₃ (80 mL) and DMF (0.5 mL) and stirred at 100° C. for 1 h. The solution was cooled, poured into ice/water, stirred for 30 minutes, filtered, washed with water (3×30 mL) and dried. The solid was suspended in DCM (150 mL), dried and the solvent evaporated. The residue was chromatographed, eluting with 5% EtOAc/DCM, to give chloride 131 (2.78 g, 45%) as a pale yellow solid, mp (EtOAc/DCM) 166-168° C.; ¹H NMR δ 8.45 (dd, J=9.5, 5.3 Hz, 1H, H-8), 7.61 (dd, J=8.3, 2.6 Hz, 1H, H-5), 7.45-7.52 (m, 1H, H-7); ¹³C NMR δ 167.1 (q, J=264 Hz), 158.4, 149.2, 131.0, 123.4 (d, J=11 Hz), 120.1 (d, J=26 Hz), 112.9 (d, J=23 Hz). Anal. calcd for C₇H₃CIFN₃O: C, 42.1; H, 1.5; N, 21.1; Cl, 17.8; found C, 42.4; H, 1.6; N, 21.2; Cl, 17.8%.

### Example 139

N¹-(6-Fluoro-1-oxido-1,2,4-benzotriazin-3-yl)-N²,N²-dipropyl-1,2-ethanediamine (132). N,N-Dipropylethanediamine (0.76 g, 5.3 mmol) was added to a stirred solution of chloride 131 (455 mg, 2.1 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 132 (645 mg, 96%) as a yellow solid, mp (MeOH) 77-79° C.; ¹H NMR δ 8.18 (d, J=9.1 Hz, 1H, H-8), 7.58 (d, J=2.1 Hz, 1H, H-5), 7.19 (dd, J=9.1, 2.1 Hz, 1H, H-7), 6.04 (br s, 1H, NH), 3.47-3.52 (m, 2H, CH₂N), 2.69 (dd, J=6.0, 5.8 Hz, 2H, CH₂N), 2.38-2.44 (m, 4H, 2×CH₂N), 1.42-1.51 (m, 4H, 2×CH₂), 0.89 (t, J=7.3 Hz, 6H, 2×CH₃); ¹³C NMR δ 159.4, 149.6, 141.9, 129.4, 124.5 (2), 121.9, 55.8 (2), 52.4, 38.9, 20.3 (2), 11.9 (2). Anal. calcd for C₁₅H₂₂ClN₅O: C, 55.6; H, 6.9; N, 21.6; found C, 55.8; H, 7.0; N, 21.7%.

### Example 140

3,6-Dichloro-1,2,4-benzotriazine 1-Oxide (133).). NaNO₂ (2.78 g, 40.3 mmol) was added in small portions to a stirred solution of amine 3u (3.96 g, 20.1 mmol) in trifluoroacetic acid (70 mL) at 5° C. and the solution stirred at 20° C. for 3 h. The solution was poured into ice/water, stirred 30 minutes, filtered, washed with water (3×30 mL) and dried. The solid was suspended in POCl₃ (60 mL) and DMF (0.5 mL) and stirred at 100° C. for 1 h. The solution was cooled, poured into ice/water, stirred for 30 minutes, filtered, washed with water (3×30 mL) and dried. The solid was suspended in DCM (150 mL), dried and the solvent evaporated. The residue was chromatographed, eluting with 5% EtOAc/DCM, to give dichloride 133 (3.59 g, 83%) as a pale yellow solid, mp (DCM) 149-151° C.; ¹H NMR δ 8.36 (d, J=9.2 Hz, 1H, H-8), 7.98 (d, J=2.1 Hz, 1H, H-5), 7.69 (dd, J=9.2, 2.1 Hz, 1H, H-7); ¹³C NMR δ 158.3, 147.8, 143.7, 132.4, 131.9, 127.5, 121.7. Anal. calcd for C₇H₃Cl₂N₃O: C, 38.9; H, 1.4; N, 19.5; Cl, 32.8; found C, 39.1; H, 1.2; N, 19.6; Cl, 32.9%.

### Example 141

N¹-(6-Chloro-1-oxido-1,2,4-benzotriazin-3-yl)-N²,N²-diethyl-1,2-ethanediamine (134). N,N-Diethylethanediamine (0.55 mL, 3.9 mmol) was added to a stirred solution of chloride 133 (425 mg, 2.0 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 134 (566 mg, 97%) as a yellow solid, mp (MeOH) 128-130° C.; ¹H NMR δ 8.18 (d, J=9.1 Hz, 1H, H-8), 7.58 (br s, 1H, H-5), 7.19 (dd, J=9.1, 2.1 Hz, 1H, H-7), 6.09 (br s, 1H, NH), 3.48-3.53 (m, 2H, CH₂N), 2.68 (dd, J=6.1, 5.9 Hz, 2H, CH₂N), 2.56 (q, J=7.1 Hz, 4H, 2×CH₂N), 1.03 (t, J=7.1 Hz, 6H, 2×CH₃); ¹³C NMR δ 158.9, 149.0, 141.4, 128.9, 124.8 (2), 121.4, 50.5, 46.0 (2), 38.2, 11.3 (2). Anal. calcd for C₁₃H₁₈ClN₅O: C, 52.8; H, 6.1; N, 23.7; found C, 53.1; H, 6.3; N, 23.9%.

### Example 142

6-Chloro-N-[2-(1-piperidinyl)ethyl]-1,2,4-benzotriazin-3-amine 1-Oxide (135). N,N-2-(1-Piperidinyl)ethanamine (0.62 mL, 4.4 mmol) was added to a stirred solution of chloride 133 (382 mg, 1.8 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 135 (518 mg, 95%) as a yellow solid, mp (MeOH) 178-181° C.; ¹H NMR δ 8.17 (d, J=9.1 Hz, 1H, H-8), 7.57 (br s, 1H, H-5), 7.18 (dd, J=9.1, 2.1 Hz, 1H, H-7), 6.15 (br s, 1H, NH), 3.51-3.58 (m, 2H, CH₂N), 2.57 (dd, J=6.0, 5.9 Hz, 2H, CH₂N), 2.39-2.45 (m, 4H, 2×CH₂N), 1.55-1.61 (m, 4H, 2×CH₂), 1.42-1.48 (m, 2H, CH₂); ¹³C NMR δ 159.4, 149.6, 141.9, 129.4, 125.4 (2), 121.9, 56.7, 54.3 (2), 37.9, 26.0 (2), 24.4. Anal. calcd for C₁₄H₁₈ClN₅O: C, 54.6; H, 5.9; N, 22.7; found C, 54.6; H, 5.9; N, 22.7%.

### Example 143

N¹-(6-Chloro-1-oxido-1,2,4-b nzotriazin-3-yl)-N²,N²-dipropyl-1,2-ethanediamine (136). N,N-Dipropylethanediamine (0.76 g, 5.3 mmol) was added to a stirred solution of chloride 133 (455 mg, 2.1 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 136 (645 mg, 96%) as a yellow solid, mp (MeOH) 77-79° C.; ¹H NMR δ 8.18 (d, J=9.1 Hz, 1H, H-8), 7.58 (d, J=2.1 Hz, 1H, H-5), 7.19 (dd, J=9.1, 2.1 Hz, 1H, H-7), 6.04 (br s, 1H, NH), 3.47-3.52 (m, 2H, CH₂N), 2.69 (dd, J=6.0, 5.8 Hz, 2H, CH₂N), 2.38-2.44 (m, 4H, 2×CH₂N), 1.42-1.51 (m, 4H, 2×CH₂), 0.89 (t, J=7.3 Hz, 6H, 2×CH₃); ¹³C NMR δ 159.4, 149.6, 141.9, 129.4, 124.5 (2), 121.9, 55.8 (2), 52.4, 38.9, 20.3 (2), 11.9 (2). Anal. calcd for C₁₅H₂₂ClN₅O: C, 55.6; H, 6.9; N, 21.6; found C, 55.8; H, 7.0; N, 21.7%.

### Example 144

N¹,N¹-Diethyl-N²-(6-methyl-1-oxido-1,2,4-benzotriazin-3-yl)-1,2-ethanediamine (137). N,N-Diethylethanediamine (0.68 mL, 4.9 mmol) was added to a stirred solution of chloride 73 (380 mg, 1.9 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) MeOH/DCM, to give 1-oxide 137 (502 mg, 94%) as a yellow solid, mp (MeOH/EtOAc) 78-80° C.; ¹H NMR δ 8.13 (d, J=8.7 Hz, 1H, H-8), 7.36 (br s, 1H, H-5), 7.08 (dd, J=8.7, 1.6 Hz, 1H, H-7), 5.98 (br s, 1H, NH), 3.50-3.55 (m, 2H, CH₂N), 2.60-2.73 (m, 2H, CH₂N), 2.59 (q, J=7.1 Hz, 4H, 2×CH₂N), 2.46 (s, 3H, CH₃), 1.05 (t, J=7.1 Hz, 6H, 2×CH₃); ¹³C NMR δ 159.1, 149.1, 146.9, 129.1, 126.9, 125.3, 120.1, 51.2, 46.7 (2), 38.7, 22.0, 11.7 (2). Anal. calcd for C₁₄H₂₁N₅O: C, 61.1; H, 7.7; N, 25.4; found C, 60.8: H, 8.0; N, 25.2%.

### Example 145

6-Methyl-N-[2-(4-morpholinyl)ethyl]-1,2,4-benzotriazin-3-amine 1-Oxide (138). 2-(1-Morpholinyl)ethylamine (1.57 mL, 12.0 mmol) was added to a stirred solution of chloride 73 (781 mg, 4.0 mmol) in DME (80 mL) and the solution stirred at reflux temperature for 2 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give 1-oxide 138 (1.01 g, 87%) as a yellow powder, mp (EtOAc) 182-184° C.; ¹H NMR δ 8.13 (d, J=8.8 Hz, 1H, H-8), 7.36 (br s, 1H, H-5), 7.10 (dd, J=8.8, 1.6 Hz, 1H, H-7), 5.85 (brs, 1H, NH), 3.70-3.74 (m, 4H, 2×CH₂O), 3.54-3.59 (m, 2H, CH₂N), 2.60-2.64 (m, 2H, CH₂N), 2.48-2.52 (m, 4H, 2×CH₂N), 2.46 (s, 3H, CH₃); ¹³C NMR δ 159.1, 149.1, 147.2, 129.2, 127.1, 125.3, 120.2, 67.0 (2), 56.7, 53.3 (2), 37.5, 22.0. Anal. calcd for C₁₄H₁₉N₅O₂: C, 58.1; H, 6.6; N, 24.2; found C, 58.1; H, 6.7; N, 24.2%.

### Example 146

6-Methyl-N-[3-(4-morpholinyl)propyl]-1,2,4-benzotriazin-3-amine 1-Oxide (139). 3-(1-Morpholinyl)propylamine (0.73 mL, 5.0 mmol) was added to a stirred solution of chloride 73 (327 mg, 1.7 mmol) in DME (50 mL) and the solution stirred at reflux temperature for 4 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give 1-oxide 139 (347 mg, 68%) as a yellow powder, mp (EtOAc) 131-132° C.; ¹H NMR δ 8.13 (d, J=8.8 Hz, 1H, H-8), 7.36 (br s, 1H, H-5), 7.09 (dd, J=8.8, 1.6 Hz, 1H, H-7), 6.25 (br s, 1H, NH), 3.75-3.77 (m, 4H, 2×CH₂O), 3.57-3.62 (m, 2H, CH₂N), 2.46-2.53 (m, 9H, 3×CH₂N, CH₃), 1.81-1.87 (m, 2H, CH₂N); ¹³C NMR δ 159.2, 146.9, 147.0, 129.5, 126.9, 125.4, 120.2, 67.0 (2), 57.3, 53.8 (2), 40.9, 25.2, 22.0. Anal. calcd for C₁₅H₂₁N₅O₂: C, 59.4; H, 7.0; N, 23.1; found C, 59.5; H, 7.0; N, 22.8%.

### Example 147

N¹-(6-Methyl-1-oxido-1,2,4-benzotriazin-3-yl)-N²,N²-dipropyl-1,2-ethanediamine (140). N,N-dipropylethylenediamine (0.83 mL, 5.7 mmol) was added to a stirred solution of chloride 73 (448 mg, 2.3 mmol) in DME (50 mL) and the solution stirred at reflux temperature for 3 h. The solvent was evaporated and the residue partitioned between DCM (100 mL) and dilute aqueous NH₃ (50 mL). The organic fraction was dried and the solvent evaporated. The residue was chromatographed, eluting with a gradient (0-6%) of MeOH/DCM, to give 1-oxide 140 (697 mg, 100%) as a yellow powder, mp (EtOAc/pet ether) 88-90° C.; ¹H NMR δ 8.13 (d, J=8.8 Hz, 1H, H-8), 7.36 (br s, 1H, H-5), 7.08 (dd, J=8.8, 1.8 Hz, 1H, H-7), 5.89 (br s, 1H, NH), 3.48-3.52 (m, 2H, CH₂N), 2.66-2.69 (m, 2H, CH₂N), 2.46 (s, 3H, CH₃), 2.39-2.43 (m, 4H, 2×CH₂N), 1.41-1.50 (m, 4H, 2×CH₂), 0.88 (t, J=7.3 Hz, 6H, 2×CH₃); ¹³C NMR δ 159.2, 149.1, 146.8, 129.1, 126.9, 125.3, 120.2, 55.9 (2), 52.5, 38.9, 22.0, 20.3 (2), 11.9 (2). Anal. calcd for C₁₆H₂₅N₅O: C, 63.3; H, 8.3; N, 23.0; found C, 63.3; H, 8.6; N, 23.3%.

### Example 148

N-[2-(2,6-Dimethyl-1-piperidi nyl)ethyl]-6-methoxy-1,2,4-benzotriazin-3-amine 1-oxide (141). 2-(2,6-Dimethyl-1-piperidinyl)ethylamine (580 mg, 3.7 mmol) was added to a stirred solution of chloride 83 (314 mg, 1.5 mmol) in DME (40 mL) and the solution stirred at reflux temperature for 4 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) MeOH/DCM, to give 1-oxide 141 (416 mg, 85%) as a yellow solid, mp (MeOH) 170-171° C.; ¹H NMR δ 8.15 (d, J=9.4 Hz, 1H, H-8), 6.87 (dd, J=9.4, 2.6 Hz, 1H, H-7), 6.83 (d, J=2.6 Hz, 1H, H-5), 5.60 (br s, 1H, NH), 3.93 (s, 3H, OCH₃), 3.51-3.56 (m, 2H, CH₂N), 2.87-2.91 (m, 2H, CH₂N), 2.49-2.57 (m, 2H, 2×CHN), 1.65-1.70 (m, 1H, CH₂), 1.53-1.59 (m, 2H, CH₂), 1.23-1.38 (m, 3H, CH₂), 1.19 (d, J=6.3 Hz, 6H, 2×CH₃); ¹³C NMR δ 165.4, 159.3, 151.5, 126.0, 122.0, 117.7, 104.0, 57.2, 56.0 (2), 47.4, 39.4, 34.2 (2), 24.4, 22.0 (2). Anal. calcd for C₁₇H₂₅N₅O₂: C, 61.6; H, 7.6; N, 21.1; found C, 61.6: H, 7.6; N, 21.1%.

### Example 149

3-Chloro-6-trifluoromethyl-1,2,4-benzotriazine 1-Oxide (142). Sodium nitrite (690 mg, 10.0 mmol) was added in portions to a stirred solution of 1-oxide 3v (1.15 g, 6.6 mmol) in trifluoroacetic acid (50 mL) at 5° C. and the solution stirred at 20° C. for 1 h. The solution was poured into ice/water, filtered, washed with water (2×50 mL) and dried. The solid was suspended in POCl₃ (20 mL), DMF (2 drops) added, and the mixture stirred at 100° C. for 3 h. The solution was poured into ice/water, stirred for 20 minutes and filtered. The solid was dissolved in DCM (150 mL), dried, and the solvent evaporated. The residue was purified by chromatography, eluting with 5% EtOAc/DCM, to give chloride 142 (375 mg, 33%) as a pale yellow solid, mp (DCM/pet. ether) 118-120° C.; ¹H NMR [(CD₃)₂SO] δ 8.52-8.57 (m, 2H, H-5, H-8), 8.15 (dd, J=9.0, 1.8 Hz, 1H, H-7); ¹³C NMR [(CD₃)₂SO] δ 156.6, 146.6, 135.8, 135.5 (q, J=33 Hz), 126.6 (q, J=3 Hz), 126.1 (q, J=4 Hz), 126.6 (q, J=274 Hz), 122.0. Anal. calcd for C₈H₃ClF₃N₃O: C, 38.5; H, 1.2; N, 16.8; F, 22.8; found C, 38.5; H, 1.1; N, 16.7; F, 14.4%.

### Exampl 150

N¹-(6-Trifluoromethyl-1-oxido-1,2,4-benzotriazin-3-yl)-N²,N²-dimethyl-1,2-than diamin (143). N,N-Dimethyl-1,2-ethanediamine (0.5 mL, 4.6 mmol) was added to a stirred solution of chloride 142 (305 mg, 1.2 mmol) in DME (20 mL) and the solution stirred at reflux temperature for 3 h. The solvent was evaporated and the residue partitioned between DCM (100 mL) and dilute aqueous NH₃ (50 mL). The organic fraction was dried and the solvent evaporated. The residue was purified by chromatography, eluting with a gradient (0-5%) of MeOH/DCM, to give the amine 143 (367 mg, 100%) as a yellow solid, mp (MeOH/DCM) 159-161° C.; ¹H NMR δ 8.33 (d, J=8.9 Hz, 1H, H-8), 7.87 (br s, 1H, H-5), 7.40 (dd, J=8.9, 1.5 Hz, 1H, H-7), 6.24 (br s, 1H, NH), 3.53-3.58 (m, 2H, CH₂N), 2.57-2.60 (m, 2H, CH₂N), 2.28 [s, 6H, N(CH₃)₂]; ¹³C NMR δ 159.4, 148.5, 136.8 (q, J=33 Hz), 132.0, 127.1, 124.3 (q, J=4 Hz), 121.9, 120.0 (q, J=3 Hz), 57.3, 45.0 (2), 38.7. Anal. calcd for C₁₂H₁₄F₃N₅O: C, 47.8; H, 4.7; N, 23.3; found C, 48.0; H, 4.4; N, 23.1%.

### Example 151

6-Isopropyl-1,2,4-benzotriazin-3-amine 1-oxide (145). A mixture of 5-isopropyl-2-nitroaniline (144) (Prasad, J. V. N. V. Org. Lett. 2000, 2, 1069) (4.53 g, 25.1 mmol) and cyanamide (4.23 g, 100 mmol) were mixed together at 100° C., cooled to 50° C., cHCl (15 mL) added carefully and the mixture heated at 100° C. for 4 h. The mixture was cooled to 50° C., 7.5 M NaOH solution added until the mixture was strongly basic and the mixture stirred at 100° C. for 3 h. The mixture was cooled, diluted with water (200 mL), filtered, washed with water (3×50 mL), washed with ether (3×30 mL) and dried. The residue was chromatographed, eluting with a gradient (0-10%) of MeOH/DCM, to give 1-oxide 145 (2.64 g, 52%) as a yellow powder, mp (CHCl₃) 220-222° C.; ¹H NMR [(CD₃)₂SO] δ 8.04 (d, J=8.9 Hz, 1H, H-8), 7.32 (d, J=1.8 Hz, 1H, H-5), 7.27 (dd, J=8.9, 1.8 Hz, 1H, H-7), 7.24 (br s, 2H, NH₂), 3.01 (sept, J=6.9 Hz, 1H, CH), 1.24 (d, J=6.9 Hz, 6H, 2×CH₃); ¹³C NMR [(CD₃)₂SO] δ 160.3, 156.7, 149.0, 128.3, 124.2, 121.9, 119.7, 33.5, 22.9 (2).

### Example 152

3-Chloro-6-isopropyl-1,2,4-benzotriazine 1-oxide (146). Sodium nitrite (1.71 g, 12.4 mmol) was added in small portions to a stirred solution of amine 145 (2.53 g, 12.4 mmol) in trifluoroacetic acid (80 mL) at 5° C. and the solution stirred at 20° C. for 3 h. The solution was poured into ice/water, stirred 30 minutes, filtered, washed with water (3×30 mL) and dried. The solid was suspended in POCl₃ (70 mL) and DMF (0.5 mL) and stirred at 100° C. for 1 h. The solution was cooled, poured into ice/water, stirred for 30 minutes, filtered, washed with water (3×30 mL) and dried. The solid was suspended in DCM (150 mL), dried and the solvent evaporated. The residue was chromatographed, eluting with 5% EtOAc/DCM, to give chloride 146 (1.74 g, 63%) as a pale yellow solid, mp (EtOAc/DCM) 81-83° C.; ¹H NMR δ 8.32 (d, J=8.9 Hz, 1H, H-8), 7.78 (d, J=1.8 Hz, 1H, H-5), 7.63 (dd, J=8.9, 1.8 Hz, 1H, H-7), 3.15 (sept, J=6.9 Hz, 1H, CH), 1.35 (d, J=6.9 Hz, 6H, 2×CH₃); ¹³C NMR δ 159.4, 157.0, 147.8, 132.2, 131.0, 124.6, 120.1, 34.6, 23.2 (2). Anal. calcd for C₁₀H₁₀CIN₃O: C, 53.7; H, 4.5; N, 18.8; found C, 53.7; H, 4.4; N, 19.0%.

### Example 153

N¹-(6-Isopropyl-1-oxido-1,2,4-benzotriazin-3-yl)-N²,N²-dimethyl-1,2-ethanediamine (147). N,N-Dimethylethanediamine (0.37 mL, 3.4 mmol) was added to a stirred solution of chloride 146 (300 mg, 1.3 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 147 (348 mg, 94%) as a yellow solid, mp (MeOH/EtOAc) 87-89° C.; ¹H NMR δ 8.16 (d, J=8.9 Hz, 1H, H-8), 7.40 (br s, 1H, H-5), 7.16 (dd, J=8.9, 1.7 Hz, 1H, H-7), 5.91 (br s, 1H, NH), 3.52-3.56 (m, 2H, CH₂N), 3.00 (sept, J=6.9 Hz, 1H, CH), 2.54-2.58 (m, 2H, CH₂N), 2.27 [s, 6H, N(CH₃)₂], 1.29 (d, J=6.9 Hz, 6H, 2×CH₃); ¹³C NMR δ 159.2, 157.5, 149.3, 129.3, 124.8, 122.6, 120.3, 57.5, 45.1 (2), 38.7, 34.5, 22.3 (2). Anal. calcd for C₁₄H₂₁N₅O.¼CH₃OH: C, 60.4; H, 7.8; N, 24.7; found C, 60.5: H, 7.9; N, 25.0%.

### Example 154

N¹-(6-Isopropyl-1-oxido-1,2,4-benzotriazi n-3-yl)-N²,N²-diethyl-1,2-ethanediami ne (148). N,N-Diethylethanediamine (0.47 mL, 3.4 mmol) was added to a stirred solution of chloride 146 (308 mg, 1.4 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 148 (390 mg, 93%) as a yellow solid, mp (MeOH/EtOAc) 60-62° C.; ¹H NMR δ 8.16 (d, J=8.9 Hz, 1H, H-8), 7.40 (br s, 1H, H-5), 7.16 (dd, J=8.9, 1.8 Hz, 1H, H-7), 5.98 (br s, 1H, NH), 3.50-3.55 (m, 2H, CH₂N), 3.00 (sept, J=6.9 Hz, 1H, CH), 2.71 (dd, J=6.0, 5.9 Hz, 2H, CH₂N), 2.58 (q, J=7.1 Hz, 4H, 2×CH₂), 1.30 (d, J=6.9 Hz, 6H, 2×CH₃), 1.04 (t, J=7.1 Hz, 6H, 2×CH₃); ¹³C NMR δ 159.1, 157.5, 149.3, 129.3, 124.8, 122.6, 120.3, 51.2, 46.6 (2), 38.7, 34.5, 23.3 (2), 11.7 (2). Anal. calcd for C₁₆H₂₅N₅O.½H₂O: C, 62.4; H, 8.4; N, 22.7; found C, 62.5: H, 8.5; N, 22.9%.

### Example 155

6-Isopropyl-N-[2-(4-morpholinyl)ethyl]-1,2,4-benzotriazin-3-amine 1-Oxide (149). 2-(4-Morpholinyl)ethylamine (0.45 mL, 3.4 mmol) was added to a stirred solution of chloride 146 (306 mg, 1.4 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 149 (429 mg, 99%) as a yellow solid, mp (MeOH) 145-147° C.; ¹H NMR δ8.17 (d, J=8.9 Hz, 1H, H-8), 7.40 (br s, 1H, H-5), 7.17 (dd, J=8.9, 1.8 Hz, 1H, H-7), 5.86 (br s, 1H, NH), 3.69-3.74 (m, 4H, 2×CH₂O), 3.56-3.60 (m, 2H, CH₂N), 3.01 (sept, J=6.9 Hz, 1H, CH), 2.63 (dd, J=6.0, 5.8 Hz, 2H, CH₂N), 2.48-2.53 (m, 4H, 2×CH₂N), 1.30 (d, J=6.9 Hz, 6H, 2×CH₃); ¹³C NMR δ159.0, 157.6, 149.2, 129.3, 125.0, 122.6, 120.3, 67.0 (2), 56.7, 53.3 (2), 37.5, 34.5, 23.3 (2). Anal. calcd for C₁₆H₂₃N₅O₂.¼H₂O: C, 59.7; H, 7.4; N, 21.8; found C, 59.7: H, 7.5; N, 21.8%.

### Example 156

6-Isopropyl-N-[2-(1-piperidinyl)ethyl]-1,2,4-benzotriazin-3-amine 1-Oxide (150). 2-(1-Piperidinyl)ethylamine (0.47 mL, 3.4 mmol) was added to a stirred solution of chloride 146 (297 mg, 1.3 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 150 (394 mg, 94%) as a yellow solid, mp (MeOH/EtOAc) 124-127° C.; ¹H NMR δ 8.16 (d, J=8.9 Hz, 1H, H-8), 7.41 (br s, 1H, H-5), 7.15 (dd, J=8.9, 1.8 Hz, 1H, H-7), 5.79 (br s, 1H, NH), 3.51-3.55 (m, 2H, CH₂N), 3.00 (sept, J=6.9 Hz, 1H, CH), 2.56 (dd, J=6.1, 5.9 Hz, 2H, CH₂N), 2.39-2.44 (m, 4H, 2×CH₂), 1.54-1.60 (m, 4H, 2×CH₂), 1.41-1.47 (m, 2H, CH₂), 1.30 (d, J=6.9 Hz, 6H, 2×CH₃); ¹³C NMR δ 159.1, 157.5, 149.3, 129.3, 124.8, 122.6, 120.3, 56.8, 54.3 (2), 37.9, 34.5, 26.0 (2), 24.4, 23.3 (2). Anal. calcd for C₁₇H₂₅N₅O: C, 64.7; H, 8.0; N, 22.2; found C, 64.5: H, 8.3; N, 22.4%.

### Example 157

N¹-(6-Isopropyl-1-oxido-1,2,4-benzotriazi n-3-yl)-N²,N²-dipropyl-1,2-ethanediamine (151). N¹,N¹-Dipropyl-1,2-ethanediamine (0.49 mL, 3.4 mmol) was added to a stirred solution of chloride 146 (305 mg, 1.4 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-5%) of MeOH/DCM, to give 1-oxide 151 (434 mg, 96%) as a yellow solid, mp (MeOH) 79-81° C.; ¹H NMR δ 8.17 (d, J=8.9 Hz, 1H, H-8), 7.41 (br s, 1H, H-5), 7.16 (dd, J=8.9, 1.8 Hz, 1H, H-7), 5.89 (br s, 1H, NH), 3.48-3.53 (m, 2H, CH₂N), 3.00 (sept, J=6.9 Hz, 1H, CH), 2.67 (dd, J=6.0, 5.8 Hz, 2H, CH₂N), 2.39-2.45 (m, 4H, 2×CH₂N), 1.41-1.50 (m, 4H, 2×CH₂), 1.30 (d, J=6.9 Hz, 6H, 2×CH₃), 0.88 (t, J=7.3 Hz, 6H, 2×CH₃); ¹³C NMR δ 159.2, 157.5, 149.3, 129.3, 124.8, 122.6, 120.3, 55.9 (2), 52.5, 38.9, 34.5, 23.3 (2), 20.4 (2), 11.9 (2). Anal. calcd for C₁₈H₂₉N₅O.¼H₂O: C, 64.4; H, 8.9; N, 20.9; found C, 64.6; H, 8.9; N, 21.3%.

### Example 158

6-tert-Butyl-1,2,4-benzotriazin-3-amine 1-Oxide (153). A mixture of 4-tert-butyl-2-nitroaniline (152) (Seko, S.; et. al. J. Chem. Soc. Perkin Trans. 1 1999, 1437.) (4.11 g, 21.2 mmol) and cyanamide (3.56 g, 84.6 mmol) were mixed together at 100° C., cooled to 50° C., cHCl (15 mL) added carefully and the mixture heated at 100° C. for 4 h. The mixture was cooled to 50° C., 7.5 M NaOH solution added until the mixture was strongly basic and the mixture stirred at 100° C. for 3 h. The mixture was cooled, diluted with water (200 mL), filtered, washed with water (3×50 mL), washed with ether (3×30 mL) and dried. The residue was chromatographed, eluting with a gradient (0-10%) of MeOH/DCM, to give 1-oxide 153 (2.56 g, 62%) as a yellow powder, mp (MeOH/EtOAc) 209-212° C.; ¹H NMR [(CD₃)₂SO] δ 8.04 (d, J=9.0 Hz, 1H, H-8), 7.45 (dd, J=9.0, 2.0 Hz, 1H, H-7), 7.22 (br s, 2H, NH₂), 7.39 (d, J=2.0 Hz, 1H, H-5), 1.32 [s, 9H, C(CH₃)₃]; ¹³C NMR [(CD₃)₂SO] δ 160.4, 158.9, 148.8, 127.9, 123.4, 120.8, 119.4, 35.0, 30.2 (3). Anal. calcd for C₁₁H₁₄N₄O: C, 60.5; H, 6.5; N, 25.7; found C, 60.8; H, 6.6; N, 25.8%.

### Example 159

6-tert-Butyl-3-chloro-1,2,4-benzotriazine 1-oxide (154). Sodium nitrite (285 mg, 4.1 mmol) was added in small portions to a stirred solution of amine 153 (0.45 g, 2.1 mmol) in trifluoroacetic acid (20 mL) at 5° C. and the solution stirred at 20° C. for 3 h. The solution was poured into ice/water, stirred 30 minutes, filtered, washed with water (3×30 mL) and dried. The solid was suspended in POCl₃ (50 mL) and DMF (0.5 mL) and stirred at 100° C. for 1 h. The solution was cooled, poured into ice/water, stirred for 30 minutes, filtered, washed with water (3×30 mL) and dried. The solid was suspended in DCM (150 mL), dried and the solvent evaporated. The residue was chromatographed, eluting with 5% EtOAc/DCM, to give chloride 154 (382 mg, 78%) as a pale yellow solid, mp (EtOAc/DCM) 91-94° C.; ¹H NMR δ 8.33 (d, J=9.1 Hz, 1H, H-8), 7.93 (d, J=2.0 Hz, 1H, H-5), 7.83 (dd, J=9.1, 2.0 Hz, 1H, H-7), 1.46 [s, 9H, C(CH₃)₃]; ¹³C NMR δ 161.2, 156.5, 147.1, 131.3, 129.4, 123.3, 119.2, 35.4, 30.1 (3). Anal. calcd for C₁₁H₁₂ClN₃O: C, 55.6; H, 5.1; N, 17.7; Cl, 14.9; found C, 55.3; H, 4.9; N, 17.5; Cl, 15.0%.

### Example 160

N¹-(6-tert-Butyl-1-oxido-1,2,4-benzotriazin-3-yl)-N²,N²-dimethyl-1,2-ethanediamine (155). N,N-Dimethylethanediamine (0.66 mL, 6.0 mmol) was added to a stirred solution of chloride 154 (476 mg, 2.0 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 155 (536 mg, 93%) as a yellow solid, mp (MeOH/EtOAc) 140-144° C.; ¹H NMR δ 8.16 (d, J=9.0 Hz, 1H, H-8), 7.53 (d, J=2.0 Hz, 1H, H-5), 7.35 (dd, J=9.0, 2.0 Hz, 1H, H-7), 6.01 (br s, 1H, NH), 3.56-3.61 (m, 2H, CH₂N), 2.62-2.65 (m, 2H, CH₂N), 2.33 [s, 6H, N(CH₃)₂], 1.38 [s, 9H, C(CH₃)₃]; ¹³C NMR δ 159.8, 159.2, 149.0, 129.0, 123.8, 121.8, 119.9, 57.5, 45.0 (2), 38.5, 35.5, 30.7 (3). Anal. calcd for C₁₅H₂₃N₅O.¼H₂O: C, 61.3; H, 8.1; N, 23.8; found C, 61.1: H, 8.1; N, 23.8%.

### Example 161

N¹-(6-tert-Butyl-1-oxido-1,2,4-benzotriazin-3-yl)-N²,N²-diethyl-1,2-ethanediamin (156). N¹,N¹-Diethyl-1,2-ethanediamine (0.52 mL, 3.7 mmol) was added to a stirred solution of chloride 154 (350 mg, 1.5 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-5%) of MeOH/DCM, to give 1-oxide 156 (412 mg, 88%) as a yellow gum, ¹H NMR 68.16 (d, J=9.1 Hz, 1H, H-8), 7.53 (d, J=2.0 Hz, 1H, H-5), 7.34 (dd, J=9.1, 2.0 Hz, 1H, H-7), 5.97 (br s, 1H, NH), 3.50-3.55 (m, 2H, CH₂N), 2.68-2.72 (m, 2H, CH₂N), 2.55-2.60 (m, 4H, 2×CH₂N), 1.37 [s, 9H, C(CH₃)₃], 1.04 (t, J=7.1 Hz, 6H, 2×CH₃); ¹³C NMR δ 159.8, 159.2, 149.1, 128.9, 123.7, 121.8, 119.9, 51.2, 46.6 (2), 38.7, 35.5, 30.7 (3), 11.8 (2); MS (EI+) m/z 317 (M+, 1%), 300 (10), 86 (100); HRMS (EI⁺) calcd for C₁₇H₂₇N₅O (M⁺) m/z317.2216, found 317.2215.

### Example 162

6-tert-Butyl-N-[2-(4-morpholinyl)ethyl]-1,2,4-benzotriazin-3-amine 1-Oxide (157). 2-(4-Morpholinyl)ethylamine (0.66 mL, 5.0 mmol) was added to a stirred solution of chloride 154 (476 mg, 2.0 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-5%) of MeOH/DCM, to give 1-oxide 157 (642 mg, 97%) as a yellow powder, mp (MeOH/EtOAc) 132-134° C.; ¹H NMR δ 8.18 (d, J=9.1 Hz, 1H, H-8), 7.55 (d, J=2.0 Hz, 1H, H-5), 7.37 (dd, J=9.1, 2.0 Hz, 1H, H-7), 5.86 (br s, 1H, NH), 3.69-3.75 (m, 4H, 2×CH₂O), 3.56-3.61 (m, 2H, CH₂N), 2.60-2.64 (m, 2H, CH₂N), 2.48-2.53 (m, 4H, 2×CH₂N), 1.38 [s, 9H, C(CH₃)₃]; ¹³C NMR δ 159.9, 159.1, 149.0, 129.0, 123.9, 121.8, 120.0, 67.0 (2), 56.7, 53.3 (2), 37.4, 35.6, 30.7 (3). Anal. calcd for C₁₇H₂₅N₅O₂: C, 61.1; H, 7.6; N, 21.1; found C, 61.2; H, 7.8; N, 21.2%.

### Example 163

6-tert-Butyl-N-[2-(1-piperidinyl)ethyl]-1,2,4-benzotriazin-3-amine 1-Oxide (158). 2-(1-Piperidinyl)ethylamine (0.57 mL, 4.0 mmol) was added to a stirred solution of chloride 154 (379 mg, 1.6 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-5%) of MeOH/DCM, to give 1-oxide 158 (500 mg, 95%) as a yellow powder, mp (MeOH/EtOAc) 145-148° C.; ¹H NMR δ 8.16 (d, J=9.1 Hz, 1H, H-8), 7.53 (d, J=2.0 Hz, 1H, H-5), 7.34 (dd, J=9.1, 2.0 Hz, 1H, H-7), 5.99 (br s, 1 H, NH), 3.52-3.57 (m, 2H, CH₂N), 2.56-2.60 (m, 2H, CH₂N), 2.38-2.45 (m, 4H, 2×CH₂N), 1.55-1.61 (m, 4H, 2×CH₂), 1.41-1.47 (m, 2H, CH₂), 1.37 [s, 9H, C(CH₃)₃];

¹³C NMR δ 159.8, 159.1, 149.1, 128.9, 123.7, 121.8, 119.9, 56.8, 54.3 (2), 37.9, 35.5, 30.7 (3), 26.0 (2), 24.4. Anal. calcd for C₁₈H₂₇N₅O.¼H₂O: C, 64.7; H, 8.3; N, 21.0; found C, 64.5; H, 8.5; N, 21.0%.

### Example 164

N¹-(6-tert-Butyl-1-oxido-1,2,4-benzotriazi n-3-yl)-N²,N²-dipropyl-1,2-ethanediamine (159). N¹,N¹-Dipropyl-1,2-ethanediamine (0.43 mL, 3.0 mmol) was added to a stirred solution of chloride 154 (283 mg, 1.2 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-5%) of MeOH/DCM, to give 1-oxide 159 (393 mg, 96%) as a pale yellow oil, ¹H NMR δ 8.17 (d, J=9.1 Hz, 1H, H-8), 7.54 (d, J=1.8 Hz, 1H, H-5), 7.35 (dd, J=9.1, 1.8 Hz, 1H, H-7), 5.90 (br s, 1H, NH), 3.49-3.55 (m, 2H, CH₂N), 2.67-2.71 (m, 2H, CH₂N), 2.38-2.45 (m, 4H, 2×CH₂N), 1.43-1.52 (m, 4H, 2×CH₂), 1.37 [s, 9H, C(CH₃)₃], 0.88 (t, J=7.3 Hz, 6H, 2×CH₃); ¹³C NMR δ 159.2, 158.7, 148.6, 128.4, 123.1, 121.3, 119.4, 55.3 (2), 52.0, 38.4, 35.0, 30.7 (3), 19.8 (2), 11.4 (2); MS (EI⁺) m/z345 (M⁺, 1%), 328 (10), 114 (100); HRMS (EI⁺) calcd for C₁₉H₃₁N₅O (M⁺) m/z345.2529, found 345.2528.

### Example 165

3-Ethyl-6-fluoro-1,2,4-benzotriazine 1-oxide (160). Pd(PPh₃)₄ (196 mg, 0.17 mmol) was added to a stirred solution of chloride 131 (329 mg, 1.7 mmol) and tetraethyltin (0.7 mL, 3.3 mmol) in DME (20 mL), the solution degassed, and stirred under N₂ at reflux temperature for 16 h. The solvent was evaporated and the residue purified by chromatography, eluting with 20% EtOAc/pet. ether to give an oil which was further purified by chromatography, eluting with 5% EtOAc/DCM, to give 1-oxide 160 (295 mg, 93%) as a white solid, mp (EtOAc/pet. ether) 122-124° C.; ¹H NMR δ 8.48 (dd, J=9.5, 5.5 Hz, 1H, H-8), 7.60 (dd, J=8.7, 2.6 Hz, 1H, H-5), 7.38 (m, 1H, H-7), 3.04 (q, J=7.6 Hz, 2H, CH₂), 1.43(t, J=7.6 Hz, 3H, CH₃); ¹³C NMR δ 168.6(q, J=175 Hz), 165.1, 149.5 (d, J=15 Hz), 130.5, 123.2 (d, J=11 Hz), 120.0 (d, J=26 Hz), 112.7 (d, J=22 Hz), 30.7, 12.2. Anal. calcd for C₉H₈FN₃O: C, 56.0; H, 4.2; N, 21.8; found C, 56.0; H, 4.2; N, 21.8%.

### Exampl 166

3-Ethyl-6-m thoxy-1,2,4-benzotriazin 1-oxid (161). Sodium (55 mg, 2.4 mmol) was added to a stirred solution of fluoride (310 mg, 1.6 mmol) in MeOH (10 mL) and the solution was stirred at 20° C. for 4 h under N₂. The solvent was evaporated and the residue partitioned between DCM (20 mL) and water (20 mL). The organic fraction was dried, and the solvent evaporated. The residue was purified by chromatography, eluting with 5% EtOAc/pet. ether, to give 1-oxide 161 (156 mg, 56%) as a white solid, mp (EtOAc/pet. ether) 109-111° C.; ¹H NMR δ 8.32 (d, J=9.5 Hz, 1H, H-8), 7.24 (dd, J=9.5, 2.6 Hz, 1H, H-7), 7.19 (d, J=2.6 Hz, 1H, H-5), 3.98 (s, 3H, OCH₃), 3.00 (q, J=7.6 Hz, 2H, CH₂), 1.43 (t, J=7.6 Hz, 3H, CH₃); ¹³C NMR δ 168.8, 165.3, 150.3, 128.5, 122.9, 121.7, 105.8, 56.2, 30.7, 12.2. Anal. calcd for C₁₀H₁₁N₃O₂: C, 58.5; H, 5.4; N, 20.5; found C, 58.6; H, 5.4; N, 20.5%.

### Example 167

N¹,N¹-Diethyl-N²-(8-methyl-1-oxido-1,2,4-benzotriazin-3-yl)-1,2-ethanediamine (162). N,N-Diethylethanediamine (0.38 mL, 2.7 mmol) was added to a stirred solution of chloride 100 (214 mg, 1.1 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) MeOH/DCM, to give 1-oxide 162 (297 mg, 99%) as a yellow solid, mp (MeOH/EtOAc) 89-91° C.; ¹H NMR δ 7.49 (dd, J=8.4, 7.1 Hz, 1H, H-6), 7.42 (d, J=8.4 Hz, 1H, H-5), 7.01 (d, J=7.1 Hz, 1H, H-7), 5.86 (br s, 1H, NH), 3.49-3.53 (m, 2H, CH₂N), 2.90 (s, 3H, CH₃), 2.69 (dd, J=6.1, 5.9 Hz, 2H, CH₂N), 2.57 (q, J=7.1 Hz, 4H, 2×CH₂N), 1.04 (t, J=7.1 Hz, 6H, 2×CH₃); ¹³C NMR δ 158.4, 150.7, 134.5, 134.2, 131.1, 127.4, 124.7, 51.3, 46.6 (2), 38.7, 24.0 11.8 (2). Anal. calcd for C₁₄H₂₁N₅O: C, 61.1; H, 7.7; N, 25.4; found C, 61.4; H, 7.8; N, 25.3%.

### Example 168

8-Methyl-N-[2-(4-morpholinyl)ethyl]-1,2,4-benzotriazin-3-amine 1-Oxide (163). N,N-2-(4-Morpholinyl)ethylamine (0.94 mL, 7.1 mmol) was added to a stirred solution of chloride 100 (465 mg, 2.4 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) MeOH/DCM, to give 1-oxide 163 (545 mg, 79%) as a yellow solid, mp (MeOH/EtOAc) 168-170° C.; ¹H NMR δ 7.50 (dd, J=8.3, 7.1 Hz, 1H, H-6), 7.42 (d, J=8.3 Hz, 1H, H-5), 7.02 (d, J=7.1 Hz, 1H, H-7), 5.76 (br s, 1H, NH), 3.69-3.73 (m, 4H, 2×CH₂O), 3.55-3.59 (m, 2H, CH₂N), 2.89 (s, 3H, CH₃), 2.60-2.63 (m, 2H, CH₂N), 2.47-2.51 (m, 4H, 2×CH₂N); ¹³C NMR δ 158.4, 150.7, 134.6, 134.3, 131.2, 127.6, 124.7, 66.9 (2), 56.8, 55.3 (2), 37.4, 24.0. Anal. calcd for C₁₄H₁₉N₅O₂: C, 58.1; H, 6.6; N, 24.2; found C, 58.3; H, 6.7; N, 24.4%.

### Example 169

N-[2-(2,6-Dimethyl-1-piperidinyl)ethyl]-8-methyl-1,2,4-benzotriazin-3-amine 1-Oxide (164). 2-(2,6-Dimethyl-1-piperidinyl)ethylamine (0.70 mL, 4.5 mmol) was added to a stirred solution of chloride 100 (352 mg, 1.8 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) of MeOH/DCM, to give 1-oxide 164 (489 mg, 82%) as a yellow solid, mp (MeOH/EtOAc) 169-171° C.;

¹H NMR δ 7.48 (dd, J=8.4, 7.1 Hz, 1H, H-6), 7.37 (d, J=8.4 Hz, 1H, H-5), 7.00 (d, J=7.1 Hz, 1H, H-7), 5.56 (br s, 1H, NH), 3.50-3.55 (m, 2H, CH₂N), 2.86-2.90 (m, 5H, CH₂N, CH₃), 2.48-2.56 (m, 2H, CH₂N), 1.63-1.68 (m, 1H, CH), 1.52-1.58 (m, 2H, CH₂), 1.32-1.38 (m, 1H, CH), 1.23-1.30 (m, 2H, CH₂), 1.20 (d, J=6.3 Hz, 6H, 2×CH₃); ¹³C NMR δ 158.5, 150.7, 134.5, 134.2, 131.1, 127.5, 124.8, 57.2 (2), 47.5, 39.3, 34.3 (2), 24.4, 24.0 (2), 21.6. Anal. calcd for C₁₇H₂₅N₅O: C, 64.7; H, 8.0; N, 22.2; found C, 64.7; H, 8.2; N, 22.3%.

### Example 170

N¹-(8-Methyl-1-oxido-1,2,4-benzotriazi n-3-yl)-N²,N²-dipropyl-1,2-ethanediami ne (165). N¹,N¹-Dipropyl-1,2-ethanediamine (0.77 mL, 5.3 mmol) was added to a stirred solution of chloride 100 (415 mg, 2.1 mmol) in DME (50 mL) and the solution stirred at reflux temperature for 2 h. The solution was cooled, the solvent evaporated and the residue partitioned between dilute aqueous NH₃ (100 mL) and DCM (100 mL). The organic fraction was dried and the solvent evaporated. The residue was chromatographed, eluting with a gradient (0-10%) MeOH/DCM, to give 1-oxide 165 (597 mg, 93%) as a yellow solid, mp (MeOH/EtOAc) 91-93° C.; ¹H NMR δ 7.48 (dd, J=8.4, 7.1 Hz, 1H, H-6), 7.41 (d, J=8.4 Hz, 1H, H-5), 7.00 (d, J=7.1 Hz, 1H, H-7), 5.80 (br s, 1H, NH), 3.47-3.52 (m, 2H, CH₂N), 2.89 (s, 3H, CH₃), 2.65-2.69 (m, 2H, CH₂N), 2.39-2.43 (m, 4H, 2×CH₂N), 1.42-1.51 (m, 4H, 2×CH₂), 0.89 (t, J=7.3 Hz, 6H, 2×CH₃); ¹³C NMR δ 158.5, 150.7, 134.4, 134.2, 131.1, 127.4, 124.7, 55.9 (2), 52.6, 38.8, 24.0, 20.3 (2), 11.9 (2). Anal. calcd for C₁₆H₂₅N₅O: C, 63.3; H, 8.3; N, 23.1; found C, 63.4; H, 8.3; N, 22.7%.

### Example 171

N¹-(6,7-Dimethyl-1-oxido-1,2,4-benzotriazin-3-yl)-N²,N²-diethyl-1,2-ethanediamine (166). N,N-Diethyl-1,2-ethanediamine (0.54 mL, 3.8 mmol) was added to a stirred solution of chloride 104 (322 mg, 1.5 mmol) in DME (30 mL) and the solution stirred at reflux temperature for 16 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give 1-oxide 166 (438 mg, 98%), mp (MeOH/EtOAc) 130-132° C.; ¹H NMR δ 8.01 (s, 1H, H-8), 7.36 (s, 1H, H-5), 5.87 (br s, 1H, NH), 3.47-3.52 (m, 2H, CH₂N), 2.68 (dd, J=6.1, 5.9 Hz, 2H, CH₂N), 2.57 (g, J=7.1 Hz, 4H, 2×CH₂N), 2.39 (s, 3H, CH₃), 2.35 (s, 3H, CH₃), 1.03 (t, J=7.1 Hz, 6H, 2×CH₃); ¹³C NMR δ 158.9, 147.8, 146.9, 135.3, 129.1, 125.7, 119.4, 51.2, 46.6 (2), 38.7, 20.5, 19.8, 11.8 (2). Anal. calcd for C₁₅H₂₃N₅O: C, 62.3; H, 8.0; N, 24.2; found C, 62.4; H, 8.1; N, 24.5%.

### Example 172

6,7-Dimethyl-N-[2-(4-morpholinyl)ethyl]-1,2,4-benzotriazi n-3-amine 1-Oxide (167). 2-(4-Morpholinyl)ethylamine (0.76 mL, 5.8 mmol) was added to a stirred solution of chloride 104 (406 mg, 1.9 mmol) in DME (50 mL) and the solution stirred at reflux temperature for 6 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give 1-oxide 167 (552 mg, 94%), mp (MeOH/EtOAc) 211-213° C.; ¹H NMR δ 8.01 (s, 1H, H-8), 7.36 (s, 1H, H-5), 5.79 (br s, 1H, NH), 3.69-3.73 (m, 4H, 2×CH₂O), 3.54-3.58 (m, 2H, CH₂), 2.62 (dd, J=6.0, 5.9 Hz, 2H, CH₂N), 2.48-2.52 (m, 4H, 2×CH₂N), 2.38 (s, 3H, CH₃), 2.36 (s, 3H, CH₃); ¹³C NMR δ 158.8, 147.8, 147.9, 135.3, 129.2, 125.7, 119.4, 66.9 (2), 56.8, 53.3 (2), 37.5, 20.5, 19.8. Anal. calcd for C₁₅H₂₁N₅O₂: C, 59.4; H, 7.0; N, 23.1; found C, 59.3; H, 6.9; N, 23.1%.

### Exampl 173

6,7-Dimethyl-A[3-(4-morpholinyl)propyl]-1,2,4-benzotriazin-3-amin 1-Oxide (168). 3-(4-Morpholinyl)propylamine (1.16 mL, 7.9 mmol) was added to a stirred solution of chloride 104 (555 mg, 2.7 mmol) in DME (50 mL) and the solution stirred at reflux temperature for 6 h. The solvent was evaporated and the residue partitioned between DCM (100 mL) and dilute aqueous NH₃ (50 mL). The organic fraction was dried and the solvent evaporated. The residue was chromatographed, eluting with a gradient (0-10%) of MeOH/DCM, to give 1-oxide 168 (788 mg, 94%), mp (MeOH/EtOAc) 145-147° C.; ¹H NMR δ 8.00 (s, 1H, H-8), 7.34 (s, 1H, H-5), 6.16 (br s, 1H, NH), 3.72-3.76 (m, 4H, 2×CH₂O), 3.55-3.60 (m, 2H, CH₂), 2.44-2.52 (m, 6H, 3×CH₂N), 2.47 (s, 3H, CH₃), 2.35 (s, 3H, CH₃), 1.79-1.87 (m, 2H, CH₂N); ¹³C NMR δ 159.0, 147.8, 146.9, 135.3, 129.2, 125.7, 119.4, 67.0 (2), 57.3, 53.8 (2), 40.8, 25.2, 20.5, 19.8. Anal. calcd for C₁₆H₂₃N₅O₂: C, 60.6; H, 7.3; N, 22.0; found C, 60.6; H, 7.2; N, 22.2%.

Wherein the foregoing description reference has been made to reagents or integers having known equivalents thereof, then those equivalents are herein incorporated as if individually set forth.

While this invention has been described with reference to certain embodiments and examples, it is to be appreciated that further modifications and variations may be made thereto without departing from the spirit or scope of the invention.

1. A cytotoxic synergistic composition, comprising an effective amount of a benzoazine N-mono oxide compound of Formula A or a pharmacologically acceptable salt thereof and an effective amount of a benzoazine 1,4 dioxide compound of Formula B or a pharmacologically acceptable salt thereof 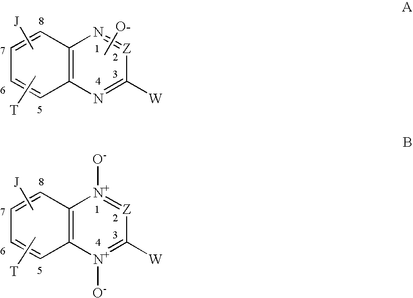
wherein in formulae A or B
Z is N or C—CN, and
wherein in formula A when Z represents N, the N-oxide moiety occupies one of the 1-, 2-, or 4-positions; and when Z represents C—CN, the N-oxide moiety occupies one of the 1-, or 4-positions;
wherein J in formulae A or B represents at one or more of the available carbons 5-8 on the benzo ring the following groups:
halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;
wherein each R is independently selected from an optionally substituted C₁ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and
wherein W in Formulae A or B can represent —X-A, wherein —X-A together can represent H, or halogen; or
X represents O, S, NH, NMe, CH₂, SO, SO₂, CONH, NHCO, CO or CO₂, and
A represents H, an optionally substituted C₁₋₁₂alkyl group wherein the optional substituents are each independently selected from OH, OR³, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂ alkyl chain can optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, or a pharmacologically acceptable salt thereof, or
W can represent a group of Formula C 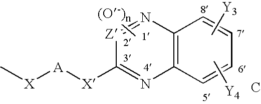
wherein in a group of Formula C
n represents either 1 or 2,
Z′ is selected from N or C—CN, and when Z′ represents N, and n represents 1 the N-oxide moiety occupies one of the 1′-, 2′-, or 4‘-positions and when Z’ represents C—CN, the N-oxide moiety occupies one of the 1′-, or 4′-positions; and when Z′ represents N or C—CN, and n represents 2 the N-oxide moieties occupy the 1′ and 4′-positions
Y₃ and Y₄ each represent at one or more of the available carbons 5′-8′ on the benzo ring the following groups:
halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;
wherein each R is independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and
X′ represents O, NH, NMe, or CH₂,
A represents an optionally substituted C₁₋₁₂alkyl group wherein the optional substituents are each independently selected from OH, OR³, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂ alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, or
W can represent a group of Formula D 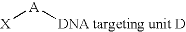
wherein X represents NH, NMe, CH₂, or O;
A represents an optionally substituted C₁₋₁₂alkyl group wherein the optional substituents are each independently selected from OH, OR, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, and
wherein the DNA-targeting unit is any moiety of a molecular weight below 700 Daltons that has an association constant (K) for binding to double-stranded random sequence DNA of >10³ M⁻¹ at an ionic strength of 0.01 M at 20° C.,
wherein T in Formulae A or B, represent at one of carbons 5-8 on the benzo ring the following groups:
halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;
wherein each R is independently selected from an optionally substituted C₁ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, or
T represents a group of Formula E 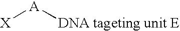
wherein X represents O, S, NH, NMe, CH₂, SO, SO₂, CONH, NHCO, CO, CO₂, or O and
A represents an optionally substituted C₁₋₁₂alkyl group wherein the optional substituents are each independently selected from OH, OR, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, and
wherein the DNA-targeting unit is any moiety of a molecular weight below 700 Daltons that has an association constant (K) for binding to double-stranded random-sequence DNA of >10³ M⁻¹ at an ionic strength of 0.01 M at 20° C.

2 A composition according to claim 1 wherein the DNA targeting agent defined in claim 1 for a group of Formula D or Formula E is independently selected from any one of the formulae III-XVII, 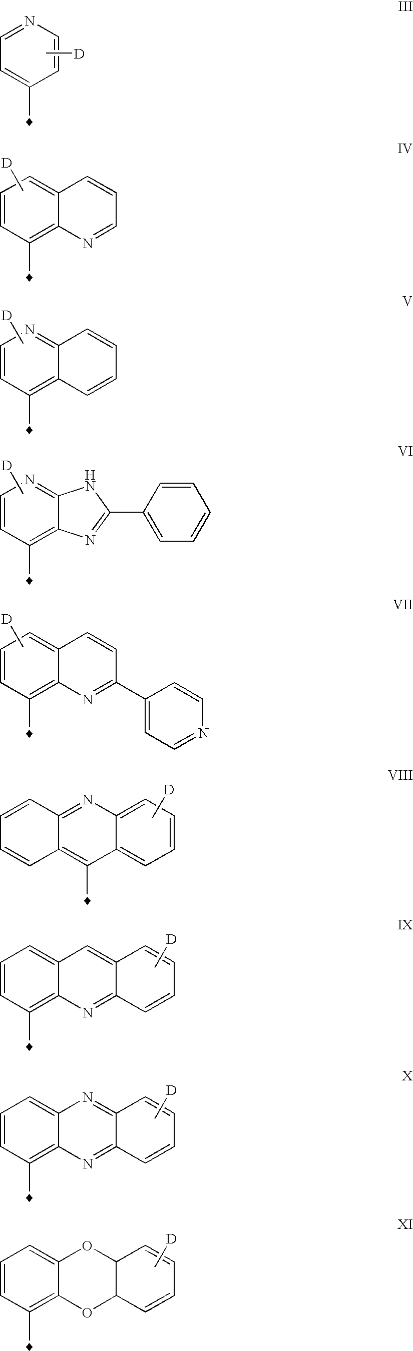 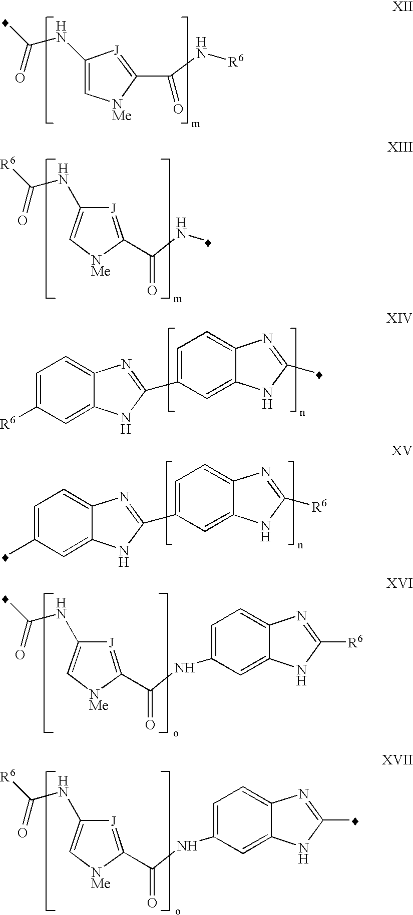
wherein in structures XII-XVII R⁶ is independently selected from an optionally substituted C₁₋₆ alicyclic or an optionally substituted C₃₋₆ cyclic alkyl group, and wherein the optional substituents are each independently selected from; halo, OH, OR⁷ NO₂, NH₂, NHR⁷, NR⁷R⁷, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷;
R⁶ can also represent an optionally substituted aryl or an optionally substituted heteroaryl group having up to 12 carbon atoms, and wherein the optional substituents are each independently selected from; halo, OH, OR⁷, NH₂, NHR⁷, NR⁷R⁷, SH, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷, and each heteroaryl group contains one or more heteroatoms in its ring system which are each independently selected from O, N or S;
wherein each R⁷ is independently selected from an optionally substituted C₁₋₄ alkyl or an optionally substituted C₂₋₄ alkenyl group and wherein the optional substituents are each independently selected from OH, OR⁸, NH₂, NHR⁸, NR⁸₂ or N(OH)R⁸ wherein each R⁸ is independently selected from C₁₋₄ alkyl, C₂₋₄ alkenyl, OH, NO₂, NH₂, CF₃, CN, CO₂H or SH;
D represents up to four of the following groups as substituents at any available ring carbon position; H, R⁹, hydroxy, alkoxy, halogen, NO₂, NH₂, NHR⁹, NR⁹₂, SH, SR⁹, SO₂R⁹, CF₃, CN, CO₂H, CO₂R⁹, CHO, COR⁹, CONH₂, CONHR⁹ or CONR⁹R⁹, cyclic alkylamino, imidazolyl, alkylpiperazinyl, morpholino, wherein each R⁹ is independently selected from an optionally substituted C₁₋₄ alkyl or an optionally substituted C₂₋₄ alkenyl group and wherein the optional substituents are each independently selected from OH, OR¹⁰, NH₂, NHR¹⁰, NR10 ₂ or N(OH)R¹⁰ wherein each R¹⁰ is independently selected from C₁₋₄ alkyl, C₂₋₄ alkenyl, OH, NO₂, NH₂, CF₃, CN, CO₂H or SH;
and wherein any available ring carbon position of formulae III-XVII can also be optionally replaced by —N— when the valency and configuration of the formula allows, the point of attachment of formulae III-XVII to the A group defined above is represented by ♦; and
wherein in formulae XII, XIII, m is selected from 2, 3 or 4, and
wherein in formulae XII, XIII, XVI and XVII, J is selected from CH or N;
and wherein in formulae XIV and XV n is selected from 0, 1 or 2;
and wherein in formulae XVI and XVII o is selected from 1 and 2.

3 A composition according to claim 2 wherein the DNA targeting unit of Formula D or Formula E is selected from one of formulae V, VI, VII, VIII, IX or X.

4 A composition according to claim 2 wherein substituent D of the DNA targeting unit of Formulae III-XI is H or Me.

5 A composition according to claim 1 wherein W in the compound of Formula A as defined in claim 1 represents a NH(C₀-C₁₂) optionally substituted alkyl or a O(C₀-C₁₂) optionally substituted alkyl.

6 A composition according to claim 5 wherein W represents NH₂, NHCH₂CH₂NHCH₃, NHCH₂CH₂N(CH₃)₂ or OCH₃.

7 A method of treating a subject in need of cancer therapy, said method comprising the steps of administering to said subject a cytotoxic effective amount of a composition including an effective amount of one or more compounds of Formula A and one or more compounds of formula B as defined in claim 1 or claim 2 to the tumour cells in said subject.

8 The method according to claim 7 wherein the steps of administration of a compound of Formula A and B are simultaneous or sequential.

9 The method according to claim 7 wherein the tumour cells are in a hypoxic environment.

10 The method according to claim 7 including the further step of administering the composition as defined in claim 7 in combination with one or more other chemotherapeutic agents or treatments, including radiotherapy, either simultaneously, or sequentially, depending on the cancer therapy required.

11 The method according to claim 10 wherein radiotherapy is administered to the tumour cells before, during or after the administration of the composition.

12 The method according to claim 10 wherein the chemotherapeutic agents are selected from Cisplatin or other platinum-based derivatives, Temozolomide or other DNA methylating agents, cyclophosphamide or other DNA alkylating agents, doxorubicin, mitoxandrone, camptothecin or other topoisomerase inhibitors, methotrexate, gemcitabine or other antimetabolites.

13 A compound of Formula I, 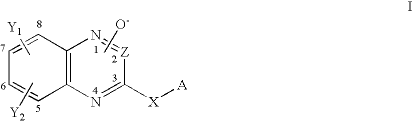
wherein
Z is selected from N or C—CN, and when Z represents N, the N-oxide moiety occupies one of the 1-, 2-, or 4-positions; and when Z represents C—CN, the N-oxide moiety occupies one of the 1-, or 4-positions;
Y₁ and Y₂ each represent at one or more of the available carbons 5-8 on the benzo ring the following groups:
halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;
wherein each R is independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R1-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and
wherein A and X together represent H, or halogen; or
X represents O, S, NH, NMe or CH₂ and
A represents H, an optionally substituted C₁₋₁₂alkyl group wherein the optional substituents are each independently selected from OH, OR³, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂ alkyl chain can be optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, or a pharmacologically acceptable salt thereof,
with the proviso that the following compounds are excluded
3-Amino-1,2,4-benzotriazine-1-oxide,
3-Amino-7-trifluoromethyl-1,2,4-benzotriazine-1-oxide,
3-Amino-7-carbamyl-1,2,4-benzotriazine-1-oxide,
3-Amino-7-chloro-1,2,4-benzotriazine-1-oxide,
3-Amino-7-nitro-1,2,4-benzotriazine-1-oxide
3-Chloro-1,2,4-benzotriazine-1-oxide,
3-(3-N,N-Diethylaminopropylamino)-3-amino-1,2,4-benzotriazine-1-oxide,
3-Chloro-7-nitro-1,2,4-benzotriazine-1-oxide,
7-Nitro-(3-(2-N,N-diethylamino-ethylamino)-1,2,4-benzotriazine-1-oxide,
8-Methoxy-1,2,4-benzotriazin-3-amine 1-oxide,
8-Methyl-1,2,4-benzotriazin-3-amine 1-oxide,
8-Fluoro-1,2,4-benzotriazin-3-amine 1-oxide,
8-Chloro-1,2,4-benzotriazin-3-amine 1-oxide,
8-Trifluoromethyl-1,2,4-benzotriazin-3-amine 1-oxide,
8-(Methylsulfanyl)-1,2,4-benzotriazin-3-amine 1-oxide,
8-(Butylsulfanyl)-1,2,4-benzotriazin-3-amine 1-oxide,
3-Amino-1,2,4-benzotriazin-7-ol 1-oxide,
3-Amino-1,2,4-benzotriazin-7-ol 1-oxide,
7-Methyl-1,2,4-benzotriazin-3-amine 1-oxide,
7-Fluoro-1,2,4-benzotriazin-3-amine 1-oxide,
7-Chloro-1,2,4-benzotrazin-3-amine 1-oxide,
7-Trifluoromethyl-1,2,4-benzotriazin-3-amine 1-oxide,
7-(Methylsulfanyl)-1,2,4-benzotriazin-3-amine 1-oxide,
7-(Butylsulfanyl)-1,2,4-benzotriazin-3-amine 1-oxide,
7-Nitro-1,2,4-benzotriazin-3-amine 1-oxide,
6-Methoxy-1,2,4-benzotriazin-3-amine 1-oxide,
6-Methyl-1,2,4-benzotriazin-3-amine 1-oxide,
6-Phenyl-1,2,4-benzotriazin-3-amine 1-oxide,
6-Fluoro-1,2,4-benzotriazin-3-amine 1-oxide,
6-Chloro-1,2,4-benzotrazin-3-amine 1-oxide,
6-Trifluoromethyl-1,2,4-benzotriazin-3-amine 1-oxide,
6-(Methylsulfanyl)-1,2,4-benzotriazin-3-amine 1-oxide,
6-(Butylsulfanyl)-1,2,4-benzotriazin-3-amine 1-oxide,
5-Methoxy-1,2,4-benzotriazin-3-amine 1-oxide,
5-Methyl-1,2,4-benzotriazin-3-amine 1-oxide,
5-Chloro-1,2,4-benzotriazin-3-amine 1-oxide,
5-Fluoro-1,2,4-benzotriazin-3-amine 1-oxide,
N⁷, N⁷-Dimethyl-1,2,4-benzotriazine-3,7-diamine 1-oxide,
3-Chloro-1,2,4-benzotriazine 1-oxide,
3-Methyl-1,2,4-benzotriazine 1-oxide,
3-Ethyl-1,2,4-benzotriazine 1-oxide,
3-Phenyl-1,2,4-benzotriazine 1-oxide,
3-(4-Methoxyphenyl)-1,2,4-benzotriazine 1-oxide,
3-Vinyl-1,2,4-benzotriazine 1-oxide,
3-Allyl-1,2,4-benzotriazine 1-oxide,
3-(2-Hydroxyethyl)-1,2,4-benzotriazine 1-oxide,
3-(2-Methoxyethyl)-1,2,4-benzotriazine 1-oxide,
N-Phenyl-1,2,4-benzotriazin-3-amine 1-oxide,
3-Methoxy-1,2,4-benzotriazine 1-oxide,
3-Chloro-7-methyl-1,2,4-benzotriazine 1-oxide,
3-Chloro-7-methoxy-1,2,4-benzotriazine 1-oxide,
1,2,4-benzotriazine 1-oxide,
1,2,4-benzotriazin-3-amine 2-oxide, and
1,2,4-benzotriazin-3-amine 4-oxide.

14 The compound of Formula I according to claim 13 wherein Z is N.

15 The compound of Formula I according to claim 13 wherein X is NH or CH₂.

16 The compound of Formula I according to claim 13 wherein —X-A represents a NH(CO-Cl₂) optionally substituted alkyl or an O(C₀-C₁₂) optionally substituted alkyl, such as NHCH₂CH₂NHCH₃, NHCH₂CH₂N(CH₃)₂ or OCH₃.

17 The compound of Formula I according to claim 13 wherein Y₁ and Y₂ each represent H.

18 The compound of Formula I according to claim 13 in which the N-oxide moiety occupies the 1-position.

19 A method of treating a subject in need of cancer therapy, said method comprising the steps of administering to said subject a cytotoxic effective amount of a compound of Formula I 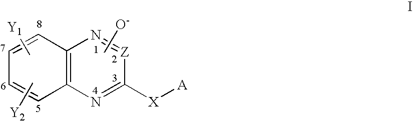
wherein
Z is selected from N or C—CN, and when Z represents N, the N-oxide moiety occupies one of the 1-, 2-, or 4-positions; and when Z represents C—CN, the N-oxide moiety occupies one of the 1-, or 4-positions;
Y₁ and Y₂ each represent at one or more of the available carbons 5-8 on the benzo ring the following groups:
halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;
wherein each R can be independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R1-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and
wherein A and X together represent H, or halogen; or
X represents O, S, NH, NMe or CH₂ and
A represents H, an optionally substituted C₁₋₁₂alkyl group wherein the optional substituents are each independently selected from OH, OR, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C-₁₂ alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, or a pharmacologically acceptable salt thereof
to the tumour cells in said subject.

20 The method according to claim 19 wherein the tumour cells are in a hypoxic environment.

21 The method according to claim 19 further including the step of administering the compound of Formula I as defined in claim 19 in combination with one or more other chemotherapeutic agents or treatments, including radiotherapy, either simultaneously, or sequentially, depending on the cancer therapy required.

22 The method according to claim 19 wherein radiotherapy is administered to the tumour cells before, during or after the administration of the compound of Formula I.

23 The method according to claim 21 wherein the chemotherapeutic agents are selected from Cisplatin or other platinum-based derivatives, Temozolomide or other DNA methylating agents, cyclophosphamide or other DNA alkylating agents, doxorubicin, mitoxandrone, camptothecin or other topoisomerase inhibitors, methotrexate, gemcitabine or other antimetabolites.

24 A compound of Formula I′, 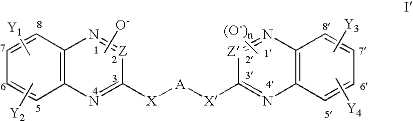
wherein
n represents either 1 or 2,
Z or Z′ is selected from N or C—CN, and when Z or Z′ represents N, and n represents 1 each N-oxide moiety occupies one of the 1-, 2-, or 4-positions or 1′-, 2′-, or 4′-positions respectively and when Z or Z′ represents C—CN, each N-oxide moiety occupies one of the 1-, or 4-positions or 1′-, or 4′-positions respectively; and when Z′ represents N, and n represents 2, the N′-oxide moieties occupy the 1′- and 4‘-positions and when Z’ represents C—CN, and n represents 2 the N′-oxide moieties occupy the 1′-, and 4′-positions;
Y₁, Y₂, Y₃ and Y₄ each represent at one or more of the available carbons 5-8 or one or more of the available carbons 5′-8′ on the respective benzo ring the following groups:
halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;
wherein each R is independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁ alkyl, C₂₋₄ alkenyl, OH, NO₂, NH₂, CF₃, CN, CO₂H or SH, and
wherein X represents NH, NMe, CH₂, or O;
A represents an optionally substituted C₁₋₁₂alkyl group wherein the optional substituents are each independently selected from OH, OR³, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH, or a pharmacologically acceptable salt thereof.

25 The compound of Formula I′ as claimed in claim 24 in which X is NH or CH₂.

26 The compound of Formula I′ as claimed in claim 24 in which Y₁ and Y₂ each represent H.

27 The compound of Formula I′ as claimed in claim 24 in which A is —(CH₂)₂NMe(CH₂)₂—

28 The compound of Formula I′ as claimed in claim 24 in which the N-oxides are positioned at the 1-position and the 1′-position.

29 A method of treating a subject in need of cancer therapy, said method comprising the steps of administering to said subject a cytotoxic effective amount of a compound of Formula I′ as defined in claim 24 to the tumour cells in said subject.

30 The method as claimed in claim 29 wherein the tumour cells are in a hypoxic environment.

31 The method as claimed in claim 29 which includes the further step of administering the compound of Formula I′ as defined in claim 24 in combination with one or more other chemotherapeutic agents or treatments, including radiotherapy, either simultaneously, or sequentially, depending on the cancer therapy required.

32 The method as claimed in claim 31 wheren radiotherapy is administered to the tumour cells before, during or after the administration of the compound of Formula I′.

33 The method as claimed in claim 31 wherein the chemotherapeutic agents are selected from Cisplatin or other platinum-based derivatives, Temozolomide or other DNA methylating agents, cyclophosphamide or other DNA alkylating agents, doxorubicin, mitoxandrone, camptothecin or other topoisomerase inhibitors, methotrexate, gemcitabine or other antimetabolites.

34 A compound of Formula II, 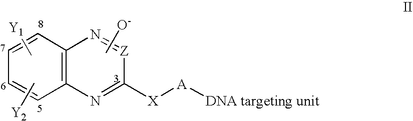
wherein
Z is selected from N or C—CN, and when Z represents N, the N-oxide moiety occupies one of the 1-, 2-, or 4-positions; and when Z represents C—CN, the N-oxide moiety occupies one of the 1-, or 4-positions;
Y₁ and Y₂ each represent at one or more of the available carbons 5-8 on the benzo ring the following groups:
halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;
wherein each R is independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and
wherein X represents NH, NMe, CH₂, or O;
A represents an optionally substituted C₁₋₁₂alkyl group wherein the optional substituents are each independently selected from OH, OR³, NH₂, NHR³, NR³₂, or N(OH)R³ wherein each R³ is independently selected from C₁ alkyl, C₂₋₄ alkenyl, OH, NO₂, NH₂, CF₃, CN, CO₂H or SH; and wherein the optionally substituted C₁₋₁₂alkyl chain can be optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH; and
wherein the DNA-targeting unit is any moiety of a molecular weight below 700 Daltons that has an association constant (K) for binding to double-stranded random-sequence DNA of >10³ M⁻¹ at an ionic strength of 0.01 M at 20° C.,
or a pharmacologically acceptable salt thereof.

35 The compound of Formula II as claimed in claim 34, wherein Z is N.

36 The compound of Formula II as claimed in claim 34 wherein X is NH or CH₂.

37 The compound of Formula II as claimed in claim 34 wherein the N-oxide is at the 1-position.

38 The compound of Formula II as claimed in claim 34 wherein Y₁ and Y₂ each represent H.

39 The compound of Formula II as claimed in claim 34 wherein Y₁ represents Me.

40 The compound of Formula II as claimed in claim 34 wherein A is selected from —(CH₂)₆NH—, —(CH₂)₃NH(CH₂)₃NHCO—, —(CH₂)₃NMe(CH₂)₃NHCO—, —(CH₂)₃NH—, —(CH₂)₂NH(CH₂)₂NHCO— or —(CH₂)₂NMe(CH₂)₂NHCO—.

41 The compound of Formula II as claimed in claim 34 wherein the DNA-targeting unit is selected from one of formulae III-XVII, 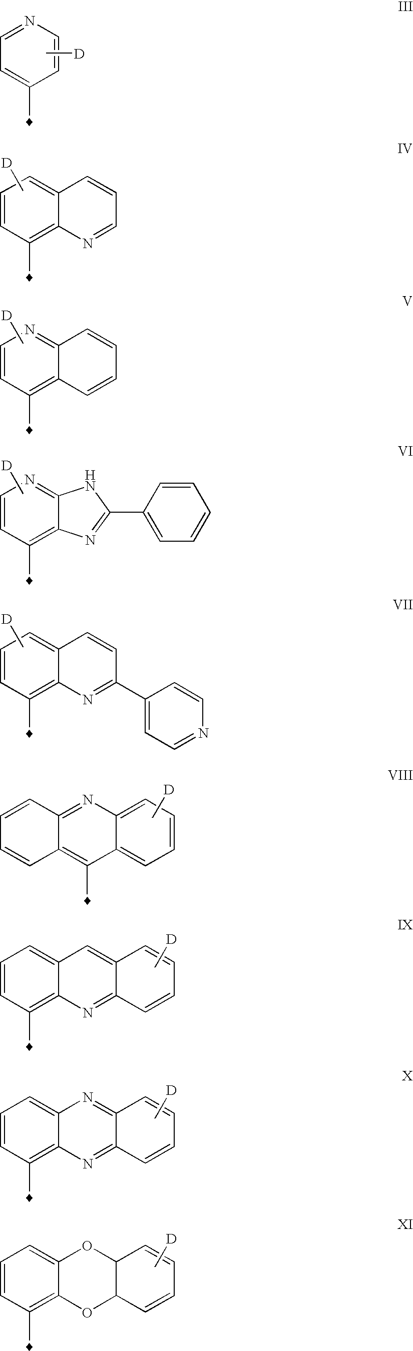 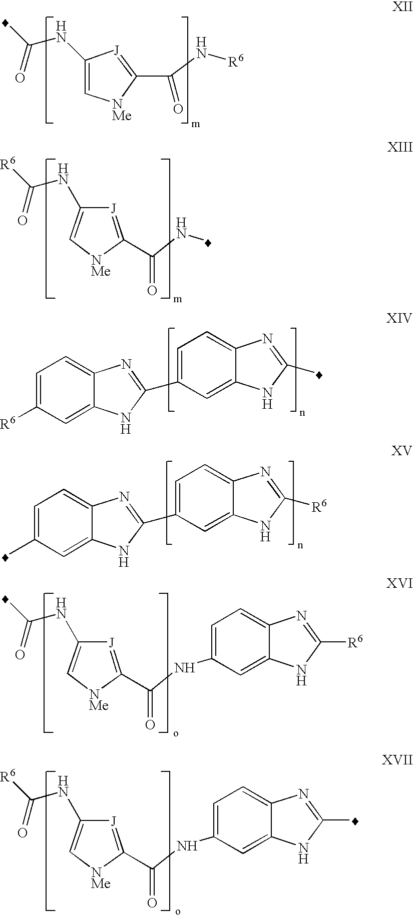
wherein in structures XII-XVII R⁶ is independent ly selected from an optionally substituted C₁₋₆ alicyclic or an optionally substituted C₃₋₆ cyclic alkyl group, and wherein the optional substituents are each independently selected from; halo, OH, OR⁷ NO₂, NH₂, NHR⁷, NR⁷R⁷, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷;
R⁶ can also be represent an optionally substituted aryl or an optionally substituted heteroaryl group having up to 12 carbon atoms, and wherein the optional substituents are each independently selected from; halo, OH, OR⁷, NH₂, NHR¹, NR¹R¹, SH, SR¹, imidazolyl, R⁷-iperazinyl, morpholino, SO₂R⁷, CF₃, CN, CO₂H, CO₂R⁷, CHO, COR⁷, CONH₂, CONHR⁷, CONR⁷R⁷, and each heteroaryl group contains one or more heteroatoms in its ring system which are each independently selected from O, N or S;
wherein each R⁷ is independently selected from an optionally substituted C₁₋₄ alkyl or an optionally substituted C₂₋₄ alkenyl group and wherein the optional substituents are each independently selected from OH, OR⁸, NH₂, NHR⁸, NR⁸₂ or N(OH)R⁸ wherein each R⁸ is independently selected from C₁₋₄ alkyl, C₂₋₄ alkenyl, OH, NO₂, NH₂, CF₃, CN, CO₂H or SH;
D represents up to four of the following groups as substituents at any available ring carbon position; H, R⁹, hydroxy, alkoxy, halogen, NO₂, NH₂, NHR⁹, NR⁹₂, SH, SR⁹, SO₂R⁹, CF₃, CN, CO₂H, CO₂R⁹, CHO, COR⁹, CONH₂, CONHR⁹ or CONR⁹R⁹, cyclic alkylamino, imidazolyl, alkylpiperazinyl, morpholino, wherein each R⁹ is independently selected from an optionally substituted C₁₋₄ alkyl or an optionally substituted C₂₋₄ alkenyl group and wherein the optional substituents are each independently selected from OH, OR¹⁰, NH₂, NHR¹⁰, NR¹⁰₂ or N(OH)R¹⁰ wherein each R¹⁰ is independently selected from C₁₋₄ alkyl, C₂₋₄ alkenyl, OH, NO₂, NH₂, CF₃, CN, CO₂H or SH;
and wherein any available ring carbon position of formulae III-XVII can also be optionally replaced by —N— when the valency and configuration of the formula allows, the point of attachment of formulae III-XVII to the A group defined above is represented by ♦; and
wherein in formulae XII, XIII, m is selected from 2, 3 or 4, and
wherein in formulae XII, XII, XVI and XVII, J is selected from CH or N;
and wherein in formulae XIV and XV n is selected from 0, 1 or 2;
and wherein in formulae XVI and XVII o is selected from 1 and 2.

42 The compound of formula II as claimed in claim 41 wherein the DNA targeting unit is selected from one of formulae V, VI, VII, VIII, IX or X.

43 The compound of formula II as claimed in claim 41 wherein D of the DNA targeting unit of Formulae III-XI is H or Me.

44 A compound of formula II as claimed in claim 41 selected from a compound;
wherein X is NH—, Y is H, Z is N, position 1-oxide, A is —(CH₂)₂NH(CH₂)₂NHCO—, the DNA targeting unit represents formula IX and D is H;
wherein X is NH—, Y is H, Z is N, position 1-oxide, A is —(CH₂)₃NH(CH₂)₃NHCO—, the DNA targeting unit represents formula IX and D is H;
wherein X is NH—, Y is H, Z is N, position 1-oxide, A is —(CH₂)₂NMe(CH₂)₂NHCO—, the DNA targeting unit represents formula IX and D is H;
wherein X is NH—, Y is 6-Me, Z is N, position 1-oxide, A is —(CH₂)₂NMe(CH₂)₂NHCO—, the DNA targeting unit represents formula IX and D is H;
wherein X is NH—, Y is H, Z is N, position 1-oxide, A is —(CH₂)₃NMe(CH₂)₃NHCO—, the DNA targeting unit represents formula IX and D is H;
wherein X is NH—, Y is 6-Me, Z is N, position 1-oxide, A is —(CH₂)₃NMe(CH₂)₃NHCO—, the DNA targeting unit represents formula IX and D is H;
wherein X is NH—, Y is H, Z is N, position 1-oxide, A is —(CH₂)₂NMe(CH₂)₂NHCO—, the DNA targeting unit represents formula IX and D is Me; and
wherein X is NH—, Y is H, Z is N, position 1-oxide, A is —(CH₂)₃NMe(CH₂)₃NHCO—, the DNA targeting unit represents formula IX and D is Me.

45 A method of treating a subject in need of cancer therapy, said method comprising the steps of administering to said subject a cytotoxic effective amount of a compound of Formula II as defined in claim 34 to the tumour cells in said subject.

46 The method according to claim 45 wherein the tumour cells are in a hypoxic environment.

47 The method according to claim 45 which includes the further step of administering the compound of Formula II in combination with one or more other chemotherapeutic agents or treatments, including radiotherapy, either simultaneously, or sequentially, depending on the cancer therapy.

48 The method according to claim 45 radiotherapy is administered to the tumour cells before, during or after the administration of the compound of Formula II.

49 The method according to claim 47 wherein the chemotherapeutic agents are selected from Cisplatin or other platinum-based derivatives, Temozolomide or other DNA methylating agents, cyclophosphamide or other DNA alkylating agents, doxorubicin, mitoxandrone, camptothecin or other topoisomerase inhibitors, methotrexate, gemcitabine or other antimetabolites.

50 A compound of Formula II′, 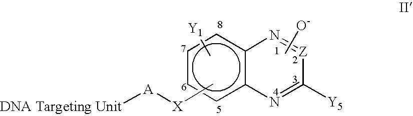
wherein
Z is selected from N or C—CN, and when Z represents N, the N-oxide moiety occupies one of the 1-, 2-, or 4-positions; and when Z represents C—CN, the N-oxide moiety occupies one of the 1-, or 4-positions;
Y₁ represents at one or more of the available carbons 5-8 on the benzo ring the following groups:
halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl, morpholino;
Y₅ is selected from the following groups halo, H, R, OR, NH₂, NHR, NR₂, SO₂R, CF₃, CN, CO₂H, CO₂R, CHO, COR, CONH₂, CONHR or CONRR, cyclic alkylamino, imidazolyl, alkylpiperazinyl, morpholino;
wherein each R of groups Y₁ and Y₅ is independently selected from an optionally substituted C₁₋₆ alicyclic or an optionally substituted C₃₋₆ cyclic alkyl group, and wherein the optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and
wherein X represents NH, NMe, CH₂, S, SO, SO₂, CONH, NHCO, CO, CO₂, or O;
A represents an optionally substituted C₁₋₁₂alkyl group wherein the optional substituents are each independently selected from OH, OR³, NH₂, NHR³, NR³₂ or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₂₋₁₂alkyl chain can be optionally interrupted by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH; and
wherein the DNA-targeting unit is any moiety of a molecular weight below 700 Daltons that has an association constant (K) for binding to double-stranded random-sequence DNA of >10³ M⁻¹ at an ionic strength of 0.01 M at 20° C.,
or a pharmacologically acceptable salt thereof.

51 A compound of Formula II′ as claimed in claim 50 wherein Z is N.

52 A compound of Formula II′ as claimed in claim 50 wherein X is O or CH₂.

53 A compound of Formula II′ as claimed in claim 50 wherein the N-oxide is at the 1-position.

54 A compound of Formula II′ as claimed in claim 50 wherein Y₁ represents H.

55 A compound of Formula II′ as claimed in claim 50 wherein Y₅ represents NHR.

56 A compound of Formula II as claimed in claim 50 wherein A is selected from —(CH₂)₆NH—, —(CH₂)₃NH(CH₂)₃NHCO—, —(CH₂)₃NMe(CH₂)₃NHCO—, —(CH₂)₃NH—, —(CH₂)₂NH(CH₂)₂NHCO— or —(CH₂)₂NMe(CH₂)₂NHCO—.

57 A compound of Formula II as claimed in claim 50 wherein the DNA-targeting unit is selected from one of formulae III-XVII, 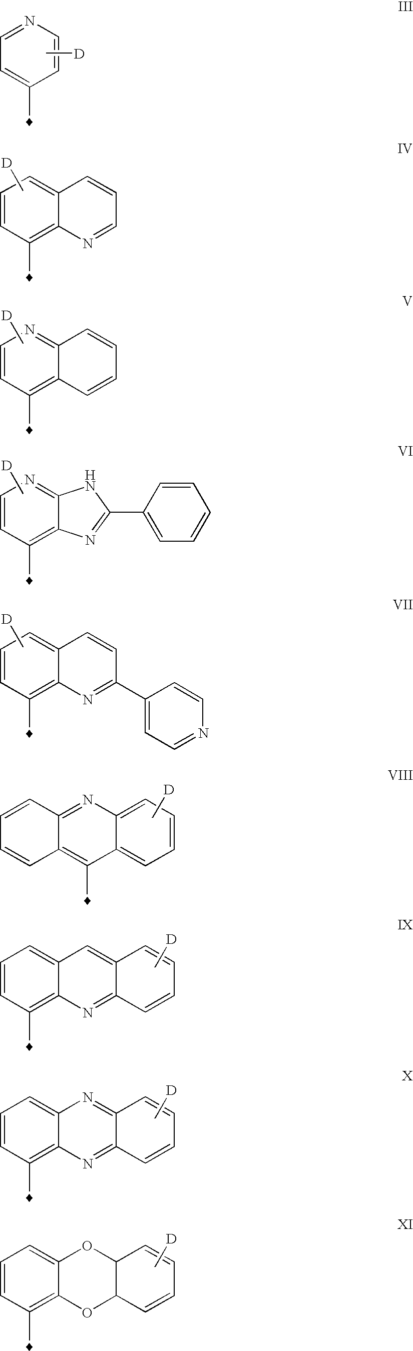 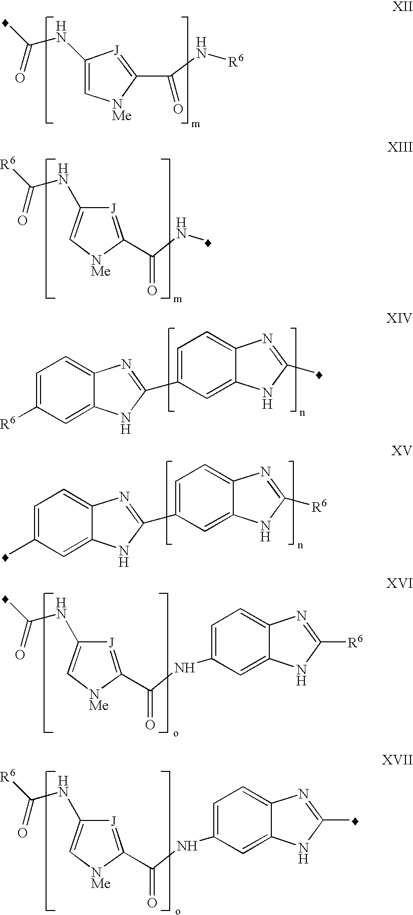
wherein in structures XII-XVII R⁶ is independently selected from an optionally substituted C₁₋₆ alicyclic or an optionally substituted C₃₋₆ cyclic alkyl group, and wherein the optional substituents are each independently selected from; halo, OH, OR⁷ NO₂, NH₂, NHR⁷, NR⁷R⁷, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷;
R⁶ represents an optionally substituted aryl or an optionally substituted heteroaryl group having up to 12 carbon atoms, and wherein the optional substituents are each independently selected from; halo, OH, OR⁷, NH₂, NHR⁷, NR⁷R⁷, SH, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷, and each heteroaryl group contains one or more heteroatoms in its ring system which are each independently selected from O, N or S;
wherein each R⁷ is independently selected from an optionally substituted C₁₋₄ alkyl or an optionally substituted C₂₋₄ alkenyl group and wherein the optional substituents are each independently selected from OH, OR⁸, NH₂, NHR₃, NR⁸₂ or N(OH)R⁹⁸ wherein each R⁸ is independently selected from C₁₋₄ alkyl, C₂₋₄ alkenyl, OH, NO₂, NH₂, CF₃, CN, CO₂H or SH;
D represents up to four of the following groups as substituents at any available ring carbon position; H, R⁹, hydroxy, alkoxy, halogen, NO₂, NH₂, NHR⁹, NR92, SH, SR⁹, SO₂R⁹, CF₃, CN, CO₂H, CO₂R⁹, CHO, COR⁹, CONH₂, CONHR⁹ or CONR⁹R⁹, cyclic alkylamino, imidazolyl, alkylpiperazinyl, morpholino, wherein each R⁹ independently selected from an optionally substituted C₁₋₄ alkyl or an optionally substituted C₂₋₄ alkenyl group and wherein the optional substituents are each independently selected from OH, OR¹⁰, NH₂, NHR¹⁰, NR¹⁰₂ or N(OH)R¹⁰ wherein each R¹⁰ is independently selected from C₁₋₄ alkyl, C₂₋₄ alkenyl, OH, NO₂, NH₂, CF₃, CN, CO₂H or SH;
and wherein any available ring carbon position of formulae III-XVII can be optionally replaced by —N— when the valency and configuration of the formula allows, the point of attachment of formulae III-XVII to the A group defined above is represented by ♦; and
wherein in formulae XII and XIII, m is selected from 2, 3 or 4,
and wherein in formulae XII, XIII, XVI or XVII J is selected from CH or N; and
wherein in formulae XIV and XV n is selected from 0, 1 or 2, and
wherein in formulae XVI and XVII o is selected from 1 or 2.

58 A compound of Formula II′ as claimed in claim 57 wherein the DNA targeting unit is selected from one of formulae IV-X.

59 A compound of formula II′ as claimed in claim 57 wherein D of the DNA targeting unit of Formulae III-XI is H or Me.

60 A compound of formula 11′ as claimed in claim 57 selected from a compound;
wherein X is CH₂—, Y₁ is H, Y₅ is NHCH₂CH₂OMe, Z is —N—, A is —(CH₂)NMe(CH₂)₂NHCO—, the DNA targeting unit represents formula IX and D is H;
wherein X is CH₂—, Y₁ is H, Y₅ is NHCH₂CH₂OMe, Z is —N—, A is —(CH₂)₂NH(CH₂)₃NHCO—, the DNA targeting unit represents formula IX and D is H;
wherein X is CH₂—, Y₁ is H, Y₅ is NHCH₂CH₂OMe, Z is —N—, A is —(CH₂)NMe(CH₂)₂NHCO—, the DNA targeting unit represents formula IX and D is Me;
wherein X is CH₂—, Y₁ is H, Y₅ is NHCH₂CH₂OMe, Z is —N—, A is —(CH₂)₂NMe(CH₂)₃NHCO—, the DNA targeting unit represents formula IX and D is Me;
wherein X is CH₂—, Y₁ is H, Y₅ is NHCH₂CH₂OMe, Z is —N—, A is —(CH₂)NMe(CH₂)₂NHCO—, the DNA targeting unit represents formula X and D is Me; and
wherein X is CH₂—, Y₁ is H, Y₅ is NHCH₂CH₂OMe, Z is —N—, A is —(CH₂)₂NH(CH₂)₃NHCO—, the DNA targeting unit represents formula X and D is Me.

61 A method of treating a subject in need of cancer therapy, said method comprising the steps of administering to said subject a cytotoxic effective amount of a compound of Formula II′ as defined in claim 50 to the tumour cells in said subject.

62 The method according to claim 61 wherein the tumour cells are in a hypoxic environment.

63 The method according to claim 61 which includes the further step of administering the compound of Formula II′ in combination with one or more other chemotherapeutic agents or treatments, including radiotherapy, either simultaneously, or sequentially, depending on the cancer therapy required.

64 The method according to claim 63 wherein radiotherapy is administered to the tumour cells before, during or after the administration of the compound of Formula II′.

65 The method according to claim 63 wherein the chemotherapeutic agents are selected from Cisplatin or other platinum-based derivatives, Temozolomide or other DNA methylating agents, cyclophosphamide or other DNA alkylating agents, doxorubicin, mitoxandrone, camptothecin or other topoisomerase inhibitors, methotrexate, gemcitabine or other antimetabolites.

66 A method of potentiating the cytotoxicity of an amount of a compound of Formula B as defined in claim 1 or a composition including Formula B as defined in claim 1, which has been administered to a subject in need of cancer therapy, by administering to said subject a compound of Formula A as defined in claim 1 or a composition including Formula A as defined in claim 1.

67 The method as claimed in claim 66 which potentiates the hypoxic cytotoxicity of an amount of a compound of Formula B.

68 The method as claimed in claim 66 which includes the further step of administering to said subject the compound of Formula A as defined in claim 1 or a composition including Formula A as defined in claim 1 in combination with one or other chemotherapeutic agents or treatments defined above, including radiotherapy, either simultaneously, or sequentially depending on the cancer therapy required.

69 The method as claimed in claim 68 wherein radiotherapy is administered to the subject, before, during or after the administration of said compound of Formula A or said composition including Formula A.

70 A method of potentiating the cytotoxicity of one or more chemotherapeutic agents as defined above, administered to a subject, by further administering to said subject a compound of Formula A as defined in claim 1 or a composition including Formula A as defined in claim 1.

71 The method as claimed in claim 68 which potentiates the hypoxic cytotoxicity of the one or more chemotherapeutic agents.

72 The method as claimed in claim 71 which includes the further step of administering radiotherapy to said subject, either simultaneously or sequentially depending on the cancer therapy required.

73 The method as claimed in claim 72 wherein the step of administering radiotherapy to the subject, occurs before, during or after the administration of said compound of Formula A or said composition including Formula A.
